(19) United States
(12) Patent Application Publication
(10) Pub. No.: US 2025/0144253 A1
(43) Pub. Date: May 8, 2025

(54) 3-((3-([1,1'-BIPHENYL]-3-YLMETHOXY)PHENOXY)METHYL)BENZONITRILE DERIVATIVES AND THE USE THEREOF

(71) Applicant: HELMHOLTZ-ZENTRUM DRESDEN-ROSSENDORF E.V., Dresden (DE)

(72) Inventors: Sven STADLBAUER, Dresden (DE); Fabian KRUTZEK, Dresden (DE); Klaus KOPKA, Dresden (DE)

(73) Assignee: HELMHOLTZ-ZENTRUM DRESDEN-ROSSENDORF E.V., Dresden (DE)

(21) Appl. No.: 18/716,251

(22) PCT Filed: Dec. 5, 2022
(86) PCT No.: PCT/EP2022/084480

§ 371 Date: Jun. 4, 2024

(30) Foreign Application Priority Data
Dec. 6, 2021 (EP) 21212444.0

(51) Int. Cl. A61K 51/04

(52) U.S. Cl. CPC A61K 51/0459; A61K 51/0497

(57) ABSTRACT

A compound of general formula I

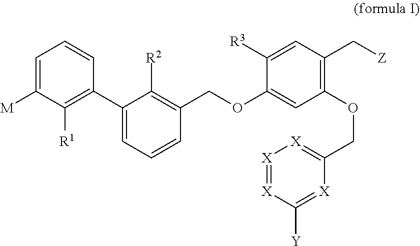

where Z is selected from the group containing a group of general formula II, a group of general formula III, a group of general formula IV, a group of general formula V, a group of general formula VI, and a group of general formula VIa:

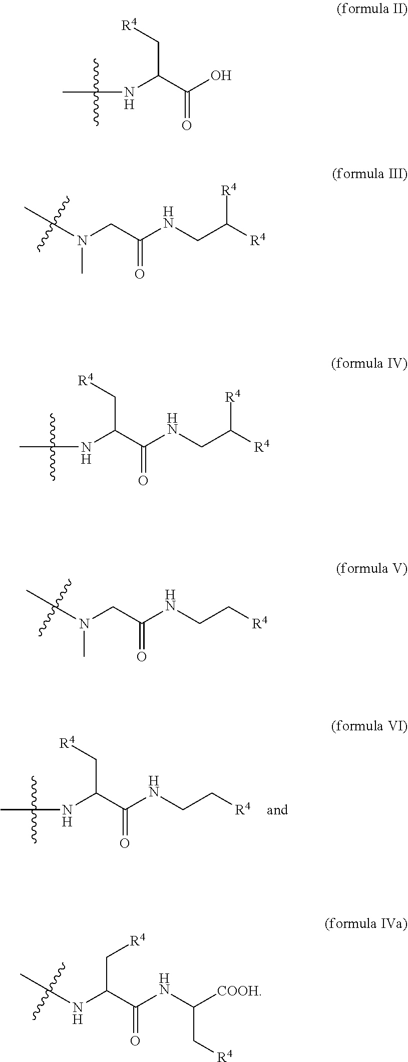

### FIELD

The invention relates to 3-((3-([1,1′-biphenyl]-3-ylmethoxy)phenoxy)methyl)benzonitrile derivatives. Further, it relates to the uses of the 3-((3-([1,1′-biphenyl]-3-ylmethoxy)phenoxy)methyl)benzonitrile derivatives.

### BACKGROUND

The Programmed Death Receptor 1 (CD279, PD-1) is a receptor on T cells that is deactivated by binding to its ligand (PD-L1, CD274, B7-H1). Interactions between PD-1 and its ligand PD-L1 regulate the immune responses, for example during the resolution of an infection or a tumor or during the development of an immunotolerance. A stimulation, as occurring for example with tumor diseases or chronic infections, with T cells results in an increased expression of PD-1 in order to be able to control the virus or tumor cell. As a result, also the tumor cells overexpress the corresponding ligand that again deactivates the PD-1. If the T cells over a longer period of time are deactivated with the corresponding antigen this results in a so-called “T cell exhaustion”. Blocking of the PD-1-/PD-L1 interaction again can be broken up using antibodies directed against PD-1 or PD-L1 and the T cells are reactivated. Ideally, said reactivation of the immune system results in an effective control of the tumor cells. Cancer patients can profit from an antibody therapy directed against PD-1 or PD-L1 as well as patients having a virus infection. However, the response rate in an antibody monotherapy is only 30%. In addition to the antibodies in clinical use also peptides and organic active ingredients of low molecular weight have been developed that bind to and inhibit PD-L1. Said substances are in the pre-clinical testing [19-22]. Examples of such substances are compounds S1, S2, and S3.

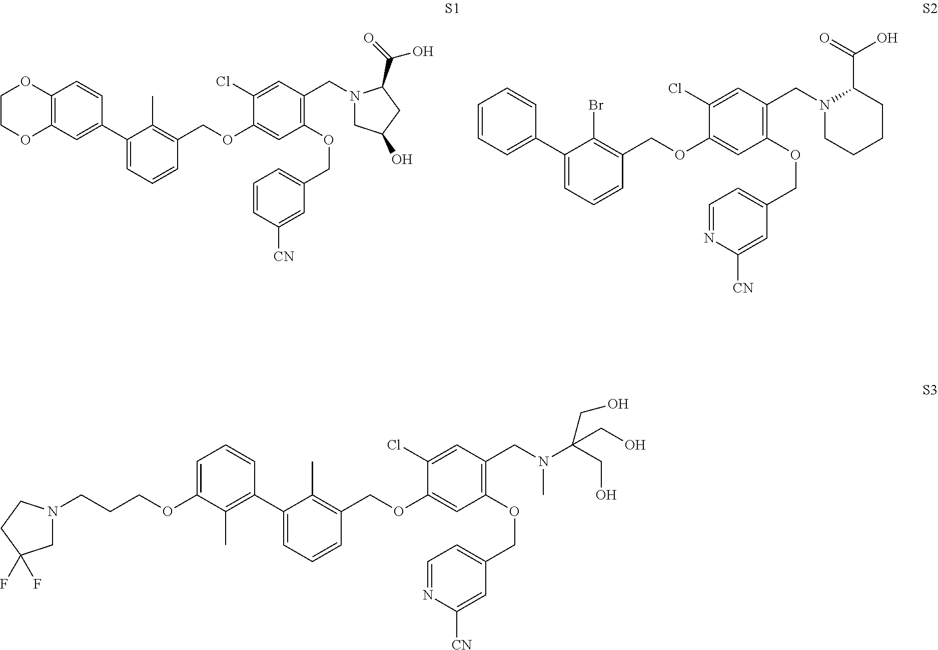 [SMILES: Cc1c(COc2cc(OCc3cccc(C#N)c3)c(CN3C[C@H](O)C[C@@H]3C(=O)O)cc2Cl)cccc1-c1ccc2c(c1)OCCO2 ; N#Cc1cc(COc2cc(OCc3cccc(-c4ccccc4)c3Br)c(Cl)cc2CN2CCCC[C@H]2C(=O)O)ccn1 ; Cc1c(COc2cc(OCc3ccnc(C#N)c3)c(CN(C)C(CO)(CO)CO)cc2Cl)cccc1-c1cccc(OCCCN2CCC(F)(F)C2)c1C]

Compounds S1, S2, and S3 are organic compounds of low molecular weight to be used for checkpoint inhibitor therapy.

The described organic molecules of low molecular weight are all similar in structure [23], as is explained in more detail with respect to scheme S1 by means of example of compound S3. They have a biaryl moiety (section 1 in scheme S1) that is of particular importance for the binding affinity. In addition, also the central chloroaromatic (section 2) and an ether-bridged cyanopyridyl group (section 3) play a role for the binding affinity. Amino alcohols or amino acids provide for water solubility (section 4). An optional extension moiety (section 5) may further have positive influence on the binding affinity. The substances known so far despite the present amino acids do not have water solubility, but a very high lipophilicity.

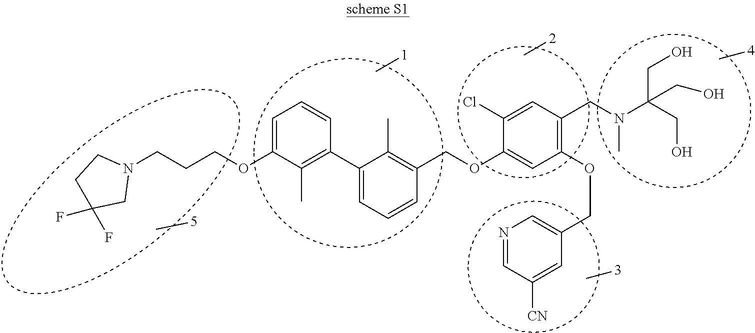

The decision to use a checkpoint inhibitor therapy is made by determining the PD-L1 protein expression by means of immunohistochemical methods of biopsic resectates. PD-L1 expression is very heterogenous both between and within tumor lesions, which is why it is difficult for the attending oncologist to decide for a therapy. Non-invasive, molecular imaging techniques such as PET and SPECT are better suited to select the patients prior to the therapy who probably will respond, since these can solve the problem of the heterogenous expression of PD-L1 over time. To this end, PD-L1-affine organic molecules of low molecular weight, peptides, and antibodies can be labelled with a radionuclide (e.g., fluorine-18, gallium-68, iodine-123) which allows an imaging of the PD-L1 expression. So far, PD-L1-affine antibodies, single-domain antibodies that are also referred to as nanobodies and affibodies have been radio-labelled and both pre-clinically and clinically investigated as PET or SPECT tracers [24-27]. Affibodies are antigen-binding peptides consisting of 58 amino acids.

Some peptidic ligands have been successfully pre-clinically tested in tumor-bearing animals [28-32]. An organic molecule of low molecular weight has also been pre-clinically tested after labelling by means of fluorine-18 in animals [33]. CN 111662270 A [34] discloses the radioactive labelling of different substances of low molecular weight with different radioactive iodine isotopes. For example, compound S5 is shown there:

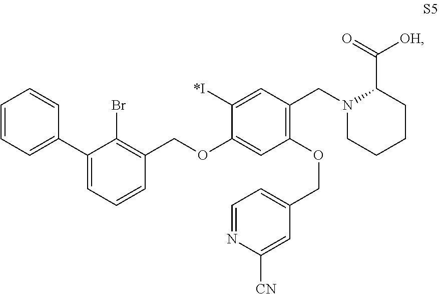

wherein *I is I-123, I-124, I-125, I-129, or I-131.

A drawback of the antibodies, affibodies, and nanobodies is their relatively high immunogenicity which can cause detrimental immunologic effects such as cytokine storms [35]. Moreover, the antibody-based radiotracers have long circulation times of several days in the body until the enrichment maximum in the tumor for a good imaging contrast has been reached. In contrast, peptides and small organic molecules have the advantage that they can enrich more rapid in the tumor tissue and achieve their enrichment maximum in the range of minutes to hours. In addition, these short circulation times allow the labelling with short-lived PET nuclides such as carbon-II and fluorine-18 that allow a particularly good resolving power of the PET images. However, the small molecules described so far exhibit a low enrichment in the tumor and due to the high lipophilicity a high enrichment in the liver [33].

The problem of the invention is to eliminate the drawbacks of the prior art. In particular, 3-((3-([1,1′-biphenyl]-3-ylmethoxy)phenoxy)methyl)benzonitrile derivatives shall be provided that bind to PD-L1, but have a lower lipophilicity. In particular, 3-((3-([1,1′-biphenyl]-3-ylmethoxy)phenoxy)methyl)benzonitrile derivatives shall be provided that after radioactive labelling with a radionuclide are suitable for the molecular non- and minimum-invasive nuclear-medical imaging and endoradiotherapy of PD-L1-overexpressing tumor diseases. Moreover, 3-((3-([1,1′-biphenyl]-3-ylmethoxy)phenoxy)methyl)benzonitrile derivatives shall be provided that are suitable for the therapy of PD-L1-positive tumors and COVID-19 infections.

### SUMMARY

According to the invention a compound of general formula I is provided:

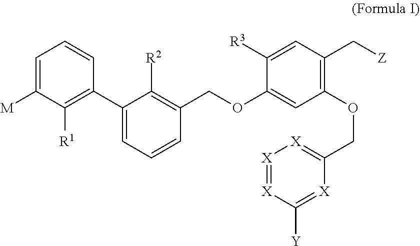

wherein
R¹ is selected from the group consisting of hydrogen, a substituted or unsubstituted C₁-C₆ alkyl group and bromine;
R² is selected from the group consisting of a substituted or unsubstituted C₁-C₆ alkyl group and bromine or iodine;
R³ is selected from the group consisting of chlorine, bromine, iodine and a first leaving group AG¹;
M is hydrogen or a group R⁵;
X is C—H or N, with the provision that at most two of the moieties X are N and the remaining moieties X are C—H;
Y is selected from the group consisting of —CN, —S(O)₂—CH₃, fluorine, iodine and a second leaving group AG²;
Z is selected from the group consisting of a group of general formula II, a group of general formula III, a group of general formula IV, a group of general formula V, a group of general formula VI and a group of general formula VIa

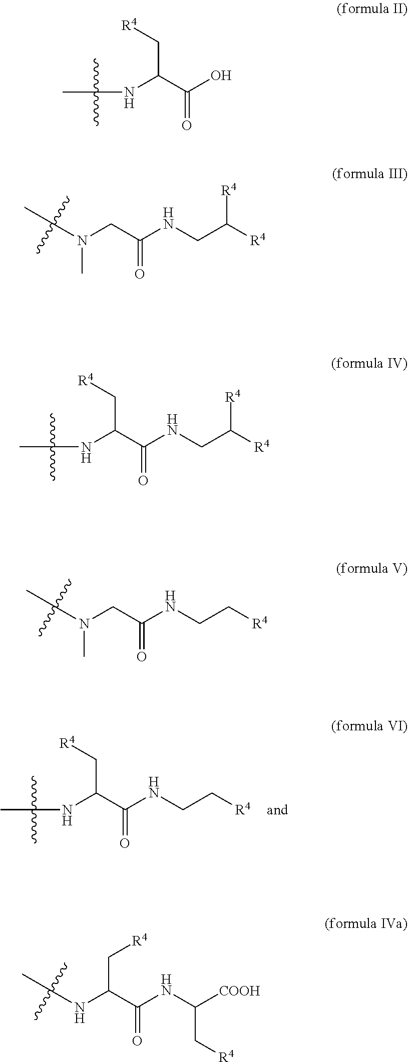

AG¹ is selected from the group consisting of an organotin group, a boronic acid and a boronic acid ester;
AG² is selected from the group consisting of an organotin group, a fluorosulphate group, a boronic acid and a boronic acid ester;
R⁴ is a sulphonic acid group or a phosphonic acid group;
R⁵ is selected from a first group or a second group, wherein
the first group consists of a group of general formula VII or a group of general formula VIII:

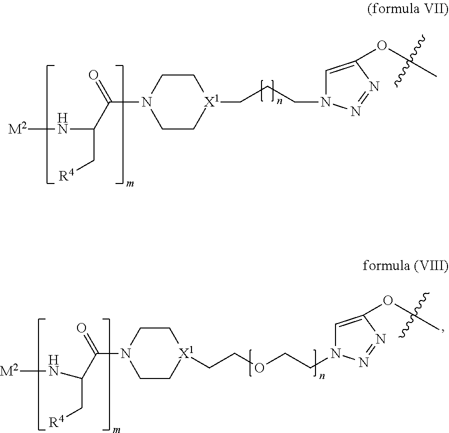

X¹ is C—H or N,
M² is a chelate ligand or a chelate complex consisting of the chelate ligand and a central atom, wherein the central atom is selected from the group consisting of Sc-43, Sc-44, Sc-47, Cu-61, Cu-62, Cu-64, Cu-67, Ga-68, Y-86, Zr-89, Y-90, Tc-99m, In-111, La-133, Gd-153, Gd-155, Gd-157, Tb-149, Tb-152, Tb-155, Tb-161, Lu-177, Pd-197m, Pb-209, Pb-212, Bi-213, and Ac-225, m is an integer of 0 to 3, n is an integer of 1 to 7, and p is an integer of 1 to 4;
the second group consists of a group of general formula IX, a group of general formula X, a group of general formula XI, and a group of general formula XII:

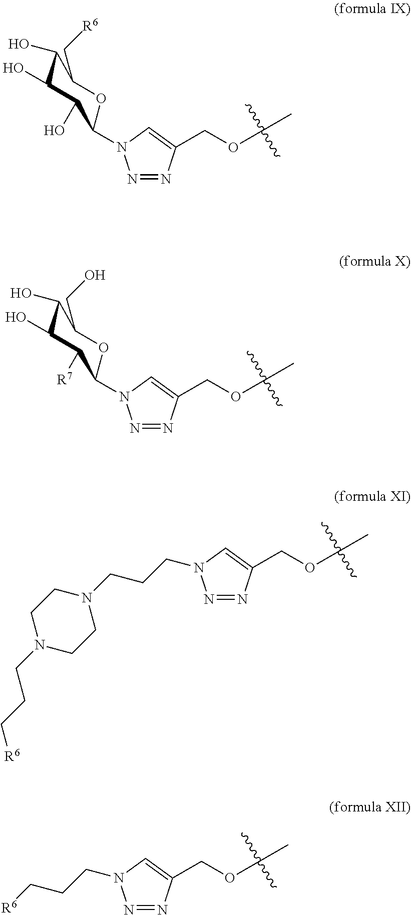

wherein
R⁶ is O-Ts or fluorine and R⁷ is O-Tf or fluorine, Ts designates a tosyl group and Tf designates a triflyl group.

The compounds according to the invention may be PD-L1-addressing compounds. The compounds according to the invention bind to PD-L1. The compounds according to the invention are particularly suitable for the diagnostics and/or therapy of cancer diseases and COVID-19 infections. PD-L1 designates the protein named “programmed cell death 1 ligand 1”.

The compounds according to the invention each have a water-soluble group. These groups are the residue Z. Residue Z is moieties having sulphonic acid groups or phosphonic acid groups. These are residues R4. Sulphonic acid groups and phosphonic acid groups replace the amino acid groups and amino alcohol groups known from the prior art. Thus, the compounds according to the invention in contrast to the compounds known from the prior art possess a low lipophilicity. It has been found that moiety Z has no detrimental influence on the binding affinity to the PD-L1 target structure.

If the compound of general formula I has a group of general formula IX or a group of general formula X then also these groups contribute to the water solubility of the compounds according to the invention. The groups of general formula IX and the groups of general formula X each have sugar moieties bound to a triazole residue on the phenyl ring of the basic structure.

In one embodiment of the invention it is provided that the compound of general formula I according to the invention contains not more than one of the following radionuclides: F-18, I*, Sc-43, Sc-44, Sc-47, Cu-61, Cu-62, Cu-64, Cu-67, Ga-68, Y-86, Zr-89, Y-90, Tc-99m, In-111, La-133, Gd-153, Gd-155, Gd-157, Tb-149, Tb-152, Tb-155, Tb-161 Lu-177, Pd-197m, Pb-209, Pb-212, Bi-213, and Ac-225. A compound containing one of the mentioned radionuclides in the following is also referred to as radiotracer. The isotopes Sc-43, Sc-44, Sc-47, Cu-61, Cu-62, Cu-64, Cu-67, Ga-68, Y-86, Y-90, Tc-99m, In-111, La-133, Gd-153, Gd-155, Gd-157, Tb-149, Tb-152, Tb-155, Tb-161 Lu-177, Pd-197m, Pb-209, Pb-212, Bi-213, and Ac-225 in the following are also referred to as radiometals. In a chelate complex the radiometals form the central atoms.

In one embodiment of the invention it is provided that the compounds of general formula I according to the invention are no radio-labelled compounds. These have none of the following radionuclides F-18, I*, Sc-43, Sc-44, Sc-47, Cu-61, Cu-62, Cu-64, Cu-67, Ga-68, Y-86, Zr-89, Y-90, Tc-99m, In-111, La-133, Gd-153, Gd-155, Gd-157, Tb-149, Tb-152, Tb-155, Tb-161 Lu-177, Pd-197m, Pb-209, Pb-212, Bi-213, and Ac-225. In the following, these compounds are referred to as not radio-labelled compounds. The not radio-labelled compounds according to the invention are suitable for the diagnostics and/or therapy of cancer diseases and COVID-19 infections. In particular, they can be used as classical active ingredients for a checkpoint inhibitor therapy of cancer or a COVID-19 therapy. The not radio-labelled compounds according to the invention in a cell test on COVID-19-infected lung cells showed that the cell viability again could be significantly enhanced and is approximately in the range of the control substance remdesivir.

The radiotracers according to the invention permit the molecular, i.e. non-invasive imaging of PD-L1-overexpressing tumors. The imaging is a nuclear-medical imaging. Here, the imaging may take place in the range of minutes to hours, so that the patient still on the day of the injection of the radiotracer can be examined by means of positron emission tomography (PET) or single photon emission computer tomography (SPECT). For example, positron emission tomography (PET) can be performed as positron emission tomography/computer tomography (PET/CT) or positron emission tomography/magnetic resonance tomography (PET/MR), single photon emission computer tomography (SPECT) as single photon emission computer tomography/computer tomography (SPECT/CT). The radiotracers according to the invention eliminate the drawback of circulation times over several days as found with antibody-based radiotracers. Moreover, intake of the substances according to the invention into the liver is decreased and their excretion via the kidney is favored.

Thus, the radiotracers according to the invention in particular differ from the prior art in that they each have a water-soluble group and a radioactive isotope, namely F-18, I* or a radiometal. Precursor compounds that can be used for the preparation of the radiotracers according to the invention each can have a water-soluble group and one of the following groups: a chelate ligand to form a chelate complex with a radiometal, the leaving group AG¹ or the leaving group AG². These precursor compounds also are subject of the invention.

In a preferred embodiment of the invention it is provided that R⁶ is [¹⁸F]fluorine. In another preferred embodiment of the invention it is provided that R⁷ is [¹⁸F]fluorine. In a further preferred embodiment of the invention it is provided that R³ or Y is a radioactive iodine isotope. The radioactive iodine isotope is preferably selected from the group consisting of iodine-123, iodine-124, iodine-125, or iodine-131. In the following a radioactive iodine isotope is referred to as I*. If R⁶ is [¹⁸F]fluorine or R⁷ is [¹⁸F]fluorine, then neither R³ nor Y is I*. If R³ or Y is I*, then M is hydrogen. If R³ is I*, then Y is not I*. If Y is I*, then R³ is not I*.

Preferably, Z is a group of general formula III or a group of general formula IV:

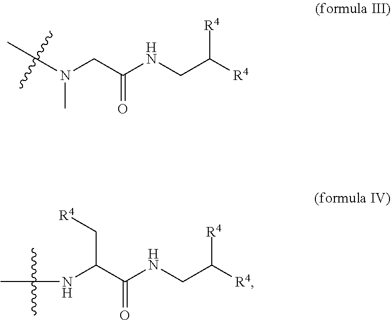

wherein R⁴ has the meaning given in connection with the compound of general formula I. Here, it is preferred that R⁴ is a sulphonic acid group.

In a preferred embodiment R⁵ is a group of general formula IX, a group of general formula X or a group of general formula XII:

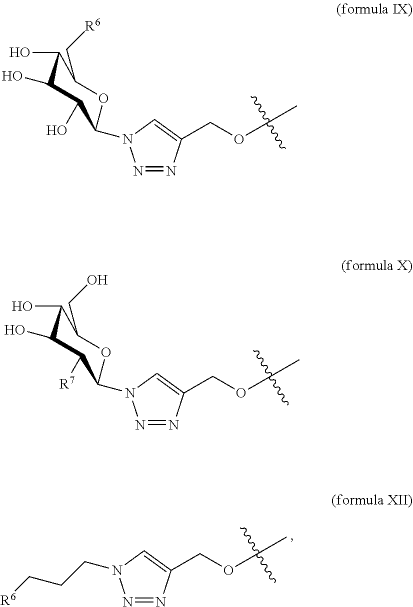

wherein R⁶ and R⁷ have the meanings given in connection with the compound of general formula I. Here, it is preferred that R⁶ is fluorine, particularly preferred [¹⁸F]fluorine. It is further preferred that R⁷ is fluorine, particularly preferred [¹⁸F]fluorine.

In another preferred embodiment R⁵ is a group of general formula VII or a group of general formula VIII:

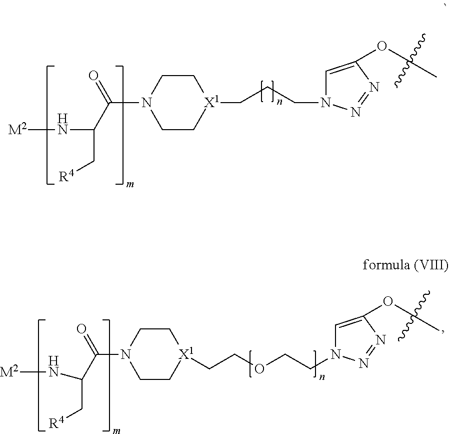

wherein R⁴, M², X¹ as well as m and n have the meanings given in connection with the compound of general formula I. Here, it is preferred that m is 0 or 1, n is 1 or 2 and p is 1 or 2. It is further preferred that M² is a chelate ligand selected from the group consisting of a 1,4,7,10-tetraazacyclododecane-1,4,7,10-tetra acetic acid group (DOTA group), a 1,4,7-triazacyclononane-1-glutaric acid-1,4,7-acetic acid group (NODAGA group), and a 6,6′-((1,4,10,13-tetraoxa-7,16-diazacyclooctadecane-7,16-diyl)bis(methylene))dipicolinic acid group (MACROPA group). Preferably, the chelate complex has a central atom selected from the group consisting of Cu-64, Ga-68, La-133, Lu-177, and Ac-225. Preferably, the chelate complex has a chelate ligand selected from the group consisting of a 1,4,7,10-tetraazacyclododecane-1,4,7,10-tetra acetic acid group (DOTA group), a 1,4,7-triazacyclononane-1-glutaric acid-1,4,7-acetic acid group (NODAGA group), and a 6,6′-((1,4,10,13-tetraoxa-7,16-diazacyclooctadecane-7,16-diyl)bis(methylene))dipicolinic acid group (MACROPA group), and a central atom selected from the group consisting of Cu-64, Ga-68, La-133, Lu-177, and Ac-225. If R⁵ is a group of general formula VII or a group of general formula VIII, then it may be provided that R³ and Y are not I*.

Preferably, R¹ is hydrogen or a methyl group. It is further preferred that R² is bromine, or a methyl group. Additionally, it is preferred that R³ is chlorine, bromine, or iodine. Moreover, it is preferred that exactly one X is N and the remaining moieties X are C—H. Furthermore, it is preferred that Y is —CN or —S(O)₂—CH₃.

There may be provided a compound of general formula I, wherein
R¹ is selected from the group consisting of hydrogen, a substituted or unsubstituted C₁-C₆ alkyl group and bromine;
R² is selected from the group consisting of a substituted or unsubstituted C₁-C₆ alkyl group and bromine;
R³ is selected from the group consisting of chlorine, bromine and iodine;
X is C—H or N, with the provision that at most two of the moieties X are N and the remaining moieties X are C—H;
Y is selected from the group consisting of —CN, —S(O)₂—CH₃, fluorine, and iodine;
M and Z have the meanings given in connection with the compound of general formula I.

There may be provided a compound of general formula I, wherein
R¹ is hydrogen or a methyl group;
R² is bromine or a methyl group;
R³ is chlorine, bromine, or iodine;
X is C—H or N, with the provision that at most two of the moieties X are N and the remaining moieties X are C—H;
Y is selected from the group consisting of —CN, —S(O)₂—CH₃, fluorine, and iodine;
M and Z have the meanings given in connection with the compound of general formula I.

There may be provided a compound of general formula I, wherein
R¹ is hydrogen or a methyl group;
R² is bromine or a methyl group;
R³ is chlorine, bromine, or iodine;
X is C—H or N, with the provision that at most two of the moieties X are N and the remaining moieties X are C—H;
Y is —CN or —S(O)₂—CH₃;
M and Z have the meanings given in connection with the compound of general formula I.

There may be provided a compound of general formula I, wherein
R¹ is hydrogen or a methyl group;
R² is bromine, or a methyl group;
R³ is chlorine, bromine, or iodine;
X is C—H or N, with the provision that only one of the moieties X is N and the remaining moieties X are C—H;
Y is —CN or —S(O)₂—CH₃;
M and Z have the meanings given in connection with the compound of general formula I.

There may further be provided a compound of general formula IA,

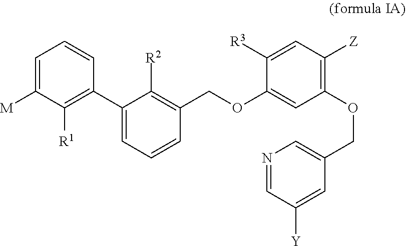

wherein
R¹ is hydrogen or a methyl group;
R² is bromine, or a methyl group;
R³ is chlorine, bromine, or iodine;
Y is —CN or —S(O)₂—CH₃;
M and Z have the meanings given in connection with the compound of general formula I.

The compound of general formula I-A corresponds to a compound of formula I in which only the X in the 3 position to the methoxy group is N and the remaining moieties X are C—H.

There may further be provided a compound of general formula IA, wherein
R¹ is hydrogen or a methyl group;
R² is bromine, or a methyl group;
R³ is chlorine, bromine, or iodine;
Y is —CN or —S(O)₂—CH₃;
Z is a group of general formula III or a group of general formula IV, wherein
R⁴ is a sulphonic acid group, and
M has the meaning given in connection with the compound of general formula I.

There may further be provided a compound of general formula IB,

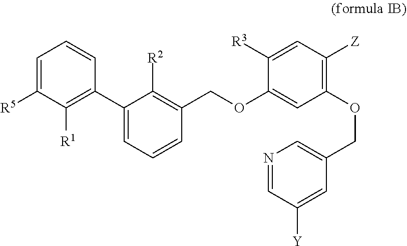

wherein
R¹ is hydrogen or a methyl group;
R² is bromine, or a methyl group;
R³ is chlorine, bromine, or iodine;
Y is —CN or —S(O)₂—CH₃;
Z is a group of general formula III or IV, wherein R⁴ is a sulphonic acid group, and
R⁵ is selected from a first group or a second group, wherein
the first group is a group of general formula VII or a group of general formula VIII, wherein X¹, M², m, n and p have the meanings given in connection with the compound of general formula I; and
the second group is selected from the group consisting of a group of general formula IX, a group of general formula X, a group of general formula XI, and a group of general formula XII, wherein R⁶ and R⁷ have the meanings given in connection with the compound of general formula I.

The compound of general formula I-B corresponds to a compound of formula IA in which M is R⁵.

There may further be provided a compound of general formula IB,

wherein
R¹ is hydrogen or a methyl group;
R² is bromine, or a methyl group;
R³ is chlorine, bromine, or iodine;
Y is —CN or —S(O)₂—CH₃;
Z is a group of general formula III or a group of general formula IV, wherein R⁴ is a sulphonic acid group, and
R⁵ is a group of general formula VII or a group of general formula VIII, wherein
m is 0 or 1, n is 1 or 2, and p is 1 or 2;
M² is a chelate ligand selected from the group consisting of a 1,4,7,10-tetraazacyclododecane-1,4,7,10-tetra acetic acid group (DOTA group), a 1,4,7-triazacyclononane-1-glutaric acid-1,4,7-acetic acid group (NODAGA group), and a 6,6′-((1,4,10,13-tetraoxa-7,16-diazacyclooctadecane-7,16-diyl)bis(methylene))dipicolinic acid group (MACROPA group); und
X¹ has the meanings given in connection with the compound of general formula I.

There may further be provided a compound of general formula IB,

wherein
R¹ is hydrogen or a methyl group;
R² is bromine, or a methyl group;
R³ is chlorine, bromine, or iodine;
Y is —CN or —S(O)₂—CH₃;
Z is a group of general formula III or IV, wherein R⁴ is a sulphonic acid group, and
R⁵ is a group of general formula VII or a group of general formula VIII, wherein
m is 0 or 1, n is 1 or 2, and p is 1 or 2; and
M² is a chelate complex consisting of
a chelate ligand selected from the group consisting of a 1,4,7,10-tetraazacyclododecane-1,4,7,10-tetra acetic acid group (DOTA group), a 1,4,7-triazacyclononane-1-glutaric acid-1,4,7-acetic acid group (NODAGA group), and a 6,6′-((1,4,10,13-tetraoxa-7,16-diazacyclooctadecane-7,16-diyl)bis(methylene))dipicolinic acid group (MACROPA group), and a central atom selected from the group consisting of Cu-64, Ga-68, La-133, Lu-177, and Ac-225, and X¹ has the meanings given in connection with the compound of general formula I.

There may further be provided a compound of general formula IB, wherein
R¹ is hydrogen or a methyl group;
R² is bromine, or a methyl group;
R³ is chlorine, bromine, or iodine;
Y is —CN or —S(O)₂—CH₃;
Z is a group of general formula III or IV, wherein R⁴ is a sulphonic acid group, and
R⁵ is selected from the group consisting of a group of general formula IX, a group of general formula X, and a group of general formula XII;
wherein R⁶ and R⁷ have the meanings given in connection with the compound of general formula I.

In one embodiment the compounds according to the invention are radiotracers. The radiotracers can be used for the diagnostics and/or therapy of cancer diseases and COVID-19 infections. The radiotracers may be PD-L1-addressing radiotracers. The compounds according to the invention comprise the five groups of radiotracers described below.

### First Group of Radiotracers

If the compound of general formula I contains a group AG¹, then it preferably contains no group AG². Thus, there may be provided a compound of the general formula IP-1

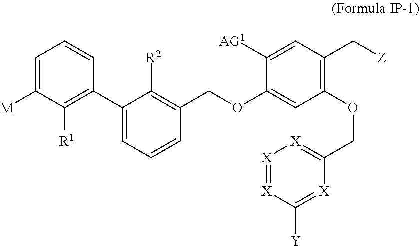

in which M is hydrogen and AG¹, R¹, R², X, Y, and Z have the meanings given in connection with the compound of general formula I, with the provision that Y is not AG² and not I*. The compound of general formula IP-1 can be used for the preparation of a radioiodinated compound of general formula [I*]I-1

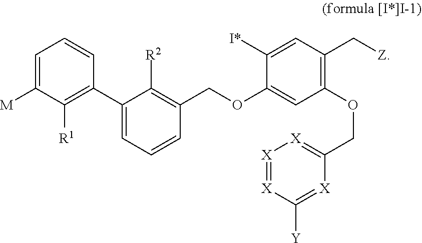

The compound of general formula [I*]I-1 corresponds to the compound of general formula IP-1, except that instead of AG¹ there is a radioactive iodine isotope I*. When using a compound of general formula IP-1 for the preparation of a compound of general formula [I*]I-1, then it is provided that the compound of general formula IP-1 contains no [¹⁸F]fluorine. The compounds of general formula [I*]I-1 are a first group of radiotracers.

The leaving group AG¹ permits a radioactive labelling with I*. Thus, the radiotracers of the first group are suitable for the diagnostics by means of imaging methods such as PET or SPECT. If I* is iodine-131, then the radiotracers of the first group are suitable for the systemic radiotherapy, in particular the endoradiotherapy.

### Second Group of Radiotracers

If the compound of general formula I contains a group AG², then it preferably contains no group AG¹. Thus, there may be provided a compound of the general formula

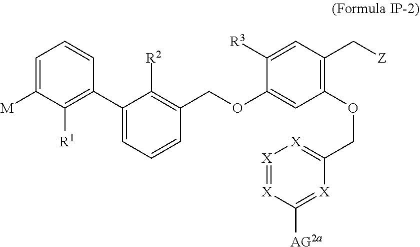

in which M is hydrogen, AG^{2a} is a leaving group AG²; R¹, R², R³, X, and Z have the meanings given in connection with the compound of general formula I, with the provision that R³ is not AG¹ and not I*. Preferably, AG^{2a} is a boronic acid ester group, particularly preferred a group of formula

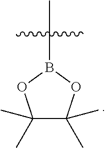

The compound of general formula IP-2 can be used for the preparation of a radioiodinated compound of general formula [I*]I-2

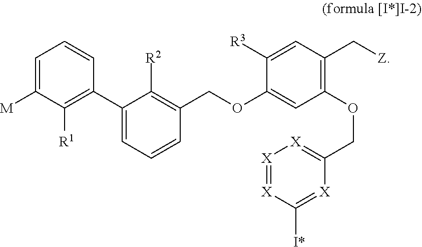

The compound of general formula [I*]I-2 corresponds to the compound of general formula IP-2, except that instead of AG^{2a} there is a radioactive iodine isotope I*. When using a compound of general formula IP-2 for the preparation of a compound of general formula [I*]I-2, then it is provided that the compound of general formula IP-2 contains no [¹⁸F]fluorine. The compounds of general formula [I*]I-2 are a second group of radiotracers.

The leaving group AG^{2a} permits a radioactive labelling with I*. Thus, the radiotracers of the second group are suitable for the diagnostics by means of imaging methods such as PET or SPECT. If I* is iodine-131, then the radiotracers of the second group are suitable for the systemic radiotherapy, in particular the endoradiotherapy.

### Third Group of Radiotracers

If the compound of general formula I contains a group AG², then it preferably contains no group AG¹. Thus, there may be provided a compound of the general formula IP-3

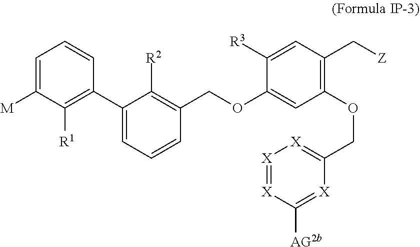

in which M is hydrogen, AG^{2b} is a leaving group AG²; R¹, R², R³, X, and Z have the meanings given in connection with the compound of general formula I, with the provision that R³ is not AG¹ and not I*. Preferably, AG^{2b} is selected from the group consisting of a fluorosulphate group, a boronic acid group, or a boronic acid ester group. Particularly preferred AG^{2b} is a group of formula

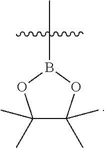

The compound of general formula IP-3 can be used for the preparation of a radiofluorinated compound of general formula [¹⁸F]I-3

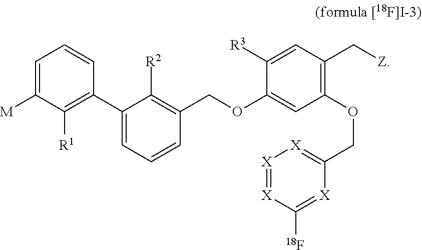

The compound of general formula [¹⁸F]I-3 corresponds to the compound of general formula IP-3, except that instead of AG^{2b} there is a [¹⁸F]fluorine. When using a compound of general formula IP-3 for the preparation of a compound of general formula [¹⁸F]I-3, then it is provided that the compound of general formula IP-3 contains no I*. The compounds of general formula [¹⁸F]I-3 are a third group of radiotracers.

The leaving group AG^{2b} permits a radiofluorination with [¹⁸F]fluorine. For example, the radiofluorination may take place via isotope exchange on a fluorosulphate group or via copper-mediated radiofluorination on boronic acids or boronic acid esters.

Thus, the radiotracers of the third group are suitable for the diagnostics by means of imaging methods such as PET or SPECT.

### Fourth Group of Radiotracers

There may be provided a compound of general formula IP-4

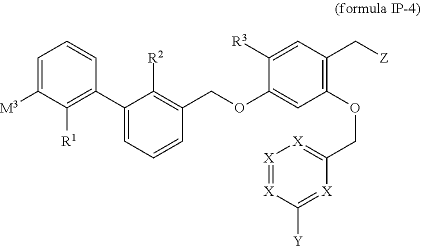

in which M³ is selected from the group consisting of a group of general formula IX, a group of general formula X, a group of general formula XI, and a group of general formula XII, R⁶ is O-Ts and R⁷ is O-Tf, and R¹, R², R³, X, and Z have the meanings given in connection with the compound of general formula I, with the provision that R³ is not AG¹ and not I* and that Y is not AG² and not I*. The compound of general formula IP-4 can be used for the preparation of a radiofluorinated compound of general formula [¹⁸F]I-4

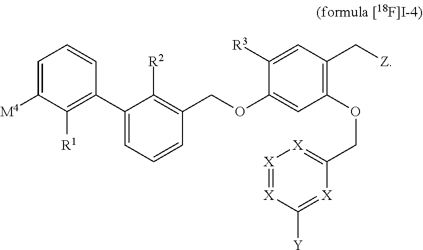

The compound of general formula [¹⁸F]I-4 corresponds to the compound of general formula IP-4, except that instead of M³ there is a group M⁴. Group M⁴ corresponds to the group M³, except that R⁶ is [¹⁸F]fluorine and that R⁷ is [¹⁸F]fluorine. The compounds of general formula [¹⁸F]I-4 are a fourth group of radiotracers.

The group of general formula IX and the group of general formula X each have a sugar moiety that is bound to the basic structure via a triazole residue. Here, the sugar moiety bears a reactive leaving group such as O-Ts or O-Tf that can be substituted by F-18. Here, the sugar residue additionally contributes to the water solubility of the compounds of general formula IP-4 and [¹⁸F]I-4.

The groups O-Ts and O-Tf permit a radioactive labelling with [¹⁸F]fluorine. Thus, the radiotracers of the fourth group are suitable for the diagnostics by means of imaging methods such as PET or SPECT.

### Fifth Group of Radiotracers

There may be provided a compound of general formula IP-5

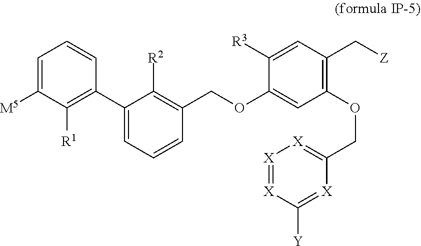

in which M⁵
is a group of general formula VII or a group of general formula VIII in which m is an integer of 0 to 3, n is an integer of 1 to 7 and p is an integer of 1 to 4, M² is a chelate ligand and
R¹, R², R³, X, Y, and Z have the meanings given in connection with the compound of general formula I, with the provision that R³ is not AG¹ and not I* and that Y is not AG² and not I*. The compound of general formula IP-5 can be used for the preparation of a radioactive compound of general formula [CK]I-5

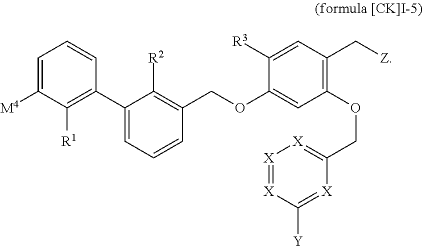

The compound of general formula [CK]I-5 corresponds to the compound of general formula IP-5, except that instead of M⁴ there is a group M⁵. The group M⁵ is a chelate complex consisting of the chelate ligand of compound IP-4 and a central atom, wherein the central atom is selected from the group consisting of Cu-64, Ga-68, La-133, Lu-177, and Ac-225. The compounds of general formula [CK]I-5 are a fifth group of radiotracers. In the compounds IP-5 and [CK]I-5 the chelate ligand is preferably selected from the group consisting of a 1,4,7,10-tetraazacyclododecane-1,4,7,10-tetra acetic acid group (DOTA group), a 1,4,7-triazacyclononane-1-glutaric acid-1,4,7-acetic acid group (NODAGA group), and a 6,6′-((1,4,10,13-tetraoxa-7,16-diazacyclooctadecane-7,16-diyl)bis(methylene))dipicolinic acid group (MACROPA group).

The group of general formula VII or the group of general formula VIII each have a linker moiety that is coupled to the chelate ligand via a piperazine moiety or piperidine moiety either directly or via a further sulphonic acid or phosphonic acid moiety. The linker moiety is a clickable linker moiety. The chelate ligands permit a radioactive labelling of the compounds of general formula IP-5 with a radiometal Cu-64, Ga68, La-133, Lu-177, and Ac-225.

The radiotracers of the fifth group are organic substances of low molecular weight that are particularly suitable for the diagnostics and therapy, especially endoradiotherapy, of PD-L1-positive tumors due to the chelate complexes. They are particularly suitable for the diagnostics by means of imaging methods such as PET or SPECT. For the PET diagnostics particularly compounds of general formula [CK]I-5 are suitable in which the central atom is Cu-64 or Ga-68. For the endoradiotherapy, for example compounds of general formula [CK]I-5 are suitable the central atom of which is a beta emitter such as e.g., Cu-67 or Lu-177, or an alpha emitter such as e.g., Ac-225.

The invention provides radiotracers which can have different radionuclides. The radiotracers according to the invention are suitable both for the PET diagnostics and for the endoradiotherapy of PD-L1-positive tumors. The compounds according to the invention have the advantage that they are water-soluble and therefore both exhibit a low intake into the liver and permit excretion via the bladder. This is a significant advantage compared to the only radiotracer of low molecular weight reported so far that is very lipophilic and thus, has shown a too high intake into the liver. The compounds according to the invention after radioactive labelling in the animal trial exhibit a very good intake in the tumor and a low intake in blood, muscles, and healthy tissue.

Moreover, the compounds according to the invention can be used as classical active ingredients for a checkpoint inhibitor therapy of cancer or a COVID-19 therapy. The not radioactive labelled compounds in a cell test on COVID-19-infected lung cells showed that the cell viability again could significantly be enhanced and is approximately in the range of the control substance remdesivir. Preferred not radioactive labelled compounds of general formula I are compounds of general formula IA and compounds of general formula IB. The not radioactive labelled compounds according to the invention of general formula I are water-soluble inhibitors for the active ingredient therapy of PD-L1-positive tumors and COVID-19 infections.

Due to the sulphonic acid- or phosphonic acid-bearing groups provided the compounds according to the invention are water-soluble. The compounds according to the invention permit the radioactive labelling with PET or SPECT radionuclides as well as beta or alpha emitters. This is attributed to suitable leaving groups or chelate ligands. The chelate ligands can be connected with the basic structure via linker moieties.

### Definitions

The term “alkyl”, unless specified otherwise, particularly relates to a monovalent, saturated, aliphatic hydrocarbon group having a branched or unbranched carbon chain with 1 to 12 carbon atoms, preferably 1 to 8 carbon atoms, and particularly preferably 1 to 6 carbon atoms. Examples of alkyl groups include, but are not limited to methyl, ethyl, propyl, isopropyl, isobutyl, sec-butyl, tert-butyl, pentyl, n-hexyl, octyl, dodecyl and the like. The alkyl group may optionally be substituted with one or more substituents, wherein each substituent independently is hydroxy, alkyl, alkoxy, aryl, halogen, haloalkyl, cyano, nitro, amino, mono-alkylamino or dialkylamino, unless specified otherwise.

The term “alkoxy”, unless specified otherwise, particularly relates to a group of formula —OR, wherein R is an alkyl group, as defined herein. Examples of alkoxy components include, but are not limited to methoxy, ethoxy, isopropoxy, and the like. The alkoxy group may optionally be substituted with one or more substituents, wherein each substituent independently is hydroxy, alkyl, alkoxy, aryl, halogen, haloalkyl, cyano, nitro, amino, mono-alkylamino or dialkylamino, unless specified otherwise.

The term “aryl”, unless specified otherwise, particularly relates to a cyclic, aromatic hydrocarbon group consisting of a mono, bi or tricyclic aromatic ring system having 5 to 10 ring atoms, preferably 5 or 6 ring atoms. The aryl group may optionally be a substituted aryl group. Examples of aryl groups include, but are not limited to phenyl, naphthyl, anthracenyl, naphthalenyl, phenanthryl, fluorenyl, indenyl, pentalenyl, azulenyl, oxydiphenyl, biphenyl, methylenediphenyl, aminodiphenyl, diphenylsulfidyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like. The term “substituted aryl group” particularly relates to an aryl group that is independently substituted with one to four substituents, preferably one or two substituents selected from hydroxy, alkyl, alkoxy, aryl, halogen, haloalkyl, cyano, nitro, amino, mono-alkylamino, or dialkylamino. Unless specified otherwise, the aryl group may be mono or multivalent, for example mono or bivalent.

The term “halogen” relates to fluorine, chlorine, bromine, or iodine. If the compound of general formula I contains [¹⁸F]fluorine, so it can be provided that the compound does not contain any further fluoro atom. If the compound of general formula I contains I*, so it can be provided that the compound does not contain any further iodine atom.

The term “sulphonic acid group”, unless otherwise specified, relates to a group —SO₂OH.

The term “sulphonate group”, unless otherwise specified, relates to a R^{S}—SO₂—O group. R^{S} may be for example a branched or unbranched substituted or unsubstituted C₁-C₆ alkyl group, an aryl group, or an alkylaryl group. Preferably, R^{S} is CH₃—, CF₃—, or CH₃—C₆H₄—. The sulphonic acid group may be for example selected from the group consisting of a toluene sulphonic acid ester group, a methyl sulphonic acid ester group, and a trifluoromethyl sulphonic acid ester group. Toluene sulphonic acid ester group means a —OTs group, wherein Ts is tosyl. Methyl sulphonic acid ester group means a —Oms group, wherein Ms is mesyl. Trifluoromethyl sulphonic acid ester group means CF₃—SO₂—O— that is referred to as —OTf, wherein Tf is triflyl.

The term “phosphonic acid group”, unless otherwise specified, relates to a group —P(O)(OH)₂.

It may be provided that AG² is an organotin compound. The organotin compound may for example be alkyl tin the alkyl group(s) of which may be substituted or unsubstituted, wherein one or more of the alkyl groups optionally have one or more hetero atoms, or aryl tin the aryl group(s) of which may be substituted or unsubstituted, wherein one or more of the aryl groups optionally have one or more hetero atoms.

One example of a boronic acid ester is 2,4,4,5,5-pentamethyl-1,3,2-dioxaborolane.

Preferred Examples of Compounds of general formula (I) are given in the following tables.

TABLE 1
Fluorine Compounds

| Com- | | |
|---|---|---|
| pound | Structure | Name |
| 85 | 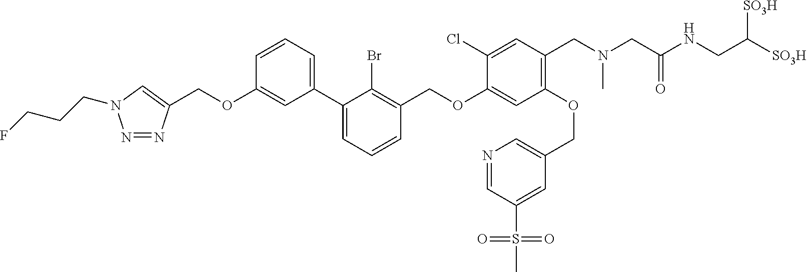 [SMILES: CN(CC(=O)NCC(S(=O)(=O)O)S(=O)(=O)O)Cc1cc(Cl)c(OCc2cccc(-c3cccc(OCc4cn(CCCF)nn4)c3)c2Br)cc1OCc1cncc(S(C)(=O)=O)c1] | 2-(2-((4-((2-Bromo-3′-((1-(3- fluoropropyl)-1H-1,2,3-triazol-4- yl)methoxy)-[1,1′-biphenyl]-3- yl)methoxy)-5-chloro-2-((5-(methyl- sulfonyl)pyridin-3-yl)methoxy) benzyl)(methyl)amino)aceta- mido)ethane-1,1-disulfonic acid |
| 151 | 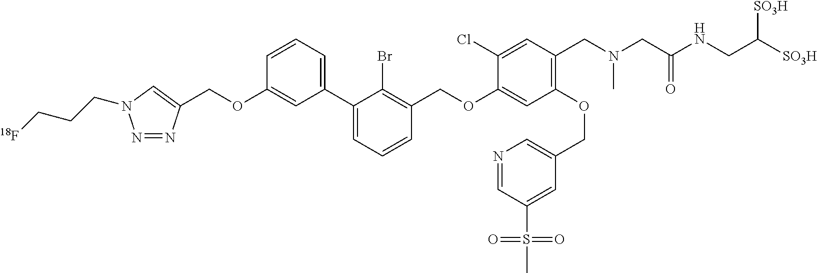 [SMILES: CN(CC(=O)NCC(S(=O)(=O)O)S(=O)(=O)O)Cc1cc(Cl)c(OCc2cccc(-c3cccc(OCc4cn(CCC[18F])nn4)c3)c2Br)cc1OCc1cncc(S(C)(=O)=O)c1] | corresponds Compound 85, wherein F is [¹⁸F] |
| 86 | 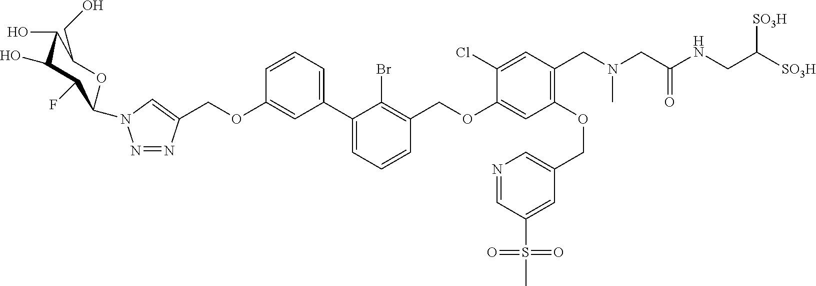 | 2-(2-((4-((2-Bromo-3′-((1- ((2R,3R,4S,5S,6R)-3-fluoro-4,5- dihydroxy-6-(hydroxymethyl)tet- rahydro-2H-pyran-2-yl)-1H-1,2,3- triazol-4-yl)methoxy)-[1,1′-bi- phenyl]-3-yl)methoxy)-5-chloro- 2-((5-(methylsulfonyl)pyridin-3- yl)methoxy)benzyl)(methyl) amino)acetamido)ethane-1,1- disulfonic acid |
| 134 | 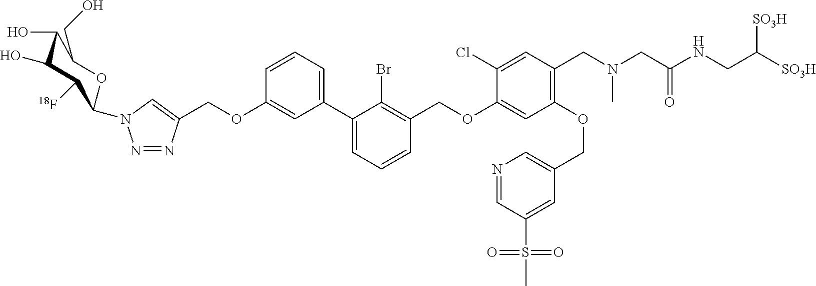 | corresponds Compound 86, wherein F is [¹⁸F] |
| 87 | 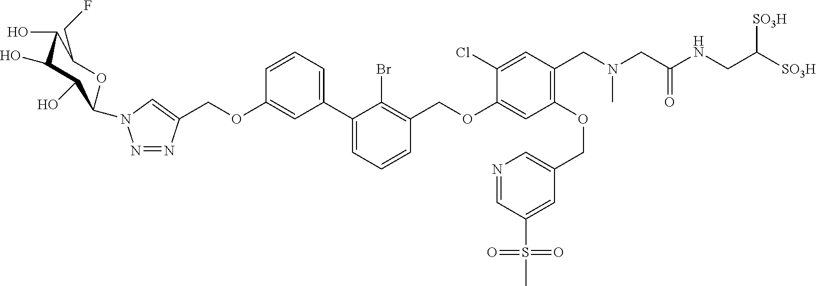 | 2-(2-((4-((2-Bromo-3′-((1- ((2R,3R,4S,5S,6S)-6-(fluoro- methyl)-3,4,5- trihydroxytetrahydro- 2H-pyran-2-yl)-1H-1,2,3-triazol- 4-yl)methoxy)-[1,1′-biphenyl]-3- yl)methoxy)-5-chloro-2-((5- (methylsulfonyl) pyridin-3-yl)methoxy) benzyl)(methyl)amino) acetamido)ethane-1,1- disulfonic acid |
| 135 | 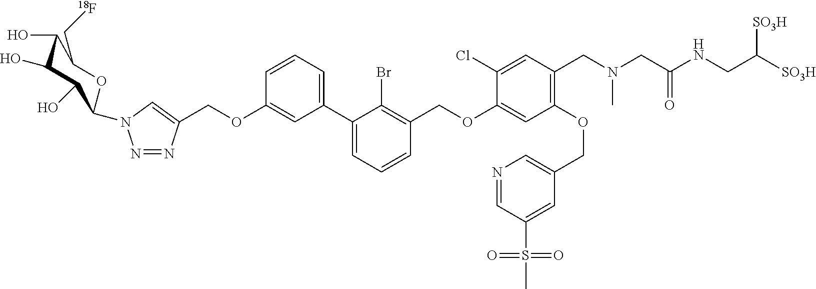 | corresponds Compound 87, wherein F is [¹⁸F] |
| 88 | 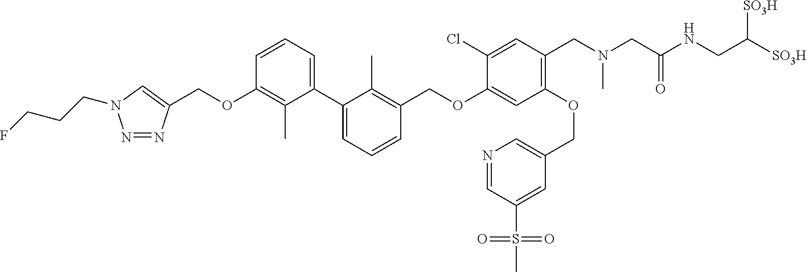 [SMILES: Cc1c(COc2cc(OCc3cncc(S(C)(=O)=O)c3)c(CN(C)CC(=O)NCC(S(=O)(=O)O)S(=O)(=O)O)cc2Cl)cccc1-c1cccc(OCc2cn(CCCF)nn2)c1C] | 2-(2-((5-Chloro-4-((3′-((1-(3- fluoropropyl)-1H-1,2,3-triazol-4- yl)methoxy)-2,2′-dimethyl-[1,1′- biphenyl]-3-yl)methoxy)-2-((5- (methylsulfonyl)27yridine-3- yl)methoxy)benzyl)(methyl) amino)acetamido)ethane-1,1- disulfonic acid |
| 136 | 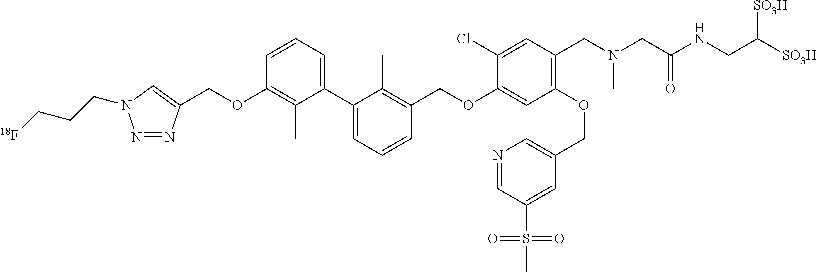 [SMILES: Cc1c(COc2cc(OCc3cncc(S(C)(=O)=O)c3)c(CN(C)CC(=O)NCC(S(=O)(=O)O)S(=O)(=O)O)cc2Cl)cccc1-c1cccc(OCc2cn(CCC[18F])nn2)c1C] | corresponds Compound 88, wherein F is [¹⁸F] |
| 89 | 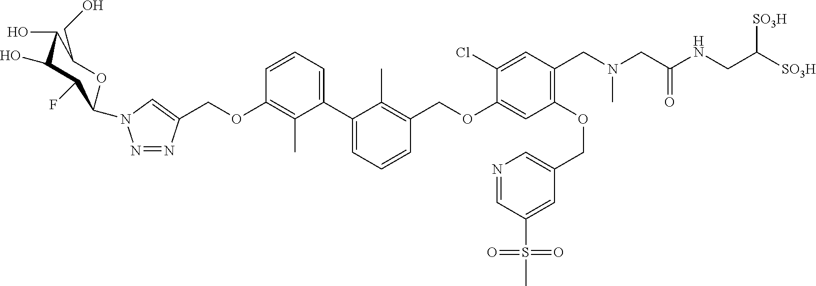 | 2-(2-((5-Chloro-4-((3′-((1- ((2R,3R,4S,5S,6R)-3-fluoro-4,5- dihydroxy-6-(hydroxymethyl)tet- rahydro-2H-pyran-2-yl)-1H-1,2,3- triazol-4-yl)methoxy)-2,2′-di- methyl-[1,1′-biphenyl]-3-yl) methoxy)-2-((5-(methylsulfonyl)pyri- din-3-yl)methoxy)benzyl)(methyl) amino)acetamido)ethane-1,1- disulfonic acid |
| 137 | 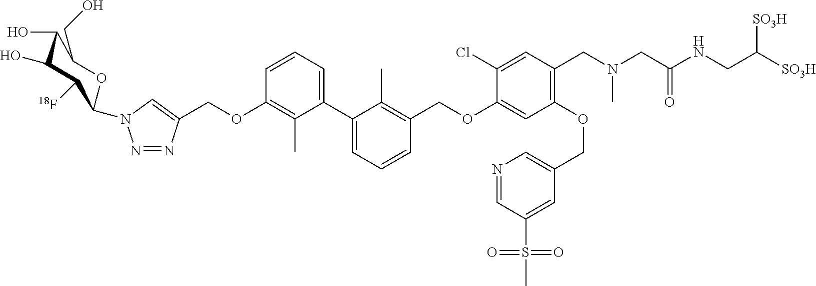 | corresponds Compound 89, wherein F is [¹⁸F] |
| 90 | 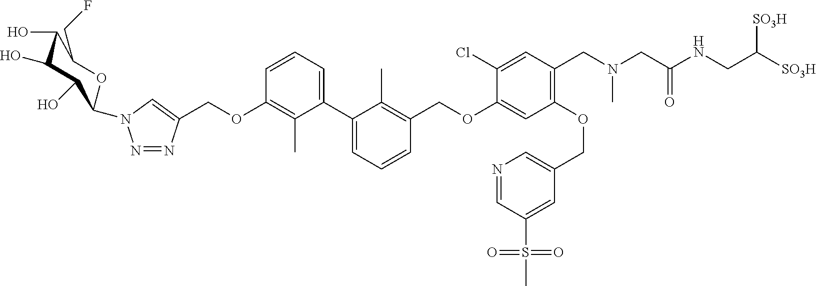 | 2-(2-((5-Chloro-4-((3′-((1- ((2R,3R,4S,5S,6S)-6-(fluorome- thyl)-3,4,5-trihydroxytetrahydro- 2H-pyran-2-yl)-1H-1,2,3-triazol- 4-yl)methoxy)-2,2′-dimethyl-[1,1′- biphenyl]-3-yl)methoxy)-2-((5- (methylsulfonyl)pyridin-3- yl)methoxy)benzyl)(methyl) amino)acetamido)ethane-1,1- disulfonic acid |
| 138 | 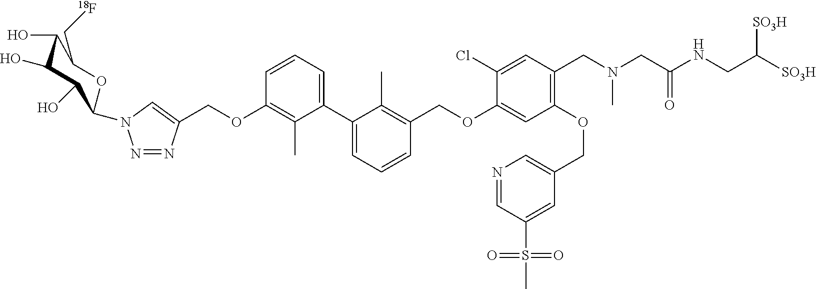 | corresponds Compound 90, wherein F is [¹⁸F] |
| 91 | 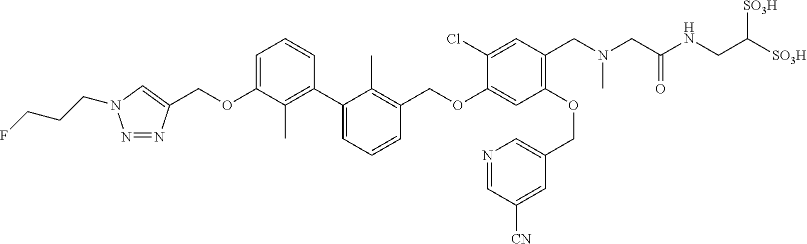 [SMILES: Cc1c(COc2cc(OCc3cncc(C#N)c3)c(CN(C)CC(=O)NCC(S(=O)(=O)O)S(=O)(=O)O)cc2Cl)cccc1-c1cccc(OCc2cn(CCCF)nn2)c1C] | 2-(2-((5-Chloro-2-((5-cyano- pyridin-3-yl)methoxy)-4-((3′-((1- (3-fluoropropyl)-1H-1,2,3-triazol- 4-yl)methoxy)-2,2′-dimethyl-[1,1′- biphenyl]-3-yl)methoxy)benzyl) (methyl)amino)acetamido) ethane-1,1-disulfonic acid |
| 139 | 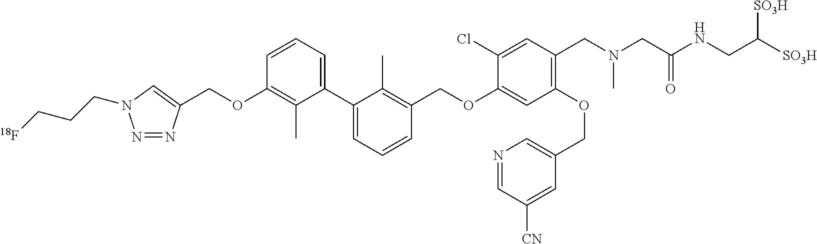 [SMILES: Cc1c(COc2cc(OCc3cncc(C#N)c3)c(CN(C)CC(=O)NCC(S(=O)(=O)O)S(=O)(=O)O)cc2Cl)cccc1-c1cccc(OCc2cn(CCC[18F])nn2)c1C] | corresponds Compound 91, where- in F is [¹⁸F] |
| 92 | 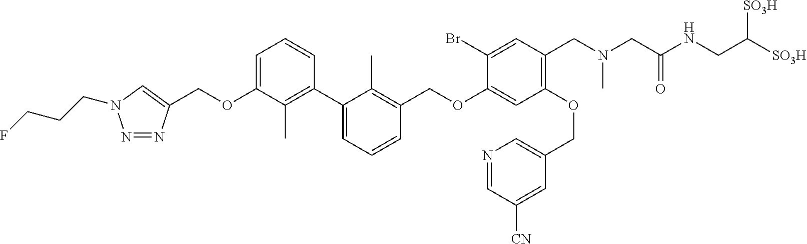 [SMILES: Cc1c(COc2cc(OCc3cncc(C#N)c3)c(CN(C)CC(=O)NCC(S(=O)(=O)O)S(=O)(=O)O)cc2Br)cccc1-c1cccc(OCc2cn(CCCF)nn2)c1C] | 2-(2-((5-bromo-2-((5-cyano- pyridin-3-yl)methoxy)-4-((3′-((1- (3-fluoropropyl)-1H-1,2,3-triazol- 4-yl)methoxy)-2,2′-dimethyl-[1,1′- biphenyl]-3-yl)methoxy)benzyl) (methyl)amino)acetamido) ethane-1,1-disulfonic acid |
| 140 | 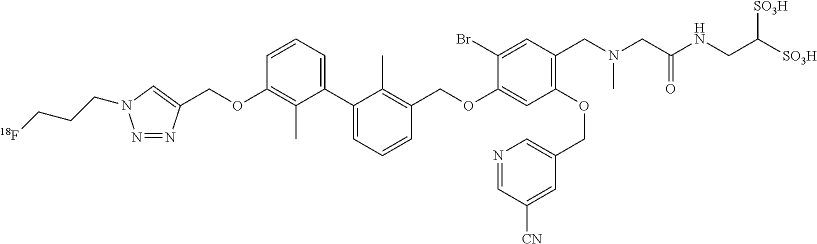 [SMILES: Cc1c(COc2cc(OCc3cncc(C#N)c3)c(CN(C)CC(=O)NCC(S(=O)(=O)O)S(=O)(=O)O)cc2Br)cccc1-c1cccc(OCc2cn(CCC[18F])nn2)c1C] | corresponds Compound 92, where- in F is [¹⁸F] |
| 93 | 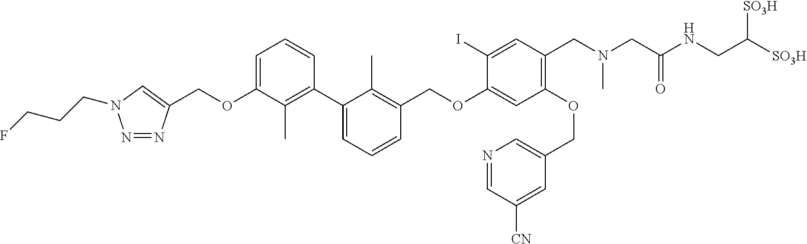 [SMILES: Cc1c(COc2cc(OCc3cncc(C#N)c3)c(CN(C)CC(=O)NCC(S(=O)(=O)O)S(=O)(=O)O)cc2I)cccc1-c1cccc(OCc2cn(CCCF)nn2)c1C] | 2-(2-((5-iodo-2-((5-cyanopyridin- 3-yl)methoxy)-4-((3′-((1-(3- fluoropropyl)-1H-1,2,3-triazol-4- yl)methoxy)-2,2′-dimethyl-[1,1′- biphenyl]-3-yl)methoxy)benzyl) (methyl)amino)acetamido) ethane-1,1-disulfonic acid |
| 141 | 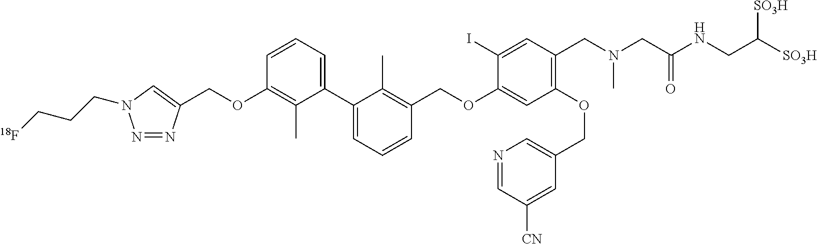 [SMILES: Cc1c(COc2cc(OCc3cncc(C#N)c3)c(CN(C)CC(=O)NCC(S(=O)(=O)O)S(=O)(=O)O)cc2I)cccc1-c1cccc(OCc2cn(CCC[18F])nn2)c1C] | corresponds Compound 93, where- in F is [¹⁸F] |
| 94 | 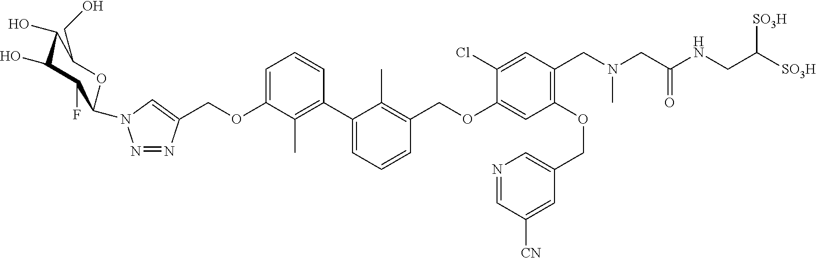 | 2-(2-((5-Chloro-2-((5-cyano- pyridin-3-yl)methoxy)-4-((3′-((1- ((2R,3R,4S,5S,6R)-3-fluoro-4,5- dihydroxy-6-(hydroxymethyl)tet- rahydro-2H-pyran-2-yl)-1H-1,2,3- triazol-4-yl)methoxy)-2,2′-di- methyl-[1,1′-biphenyl]-3-yl)methoxy) benzyl)(methyl)amino)acetamido) ethane-1,1-disulfonic acid |
| 142 | 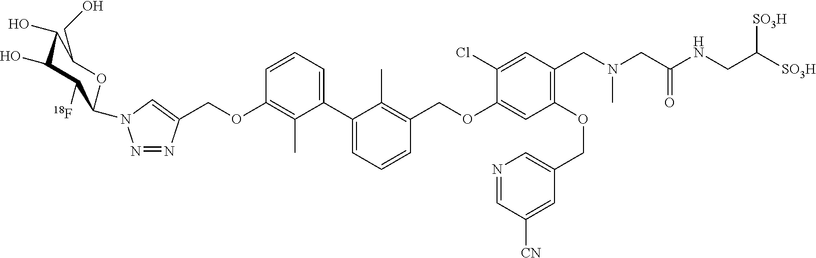 | corresponds Compound 94, where- in F is [¹⁸F] |
| 95 | 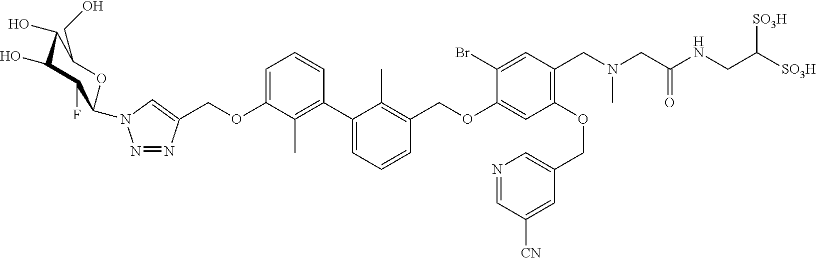 | 2-(2-((5-bromo-2-((5-cyano- pyridin-3-yl)methoxy)-4-((3′-((1- ((2R,3R,4S,5S,6R)-3-fluoro-4,5- dihydroxy-6-(hydroxymethyl)tet- rahydro-2H-pyran-2-yl)-1H-1,2,3- triazol-4-yl)methoxy)-2,2′-di- methyl-[1,1′-biphenyl]-3-yl)methoxy) benzyl)(methyl)amino)acetamido) ethane-1,1-disulfonic acid |
| 143 | 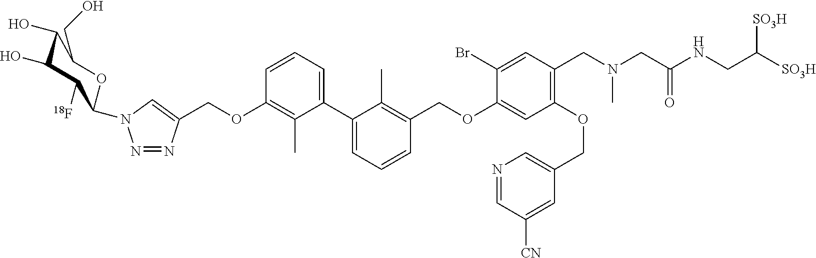 | corresponds Compound 95, where- in F is [¹⁸F] |
| 96 | 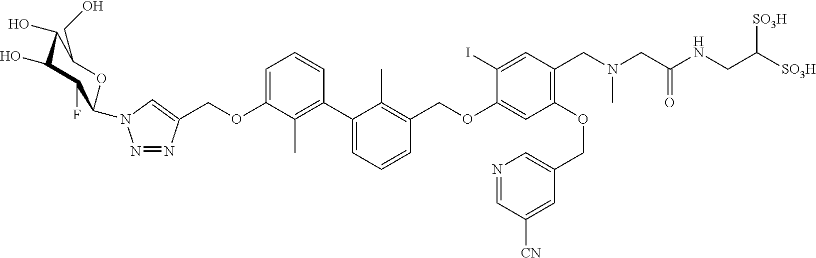 | 2-(2-((5-iodo-2-((5-cyanopyridin- 3-yl)methoxy)-4-((3′-((1- ((2R,3R,4S,5S,6R)-3-fluoro-4,5- dihydroxy-6-(hydroxymethyl)tet- rahydro-2H-pyran-2-yl)-1H-1,2,3- triazol-4-yl)methoxy)-2,2′-di- methyl-[1,1′-biphenyl]-3-yl)methoxy) benzyl)(methyl)amino)acetamido) ethane-1,1-disulfonic acid |
| 144 | 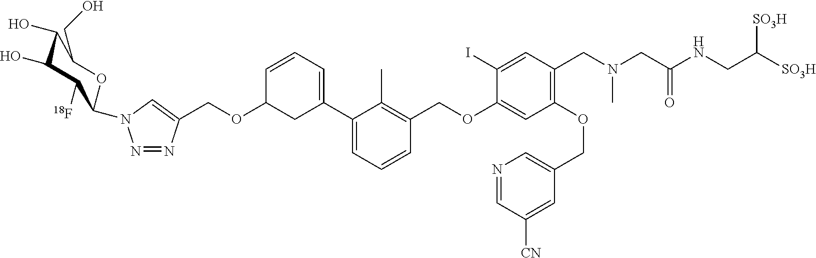 | corresponds Compound 96, where- in F is [¹⁸F] |
| 97 | 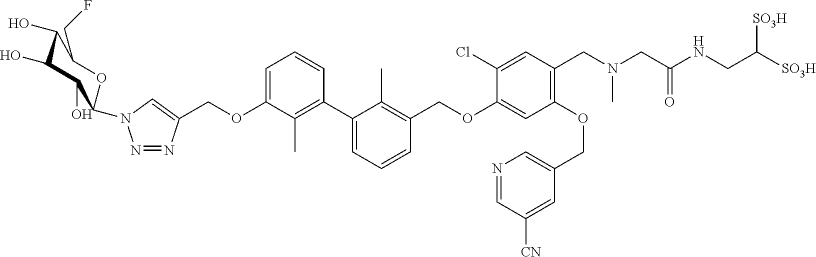 | 2-(2-((5-Chloro-2-((5-cyano- pyridin-3-yl)methoxy)-4-((3′-((1- ((2R,3R,4S,5S,6S)-6-(fluoro- methyl)-3,4,5-trihydroxytetrahydro- 2H-pyran-2-yl)-1H-1,2,3-triazol- 4-yl)methoxy)-2,2′-dimethyl-[1,1′- biphenyl]-3-yl)methoxy)benzyl) (methyl)amino)acetamido) ethane-1,1-disulfonic acid |
| 145 | 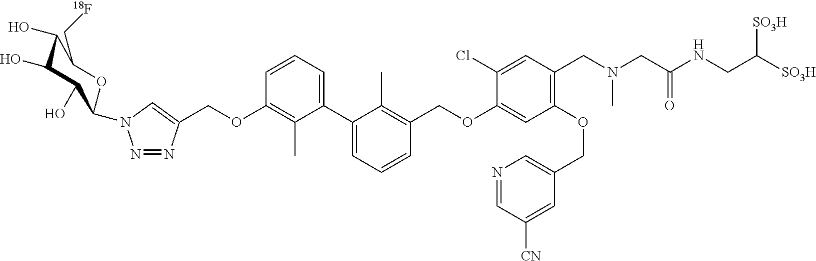 | corresponds Compound 97, where- in F is [¹⁸F] |
| 98 | 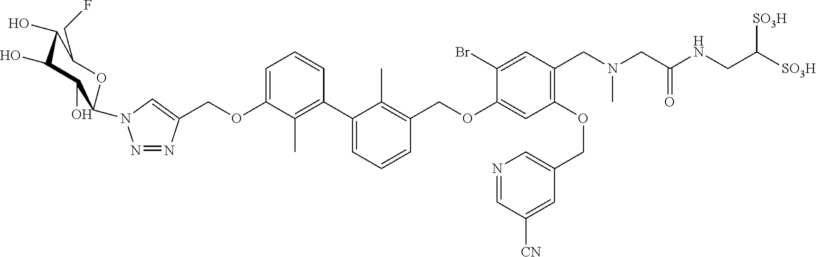 | 2-(2-((5-bromo-2-((5-cyano- pyridin-3-yl)methoxy)-4-((3′-((1- ((2R,3R,4S,5S,6S)-6-(fluoro- methyl)-3,4,5-trihydroxytetrahydro- 2H-pyran-2-yl)-1H-1,2,3-triazol- 4-yl)methoxy)-2,2′-dimethyl-[1,1′- biphenyl]-3-yl)methoxy)benzyl) (methyl)amino)acetamido) ethane-1,1-disulfonic acid |
| 146 | 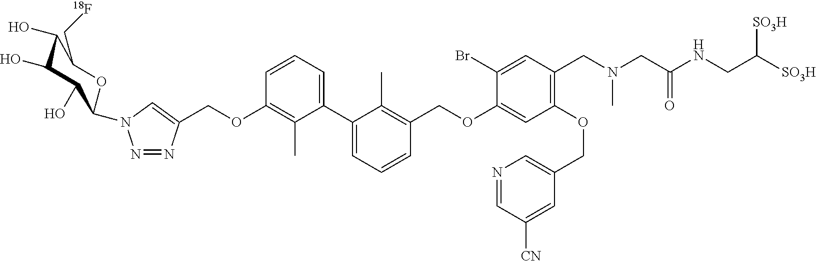 | corresponds Compound 98, where- in F is [¹⁸F] |
| 99 | 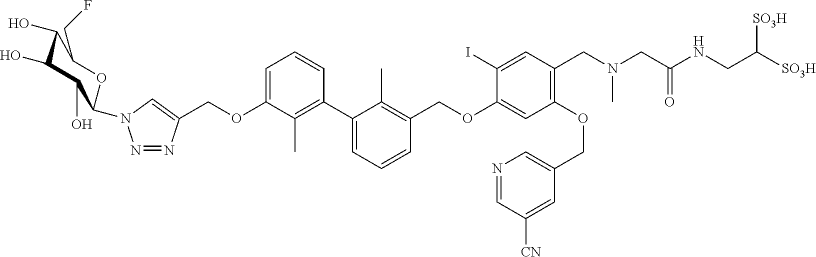 | 2-(2-((5-iodo-2-((5-cyanopyridin- 3-yl)methoxy)-4-((3′-((1- ((2R,3R,4S,5S,6S)-6-(fluoro- methyl)-3,4,5-trihydroxytetrahydro- 2H-pyran-2-yl)-1H-1,2,3-triazol- 4-yl)methoxy)-2,2′-dimethyl-[1,1′- biphenyl]-3-yl)methoxy)benzyl) (methyl)amino)acetamido) ethane-1,1-disulfonic acid |
| 147 | 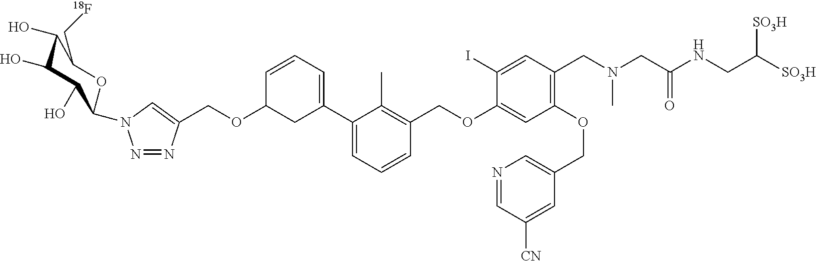 | corresponds Compound 99, where- in F is [¹⁸F] |
| 102 | 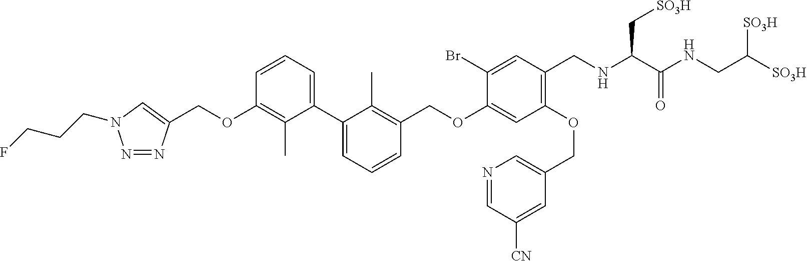 [SMILES: Cc1c(COc2cc(OCc3cncc(C#N)c3)c(CN[C@@H](CS(=O)(=O)O)C(=O)NCC(S(=O)(=O)O)S(=O)(=O)O)cc2Br)cccc1-c1cccc(OCc2cn(CCCF)nn2)c1C] | (S)-2-(2-((5-Bromo-2-((5-cyano- pyridin-3-yl)methoxy)-4-((3′-((1- (3-fluoropropyl)-1H-1,2,3-triazol- 4-yl)methoxy)-2,2′-dimethyl-[1,1′- biphenyl]-3-yl)methoxy)benzyl) amino)-3-sulfopropanamido) ethane-1,1-disulfonic acid |
| 148 | 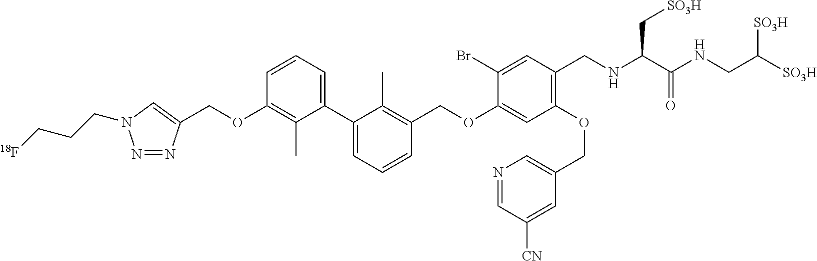 [SMILES: Cc1c(COc2cc(OCc3cncc(C#N)c3)c(CN[C@@H](CS(=O)(=O)O)C(=O)NCC(S(=O)(=O)O)S(=O)(=O)O)cc2Br)cccc1-c1cccc(OCc2cn(CCC[18F])nn2)c1C] | corresponds Compound 102, where-in F is [¹⁸F] |
| 103 | 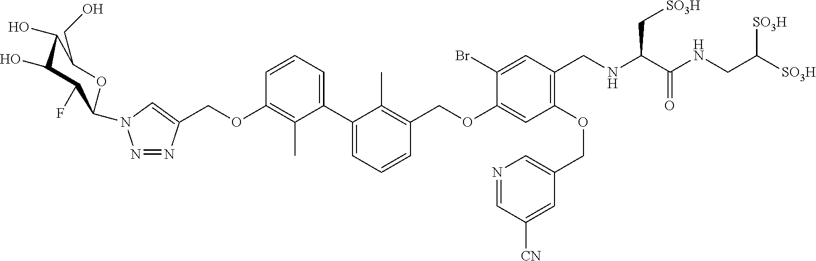 | 2-((S)-2-((5-bromo-2-((5-cyano- pyridin-3-yl)methoxy)-4-((3′-((1- ((2R,3R,4S,5S,6R)-3-fluoro-4,5- dihydroxy-6-(hydroxymethyl)tet- rahydro-2H-pyran-2-yl)-1H-1,2,3- triazol-4-yl)methoxy)-2,2′-dimethyl- [1,1′-biphenyl]-3-yl)methoxy) benzyl)amino)-3-sulfopro- panamido)ethane-1,1-disulfonic acid |
| 149 | 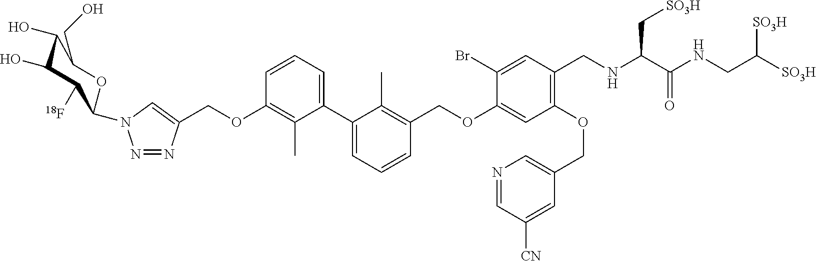 | corresponds Compound 103, wherein F is [¹⁸F] |
| 104 | 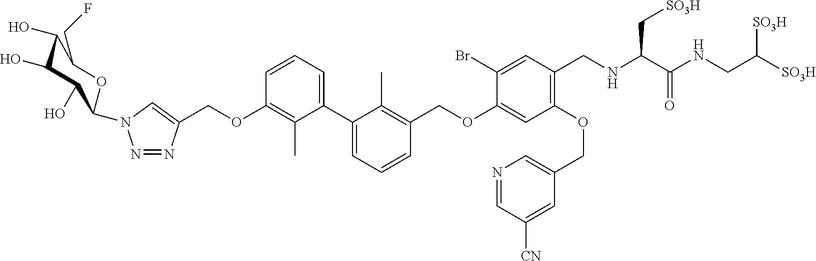 | 2-((S)-2-((5-Bromo-2-((5-cyano- pyridin-3-yl)methoxy)-4-((3′-((1- ((2R,3R,4S,5S,6S)-6-(fluoro- methyl)-3,4,5-trihydroxytetrahydro- 2H-pyran-2-yl)-1H-1,2,3-triazol- 4-yl)methoxy)-2,2′-dimethyl-[1,1′- biphenyl]-3-yl)methoxy)benzyl) amino)-3-sulfopropanamido) ethane-1,1-disulfonic acid |
| 150 | 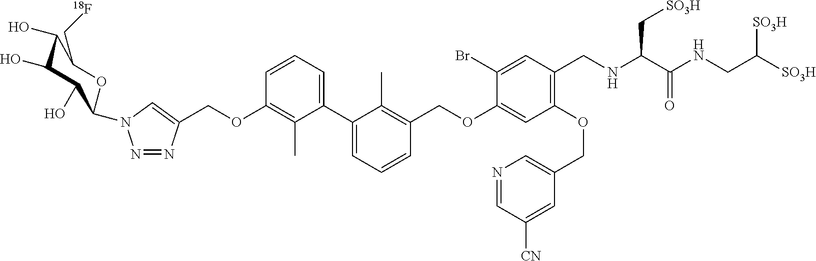 | corresponds Compound 104, wherein F is [¹⁸F] |

Compound 85 is a compound of general formula I, in which R¹ is hydrogen, R² is bromine, R³ is Cl, one X is N, the other are C—H, Y is a methyl sulphonyl group, Z is a group of formula III, wherein R⁴ are sulphonic acid groups, and M is a group of formula XII, wherein R⁶ is fluorine. Compound 151 corresponds compound 85 with the exception of R⁶ is [¹⁸F]fluorine.

Compound 86 is a compound of general formula I, in which R¹ is hydrogen, R² is bromine, R³ is Cl, one X is N, the other are C—H, Y is a methyl sulphonyl group, Z is a group of formula III, wherein R⁴ are sulphonic acid groups, and M is a group of formula X, wherein R⁷ is fluorine. Compound 134 corresponds compound 86 with the exception of R⁷ is [¹⁸F]fluorine

Compound 87 is a compound of general formula I, in which R¹ is hydrogen, R² is bromine, R³ is Cl, one X is N, the other are C—H, Y is a methyl sulphonyl group, Z is a group of formula III, wherein R⁴ are sulphonic acid groups, and M is a group of formula IX, wherein R⁶ is fluorine. Compound 135 corresponds compound 87 with the exception of R⁶ is [¹⁸F]fluorine.

Compound 88 is a compound of general formula I, in which R¹ is a methyl group, R² is a methyl group, R³ is Cl, one X is N, the other are C—H, Y is a methyl sulphonyl group, Z is a group of formula III, wherein R⁴ are sulphonic acid groups, and M is a group of formula XII, wherein R⁶ is fluorine. Compound 136 corresponds compound 88 with the exception of R⁶ is [¹⁸F]fluorine.

Compound 89 is a compound of general formula I, in which R¹ is a methyl group, R² is a methyl group, R³ is Cl, one X is N, the other are C—H, Y is a methyl sulphonyl group, Z is a group of formula III, wherein R⁴ are sulphonic acid groups, and M is a group of formula X, wherein R⁷ is fluorine. Compound 137 corresponds compound 89 with the exception of R⁷ is [¹⁸F]fluorine.

Compound 90 is a compound of general formula I, in which R¹ is a methyl group, R² is a methyl group, R³ is Cl, one X is N, the other are C—H, Y is a methyl sulphonyl group, Z is a group of formula III, wherein R⁴ are sulphonic acid groups, and M is a group of formula IX, wherein R⁶ is fluorine. Compound 138 corresponds compound 90 with the exception of R⁶ is [¹⁸F]fluorine.

Compound 91 is a compound of general formula I, in which R¹ is a methyl group, R² is a methyl group, R³ is Cl, one X is N, the other are C—H, Y is a cyano group, Z is a group of formula III, wherein R⁴ are sulphonic acid groups, and M is a group of formula XII, wherein R⁶ is fluorine. Compound 139 corresponds compound 91 with the exception of R⁶ is [¹⁸F]fluorine.

Compound 92 is a compound of general formula I, in which R¹ is a methyl group, R² is a methyl group, R³ is Br, one X is N, the other are C—H, Y is a cyano group, Z is a group of formula III, wherein R⁴ are sulphonic acid groups, and M is a group of formula XII, wherein R⁶ is fluorine. Compound 140 corresponds compound 92 with the exception of R⁶ is [¹⁸F]fluorine.

Compound 93 is a compound of general formula I, in which R¹ is a methyl group, R² is a methyl group, R³ is I, one X is N, the other are C—H, Y is a cyano group, Z is a group of formula III, wherein R⁴ are sulphonic acid groups, and M is a group of formula XII, wherein R⁶ is fluorine. Compound 141 corresponds compound 93 with the exception of R⁶ is [¹⁸F]fluorine.

Compound 94 is a compound of general formula I, in which R¹ is a methyl group, R² is a methyl group, R³ is Cl, one X is N, the other are C—H, Y is is a cyano group, Z is a group of formula III, wherein R⁴ are sulphonic acid groups, and M is a group of formula X, wherein R⁷ is fluorine. Compound 142 corresponds compound 94 with the exception of R⁷ is [¹⁸F]fluorine.

Compound 95 is a compound of general formula I, in which R¹ is a methyl group, R² is a methyl group, R³ is Br, one X is N, the other are C—H, Y is is a cyano group, Z is a group of formula III, wherein R⁴ are sulphonic acid groups, and M is a group of formula X, wherein R⁷ is fluorine. Compound 143 corresponds compound 95 with the exception of R⁷ is [¹⁸F]fluorine.

Compound 96 is a compound of general formula I, in which R¹ is a methyl group, R² is a methyl group, R³ is I, one X is N, the other are C—H, Y is a cyano group, Z is a group of formula III, wherein R⁴ are sulphonic acid groups, and M is a group of formula X, wherein R⁷ is fluorine. Compound 144 corresponds compound 96 with the exception of R⁷ is [¹⁸F]fluorine.

Compound 97 is a compound of general formula I, in which R¹ is a methyl group, R² is a methyl group, R³ is Cl, one X is N, the other are C—H, Y is a cyano group, Z is a group of formula III, wherein R⁴ are sulphonic acid groups, and M is a group of formula IX, wherein R⁶ is fluorine. Compound 145 corresponds compound 97 with the exception of R⁶ is [¹⁸F]fluorine.

Compound 98 is a compound of general formula I, in which R¹ is a methyl group, R² is a methyl group, R³ is Br, one X is N, the other are C—H, Y is a cyano group, Z is a group of formula III, wherein R⁴ are sulphonic acid groups, and M is a group of formula IX, wherein R⁶ is fluorine. Compound 146 corresponds compound 98 with the exception of R⁶ is [¹⁸F]fluorine.

Compound 99 is a compound of general formula I, in which R¹ is a methyl group, R² is a methyl group, R³ is I, one X is N, the other are C—H, Y is a cyano group, Z is a group of formula III, wherein R⁴ are sulphonic acid groups, and M is a group of formula IX, wherein R⁶ is fluorine. Compound 147 corresponds compound 99 with the exception of R⁶ is [¹⁸F]fluorine.

Compound 102 is a compound of general formula I, in which R¹ is a methyl group, R² is a methyl group, R³ is Br, one X is N, the other are C—H, Y is a cyano group, Z is a group of formula IV, wherein R⁴ are sulphonic acid groups, and M is a group of formula XII, wherein R⁶ is fluorine. Compound 148 corresponds compound 102 with the exception of R⁶ is [¹⁸F]fluorine.

Compound 103 is a compound of general formula I, in which R¹ is a methyl group, R² is a methyl group, R³ is Br, one X is N, the other are C—H, Y is a cyano group, Z is a group of formula IV, wherein R⁴ are sulphonic acid groups, and M is a group of formula X, wherein R⁷ is fluorine. Compound 149 corresponds compound 103 with the exception of R⁷ is [¹⁸F]fluorine.

Compound 104 is a compound of general formula I, in which R¹ is a methyl group, R² is a methyl group, R³ is Br, one X is N, the other are C—H, Y is a cyano group, Z is a group of formula IV, wherein R⁴ are sulphonic acid groups, and M is a group of formula IX, wherein R⁶ is fluorine. Compound 150 corresponds compound 104 with the exception of R⁶ is [¹⁸F]fluorine.

TABLE 2
Chelate compounds

| Compound | Structure | Name |
|---|---|---|
| 42 | 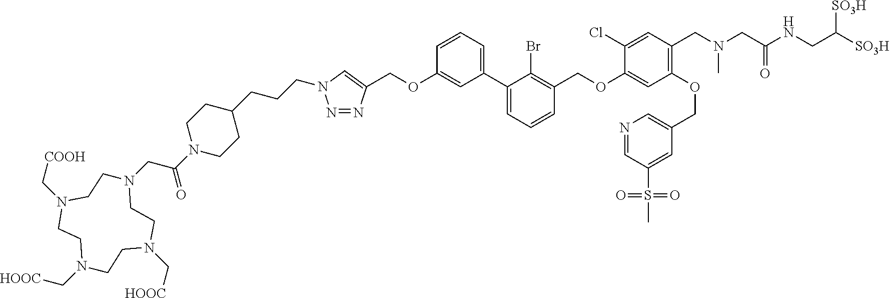 [SMILES: CN(CC(=O)NCC(S(=O)(=O)O)S(=O)(=O)O)Cc1cc(Cl)c(OCc2cccc(-c3cccc(OCc4cn(CCCC5CCN(C(=O)CN6CCN(CC(=O)O)CCN(CC(=O)O)CCN(CC(=O)O)CC6)CC5)nn4)c3)c2Br)cc1OCc1cncc(S(C)(=O)=O)c1] | 2,2′,2″-(10-(2-(4-(3-(4-(((2′- Bromo-3′-((2-chloro-4-(((2-((2,2- disulfoethyl)amino)-2-oxo- ethyl)(methyl)amino)methyl)-5- ((5-(methylsulfonyl)pyridin-3- yl)methoxy)phenoxy)methyl)- [1,1′-biphenyl]-3-yl)oxy)methyl)- 1H-1,2,3-triazol-1-yl)propyl)piper- idin-1-yl)-2-oxoethyl)-1,4,7,10- tetraazacyclododecane-1,4,7- triyl)triacetic acid, copper(II)-⁶⁴Cu salt |
| 105 | 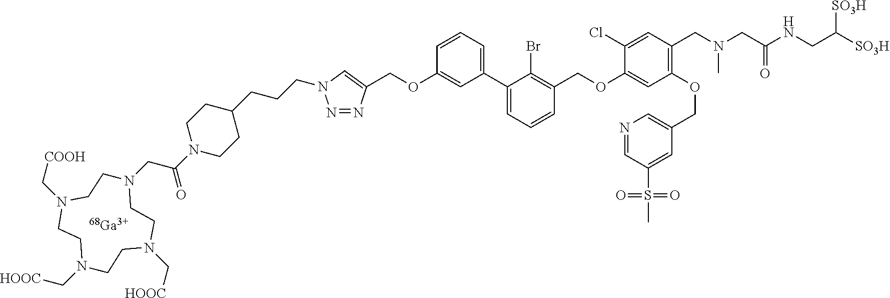 | corresponds Compound 42 with ⁶⁸Ga³⁺ as central atom of the chelate complex |
| 115 | 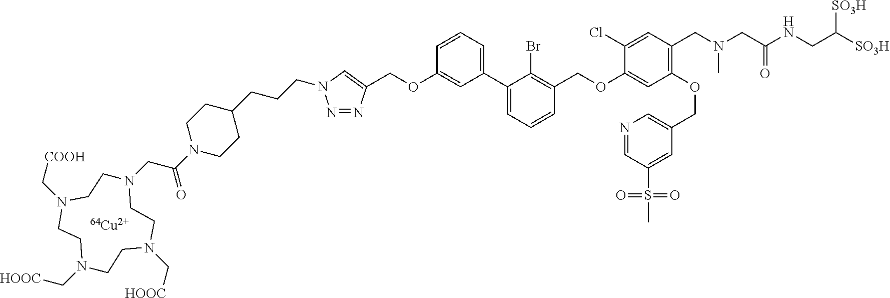 | corresponds Compound 42 with ⁶⁴Cu²⁺ as central atom of the chelate complex |
| 125 | 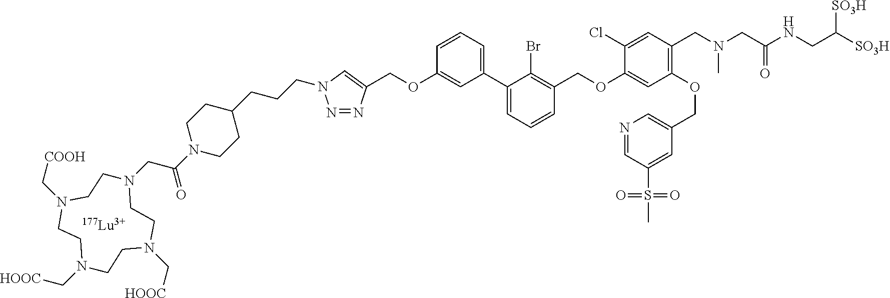 | corresponds Compound 42 with ¹⁷⁷Lu³⁺ as central atom of the che- late complex |
| 43 | 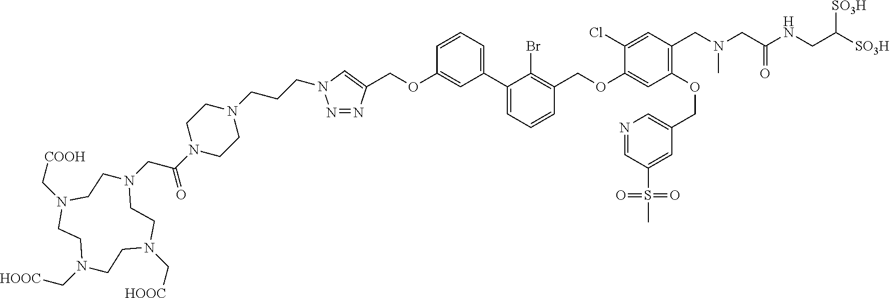 [SMILES: CN(CC(=O)NCC(S(=O)(=O)O)S(=O)(=O)O)Cc1cc(Cl)c(OCc2cccc(-c3cccc(OCc4cn(CCCN5CCN(C(=O)CN6CCN(CC(=O)O)CCN(CC(=O)O)CCN(CC(=O)O)CC6)CC5)nn4)c3)c2Br)cc1OCc1cncc(S(C)(=O)=O)c1] | 2,2′,2″-(10-(2-(4-(3-(4-(((2′- Bromo-3′-((2-chloro-4-(((2-((2,2- disulfoethyl)amino)-2-oxo- ethyl)(methyl)amino)methyl)-5- ((5-(methylsulfonyl)pyridin-3- yl)methoxy)phenoxy)methyl)- [1,1′-biphenyl]-3-yl)oxy)methyl)- 1H-1,2,3-triazol-1-yl)propyl)piper- azin-1-yl)-2-oxoethyl)-1,4,7,10- tetraazacyclododecane-1,4,7- triyl)triacetic acid |
| 106 | 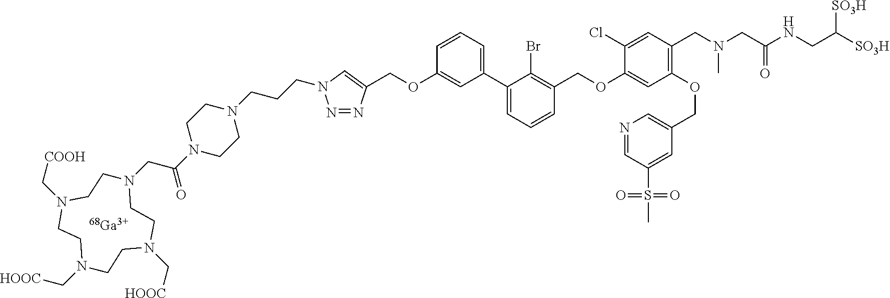 | corresponds Compound 43 with ⁶⁸Ga³⁺ as central atom of the chelate complex |
| 116 | 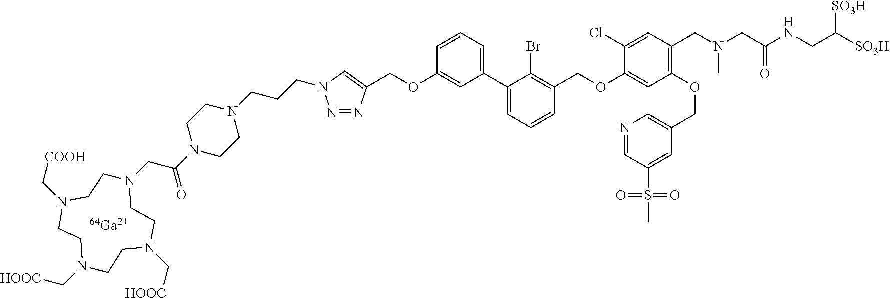 | corresponds Compound 43 with ⁶⁴Cu²⁺ as central atom of the chelate complex |
| 126 | 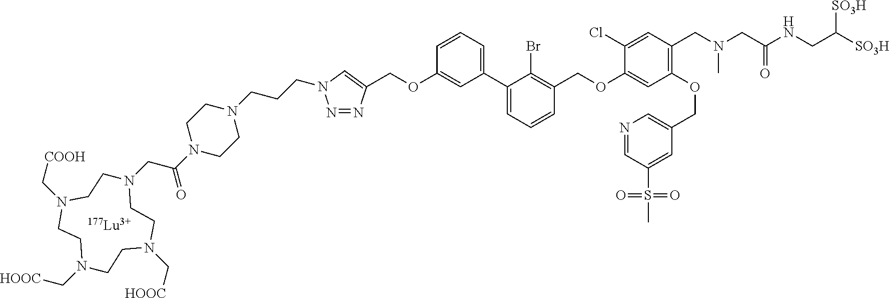 | corresponds Compound 43 with ¹⁷⁷Lu³⁺ as central atom of the che- late complex |
| 44 | 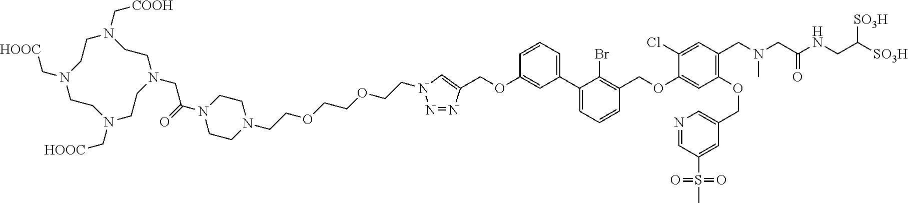 [SMILES: CN(CC(=O)NCC(S(=O)(=O)O)S(=O)(=O)O)Cc1cc(Cl)c(OCc2cccc(-c3cccc(OCc4cn(CCOCCOCCN5CCN(C(=O)CN6CCN(CC(=O)O)CCN(CC(=O)O)CCN(CC(=O)O)CC6)CC5)nn4)c3)c2Br)cc1OCc1cncc(S(C)(=O)=O)c1] | 2,2′,2″-(10-(2-(4-(2-(2-(2-(4-(((2′- Bromo-3′-((2-chloro-4-(((2-((2,2- disulfoethyl)amino)-2-oxo- ethyl)(methyl)amino)methyl)-5- ((5-(methylsulfonyl)pyridin-3- yl)methoxy)phenoxy)methyl)- [1,1′-biphenyl]-3-yl)oxy)methyl)- 1H-1,2,3-triazol-1-yl)ethoxy)eth- oxy)ethyl)piperazin-1-yl)-2-ox- oethyl)-1,4,7,10-tetraazacyclodo- decane-1,4,7-triyl)triacetic acid |
| 107 | 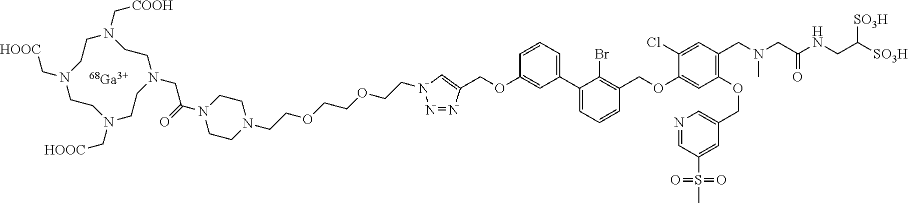 | corresponds Compound 44 with ⁶⁸Ga³⁺ as central atom of the chelate complex |
| 117 | 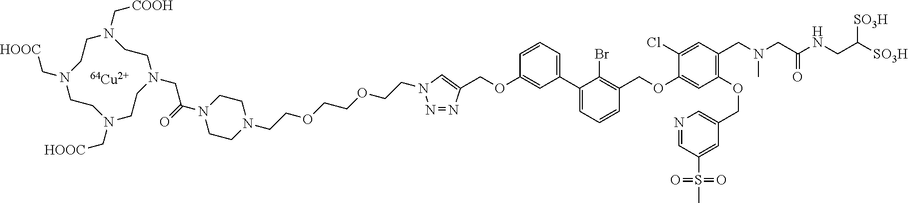 | corresponds Compound 44 with ⁶⁴Cu²⁺ as central atom of the chelate complex |
| 127 | 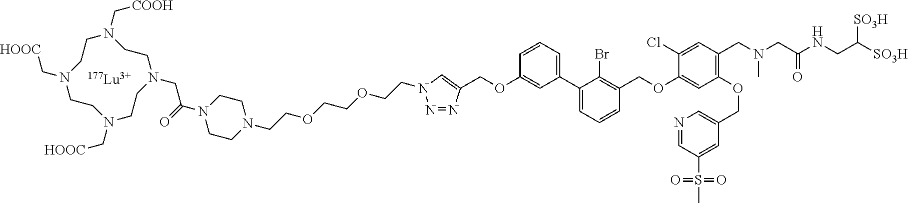 | corresponds Compound 44 with ¹⁷⁷Lu³⁺ as central atom of the che- late complex |
| 45 | 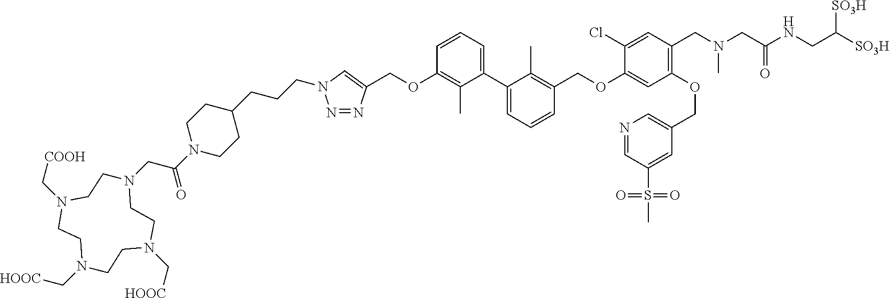 [SMILES: Cc1c(COc2cc(OCc3cncc(S(C)(=O)=O)c3)c(CN(C)CC(=O)NCC(S(=O)(=O)O)S(=O)(=O)O)cc2Cl)cccc1-c1cccc(OCc2cn(CCCC3CCN(C(=O)CN4CCN(CC(=O)O)CCN(CC(=O)O)CCN(CC(=O)O)CC4)CC3)nn2)c1C] | 2,2′,2″-(10-(2-(4-(3-(4-(((3′-((2- Chloro-4-(((2-((2,2-disul- foethyl)amino)-2-oxoethyl)(meth- yl)amino)methyl)-5-((5-(methyl- sulfonyl)pyridin-3-yl)meth- oxy)phenoxy)methyl)-2,2′-dimeth- yl-[1,1′-biphenyl]-3-yl)oxy)meth- yl)-1H-1,2,3-triazol-1-yl)pro- pyl)piperidin-1-yl)-2-oxoethyl)- 1,4,7,10-tetraazacyclododecane- 1,4,7-triyl)triacetic acid |
| 108 | 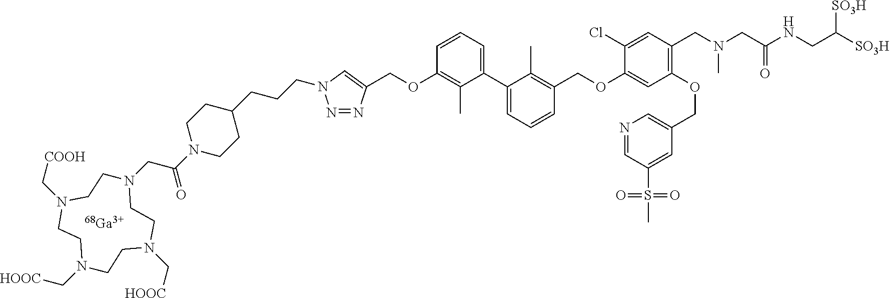 | corresponds Compound 45 with ⁶⁸Ga³⁺ as central atom of the chelate complex |
| 118 | 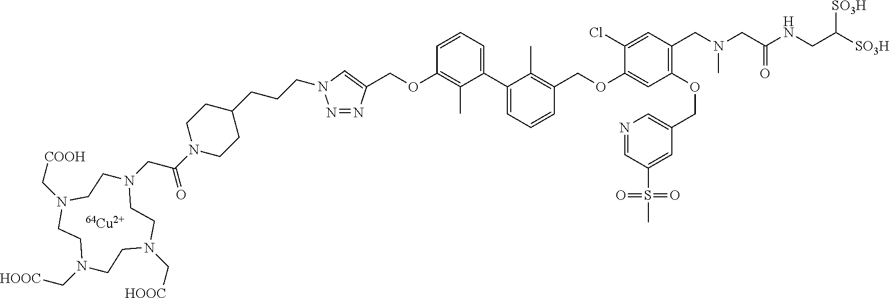 | corresponds Compound 45 with ⁶⁴Cu²⁺ as central atom of the chelate complex |
| 128 | 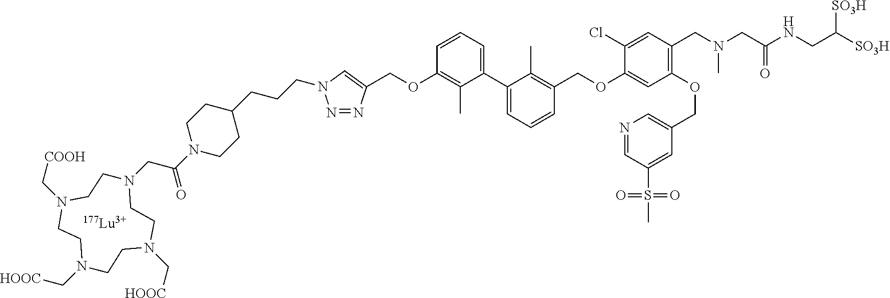 | corresponds Compound 45 with ¹⁷⁷Lu³⁺ as central atom of the che- late complex |
| 46 | 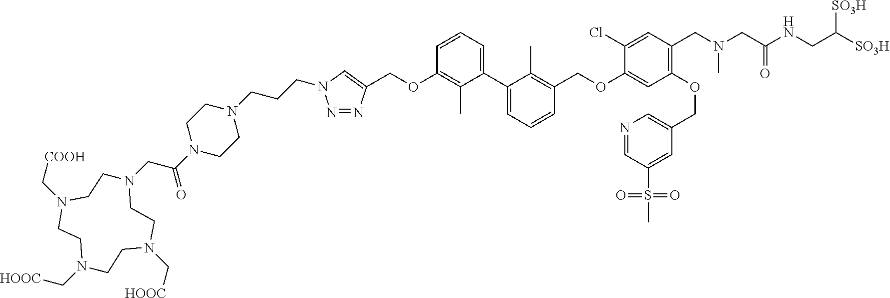 [SMILES: Cc1c(COc2cc(OCc3cncc(S(C)(=O)=O)c3)c(CN(C)CC(=O)NCC(S(=O)(=O)O)S(=O)(=O)O)cc2Cl)cccc1-c1cccc(OCc2cn(CCCN3CCN(C(=O)CN4CCN(CC(=O)O)CCN(CC(=O)O)CCN(CC(=O)O)CC4)CC3)nn2)c1C] | 2,2′,2″-(10-(2-(4-(3-(4-(((3′-((2- Chloro-4-(((2-((2,2-disul- foethyl)amino)-2-oxoethyl)(meth- yl)amino)methyl)-5-((5-(methyl- sulfonyl)pyridin-3-yl)meth- oxy)phenoxy)methyl)-2,2′-dimeth- yl-[1,1′-biphenyl]-3-yl)oxy)meth- yl)-1H-1,2,3-triazol-1-yl)pro- pyl)piperazin-1-yl)-2-oxoethyl)- 1,4,7,10-tetraazacyclododecane- 1,4,7-triyl)triazetic acid |
| 109 | 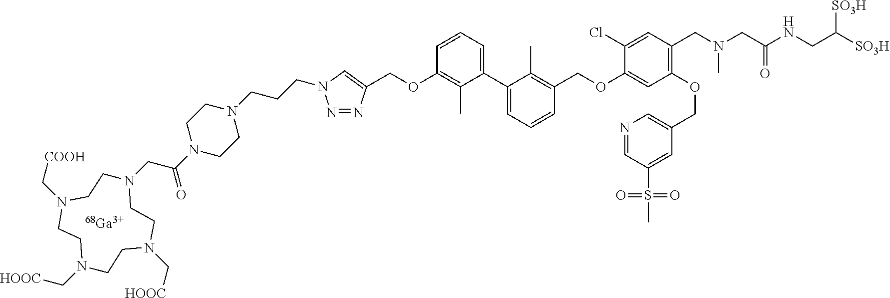 | corresponds Compound 46 with ⁶⁸Ga³⁺ as central atom of the chelate complex |
| 119 | 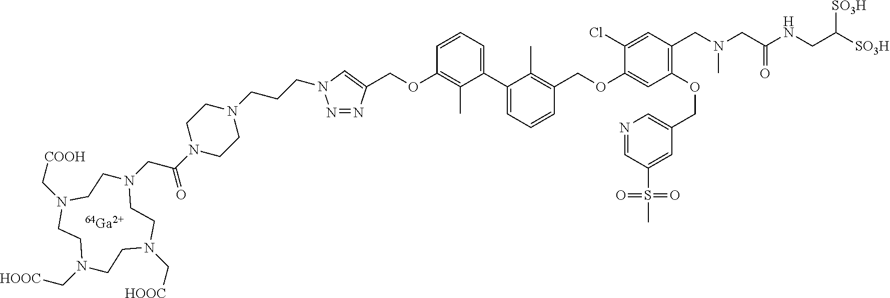 | corresponds Compound 46 with ⁶⁴Cu²⁺ as central atom of the chelate complex |
| 129 | 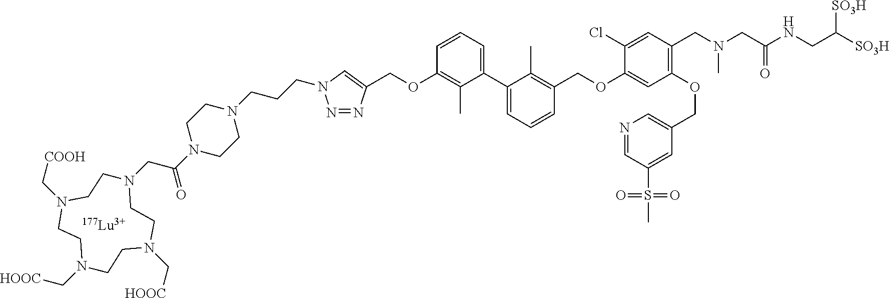 | corresponds Compound 46 with ¹⁷⁷Lu³⁺ as central atom of the che- late complex |
| 47 | 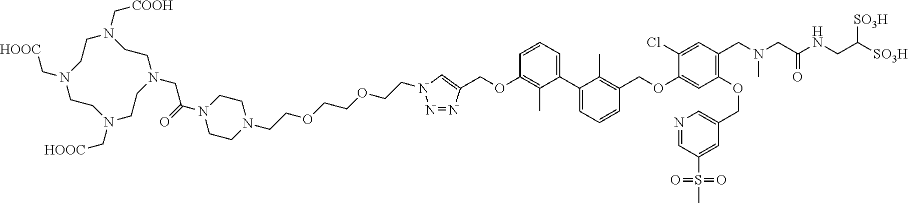 [SMILES: Cc1c(COc2cc(OCc3cncc(S(C)(=O)=O)c3)c(CN(C)CC(=O)NCC(S(=O)(=O)O)S(=O)(=O)O)cc2Cl)cccc1-c1cccc(OCc2cn(CCOCCOCCN3CCN(C(=O)CN4CCN(CC(=O)O)CCN(CC(=O)O)CCN(CC(=O)O)CC4)CC3)nn2)c1C] | 2,2′,2″-(10-(2-(4-(2-(2-(2-(4-(((3′- ((2-Chloro-4-(((2-((2,2-disul- foethyl)amino)-2-oxoethyl)(meth- yl)amino)methyl)-5-((5-(methyl- sulfonyl)pyridin-3-yl)meth- oxy)phenoxy)methyl)-2,2′-dimeth- yl-[1,1′-biphenyl]-3-yl)oxy)meth- yl)-1H-1,2,3-triazol-1-yl)eth- oxy)ethoxy)ethyl)piperazin-1-yl)- 2-oxoethyl)-1,4,7,10-tetraazacy- clododecane-1,4,7-triyl)triacetic acid |
| 110 | 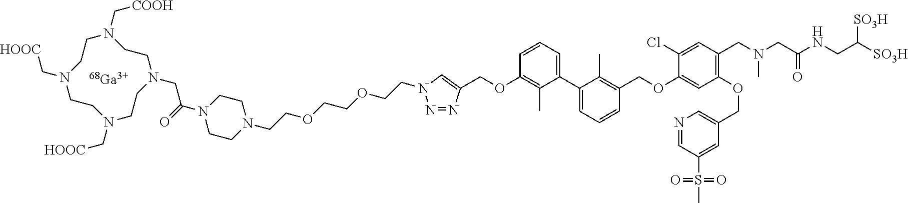 | corresponds Compound 47 with ⁶⁸Ga³⁺ as central atom of the chelate complex |
| 120 | 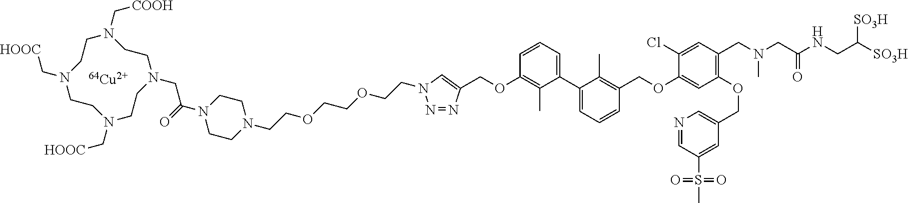 | corresponds Compound 47 with ⁶⁴Cu²⁺ as central atom of the chelate complex |
| 130 | 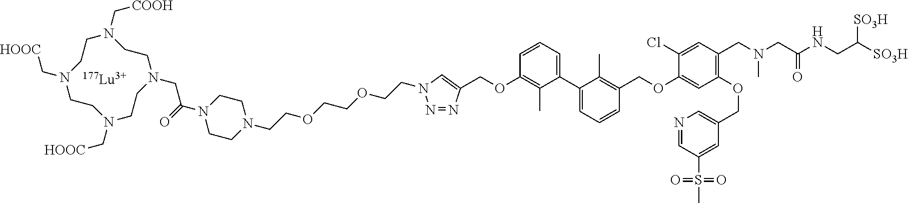 | corresponds Compound 47 with ¹⁷⁷Lu³⁺ as central atom of the che- late complex |
| 54 | 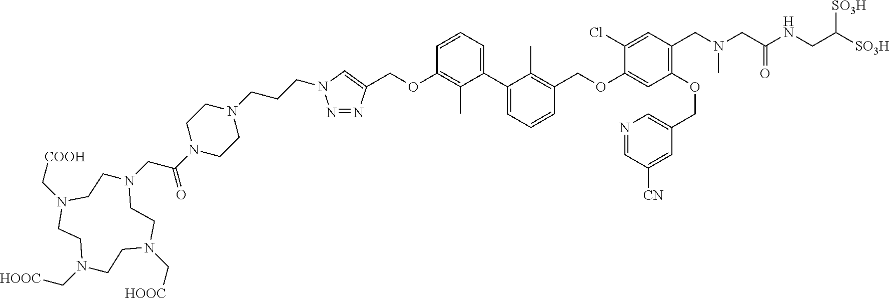 [SMILES: Cc1c(COc2cc(OCc3cncc(C#N)c3)c(CN(C)CC(=O)NCC(S(=O)(=O)O)S(=O)(=O)O)cc2Cl)cccc1-c1cccc(OCc2cn(CCCN3CCN(C(=O)CN4CCN(CC(=O)O)CCN(CC(=O)O)CCN(CC(=O)O)CC4)CC3)nn2)c1C] | 2,2′,2″-(10-(2-(4-(3-(4-(((3′-((2- Chloro-5-((5-cyanopyridin-3- yl)methoxy)-4-(((2-((2,2-disul- foethyl)amino)-2-oxoethyl)(meth- yl)amino)methyl)phenoxy)meth- yl)-2,2′-dimethyl-[1,1′-biphenyl]- 3-yl)oxy)methyl)-1H-1,2,3-triazol- 1-yl)propyl)piperazin-1-yl)-2-oxo- ethyl)-1,4,7,10-tetraazacyclodo- decane-1,4,7-triyl)triacetic acid |
| 111 | 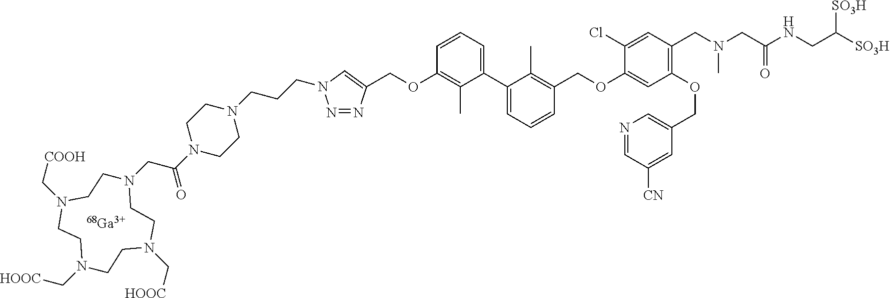 | corresponds Compound 54 with ⁶⁸Ga³⁺ as central atom of the chelate complex |
| 121 | 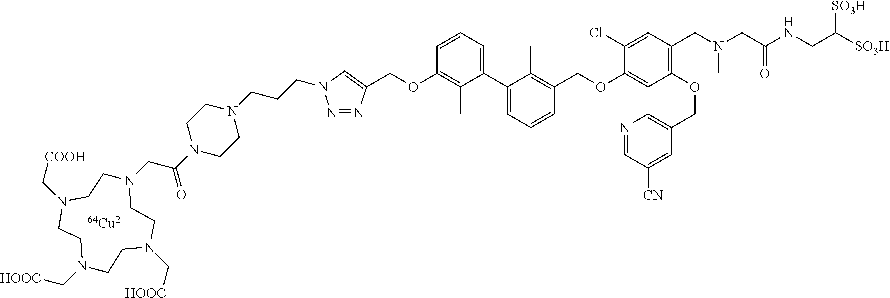 | corresponds Compound 54 with ⁶⁴Cu²⁺ as central atom of the chelate complex |
| 131 | 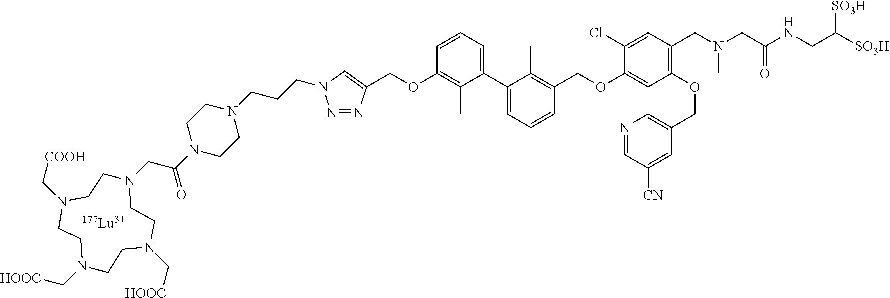 | corresponds Compound 54 with ¹⁷⁷Lu³⁺ as central atom of the che- late complex |
| 63 | 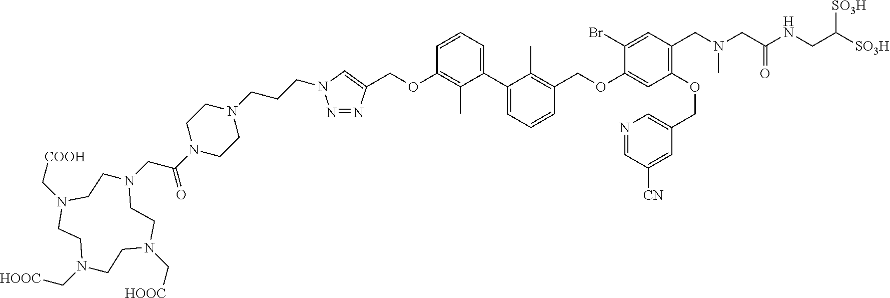 [SMILES: Cc1c(COc2cc(OCc3cncc(C#N)c3)c(CN(C)CC(=O)NCC(S(=O)(=O)O)S(=O)(=O)O)cc2Br)cccc1-c1cccc(OCc2cn(CCCN3CCN(C(=O)CN4CCN(CC(=O)O)CCN(CC(=O)O)CCN(CC(=O)O)CC4)CC3)nn2)c1C] | 2,2′,2″-(10-(2-(4-(3-(4-(((3′-((2- Bromo-5-((5-cyanopyridin-- yl)methoxy)-4-(((2-((2,2-disul- foethyl)amino)-2-oxoethyl)(meth- yl)amino)methyl)phenoxy)meth- yl)-2,2′-dimethyl-[1,1′-biphenyl]- 3-yl)oxy)methyl)-1H-1,2,3-triazol- 1-yl)propyl)piperazin-1-yl)-2-oxo- ethyl)-1,4,7,10-tetraazacyclodo- decane-1,4,7-triyl)triacetic acid |
| 112 | 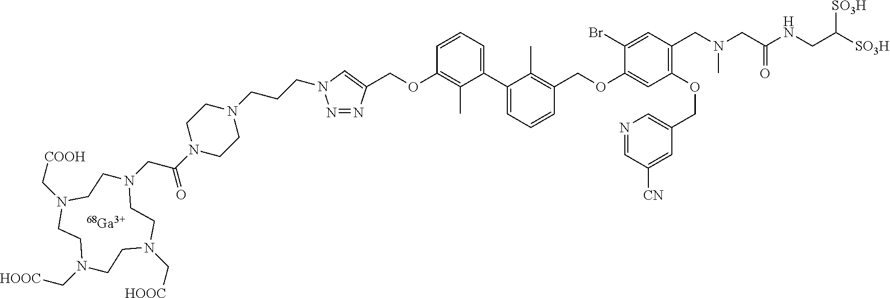 | corresponds Compound 63 with ⁶⁸Ga³⁺ as central atom of the chelate complex |
| 122 | 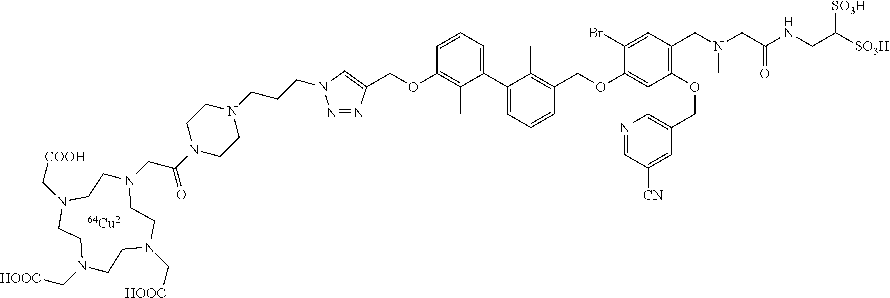 | corresponds Compound 63 with ⁶⁴Cu²⁺ as central atom of the chelate complex |
| 132 | 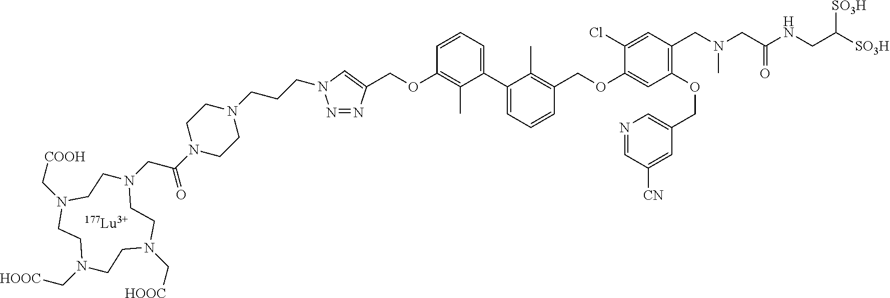 | corresponds Compound 63 with ¹⁷⁷Lu³⁺ as central atom of the che- late complex |
| 73 | 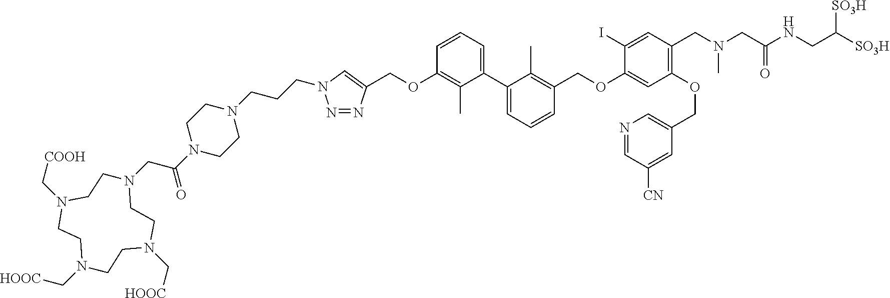 [SMILES: Cc1c(COc2cc(OCc3cncc(C#N)c3)c(CN(C)CC(=O)NCC(S(=O)(=O)O)S(=O)(=O)O)cc2I)cccc1-c1cccc(OCc2cn(CCCN3CCN(C(=O)CN4CCN(CC(=O)O)CCN(CC(=O)O)CCN(CC(=O)O)CC4)CC3)nn2)c1C] | 2,2′,2″-(10-(2-(4-(3-(4-(((3′-((5-((5- Cyanopyridin-3-yl)methoxy)-4- (((2-((2,2-disulfoethyl)amino)-2- oxoethyl)(methyl)amino)methyl)- 2-iodophenoxy)methyl)-2,2′-dimeth- yl-[1,1′-biphenyl]-3-yl)oxy)meth- yl)-1H-1,2,3-triazol-1-yl)pro- pyl)piperazin-1-yl)-2-oxoethyl)- 1,4,7,10-tetraazacyclododecane- 1,4,7-triyl)triacetic acid |
| 113 | 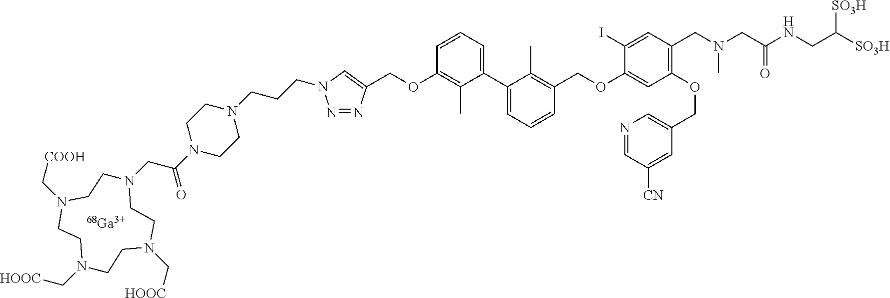 | corresponds Compound 73 with ⁶⁸Ga³⁺ as central atom of the chelate complex |
| 123 | 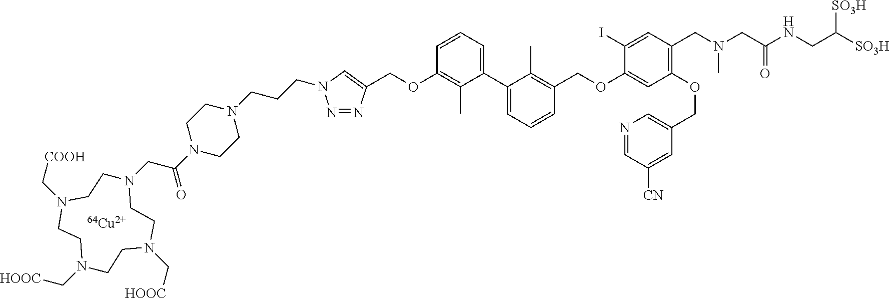 | corresponds Compound 73 with ⁶⁴Cu²⁺ as central atom of the chelate complex |
| 133 | 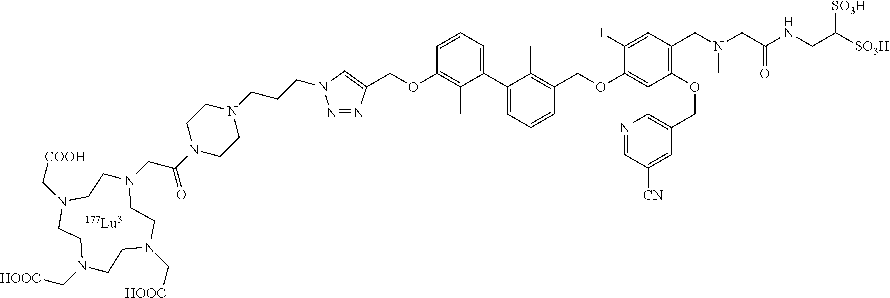 | corresponds Compound 73 with ¹⁷⁷Lu³⁺ as central atom of the che- late complex |
| 80 | 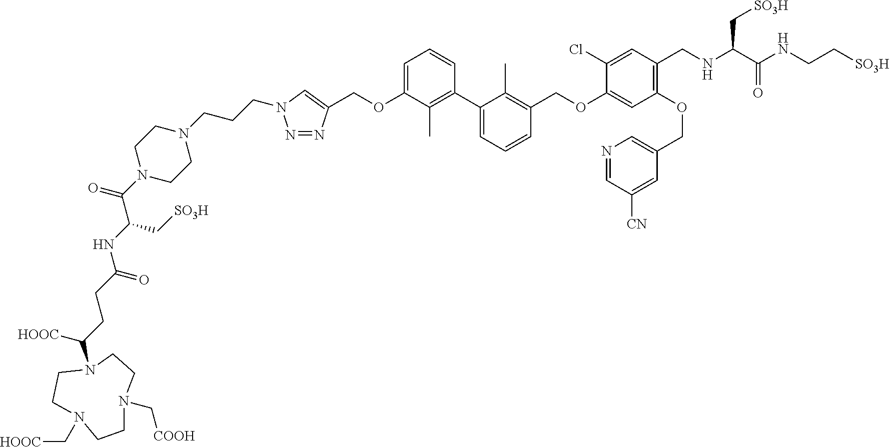 | 2,2′-(7-((S)-1-Carboxy-4-(((R)-1- (4-(3-(4-(((3′-((2-chloro-5-((5-cy- anopyridin-3-yl)methoxy)-4-((((S)- 1-oxo-3-sulfo-1-((2-sul- foethyl)amino)propan-2- yl)amino)methyl)phenoxy)meth- yl)-2,2′-dimethyl-[1,1′-biphenyl]- 3-yl)oxy)methyl)-1H-1,2,3-triazol- 1-yl)propyl)piperazin-1-yl)-1-oxo- 3-sulfopropan-2-yl)amino)-4-oxo- butyl)-1,4,7-triazonane-1,4-diyl)di- acetic acid |
| 114 | 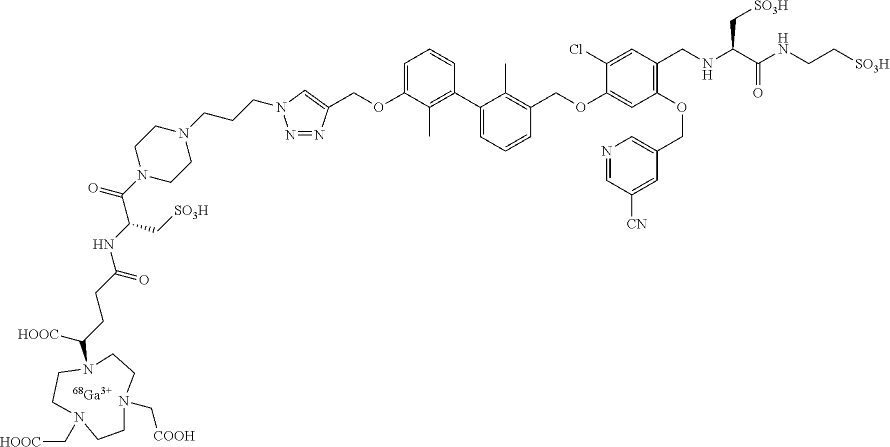 | corresponds Compound 80 with ⁶⁸Ga³⁺ as central atom of the chelate complex |
| 124 | 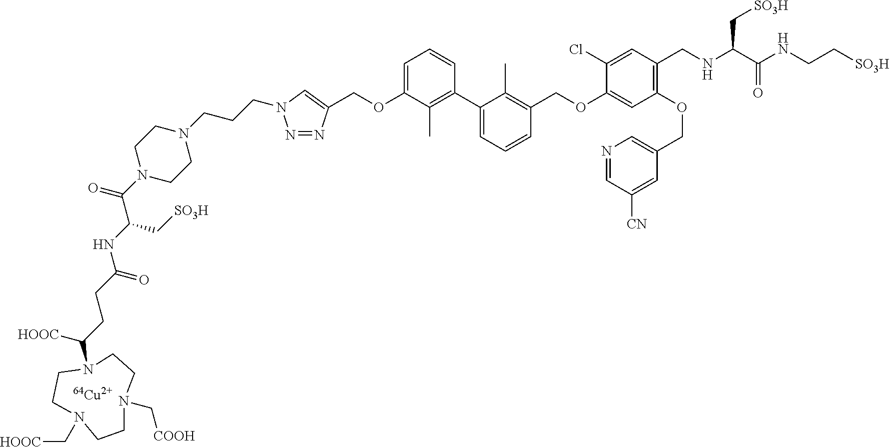 | corresponds Compound 80 with ⁶⁴Cu²⁺ as central atom of the chelate complex |

Compound 42 is a compound of general formula I, in which R¹ is hydrogen, R² is bromine, R³ is chlorine, one X is N, the other are C—H, Y is a sulphonic acid group, Z is a group of formula IV, wherein R⁴ are sulphonic acid groups, and M is a group of formula VII, wherein n=1, X¹ is C—H, m is 0, and M² is a chelate ligand [a 10-oxoethyl(1,4,7,10-tetraazacyclododecane-1,4,7-triyl)triacetic acid group].

Compound 43 is a compound of general formula I, in which R¹ is hydrogen, R² is bromine, R³ is chlorine, one X is N, the other are C—H, Y is a sulphonic acid group, Z is a group of formula IV, wherein R⁴ are sulphonic acid groups, and M is a group of formula VII, wherein n=1, X¹ is N, m is 0, and M² is a chelate ligand [a 10-oxoethyl(1,4,7,10-tetraazacyclododecane-1,4,7-triyl)triacetic acid group].

Compound 44 is a compound of general formula I, in which R¹ is hydrogen, R² is bromine, R³ is chlorine, one X is N, the other are C—H, Y is a sulphonic acid group, Z is a group of formula IV, wherein R⁴ are sulphonic acid groups, and M is a group of formula VIII, wherein p=2, X¹ is N, m is 0, and M² is a chelate ligand [a 10-oxoethyl(1,4,7,10-tetraazacyclododecane-1,4,7-triyl)triacetic acid group].

Compound 45 is a compound of general formula I, in which R¹ is a methyl group, R² is a methyl group, R³ is chlorine, one X is N, the other are C—H, Y is a sulphonic acid group, Z is a group of formula IV, wherein R⁴ are sulphonic acid groups, and M is a group of formula VII, wherein n=1, X¹ is C—H, m is 0, and M² is a chelate ligand [a 10-oxoethyl-(1,4,7,10-tetraazacyclododecane-1,4,7-triyl)triacetic acid group].

Compound 46 is a compound of general formula I, in which R¹ is a methyl group, R² is a methyl group, R³ is chlorine, one X is N, the other are C—H, Y is a sulphonic acid group, Z is a group of formula IV, wherein R⁴ are sulphonic acid groups, and M is a group of formula VII, wherein n=1, X¹ is N, m is 0, and M² is a chelate ligand [a 10-oxoethyl-(1,4,7,10-tetraazacyclododecane-1,4,7-triyl)triacetic acid group].

Compound 47 is a compound of general formula I, in which R¹ is a methyl group, R² is a methyl group, R³ is chlorine, one X is N, the other are C—H, Y is a sulphonic acid group, Z is a group of formula IV, wherein R⁴ are sulphonic acid groups, and M is a group of formula VIII, wherein p=2, X¹ is N, m is 0, and M² is a chelate ligand [a 10-oxoethyl-(1,4,7,10-tetraazacyclododecane-1,4,7-triyl)triacetic acid group].

Compound 54 is a compound of general formula I, in which R¹ is a methyl group, R² is a methyl group, R³ is chlorine, one X is N, the other are C—H, Y is a cyano group, Z is a group of formula IV, wherein R⁴ are sulphonic acid groups, and M is a group of formula VII, wherein n=1, the X¹ is N, m is 0, and M² is a chelate ligand [a 10-oxoethyl-(1,4,7,10-tetraazacyclododecane-1,4,7-triyl)triacetic acid group].

Compound 63 is a compound of general formula I, in which R¹ is a methyl group, R² is a methyl group, R³ is bromine, one X is N, the other are C—H, Y is a cyano group, Z is a group of formula IV, wherein R⁴ are sulphonic acid groups, and M is a group of formula VII, wherein n=1, the X¹ is N, m is 0, and M² is a chelate ligand [a 10-oxoethyl-(1,4,7,10-tetraazacyclododecane-1,4,7-triyl)triacetic acid group].

Compound 73 is a compound of general formula I, in which R¹ is a methyl group, R² is a methyl group, R³ is iodine, one X is N, the other are C—H, Y is a cyano group, Z is a group of formula III, wherein R⁴ are sulphonic acid groups, and M is a group of formula VII, wherein n=1, the X¹ is N, m is 1, R⁴ is a sulphonic acid group and M² is a chelate ligand [a 10-oxoethyl-(1,4,7,10-tetraazacyclododecane-1,4,7-triyl)triacetic acid group].

Compound 80 is a compound of general formula I, in which R¹ is a methyl group, R² is a methyl group, R³ is chlorine, one X is N, the other are C—H, Y is a cyano group, Z is a group of formula IV, wherein R⁴ are sulphonic acid groups, and M is a group of formula VII, wherein n=1, the X¹ is N, m is 0, and M² is a chelate ligand [a 7-oxobutyl)-1,4,7-triazonane-1,4-diyl)diacetic acid group].

### Preparation of the Lead Structure LS

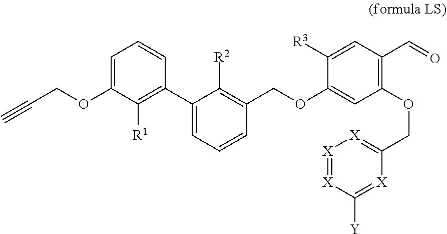

Compound of formula I, wherein instead of M

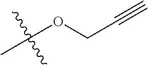 [SMILES: *OCC#C]

is present and instead of Z

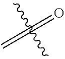 [SMILES: *=O]

is present.

The lead structure LS is prepared from a compound of general formula A and a compound of general formula B, as shown in scheme 1 above. Preferred methods for the preparation of the compound of general formula A and for the preparation of the compound of general formula B are explained in detail below.

### a) Preparation of a Compound of General Formula A

The preparation of the compound of general formula A takes place in several steps. In a first step a compound of general formula PA-2 is prepared, wherein the preparation can take place in two alternative ways depending on whether R³ is chorine, bromine, or AG¹ (see, scheme A1) or iodine (see scheme Ala). Compounds 2, 56, and 66 are examples of compound PA-2.

In scheme A1 there is shown the reaction of a compound of general formula PA-1 to a compound of general formula PA-2 in a first step a.

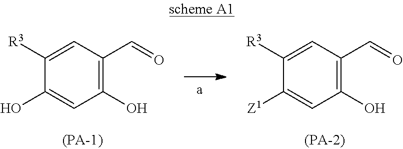

In compound PA-1 R³ is chlorine, bromine, or AG¹. If R³ is chlorine, then compound PA-1 is compound 1, if R³ is bromine, then compound PA-1 is compound 55. Compounds 1, 55, and 65 are known from literature. In compound PA-2 Z¹ is —O-MEM or —O-MOM. Here, -MEM designates a (2-methoxyethoxy)methyl group, -MOM a methoxymethyl group. The reaction can take place in the presence of (i) (2-methoxy-ethoxy)methylchloride (MEMCl) or methoxymethylchloride (MOMCl) and (ii) an amine base in an aprotic solvent. For example, the reaction can take place in the presence of MEMCl and absolute diisopropylethylamine (DIPEA) in absolute tetrahydrofurane (THF) at 0° C. to room temperature for 40 hrs.

Alternatively, compound PA-2 in which R³ is iodine can be prepared from compound 65 in an alternative step b, as is shown in scheme Ala.

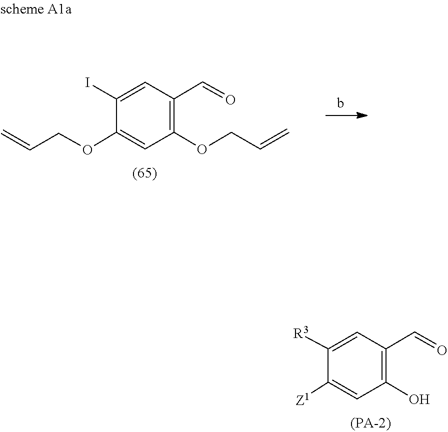

In compound PA-2 residue Z¹ is a group of formula

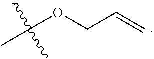 [SMILES: *OCC=C]

The reaction can take place in the presence of a Pd(0) catalyst and an inorganic base in a protic solvent. For example, the reaction can take place in the presence of Pd(PPh₃)₄ and K₂CO₃ in methanol (MeOH) at room temperature for 16 hrs. PPh₃ designates triphenylphosphane.

In a second step c a compound of general formula PA-3 is reacted to a compound of general formula PA-4, as is shown in scheme A2.

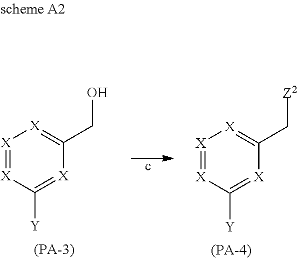

In compounds PA-3 and PA-4 residues X and Y have the meanings given in connection with the general formula I, Z² is chlorine, bromine, or iodine. The reaction can take place by means of an Appel reaction in the presence of tetrabromomethane (CBr₄), tetrachloromethane (CCl₄), or I₂/imidazolein an aprotic solvent. For example, the reaction can take place in the presence of triphenylphosphane (PPh₃) and CBr₄ in absolute tetrahydrofurane (THF) at 0° C. to room temperature for 16 hrs. Compound 3 is an example of compound PA-3. Compound 4 is an example of compound PA-4. Compound PA-3 is commercially available or known from literature.

In a third step d compound PA-2 is reacted with compound PA-4 to a compound of general formula PA-5, as is shown in scheme A3.

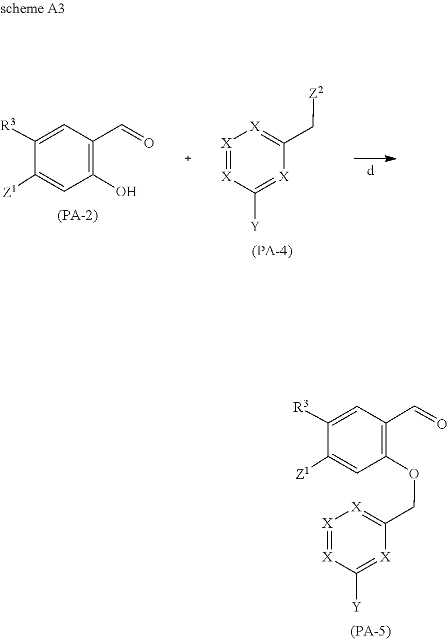

In compound PA-4 residues R³, X, Y, and Z¹ have the meanings given in connection with compounds PA-2 and PA-4. The reaction can take place in the presence of an inorganic base or an amine base in a polar aprotic solvent. For example, the reaction can take place in the presence of K₂CO₃ in absolute dimethylformamide (DMF) at room temperature for 16 hrs. Compounds 5, 48, 57, and 67 are examples of compound PA5.

In a fourth step e compound PA-5 is converted to a compound of general formula A, as is shown in scheme A4.

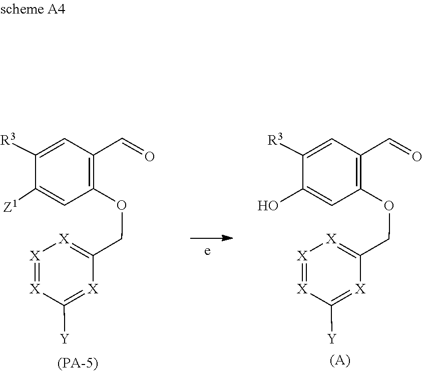

In compound A residues R³, X, and Y have the meanings given in connection with compound PA-5. The reaction can take place in a chlorine-containing solvent. For example, the reaction can take place in a mixture of trifluoroacetic acid (TFA) and dichloromethane (DCM) in a volume ratio of 1:1 at 0° C. to room temperature for 16 hrs. Compounds 6, 49, 58, 68 are examples of compound A.

### b) Preparation of a Compound of General Formula B

The preparation of the compound of general formula B takes place in several steps. The preparation can take place in two alternative ways depending on whether compound Ba or compound Bb shall be prepared (see schemes B3 and B4a). Compounds Ba and Bb are compounds of general formula B.

### aa) Synthesis Way 1 for the Preparation of Compound B

In scheme B1 there is shown the reaction of compound 8 with compound 9 to a compound of general formula PB-la and a compound of general formula PB-2b in a first step a.

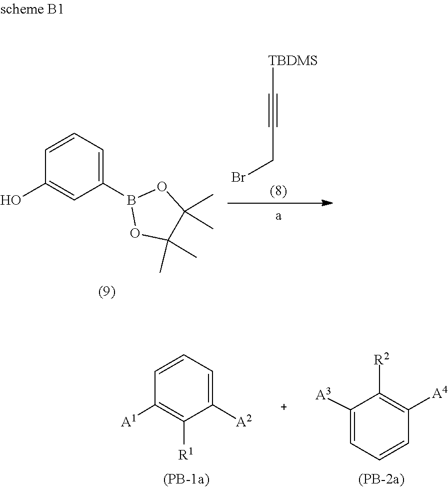

Compounds 8 and 9 are known from literature. Residue A¹ is a group of formula

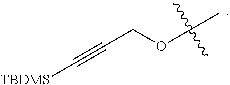

TBDMS designates a tert-butyldimethylsilyl group. Residue A² is a group of formula

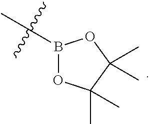 [SMILES: *B1OC(C)(C)C(C)(C)O1]

Residue A³ is iodine. Residue A⁴ is —CH₂—OH. Residue R¹ is hydrogen, residue R² is bromine. The reaction can take place in the presence of an inorganic base or an amine base in a polar aprotic solvent. For example, the reaction can take place in the presence of K₂CO₃ in absolute dimethylformamide (DMF) at room temperature for 16 hrs. Compound 10 is an example of compound PB-la. Compound 13 is an example of compound PB-2a.

In scheme B2 there is shown the reaction of compounds of general formula PB-1a and PB-2a to a compound of general formula PB-4a in a second step b.

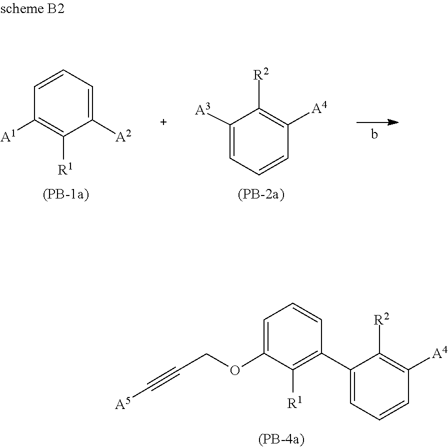

In compound PB-4a residues A⁴, R¹, and R² have the meanings in connection with compounds PB-la and PB-2a. Residue A⁵ is -TBDMS. The reaction can take place in the presence of a Pd(0) catalyst and an inorganic base in a mixture of an aromatic solvent, a protic solvent and H₂O. For example, the reaction can take place in the presence of Pd(PPh₃)₄ and K₂CO₃ under argon in a mixture of toluene, ethanol (EtOH) and H₂O in a volume ratio of 20:0.5:0.1 at 100° C. for 16 hrs. Compound 14 is an example of compound PB-4a.

In scheme B3 there is shown the reaction of the compound of general formula PB-4a to a compound of general formula Ba in a third step c.

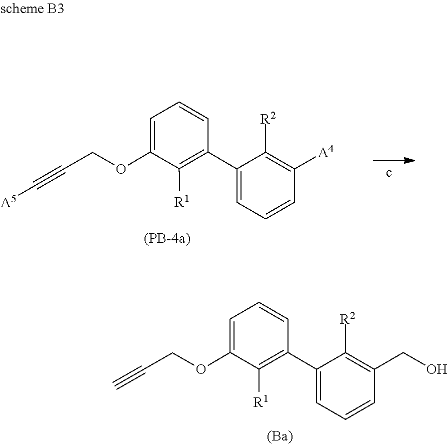

In compound Ba residues R¹ and R² have the meanings in connection with compound PB-4a. In a first variant the reaction can be carried out in the presence of a fluoride source in an aprotic solvent. For example, according to the first variant the reaction can take place in the presence of tetrabutylammonium fluoride employed as 1M solution in THF in absolute THF as the solvent at 0° C. to room temperature for 2 hrs. Alternatively, in a second variant the reaction can take place in the presence of a hydride reagent in a mixture of a protic solvent and an aprotic solvent. For example, according to the second variant the reaction can be carried out in the presence of NaBH₄ in a mixture of absolute MeOH and absolute DCM in a volume ratio of 1:1 at 0° C. to room temperature for 3 hrs. Compound 15 is an example of compound Ba.

### bb) Synthesis Way 2 for the Preparation of Compound B

Synthesis way 2 for the preparation of compound B starts with a compound of general formula PB-1b and a compound of general formula PB-2b (schema B1a).

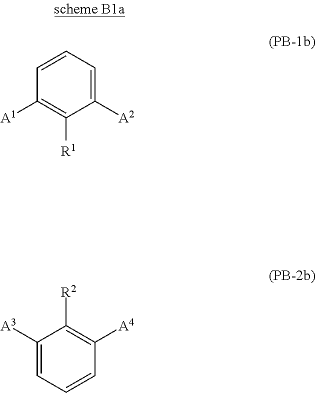

Residue A¹ is —OH. Residue A² is bromine. Residue A³ is a group of formula

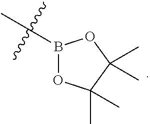 [SMILES: *B1OC(C)(C)C(C)(C)O1]

Residue A⁴ is —CHO. Residue R¹ is a C₁-C₆ alkyl group, residue R² is a C₁-C₆ alkyl group. Compound 21 is an example of compound PB-2b. Compound s PB-1b and PB-2b are known from literature.

In scheme B2a there is shown the reaction of compounds of general formula PB-1b and PB-2b to a compound of general formula PB-3b in a first step a.

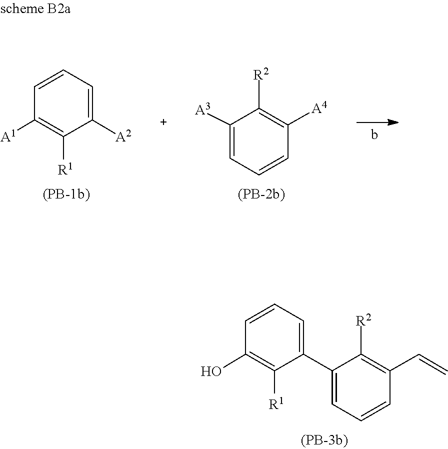

In compound PB-3b residues R¹ and R² have the meanings in connection with compounds PB-1b and PB-2b. The reaction can take place in the presence of a Pd(O) catalyst and an inorganic base in a mixture of an aromatic solvent, a protic solvent and H₂O. For example, the reaction can take place in the presence of Pd(PPh₃)₄ and K₂CO₃ under argon in a mixture of toluene, ethanol (EtOH), and H₂O in a volume ratio of 20:0.5:0.1 at 100° C. for 16 hrs. Compound 22 is an example of compound PB-3b.

In scheme B3a there is shown the reaction of a compound of general formula PB-3b with 3-bromoprop-1-yne to a compound of general formula PB-4b in a second step c.

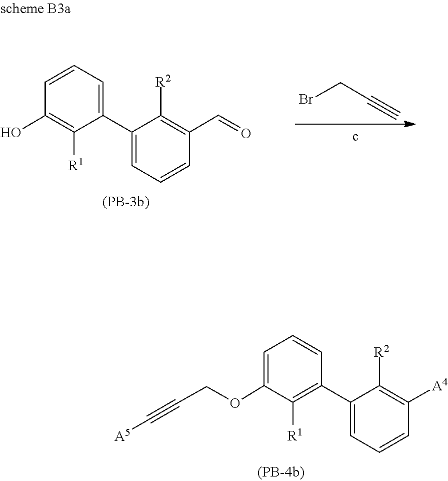

In compound PB-4b residues R¹ and R² have the meanings in connection with compound PB-3b. Residue A⁵ is hydrogen. The reaction can take place in the presence of an inorganic base or an amine base in a polar aprotic solvent. For example, the reaction can take place in the presence of K₂CO₃ in absolute dimethylformamide (DMF) at room temperature for 16 hrs. Compound 23 is an example of compound PB-4b.

In scheme B4a there is shown the reaction of the compound of general formula PB-4b to a compound of general formula Bb in a third step d.

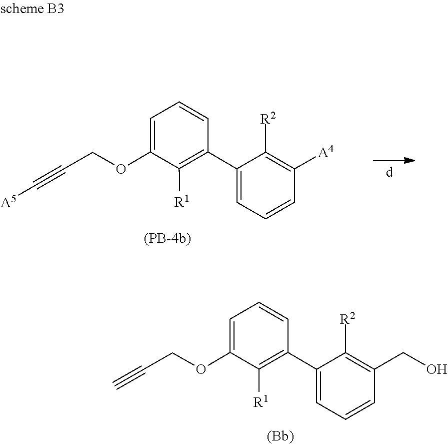

In compound Bb residues R¹ and R² have the meanings in connection with compound PB-4b. In a first variant the reaction can be carried out in the presence of a fluoride source in an aprotic solvent. For example, according to the first variant the reaction can take place in the presence of tetrabutylammonium fluoride employed as a 1M solution in THE in absolute THE as the solvent at 0° C. to room temperature for 2 hrs. Alternatively, in a second variant the reaction can take place in the presence of a hydride reagent in a mixture of a protic solvent and an aprotic solvent. For example, according to the second variant the reaction can be carried out in the presence of NaBH₄ in a mixture of absolute MeOH and absolute DCM in a volume ratio of 1:1 at 0° C. to room temperature for 3 hrs. Compound 24 is an example of compound Bb.

Compound A can be reacted with compound Ba or compound Bb to the lead structure LS. This is described in detail below in the section “Lead Structure”. The reaction can take place in the presence of a Mitsonobu reagent in a polar aprotic solvent. For example, the reaction can take place in the presence of DEAD and PPh₃ in absolute DMF at 0° C. to room temperature for 16 hrs. Compounds 16, 25, 50, 59, and 69 are examples of the lead structure LS.

Scheme 1 shows the preparation of the lead structure LS.

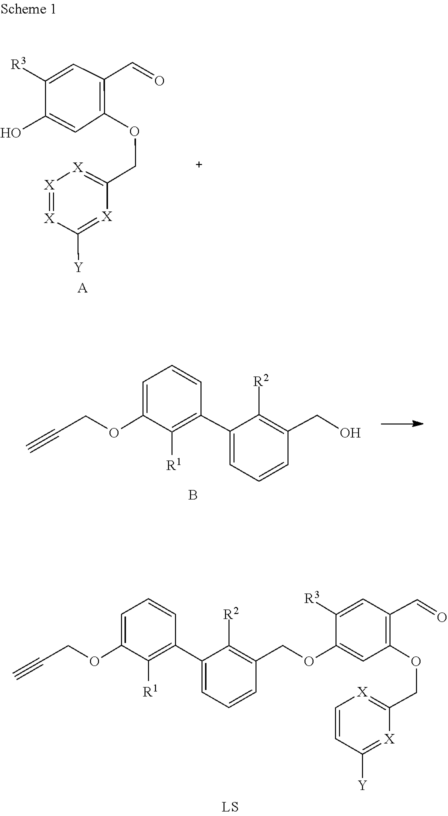

In formulae A, B and LS R¹, R², R³, X and Y each have the meanings given in context with general formula I. Here, the phenolic compound A, the alcohol B, and triphenylphosphine were reacted with DEAD (Diethylazodicarboxylat) by the general procedure 2 described later.

Scheme 2 shows the substitution of the carbaldehyde group of the lead structure LS by a water-soluble group Z.

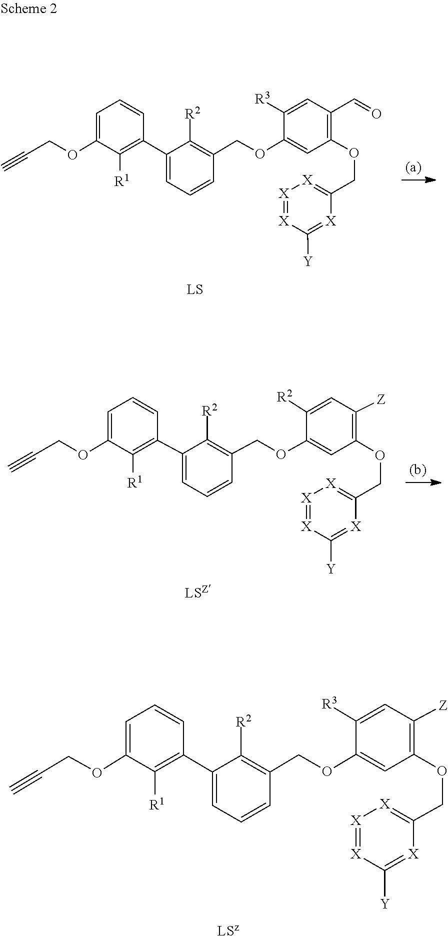

In formulae LS, LS^{Z′} and LS^{Z} R¹, R², R³, X and Y each have the meanings given in context with general formula I. Z′ and Z each have the meaning given in the following table 1. In step (a), the carbaldehyde group in the formula LS is subjected a reductive amination with a corresponding unsubstituted or substituted amino carboxylic acid given in table 1, a) to yield formula LS′. In the optional step (b), group Z′ in formula LS^{Z′} is converted into an unsubstituted or substituted amino sulphonic acid or unsubstituted or substituted amino phosphonic acid group Z by means of amide coupling with the corresponding amide given in table 1, b). The skilled person is able to carry out the particular steps of these synthesis pathways according to the general procedures 3 and 6, respectively, given below.

TABLE 1

| a) | 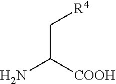 | 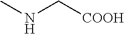 | 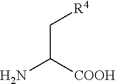 |
|---|---|---|---|
| Z′ | 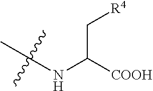 | 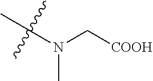 [SMILES: *N(C)CC(=O)O] | 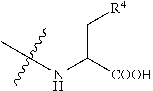 |
| b) | — | 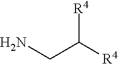 | 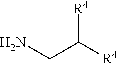 |
| Z | 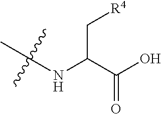 | 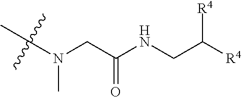 | 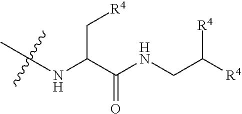 |
| | (formula II) | (formula III) | (formula IV) |
| a) | 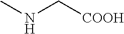 | 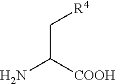 | 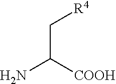 |
| Z′ | 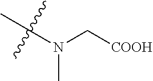 [SMILES: *N(C)CC(=O)O] | 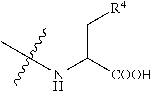 | 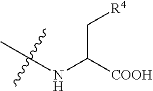 |
| b) | 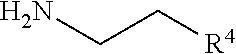 | 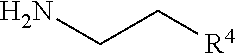 | 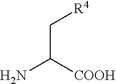 |
| Z | 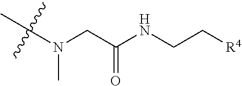 | 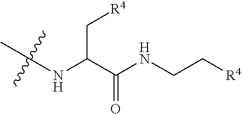 | 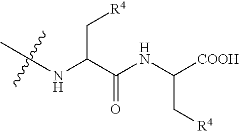 |
| | (formula V) | (formula VI) | (formula VIa) |

Scheme 3 shows the way to obtain the first group of compounds of formula I—the fluorine compounds.

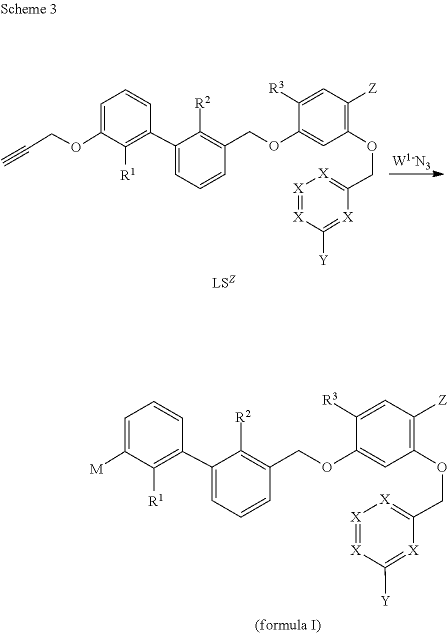

In the formulae LS^{Z} and I R¹, R², R³, X, Y and Z each have the meanings given in context with general formula I, W¹ and M the meanings given in table 2. In the formulae IXa-XIIa and IX-XII in table 2 R⁶ and R⁷ each have the meanings given in context with general formula I. This first group of compounds of formula I according to the invention is obtained by azide-alkyne cycloaddition using a Cu(I)-catalyst system according general procedure 4 described later.

TABLE 2

| | | | | |
|---|---|---|---|---|
| W¹ | 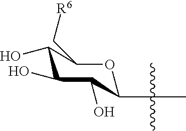 | 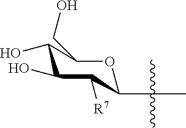 | 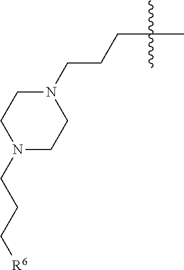 | 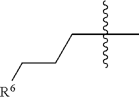 |
| | (formula IXa) | (formula Xa) | (formula XIa) | (formula XIIa) |
| M | 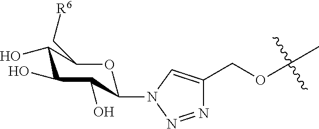 | 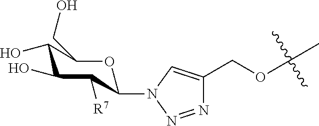 | 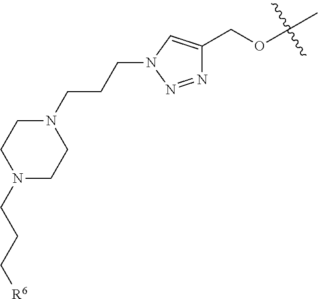 | 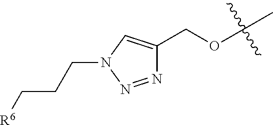 |
| | (formula IX) | (formula X) | (formula XI) | (formula XII) |

Schemes 4 and 5 show the way to obtain the second group of compounds of formula I—the chelate compounds.

Scheme 4 illustrates the synthesis of the linker structure LS^{ZL}

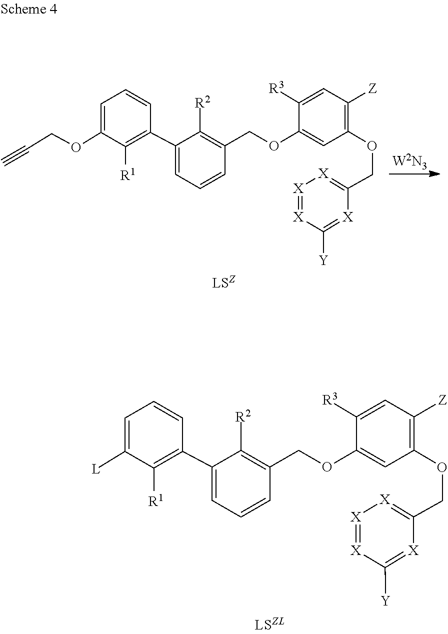

In the formulae LS^{Z} and LS^{ZL} R¹, R², R³, X, Y and Z each have the meanings given in context with general formula I, W² and L each have the meanings given in table 3. In the formulae VIIa, VIIb, VIIIa and VIIIb in table 3 R⁴, X¹, m, n and p each have the meanings given in context with general formula I. The linker structure LS^{ZL} is obtained by azide-alkyne cycloaddition using a Cu(II)-catalyst system according general procedure 5 described later.

TABLE 3

| | | |
|---|---|---|
| W² | 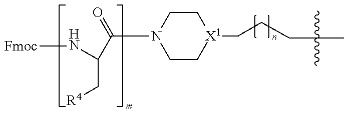 | 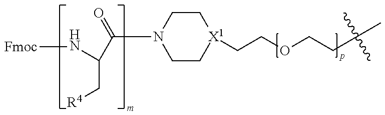 |
| | (formula VIIb) | (formula VIIIb) |
| L | 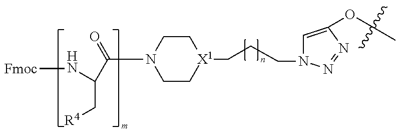 | 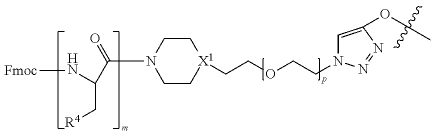 |
| | (formula VIIa) | (formula VIIIa) |

Scheme 5 shows the way of preparing a compound of general formula I with a chelate ligand.

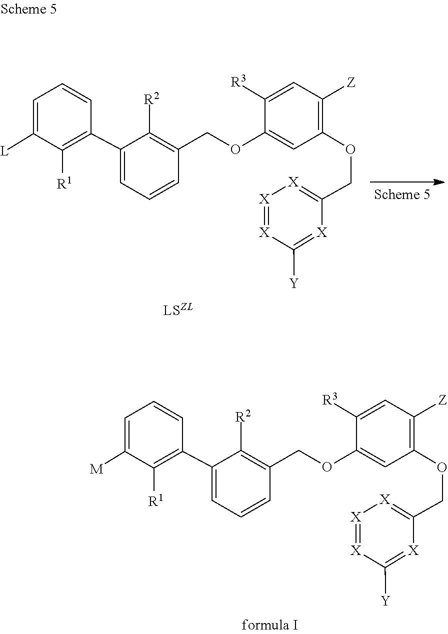

In the formulae LS^{ZL} and I R¹, R², R³, X, Y and Z each have the meanings given in context with general formula I, L has the meanings given in table 3, M² and M each have the meanings given in table 4, X¹, m, n and p in formulae in table 4 each have the meanings given in context with general formula I. The compound of formula I is obtained by conjugation of a chelate ligand M² to the linker structure LS^{ZL}. In case of the chelate ligand is DOTA, the addition is done according to general procedure 6 described later.

TABLE 4

| | | |
|---|---|---|
| M | 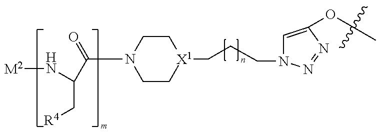 | 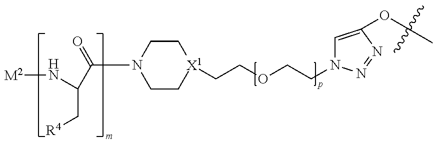 |

| | | | |
|---|---|---|---|
| M² | 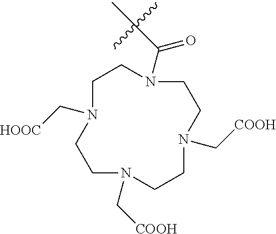 [SMILES: *C(=O)N1CCN(CC(=O)O)CCN(CC(=O)O)CCN(CC(=O)O)CC1] | 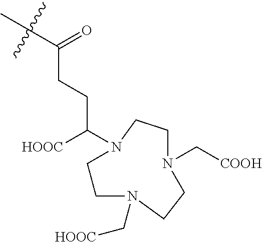 [SMILES: *C(=O)CCC(C(=O)O)N1CCN(CC(=O)O)CCN(CC(=O)O)CC1] | 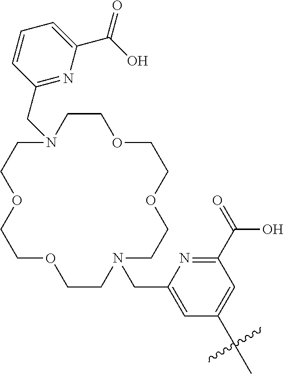 [SMILES: *c1cc(CN2CCOCCOCCN(Cc3cccc(C(=O)O)n3)CCOCCOCC2)nc(C(=O)O)c1] |

In a further embodiment of the invention, there may further be provided a compound of general formula IC

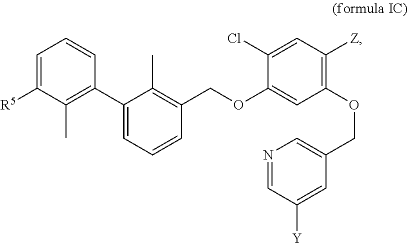

wherein
R¹ is a methyl group;
R² is a methyl group;
R³ is chlorine;
R⁴ is a sulphonic acid group or a phosphonic acid group;
Y is —CN or —S(O)₂—CH₃;
Z is a group of general formula V or a group of general formula VI;
R⁵ is a group of general formula VII or a group of general formula VIII, wherein X¹, M², m, n and p have the meanings given in connection with the compound of general formula I.

The compound of general formula IC corresponds to a compound of formula I in which only the X in the 3 position to the methoxy group is N and the remaining moieties X are C—H and M is R⁵.

Preferably, R⁵ is a group of general formula VII, wherein X¹ is N, M² is chelate ligand or a chelate complex consisting of the chelate ligand and a central atom, wherein the central atom is Cu-64 or Ga-68, R⁴ is a sulphonic acid group or a phosphonic acid group, Z is a group of general formula VI, n is 1 or 2 and m is 0 to 2. It is further preferred that n is 1.

TABLE 2a
Further chelate compounds

| | | |
|---|---|---|
| 201 | 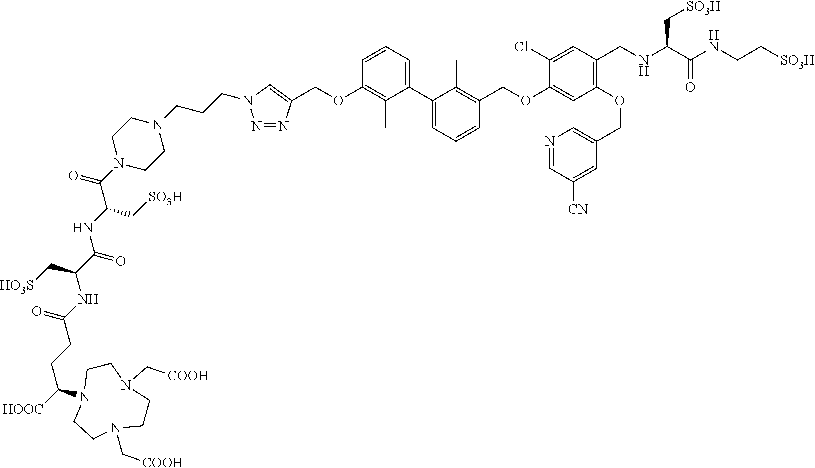 | 2,2′-(7-((R)-1-Carboxy-4-(((R)-1- (((R)-1-(4-(3-(4-(((3′-((2-chloro-5- ((5-cyanopyridin-3-yl)methoxy)- 4-((((R)-1-oxo-3-sulfo-1-((2-sul- foethyl)amino)propan-2- yl)amino)methyl)phenoxy)meth- yl)-2,2′-dimethyl-[1,1′-bi- phenyl]-3-yl)oxy)methyl)-1H- 1,2,3-triazol-1-yl)propyl)piper- azin-1-yl)-1-oxo-3-sulfopropan-2- yl)amino)-1-oxo-3-sulfopropan-2- yl)amino)-4-oxobutyl)-1,4,7-tria- zonane-1,4-diyl)diazetic acid |
| 211 | 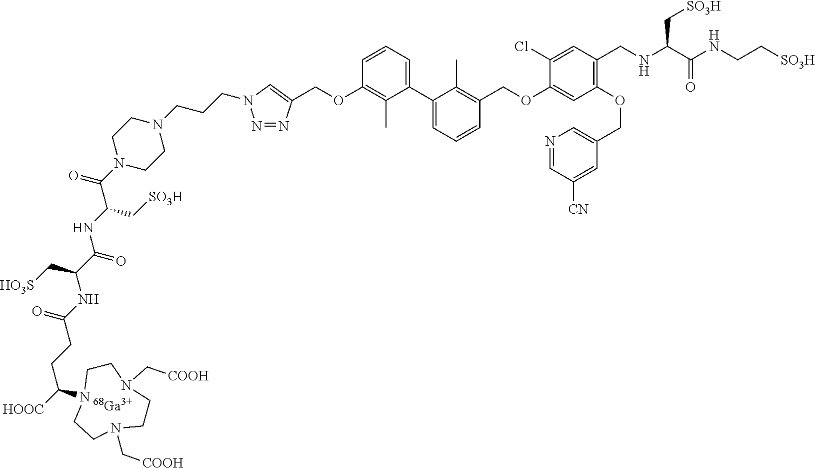 | corresponds Compound 201 with ⁶⁸Ga³⁺ as central atom of the che- late complex |
| 221 | 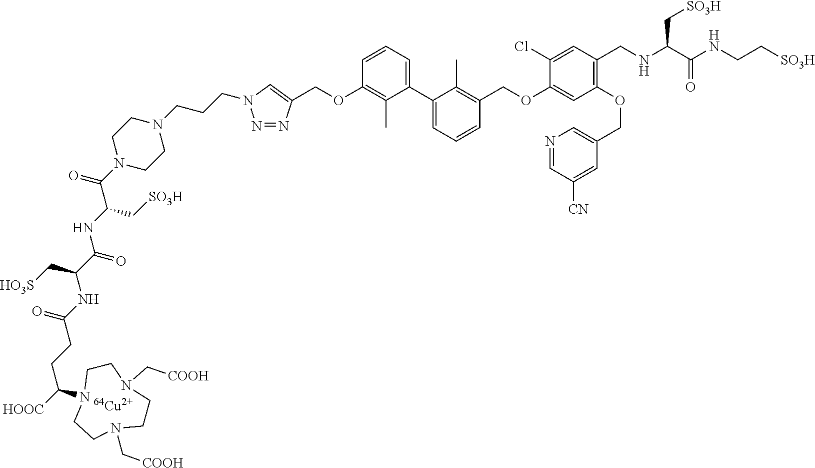 | corresponds Compound 201 with ⁶⁴Cu²⁺ as central atom of the che- late complex |
| 202 | 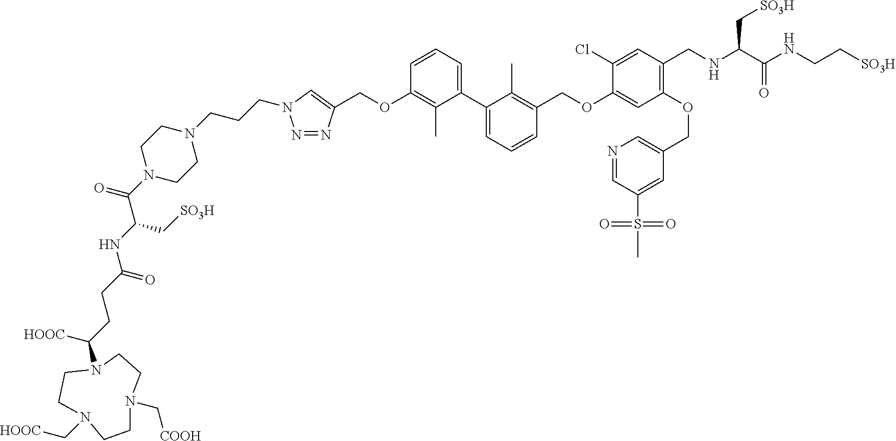 [SMILES: Cc1c(COc2cc(OCc3cncc(S(C)(=O)=O)c3)c(CN[C@@H](CS(=O)(=O)O)C(=O)NCCS(=O)(=O)O)cc2Cl)cccc1-c1cccc(OCc2cn(CCCN3CCN(C(=O)[C@H](CS(=O)(=O)O)NC(=O)CC[C@H](C(=O)O)N4CCN(CC(=O)O)CCN(CC(=O)O)CC4)CC3)nn2)c1C] | 2,2′-(7-((R)-1-Carboxy-4-(((R)-1- (4-(3-(4-(((3′-((2-chloro-5-((5- (methylsulfonyl)pyridin-3- yl)methoxy)-4-((((R)-1-oxo-3- sulfo-1-((2-sulfoethyl)amino)pro- pan-2-yl)amino)methyl)phe- noxy)methyl)-2,2′-dimethyl-[1,1′- biphenyl]-3-yl)oxy)methyl)-1H- 1,2,3-triazol-1-yl)propyl)piper- azin-1-yl)-1-oxo-3-sulfopropan-2- yl)amino)-4-oxobutyl)-1,4,7-tria- zonane-1,4-diyl)diacetic acid |
| 212 | 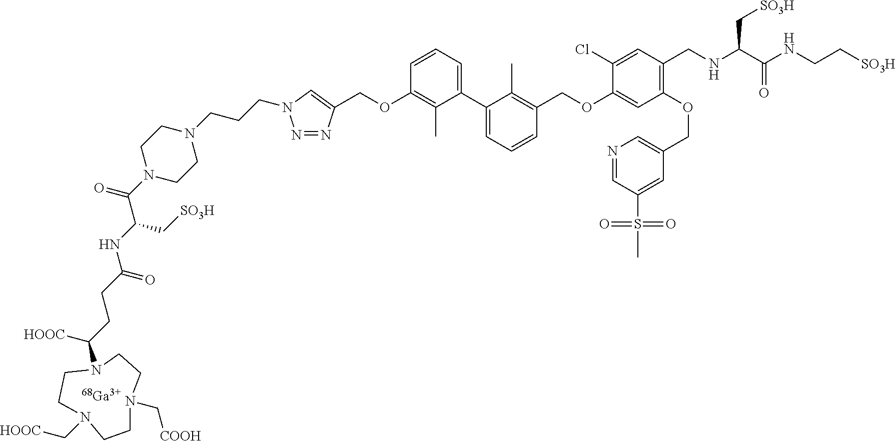 | corresponds Compound 202 with ⁶⁸Ga³⁺ as central atom of the che- late complex |
| 222 | 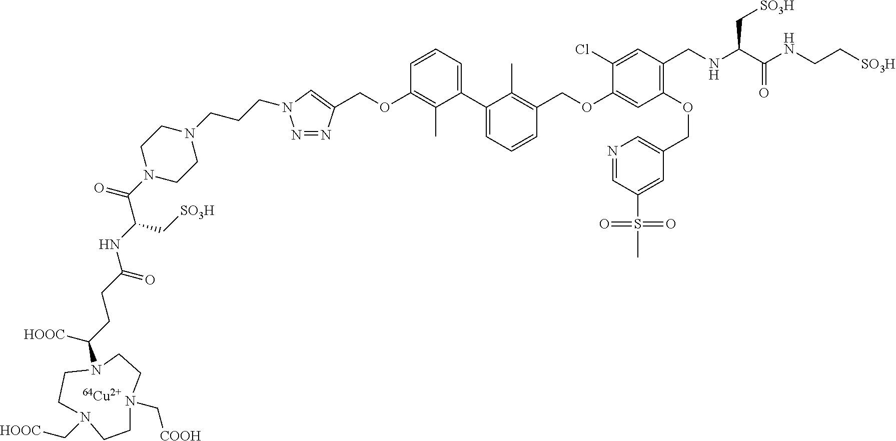 | corresponds Compound 202 with ⁶⁴Cu²⁺ as central atom of the che- late complex |
| 203 | 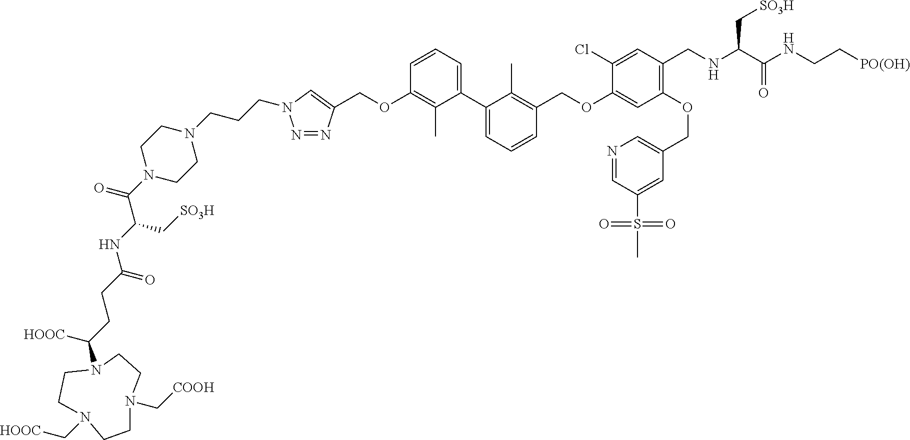 | 2,2′-(7-((R)-1-Carboxy-4-(((R)-1- (4-(3-(4-(((3′-((2-chloro-5-((5- (methylsulfonyl)pyridin-3- yl)methoxy)-4-((((R)-1-oxo-1-((2- phosphonoethyl)amino)-3-sul- fopropan-2-yl)amino)methyl)phe- noxy)methyl)-2,2′-dimethyl-[1,1′- biphenyl]-3-yl)oxy)methyl)-1H- 1,2,3-triazol-1-yl)propyl)piper- azin-1-yl)-1-oxo-3-sulfopropan-2- yl)amino)-4-oxobutyl)-1,4,7-tria- zonane-1,4-diyl)diacetic acid |
| 213 | 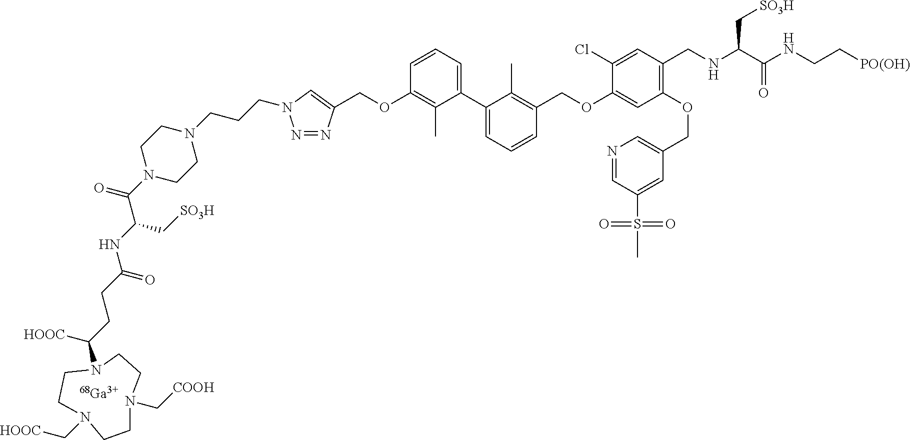 | corresponds Compound 203 with ⁶⁸Ga³⁺ as central atom of the che- late complex |
| 223 | 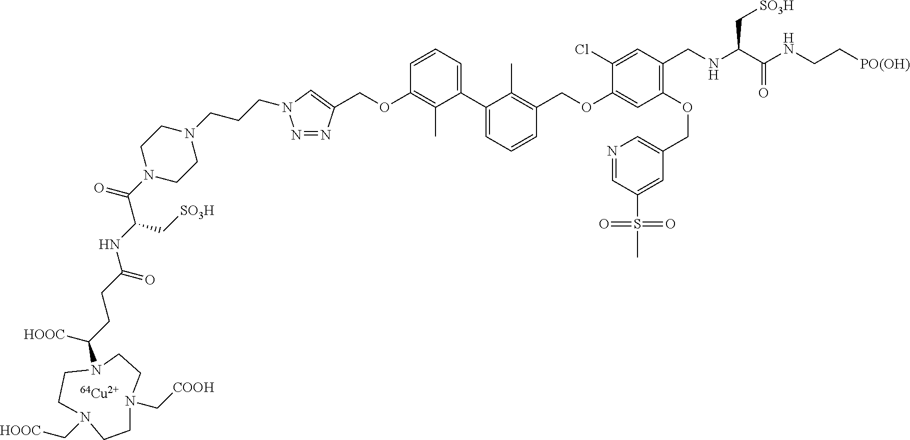 | corresponds Compound 203 with ⁶⁴Cu²⁺ as central atom of the che- late complex |
| 204 | 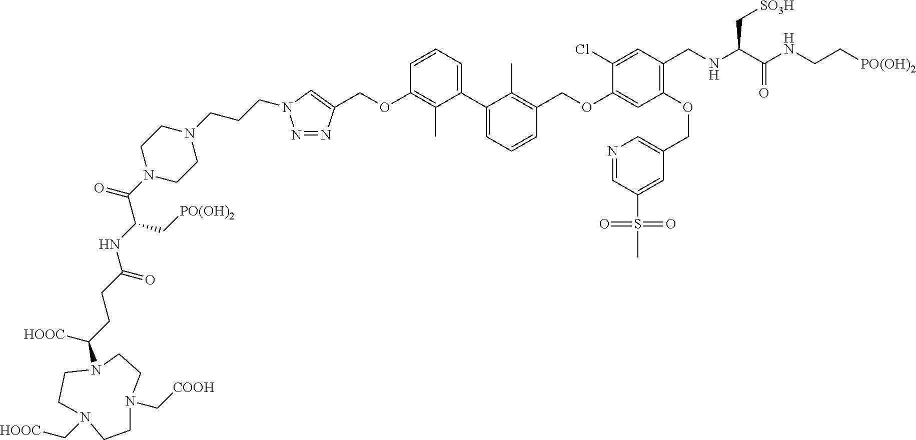 | 2,2′-(7-((R)-1-Carboxy-4-(((R)-1- (4-(3-(4-(((3′-((2-chloro-5-((5- (methylsulfonyl)pyridin-3- yl)methoxy)-4-((((R)-1-oxo-1-((2- phosphonoethyl)amino)-3-sul- fopropan-2-yl)amino)methyl)phe- noxy)methyl)-2,2′-dimethyl-[1,1′- biphenyl]-3-yl)oxy)methyl)-1H- 1,2,3-triazol-1-yl)propyl)piper- azin-1-yl)-1-oxo-3-phosphonopro- pan-2-yl)amino)-4-oxobutyl)- 1,4,7-triazonane-1,4-dihyl)diacetic acid |
| 214 | 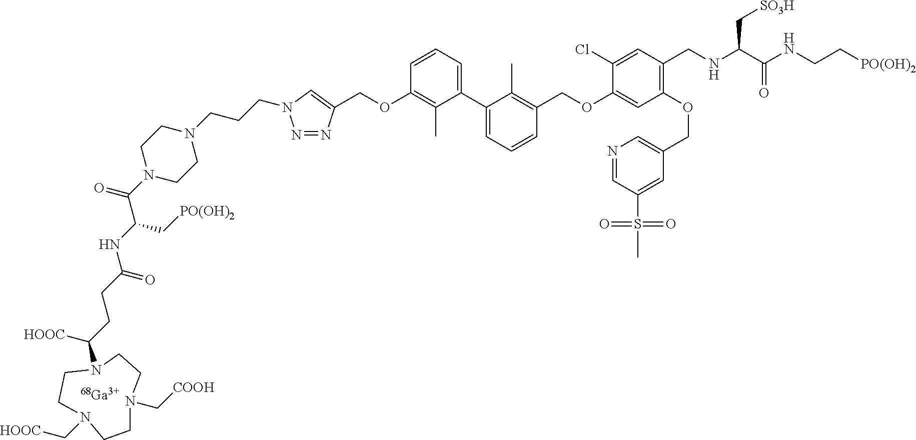 | corresponds Compoudn 204 with ⁶⁸Ga³⁺ as central atom of the che- late complex |
| 224 | 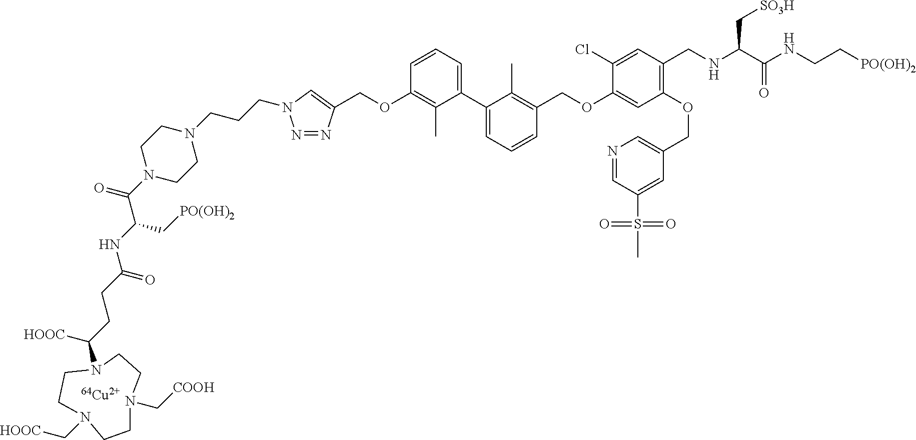 | corresponds Compound 204 with ⁶⁴Cu²⁺ as central atom of the che- late complex |
| 205 | 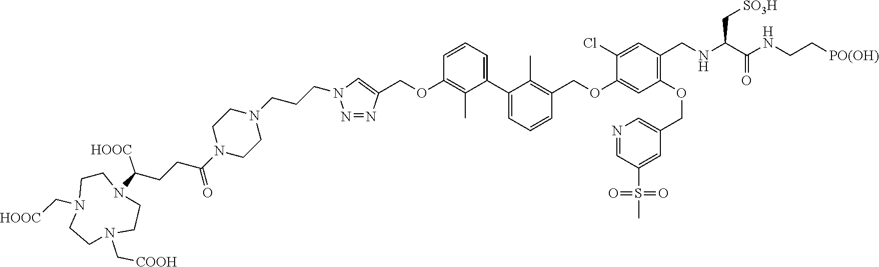 | 2,2′-(7-((R)-1-Carboxy-4-(4-(3-(4- (((3′-((2-chloro-5-((5-(methyl- sulfonyl)pyridin-3-yl)methoxy)-4- ((((R)-1-oxo-1-((2-phosphono- ethyl)amino)-3-sulfopropan-2- yl)amino)methyl)phenoxy)meth- yl)-2,2′-dimethyl-[1,1′-bi- phenyl]-3-yl)oxy)methyl)-1H- 1,2,3-triazol-1-yl)propyl)piper- azin-1-yl)-4-oxobutyl)-1,4,7-tria- zonane-1,4-diyl)diacetic acid |
| 215 | 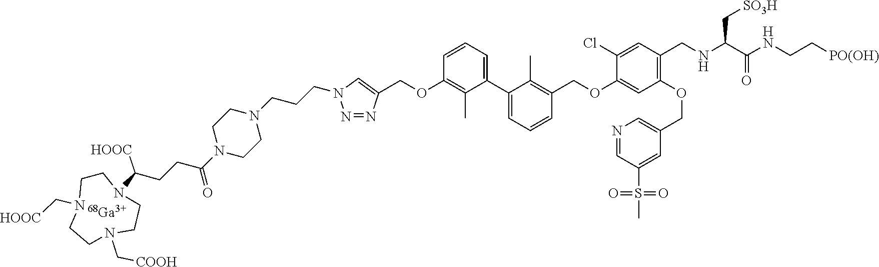 | corresponds Compound 205 with ⁶⁸Ga³⁺ as central atom of the che- late complex |
| 225 | 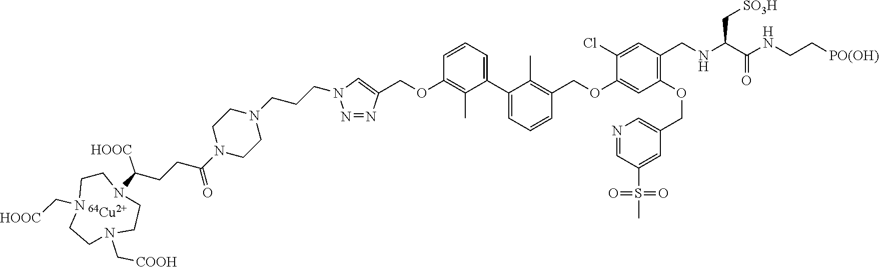 | corresponds Compound 205 with ⁶⁴Cu²⁺ as central atom of the che- late complex |

Compound 201 is a compound of general formula IC, in which Y is a cyano group, Z is a group of formula VI, wherein R⁴ are sulphonic acid groups, and R⁵ is a group of formula VII, wherein n=1, X¹ is N, R⁴ are sulphonic acid groups, m is 2, and M² is a chelate ligand.

Compound 202 is a compound of general formula IC, in which Y is methyl sulphonyl group, Z is a group of formula VI, wherein R⁴ are sulphonic acid groups, and R⁵ is a group of formula VII, wherein n=1, the X¹ is N, R⁴ is a sulphonic acid group, m is 1, and M² is a chelate ligand.

Compound 203 is a compound of general formula IC, in which Y is a methyl sulphonyl group, Z is a group of formula VI, wherein one R⁴ is a sulphonic acid group and the other R⁴ is a phosphonic acid group, and R⁵ is a group of formula VII, wherein n=1, the X¹ is N, R⁴ is a sulphonic acid group, m is 1, and M² is a chelate ligand.

Compound 204 is a compound of general formula IC, in which Y is a methyl sulphonyl group, Z is a group of formula VI, wherein one R⁴ is a sulphonic acid group and the other R⁴ is a phosphonic acid group, and R⁵ is a group of formula VII, wherein n=1, the X¹ is N, R⁴ is a phosphonic acid group, m is 1, and M² is a chelate ligand.

Compound 205 is a compound of general formula IC, in which Y is a methyl sulphonyl group, Z is a group of formula VI, wherein one R⁴ is a sulphonic acid group and the other R⁴ is a phosphonic acid group, and R⁵ is a group of formula VII, wherein n=1, the X¹ is N, m is 0, and M² is a chelate ligand.

In the following, the invention is explained in detail with the help of examples not intended to limit the invention with respect to the drawings. Here,

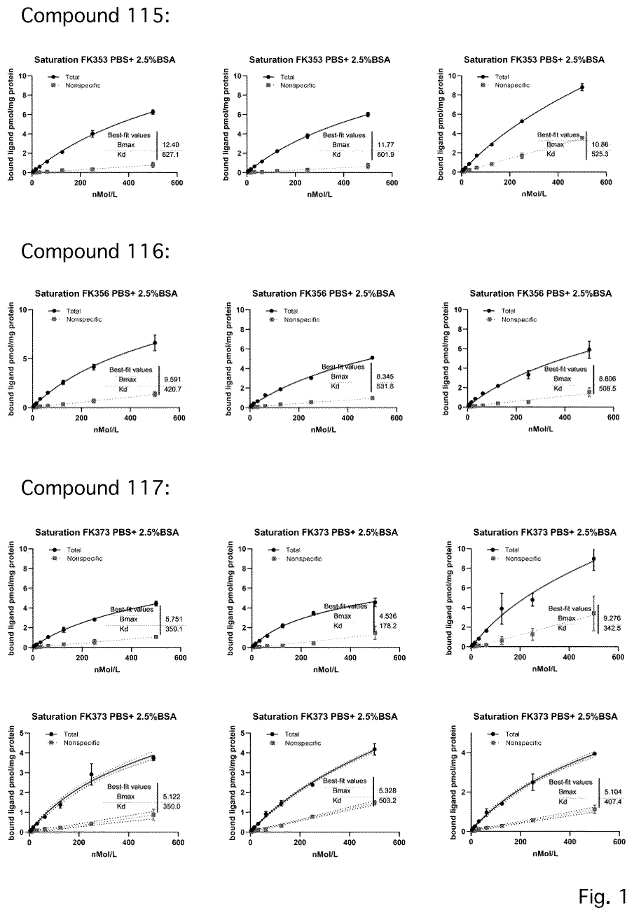

FIG. 1 shows diagrams as obtained in saturation binding experiments on a PC3 hPSCA PD-L1 overexpressing cell line for compounds 115 and 117 in triplicates as well as for compound 116 in sixfold;

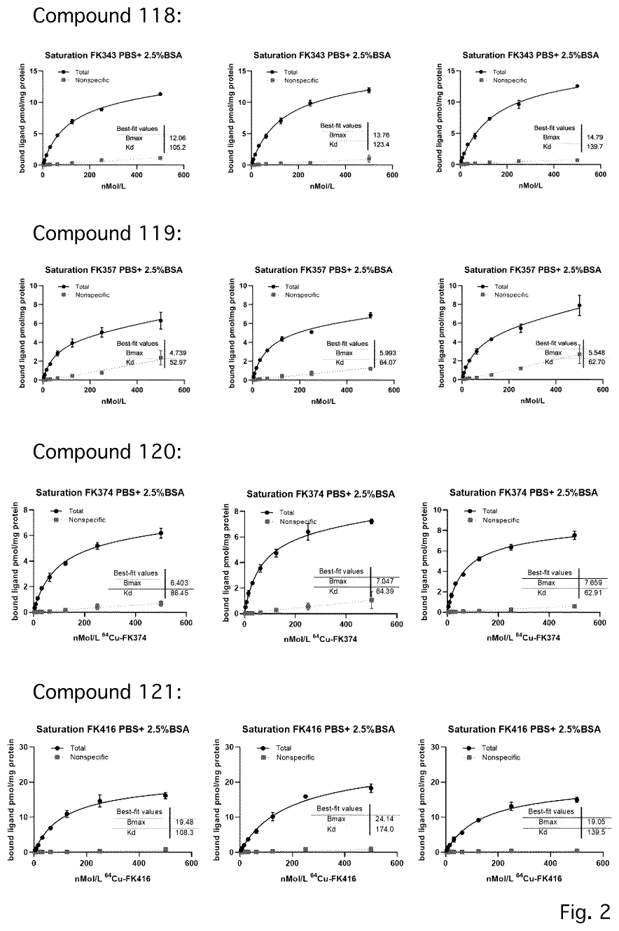

FIG. 2 shows diagrams as obtained in saturation binding experiments on a PC3 hPSCA PD-L1 overexpressing cell line for compounds 118, 119, 120 and 121 in triplicates;

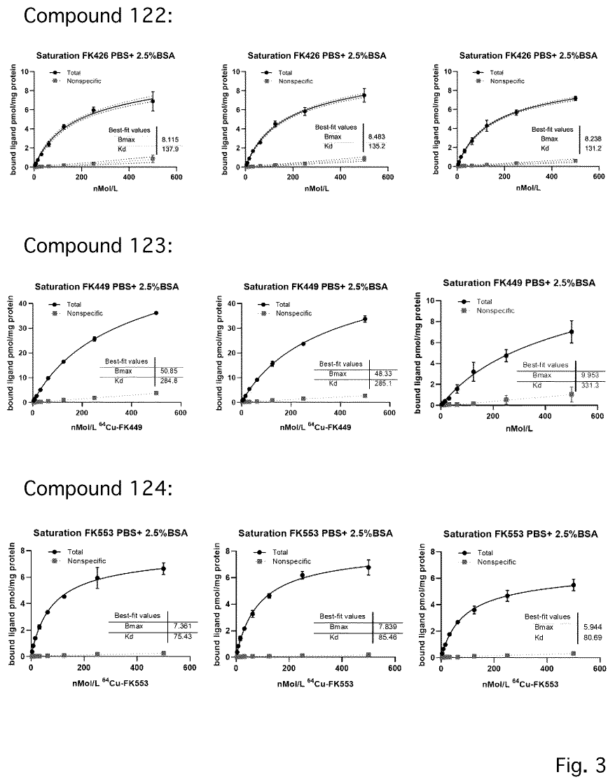

FIG. 3 shows diagrams as obtained in saturation binding experiments on a PC3 hPSCA PD-L1 overexpressing cell line for compounds 122, 123 and 124 in triplicates;

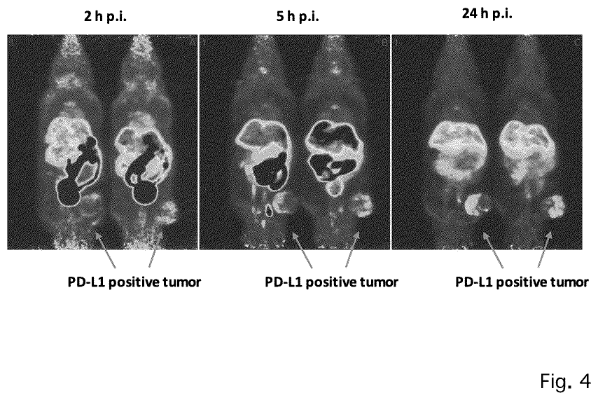

FIG. 4 shows PET images 2 h, 15 h and 24 h after injection of Compound 119 in a PD-L1 overexpressing mouse xenograft model and

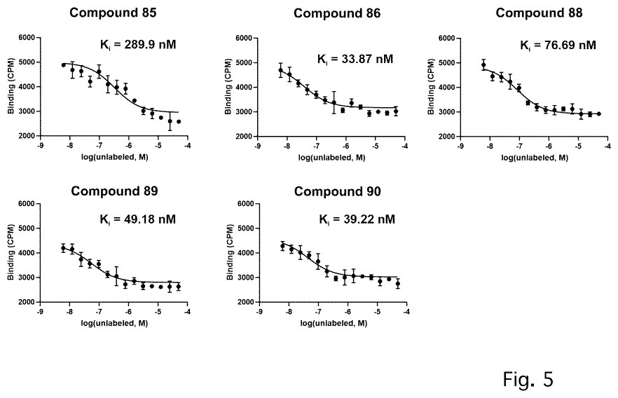

FIG. 5 shows diagrams as obtained in cell-based competitive assay for compounds 85, 86, 88 to 90 using the Lu-labelled DOTA compound 129.

### EXAMPLES

### General Procedures

### GP-1: S_{N}2-Alkylation

The corresponding alcohol (1.0 equiv.) was dissolved in anhydrous DMF under argon atmosphere and potassium carbonate (2.0 equiv.) was added. After stirring the suspension at room temperature for 10 min, then the alkyl bromide (1.2-1.5 equiv.) was added. The reaction mixture was stirred at room temperature for the corresponding time and after completion, the solvent was removed in vacuo. The residue was taken up in an organic solvent and water, the phases were separated and the aqueous phase was extracted with an organic solvent three times. The combined organic phases were dried over anhydrous sodium sulfate and after filtration, the solvent was removed in vacuo. The crude product was purified by flash column chromatography on silica gel using an appropriate eluent.

### GP-2: Mitsonobu-Reaction

The phenolic compound (1.0 equiv), the alcohol (1.1 equiv.) and triphenylphosphine (1.3 equiv) were dissolved in anhydrous DMF under argon atmosphere. The reaction solution was cooled to 0° C. and DEAD (1.3 equiv.) was added slowly by syringe. After allowing to warm room temperature, the reaction solution was stirred for the corresponding time. After completion, all volatiles were removed in vacuo and the residue was purified by flash column chromatography on silica gel using an appropriate eluent.

### GP-3: Reductive Amination

The aldehyde (1.0 equiv.) and the amine (3.0-5.0 equiv.) were dissolved in MeOH/DMF (1:1) under argon atmosphere. After stirring for 20 min at room temperature, the reaction solution was cooled to 0° C. and NaBH₃CN (1.5 equiv.) was added. The solution was warmed to room temperature and stirred for the corresponding time. After completion of the reaction, water and ethyl acetate were added. Phases were separated and the aqueous phase was extracted with ethyl acetate three times. The organic layers were combined, dried over anhydrous sodium sulfate and filtrated. After removing the solvent in vacuo, the crude product was purified by flash column chromatography on silica gel using an appropriate eluent.

GP-4: CuAAC-Reaction (Cu(I)-Catalysator system)

The alkyne (1.00 equiv.) and azide (1.50-5.00 equiv.) were dissolved in the corresponding solvent and the resulting solution was degassed with argon for 30 min. TBTA (0.01 equiv.) and [Cu(MeCN)₄]PF₆ (0.50 equiv.) were added in one portion under argon. The reaction mixture was stirred at room temperature for 16 h. Complete conversion of starting material was verified either by TLC or RP-HPLC. The solvent was removed in vacuo and the crude product was purified either by flash column chromatography on silica gel or by RP-HPLC with subsequent lyophilization.

GP-5: CuAAC-Reaction (CuSO₄/sodium ascorbate system)

The alkyne (1.00 equiv.) and azide component (1.00-5.00 equiv.) were dissolved in a 1:1 mixture of H₂O/t-BuOH. CuSO₄ (0.30 equiv.), THPTA (0.10 equiv.) and sodium ascorbate (5.00 equiv.) were premixed in H₂O/t-BuOH (1:1) and then added to the reaction solution, which was stirred at room temperature until complete conversion was achieved (monitored by RP-HPLC System A). All volatiles were removed in vacuo, the crude product was purified by RP-HPLC and after lyophilization, the desired triazol was obtained.

### GP-6: Amide-Coupling of 2-Aminoethan-1,1-Disulfonic Acid (TBA Salt)

The carboxylic acid (1.0 equiv.), 2-aminoethan-1,1-disulfonic acid (TBA salt) (5.0 equiv.), anhydrous DIPEA (2.0 equiv.) and HBTU (2.2 equiv.) were stirred in anhydrous DMF at room temperature for 40 h. The suspension was filtered over a short plug of celite and DMF was removed under reduced pressure. The crude product was purified by RP-HPLC to yield the product as a TBA salt after lyophilization. Dissolving in water and stirring with DOWEX® 50WX8 at room temperature for 2 h, followed by lyophilization led to the disulfonated product in acid form.

### GP-7: DOTA-Conjugation

The secondary amine (1.0 equiv.) was dissolved in anhydrous DMF and the reaction was initialized by the addition of anhydrous DIPEA (5.0 equiv.) and DOTA-p-nitro-phenylester (2.0 equiv.). The reaction was stirred at room temperature for 40 h and complete consumption was verified by analytical RP-HPLC (System A). The reaction mixture was then stirred at 80° C. for 4 h to decompose excess of DOTA-p-nitrophenylester. The solvents were removed under reduced pressure and the crude product was purified by RP-HPLC and lyophilized to yield the DOTA-conjugated compound.

### GP-8: General Amide-Bond Formation

The carboxylic acid, the base, the coupling reagent and HOBt (if chirality present; 1.0 equiv.) were dissolved in abs. DMF and were stirred for 10 min at room temperature. The reaction mixture was cooled down to 0° C. and the amine component was added. The reaction was stirred at room temperature whereas the progress of the reaction was monitored by analytical RP-HPLC (System A). After complete conversion, the solvent was removed and the crude product was purified by semi-preparative RP-HPLC. Followed by lyophilization, the amide was obtained.

GP-9: Fmoc-Deprotection with Sodium Azide

The Fmoc-bearing compound (1.0 equiv.) was dissolved in abs. DMF, sodium azide (5.0 equiv.) was added and the reaction mixture was heated to 60° C. for 3 h. After confirming complete conversion by analytical HPLC (System A), the solvent was removed under reduced pressure. The residue was purified by semi-preparative RP-HPLC and subsequent lyophilization provided the free amine.

GP-10: t-Butyl-Ester Deprotection

The chelator compound bearing t-butyl esters was stirred in the deprotection cocktail (TFA:DCM:TES:H₂O, 20:20:8:7, v/v) at room temperature for 40 h. The threefold deprotection was confirmed with analytical RP-HPLC (System A). The solvent was removed in vacuo, the residue was purified by semi-preparative RP-HPLC and after lyophilization, the title compound was obtained.

GP-11 Dealkylation of Phosphonate Esters and tert-Butyl-Ester Deprotection

The protected compound was dissolved in a NMR tube under argon in abs. DMF and then trimethylsilyl bromide (20.0 equiv.) was added. The reaction was monitored by ³¹P NMR and after complete formation of the bis(trimethylsilyl) phosphonate, methanol (50 μL) was added. The NMR tube was shaken and complete hydrolysis of bis(trimethylsilyl) phosphonate was confirmed by ³¹P NMR. The solvent was removed in vacuo and the residue was directly subjected to the tBu-deprotection which was performed according to GP-10.

### HPLC-Systems

System A: RP-HPLC, analytical (Agilent Zorbax 300 C-18, 5 μm, 4.6×150 mm) with 10-95% acetonitrile (0.1% TFA) in water (0.1% TFA) in a linear gradient over 15 min, 1 mL/min.
System B: RP-HPLC, semi-preparative (Zorbax SB C-18 5 μm 80 Å, 9.4×250 mm) with 35-90% acetonitrile (0.1% TFA) in water (0.1% TFA) in a linear gradient over 45 min, 6 mL/min.
System C: RP-HPLC, semi-preparative (Zorbax SB C-18 5 μm 80 Å, 9.4×250 mm) with 20-60% acetonitrile (0.1% TFA) in water (0.1% TFA) in a linear gradient over 45 min, 6 mL/min.
System D: hydro-RP-HPLC, preparative (Phenomenex Synergi hydro-RP 4 μm 80 Å, 21.2×250 mm) with 25-35% acetonitrile (0.1% TFA) in water (0.1% TFA) in a Linear gradient over 60 min, 15 mL/min.
System E: RP-HPLC, analytical (Agilent Zorbax 300 C-18, 5 μm, 4.6×250 mm) with 10-95% acetonitrile (0.1% TFA) in water (0.1% TFA) in a linear gradient over 30 min, 1 mL/min.

### Preparation of the Starting Compounds

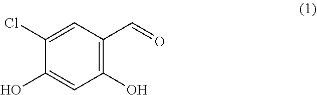 [SMILES: O=Cc1cc(Cl)c(O)cc1O]

was synthesized according to literature [1].

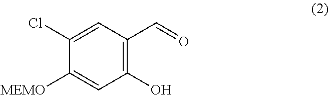

5-Chloro-2-hydroxy-4-((2-methoxyethoxy)methoxy)benzaldehyde (2). 5-Chloro-2,4-dihydroxybenzaldehyde (1) (4.30 g, 24.7 mmol, 1.00 equiv.) was dissolved in anhydrous THF (80 mL) under argon atmosphere and anhydrous triethylamine (8.81 mL, 49.5 mmol, 2.00 equiv.) was added via syringe in one portion. After cooling to 0° C., MEM chloride (3.96 mL, 34.6 mmol, 1.40 equiv.) was added in small portions. The completion of the reaction was verified by TLC after stirring at room temperature for 16 h under argon. Water (100 mL) was added and the phases were separated. The aqueous phase was extracted with DCM (3×100 mL) and the combined organic extracts were dried over anhydrous sodium sulfate, filtered and concentrated. The residue was purified by flash column chromatography on silica gel (PE:EA, 8:2, R_{f}=0.25) to afford 2 (3.87 g, 14.9 mmol, 60%) as colorless crystals. mp=53° C. ¹H NMR (600 MHz, CDCl₃) δ=11.28 (s, 1H), 9.70 (s, 1H), 7.52 (s, 1H), 6.80 (s, 1H), 5.40 (s, 2H), 3.85-3.87 (m, 2H), 3.55-3.57 (m, 2H), 3.37 ppm (s, 3H). ¹³C NMR (151 MHz, CDCl₃) δ=194.0, 162.7, 159.7, 134.2, 115.9, 115.0, 103.8, 94.1, 71.5, 68.8, 59.2 ppm. IR (ATR): \tilde{ν}=2856 (w), 1643 (s), 1622 (s), 1572 (m), 1489 (s), 1454 (m), 1354 (m), 1320 (m), 1273 (m), 1242 (w), 1223 (w), 1195 (s), 1154 (m), 1136 (m), 1105 (s), 1027 (s), 940 (s), 891 (m), 830 (s), 738 (s), 717 (s), 705 (s), 681 (m), 662 (m), 582 (w), 559 (m), 493 (w), 458 (w), 436 cm⁻¹ (w). MS (HR-ESI⁺): Exact mass calculated for [M+Na]⁺: m/z=283.0349 measured: m/z=283.0346.

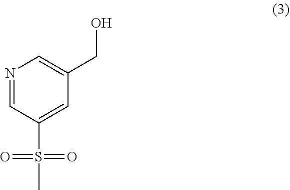

was synthesized according to literature [2].

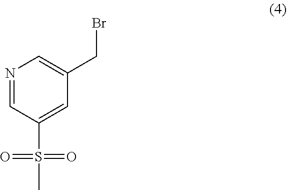

3-(Bromomethyl)-5-(methylsulfonyl)pyridine (4). (5-(Methylsulfonyl)pyridin-3-yl)methanol (3) (5.00 g, 35.1 mmol, 1.00 equiv.) was dissolved under argon in abs. THF (200 mL) and cooled to 0° C. Triphenylphosphine (9.67 g, 36.9 mmol, 1.05 equiv.) and tetrabromomethane (12.2 g, 36.90 mmol, 1.05 equiv.) were added and the solution was stirred at 0° C. for 15 min and then for 16 h at room temperature until complete consumption of starting material was observed. Subsequently, the reaction mixture was filtered, water (200 mL) was added to the filtrate, phases were separated and the organic extract was dried over sodium sulfate. After filtration and removal of the solvent, the crude product was purified by flash column chromatography on silica gel (PE:EA, 5:5, R_{f}=0.15) to afford 3-(bromomethyl)-5-(methylsulfonyl)pyridine (4) (3.73 g, 14.9 mmol, 42%) as beige crystals. mp=240° C. ¹H NMR (600 MHz, CDCl₃) δ=9.07 (d, ⁴J=2.1 Hz, 1H), 8.90 (d, ⁴J=2.1 Hz, 1H), 8.26 (t, ⁴J=2.1 Hz, 1H), 4.53 (s, 2H), 3.14 ppm (s, 3H). ¹³C NMR (151 MHz, CDCl₃) δ=154.5, 148.2, 137.2, 135.7, 134.8, 45.0, 27.8 ppm. IR (ATR): G=3024 (w), 1565 (w), 1423 (m), 1295 (s), 1246 (m), 1211 (m), 1160 (m), 1138 (s), 1099 (s), 1021 (w), 960 (m), 915 (w), 860 (w), 766 (m), 707 (m), 683 (m), 638 (w), 573 (m), 544 (m), 525 (s), 441 (w), 423 cm⁻¹ (m). MS (HR-EI⁺): Exact mass calculated for [M]′⁺: m/z=248.9459, measured: m/z=248.9467.

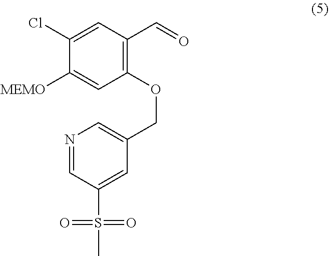

5-Chloro-4-((2-methoxyethoxy)methoxy)-2-((5-(methylsulfonyl)pyridin-3-yl)methoxy)benzaldehyde (5). 5-Chloro-2-hydroxy-4-((2-methoxyethoxy)methoxy)benzaldehyde (2) (2.09 g, 8.01 mmol, 1.00 equiv.) reacted with 3-(bromomethyl)-5-(methylsulfonyl)pyridine (4) (2.60 g, 10.4 mmol, 1.30 equiv.) and potassium carbonate (2.21 g, 16.0 mmol, 2.00 equiv.) as abase in abs. DMF (40 mL) according to GP-1. Purification by flash column chromatography on silica gel (EA:MeOH, 100:1.5, R_{f}=0.25) provided 5-chloro-4-((2-methoxyethoxy)methoxy)-2-((5-(methylsulfonyl)pyridin-3-yl)methoxy)benzaldehyde (5) (2.89 g, 6.72 mmol, 84%) as a yellowish solid. mp=134° C. ¹H NMR (600 MHz, CDCl₃) δ=10.28 (s, 1H), 9.16 (s, 1H), 8.99 (s, 1H), 8.37 (s, 1H), 7.87 (s, 1H), 7.01 (s, 1H), 5.42 (s, 2H), 5.27 (s, 2H), 3.88-3.90 (m, 2H), 3.57-3.59 (m, 2H), 3.37 (s, 3H), 3.16 ppm (s, 3H). ¹³C NMR (151 MHz, CDCl₃) (=186.7, 160.0, 158.9, 153.4, 148.6, 137.4, 134.5, 132.4, 130.7, 120.3, 117.5, 101.0, 94.3, 71.5, 68.5, 67.9, 59.1), 45.0 ppm. IR (ATR): G=2921 (w), 1667 (m), 1594 (m), 1498 (w), 1429 (w), 1398 (w), 1384 (m), 1303 (s), 1258 (m), 1188 (m), 1145 (s), 1117 (s), 1084 (m), 1059 (w), 1013 (m), 984 (m), 967 (s), 894 (w), 867 (m), 851 (m), 824 (m), 768 (s), 722 (m), 701 (m), 670 (m), 642 (w), 590 (w), 557 (m), 535 (s), 516 (m), 458 (w), 427 cm⁻¹ (w). MS (HR-ESI⁺): Exact mass calculated for [2M+Na]+: m/z=881.1190, measured: m/z=881.1190.

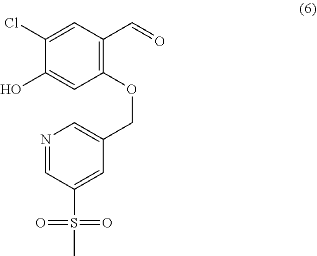 [SMILES: CS(=O)(=O)c1cncc(COc2cc(O)c(Cl)cc2C=O)c1]

5-Chloro-4-hydroxy-2-((5-(methylsulfonyl)pyridin-3-yl)methoxy)benzaldehyde (6). MEM-Protected phenol 5 (2.87 g, 6.68 mmol, 1.00 equiv.) was dissolved in abs. dichloromethane (50 mL) under argon and the solution was cooled to 0° C. After addition of trifluoracetic acid (10 mL), the reaction mixture was stirred at room temperature for 16 h. Ethyl acetate (100 mL) was added and a solid precipitated, which was isolated by filtration. The pale greenish precipitate turned out to be 5-chloro-4-hydroxy-2-((5-(methylsulfonyl)pyridin-3-yl)methoxy)benzaldehyde (6) (0.96 g, 2.81 mmol, 42%), which was used in the next step without further purification. mp=236° C. (decomposition). ¹H NMR (600 MHz, DMSO-d₆) δ=11.63 (s, 1H), 10.10 (s, 1H), 9.09 (d, 4J=2.0 Hz), 9.07 (d, ⁴J=2.0 Hz), 8.48 (d, ⁴J=2.0 Hz), 7.67 (s, 1H), 6.81 (s, 1H), 5.38 (s, 2H), 3.37 ppm (s, 3H). ¹³C NMR (151 MHz, DMSO-d₆) δ=186.5, 160.4, 159.9, 153.3, 147.4, 137.0, 134.3, 132.8, 129.5, 117.9, 113.5, 101.5, 67.1, 43.6 ppm. IR (ATR): \tilde{ν}=2915 (w), 1672 (m), 1598 (m), 1520 (w), 1452 (m), 1399 (m), 1368 (w), 1303 (s), 1214 (m), 1191 (s), 1142 (s), 1103 (m), 1029 (m), 974 (m), 902 (w), 845 (w), 816 (m), 765 (m), 725 (m), 699 (m), 665 (w), 641 (w), 556 (m), 535 (s), 458 (m), 429 cm⁻¹ (w). MS (HR-ESI⁺): Exact mass calculated for [M+H]+: m/z=342.0197, measured: m/z=342.0197.

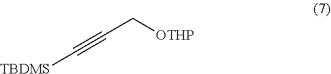

was synthesized according to literature[3].

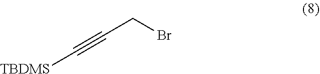

(3-Bromoprop-1-yn-1-yl)(tert-butyl)dimethylsilane (8). TBDMS-protected alkyne 7 (13.5 g, 53.1 mmol, 1.00 equiv.) was dissolved in DCM (300 mL), triphenylphosphine bromide (24.6 g, 58.4 mmol, 1.10 equiv.) was added and the reaction mixture was stirred at room temperature for 3 h. The solvent was removed under reduced pressure and the crude product was filtered over a plug of silica gel (PE) to elute (3-bromoprop-1-yn-1-yl)(tert-butyl)dimethylsilane (8) (8.57 g, 36.8 mmol, 69%) as a yellowish oil.

Analytical data are in accordance with literature[3].

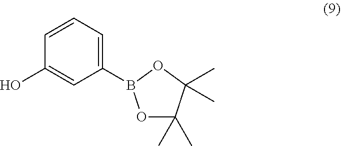 [SMILES: CC1(C)OB(c2cccc(O)c2)OC1(C)C]

was synthesized according to literature[4].

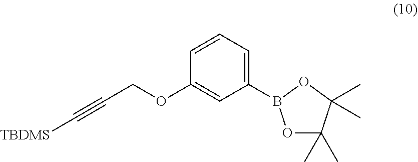

tert-butyldimethyl(3-(3-(4,4,5,5-tetramethyl-1, 3,2-dioxaborolan-2-yl)phenoxy)prop-1-yn-1-yl)silane (10). 3-(4,4,5,5-Tetramethyl-1,3,2-dioxaborolan-2-yl)phenol (9) (4.03 g, 18.3 mmol, 1.00 equiv.) reacted with 3-(TBDMS)propargyl bromide (5.13 g, 22.0 mmol, 1.20 equiv.) and potassium carbonate (3.80 g, 27.5 mmol, 1.50 equiv.) as a base in abs. DMF (50 mL) according to GP-1. Purification by flash column chromatography on silica gel (PE:DCM, 1:1, R_{f}=0.4) gave TBDMS-protected alkyne 10 (4.63 g, 12.4 mmol, 68%) as yellow oil. ¹H NMR (600 MHz, CDCl₃) δ=7.44-7.42 (m, 2H), 7.29-7.27 (m, 1H), 7.08 (ddd, ³J=8.2 Hz, ⁴J=2.7 Hz, ⁵J=1.2 Hz, 1H), 4.71 (s, 2H), 1.34 (s, 12H), 0.91 (s, 9H), 0.11 ppm (s, 6H). ¹³C NMR (151 MHz, CDCl₃) δ=157.6, 129.0, 128.2, 120.9, 119.2, 101.4, 91.2, 84.0, 57.1, 26.2, 25.1, 16.6, −4.6 ppm. IR (ATR): \tilde{ν}=2929 (m), 2857 (m), 1575 (w), 1489 (w), 1428 (m), 1349 (s), 1315 (m), 1251 (w), 1210 (s), 1143 (s), 1069 (w), 1042 (m), 964 (m), 884 (w), 825 (m), 776 (m), 705 (m), 580 cm⁻¹ (w). MS (HR-EI⁺): Exact mass calculated for [M]+: m/z=372.2292, measured: m/z=372.2275.

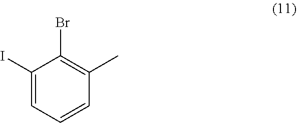

was synthesized according to the literature [5].

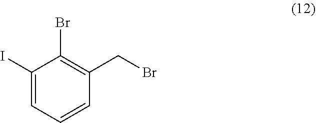

was synthesized according to the literature [6].

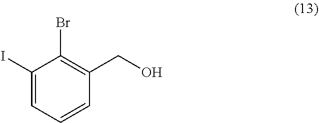

3-Iodo-2-bromobenzylalcohol (13). 3-Iodo-2-bromobenzylbromide (12) (7.80 g, 20.8 mmol, 1.00 equiv.) was suspended in an 8:2 mixture of DMF/H₂O (250 mL) and the suspension was heated to 80° C. for 16 h. After complete conversion, the mixture was cooled down and DCM (400 mL) and water (200 mL) were added. The phases were separated, the aqueous phase was extracted with DCM (3×400 mL), the combined organic phases were dried over sodium sulfate, filtered and concentrated. The residue was purified by flash column chromatography on silica gel (PE:EA, 10:1, R_{f}=0.2) to yield 3-iodo-2-bromobenzylalcohol (13) (4.89 g, 15.6 mmol, 75%) as colorless crystals. mp=97° C. ¹H NMR (400 MHz, DMSO-d₆) δ=7.85 (d, ³J=7.7 Hz, 1H), 7.50 (d, ³J=7.7 Hz, 1H), 7.18 (t, ³J=7.7 Hz, 1H), 4.50 ppm (s, 2H). ¹³C NMR (101 MHz, DMSO-d₆) δ=181.4, 175.9, 166.7, 165.0, 164.9, 141.0, 102.2 ppm. IR (ATR): G=3201 (w), 1530 (w), 1431 (w), 1400 (m), 1431 (m), 1233 (w), 1180 (w), 1133 (m), 1088 (m), 153 (s), 1012 (m), 981 (m), 814 (w), 7662 (s), 685 (s), 616 cm⁻¹ (m). MS (HR-EI⁺): Exact mass calculated for [M]′⁺: m/z=311.8638, measured: m/z=311.8647.

The analytical data are in accordance with the literature [7].

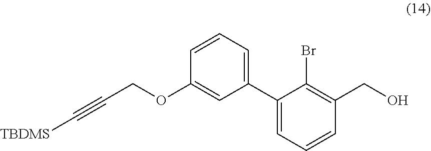

(2-Bromo-3′-((3-(tert-butyldimethylsilyl)prop-2-yn-1-yl)oxy)-[,1,1′-biphenyl]-3-yl)methanol (14). 3-Iodo-2-bromobenzylalcohol (13) (3.22 g, 10.3 mmol, 1.00 equiv.) and TBDMS-protected alkyne 10 (4.41 g, 11.8 mmol, 1.15 equiv.) were dissolved in a 20:0.5:0.1 mixture of toluene/EtOH/H₂O (130 mL) and the resulting solution was degassed with argon for 30 min. The reaction was initiated by the addition of potassium carbonate (2.84 g, 20.6 mmol, 2.00 equiv.) and tetrakis(triphenylphosphine)palladium(0) (0.59 g, 0.51 mmol, 0.05 equiv.). The mixture was stirred at 78° C. for 16 h until all starting material was consumed. It was cooled to room temperature, water (200 mL) and DCM (400 mL) were added, the phases were separated and the aqueous phase was extracted with DCM (3×300 mL). Drying over sodium sulfate, filtrating and concentrating under reduced pressure lead to the crude product, which was purified by flash column chromatography on silica gel (PE:EA, 8:2, R_{f}=0.2) to give (2-bromo-3′-((3-(TBDMS)prop-2-yn-1-yl)oxy)-[1,1′-biphenyl]-3-yl)methanol (14) (2.79 g, 6.47 mmol, 63%) as a yellowish oil. ¹H NMR (400 MHz, DMSO-d₆) δ=7.56 (d, 3J=7.6 Hz, 1H), 7.45 (t, ³J=7.6 Hz, 1H), 7.36 (t, ³J=7.9 Hz, 1H), 7.23-7.21 (m, 1H), 7.02-7.00 (m, 1H), 6.97-6.94 (m, 2H), 5.50 (t, ³J=5.1 Hz, 1H), 4.86 (s, 2H), 4.57 (s, 2H), 0.83 (s, 9H), 0.06 ppm (s, 6H). ¹³C NMR (101 MHz, DMSO-d₆) δ=156.8, 142.2, 141.9, 129.3, 129.0, 127.2, 126.8, 122.3, 115.8, 114.5, 102.0, 90.1, 63.3, 56.2, 25.7, 16.1, −4.9 ppm. IR (ATR): G=3344 (w), 3059 (w), 2952 (m), 2927 (m), 2884 (w), 2856 (m), 2177 (w), 1602 (w), 1580 (m), 1461 (m), 1409 (w), 1362 (w), 1250 (m), 1201 (m), 1076 (w), 1340 (m), 1023 (m), 989 (w), 838 (m), 824 (m), 775 (s), 697 (m), 684 cm⁻¹ (m). MS (HR-EI⁺): Exact mass calculated for [M]′⁺: m/z=430.0964, measured: m/z=430.0975.

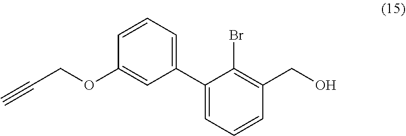 [SMILES: C#CCOc1cccc(-c2cccc(CO)c2Br)c1]

(2-Bromo-3′-(prop-2-yn-1-yloxy)-[1,1′-biphenyl]-3-yl)methanol (15). TBMDS-Protected biaryl 14 (2.56 g, 5.94 mmol, 1.00 equiv.) was dissolved in abs. THF (20 mL), cooled to 0° C. and TBAF (1 M in THF, 6.53 mL, 6.54 mmol, 1.10 equiv.) was added slowly by syringe. The reaction solution was allowed to warm to room temperature and was stirred for 2 h to ensure complete consumption of the starting material. Water (50 mL) and DCM (40 mL) were added, the phases were separated and the aqueous phase was extracted with DCM (3×30 mL). The combined organic phases were dried over sodium sulfate, filtrated and concentrated under reduced pressure. The crude product was purified by flash column chromatography on silica gel (PE:EA, 7:3, R_{f}=0.3) to obtain (2-bromo-3′-(prop-2-yn-1-yloxy)-[1,1′-biphenyl]-3-yl)methanol (15) (1.60 g, 5.04 mmol, 85%) as a colorless solid. mp=90° C. ¹H NMR (400 MHz, CDCl₃) δ=7.47 (d, ³J=7.5 Hz, 1H), 7.35-7-32 (m, 2H), 7.25-7.23 (m, 1H), 7.01-6.97 (m, 3H), 4.80 (s, 2H), 4.71-4.70 (m, 2H), 2.52 ppm (s, 1H). ¹³C NMR (101 MHz, CDCl₃) δ=157.2, 143.2, 142.8, 140.7, 130.4, 129.2, 127.9, 127.4, 122.9, 122.9, 116.2, 114.3, 78.7, 75.8, 66.0, 56.0 ppm. IR (ATR): \tilde{ν}=3279 (s), 3072 (w), 3029 (w), 2929 (w), 2855 (w), 2358 (w), 2116 (w), 1591 (m), 1463 (m), 1452 (m), 1445 (w), 1345 (w), 1318 (w), 1264 (w), 1202 (m), 1176 (w), 1161 (w), 1083 (m), 1060 (m), 1033 (m), 1021 (m), 995 (w), 854 (w), 803 (w), 794 (w), 777 (m), 716 (w), 695 (m), 639 cm⁻¹ (m). MS (HR-EI⁺): Exact mass calculated for [M]+: m/z=316.0099, measured: m/z=316.0082.

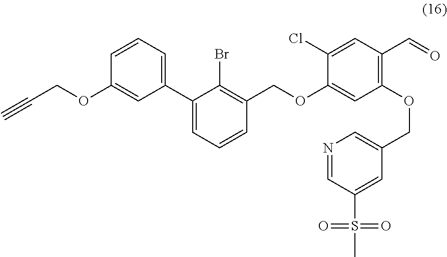 [SMILES: C#CCOc1cccc(-c2cccc(COc3cc(OCc4cncc(S(C)(=O)=O)c4)c(C=O)cc3Cl)c2Br)c1]

4-((2-Bromo-3′-(prop-2-yn-1-yloxy)-[,1,1′-biphenyl]-3-yl)methoxy)-5-chloro-2-((5-methylsulfonyl) pyridin-3-yl)methoxy)benzaldehyde (16). Biaryl 15 (500 mg, 1.58 mmol, 1.00 equiv.), phenol 6 (647 mg, 1.89 mmol, 1.20 equiv.) and triphenylphosphine (538 mg, 2.05 mmol, 1.30 equiv.) were dissolved in abs. DMF (5 mL) and reacted with DEAD (322 μL, 2.05 mmol, 1.30 equiv.) according to GP-2. Purification by flash column chromatography on silica gel (DCM:EA, 9:1, R_{f}=0.25) yielded compound 16 (808 mg, 1.26 mmol, 80%) as a colorless solid. mp=145° C. ¹H NMR (400 MHz, DMSO-d₆) δ=10.23 (s, 1H), 9.09 (d, 2H), 8.52 (s, 1H), 7.76 (s, 1H), 7.70 (d, ³J=7.5 Hz, 1H), 7.54 (t, ³J=7.6 Hz, 1H), 7.43-7.39 (m, 2H), 7.29 (s, 1H), 7.06-7.00 (m, 3H), 5.57 (s, 2H), 5.46 (s, 2H), 4.85 (d, ⁴J=2.0 Hz, 2H), 3.58 (t, ⁴J=2.0 Hz, 1H), 3.37 ppm (s, 3H). ¹³C NMR (101 MHz, DMSO-d₆) δ=186.8, 160.7, 159.3, 156.8, 153.3, 147.4, 142.8, 142.0, 137.0, 135.6, 134.3, 132.7, 131.4, 129.4, 129.2, 128.9, 127.8, 123.1, 122.5, 122.3, 118.9, 115.8, 114.9, 114.2, 100.5, 79.2, 78.4, 71.5, 67.8, 55.5, 43.6 ppm. IR (ATR): \tilde{ν}=3239 (m), 3040 (w), 2991 (m), 2917 (w), 1747 (m), 1693 (s), 1676 (m), 1595 (m), 1530 (s), 1481 (w), 1449 (w), 1367 (w), 1319 (w), 1284 (m), 1233 (s), 1206 (w), 1174 (m), 1144 (m), 1063 (m), 1021 (m), 960 (w), 900 (w), 791 (w), 756 (w), 643 (w), 593 cm⁻¹ (w). MS (HR-ESI⁺): Exact mass calculated for [M+Na]⁺: m/z=662.0010, measured: m/z=662.0018.

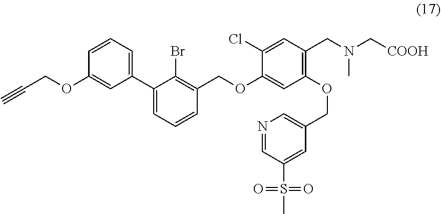 [SMILES: C#CCOc1cccc(-c2cccc(COc3cc(OCc4cncc(S(C)(=O)=O)c4)c(CN(C)CC(=O)O)cc3Cl)c2Br)c1]

N-(4-((2-Bromo-3′-(prop-2-yn-1-yloxy)-[1,1′-biphenyl]-3-yl)methoxy)-5-chloro-2-((5-(methylsulfonyl) pyridin-3-yl)methoxy)benzyl)-N-methylglycine (17). Aldehyde 16 (70.0 mg, 109 μmol, 1.00 equiv.), sarcosine (34.1 mg, 382 μmol, 3.50 equiv.) and sodium cyanoborohydride (10.3 mg, 164 μmol, 1.50 equiv.) were reacted in an 1:1 mixture of abs. MeOH/DMF (2 mL) and subsequently worked up according to GP-3. After flash column chromatography on silica gel (MeCN:MeOH:Et3N, 100:20:1, R_{f}=0.2) compound 17 (44.0 mg, 61.6 μmol, 56%) was obtained as a colorless solid. mp=175-175° C. Rₜ=11.98 min (System A), purity: 95.4%. ¹H NMR (400 MHz, DMSO-d₆) δ=9.07 (s, 1H), 9.03 (s, 1H), 8.46 (s, 1H), 7.68 (d, ³J=6.8 Hz, 1H), 7.52 (t, ³J=7.6 Hz, 1H), 7.45-7.38 (m, 3H), 7.13 (s, 1H), 7.05-6.99 (m, 3H), 5.41 (s, 2H), 5.32 (s, 2H), 4.84 (d, ⁴J=1.8 Hz, 2H), 3.75 (s, 3H), 3.58 (s, 1H), 3.21 (s, 2H), 2.34 ppm (s, 3H). ¹³C NMR (101 MHz, DMSO-d₆) δ=170.6, 156.8, 155.9, 153.5, 153.0, 147.2, 142.7, 142.0, 136.9, 136.4, 134.2, 133.3, 131.3, 131.1, 129.2, 129.0, 127.7, 122.8, 122.3, 119.4, 115.8, 114.1, 113.3, 100.8, 79.2, 78.4, 71.0, 67.3, 57.5, 55.5, 53.1, 43.7, 41.4 ppm. IR (ATR): G=3226 (w), 3047 (w), 2925 (w), 1606 (m), 1578 (w), 1506 (m), 1463 (w), 1404 (w), 1366 (w), 1302 (s), 1202 (m), 1149 (m), 1109 (w), 1042 (w), 996 (w), 977 (w), 803 (w), 774 (m), 699 cm⁻¹ (w). MS (HR-ESI⁺): Exact mass calculated for [M+H]⁺: m/z=713.0719, measured: m/z=713.0724.

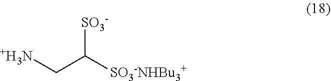

was synthesized according to literature [8].

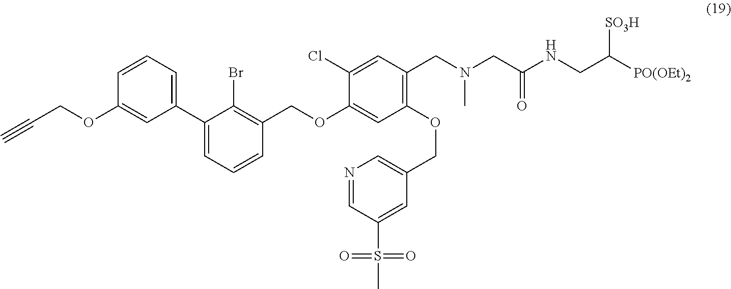

Bis(sulfonic acid) (19). Carboxylic acid 17 (369 mg, 517 μmol, 1.00 equiv.) and 2-aminoethan-1,1-disulfonic acid (TBA salt) (18) (1.01 g, 2.58 mmol, 5.00 equiv.) was dissolved in abs. DMF (5 mL) followed by addition of of DIPEA (180 μL, 1.03 mol, 2.00 equiv.) and HBTU (431 mg, 1.14 mmol, 2.20 equiv.). After completion the reaction was subsequently worked up according to GP-6. After purification by preparative RP-HPLC (System D, Rt=32 min) and desalting, alkyne 19 (334 mg, 307 μmol, 60%) was obtained as an off-white powder. mp=195-198° C. (decomposition). Rₜ=12.42 min (System A), purity: 100%. ¹H NMR (400 MHz, DMSO-d₆) δ=9.61 (bs, 1H), 9.17 (s, 1H), 9.11 (s, 1H), 8.57 (s, 1H), 8.14 (t, 3J=5.0 Hz, 1H), 7.71-7.69 (m, 1H), 7.66 (s, 1H), 7.53 (t, ³J=7.6 Hz, 1H), 7.43-7.39 (m, 2H), 7.22 (s, 1H), 7.06-7.00 (m, 3H), 5.51 (s, 2H), 5.38 (s, 2H), 4.84 (d, ⁴J=2.3 Hz, 2H), 3.93-3.89 (m, 2H), 3.71-3.64 (m, 2H), 3.59-3.54 (m, 2H), 3.39 (s, 3H), 2.72 ppm (s, 3H). ¹³C NMR (101 MHz, DMSO-d₆) δ=163.5, 156.9, 156.8, 155.6, 152.4, 146.5, 142.7, 142.0, 137.2, 136.1, 135.5, 134.0, 133.4, 131.2, 129.4, 129.2, 127.8, 123.1, 122.3, 115.8, 114.1, 113.4, 111.8, 111.4, 109.5, 100.2, 79.2, 78.4, 74.1, 71.2, 67.4, 56.0, 5.55, 52.7, 43.6, 40.6 ppm. IR (ATR): G=3288 (w), 3053 (w), 2924 (w), 2162 (w), 1682 (m), 1606 (m), 1580 (w), 1506 (w), 1462 (w), 1407 (w), 1307 (m), 1201 (s), 1174 (s), 1154 (s), 1063 (m), 1019 (m), 932 (w), 778 (w), 699 (w), 674 (w), 590 cm⁻¹ (w). MS (HR-ESI⁺): Exact mass calculated for [M+H]⁺: m/z=900.0328, measured: m/z=900.0317.

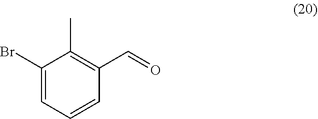

was synthesized according to literature [9].

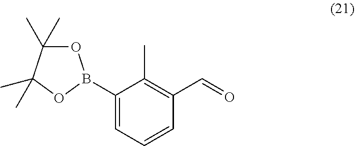 [SMILES: Cc1c(C=O)cccc1B1OC(C)(C)C(C)(C)O1]

was synthesized according to literature [10].

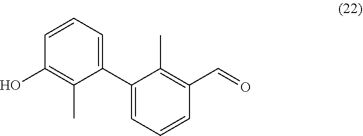 [SMILES: Cc1c(O)cccc1-c1cccc(C=O)c1C]

3′-Hydroxy-2′-methyl-[1,1′-biphenyl]-3-carbaldehyde (22). A solution of 3-bromo-2-methylphenol (3.93 g, 21.0 mmol, 1.00 equiv.) and 2-methyl-3-(4,4,5,5-tetramethyl-1,3-dioxolan-2-yl)benzaldehyde (21) (5.69 g, 23.1 mmol, 1.10 equiv.) in a 7:1 mixture of 1,4-dioxane/water (300 mL) was degassed with argon for 30 min and after addition of potassium carbonate (8.71 g, 63.0 mmol, 3.00 equiv.) and tetrakis(triphenylphosphine)palladium(0) (1.21 g, 1.05 mmol, 0.05 equiv.) in one portion, the reaction mixture was stirred under argon at 90° C. for 16 h. After complete conversion, the solvent was removed in vacuo, dichloromethane (400 mL) and water (350 mL) were added, the phases were separated and the aqueous phase was extracted with dichloromethane (3×400 mL). The combined organic extracts were dried over anhydrous sodium sulfate, filtered and after removing the solvent in vacuo, the crude product was purified by flash column chromatography on silica gel (PE:EA, 10:1, R_{f}=0.2) to afford 3′-hydroxy-2,2′-dimethyl-[1,1′-biphenyl]-3-carbaldehyde (22) (4.30 g, 19.0 mmol, 90%) as an off-white solid. mp=140° C. ¹H NMR (400 MHz, CDCl₃) (=10.38 (s, 1H), 7.84 (dd, ⁴J=1.8, ³J=7.4 Hz, 1H), 7.35-7.42 (m, 2H), 7.13 (t, ³J=7.8 Hz, 1H), 6.84 (d, ⁴J=7.8 Hz, 1H), 6.70 (d, ⁴J=7.4 Hz, 1H), 5.07 (s, 1H), 2.39 (s, 3H), 1.94 ppm (s, 3H). ¹³C NMR (101 MHz, CDCl₃) (=193.3, 154.1, 143.4, 142.1, 138.8, 135.1, 134.5, 131.2, 126.6, 126.0, 122.5, 122.1, 114.3, 15.8, 12.7 ppm. IR (ATR): \tilde{ν}=3332 (w), 2922 (w), 1681 (s), 1579 (m), 1449 (m), 1409 (w), 1380 (w), 1335 (w), 1307 (m), 1278 (s), 1213 (m), 1178 (w), 1144 (w), 1070 (m), 993 (w), 902 (m), 876 (w), 859 (w), 813 (w), 785 (s), 757 (m), 720 (s), 670 (m), 509 (w), 455 (w), 411 cm⁻¹ (w). MS (HR-ESI⁻): Exact mass calculated for [M−H]⁻: m/z=226.0994, measured: m/z=225.0920.

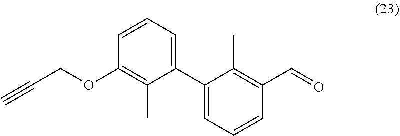 [SMILES: C#CCOc1cccc(-c2cccc(C=O)c2C)c1C]

2,2′-Dimethyl-3′-(prop-2-yn-1-yloxy)-[1,1′-biphenyl]-3-carbaldehyde (23). 3′-Hydroxy-2,2′-dimethyl-[1,1′-biphenyl]-3-carbaldehyde (22) (2.00 g, 8.83 mmol, 1.00 equiv.) reacted with propargyl bromide (80 wt. % in toluene, 1.48 mL, 13.3 mmol, 1.50 equiv.) and potassium carbonate (2.44 g, 17.7 mmol, 2.00 equiv.) as a base in abs. DMF (20 mL) according to GP-1. Purification by flash column chromatography on silica gel (PE:EA, 8:2, R_{f}=0.4) gave 2,2′-dimethyl-3′-(prop-2-yn-1-yloxy)-[1,1′-biphenyl]-3-carbaldehyde (23) (2.13 g, 8.06 mmol, 91%) as a colorless oil. ¹H NMR (600 MHz, CDCl₃) (=10.37 (s, 1H), 7.84 (d, ³J=6.5 Hz, 1H), 7.40 (t, ³J=7.5 Hz, 1H), 7.36 (d, ³J=6.5 Hz, 1H), 7.22 (t, ³J=8.1 Hz, 1H), 7.01 (d, ³J=8.1 Hz, 1H), 6.77 (d, ³J=7.5 Hz, 1H), 4.77 (d, ⁴J=2.4 Hz, 2H), 2.55 (t, ⁴J=2.4 Hz, 1H), 2.37 (s, 3H), 1.93 ppm (s, 3H). ¹³C NMR (151 MHz, CDCl₃) δ=193.2, 156.1, 143.4, 141.9, 138.7, 135.1, 131.1, 126.3, 126.0, 125.7, 122.8, 110.9, 79.0, 75.5, 56.3, 15.8, 13.1 ppm. IR (ATR): \tilde{ν}=3276 (m), 2924 (w), 2120 (w), 1678 (s), 1578 (m), 1451 (s), 1379 (w), 1306 (m), 1268 (m), 1241 (s), 1228 (m), 1200 (w), 1178 (m), 1139 (m), 1066 (m), 1007 (s), 926 (m), 868 (w), 788 (s), 724 (s), 695 (s), 646 (s), 595 (w), 565 (w), 502 (w), 486 cm⁻¹ (w). MS (HR-EI⁺): Exact mass calculated for [M]⁺: m/z=264.1150, measured: m/z=264.1133.

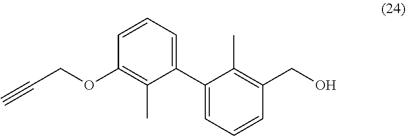 [SMILES: C#CCOc1cccc(-c2cccc(CO)c2C)c1C]

(2,2′-Dimethyl-3′-(prop-2-yn-1-yloxy)-[1,1′-biphenyl]-3-yl)methanol (24). 2,2′-Dimethyl-3′-(prop-2-yn-1-yloxy)-[1,1′-biphenyl]-3-carbaldehyde (23) (2.45 g, 9.27 mmol, 1.00 equiv.) was dissolved in an 1:1 mixture of abs. DCM/MeOH (20 mL) and was cooled down to 0° C. Sodium borohydride (0.53 g, 13.9 mmol, 1.50 equiv.) was added in small portions and after complete addition, the reaction mixture was allowed to stir at room temperature for 3 h. After complete conversion, water (50 mL) and ethyl acetate (50 mL) were added. The phases were separated, the aqueous phase was extracted with ethyl acetate (3×30 mL), the combined organic phases were dried over sodium sulfate, filtrated and concentrated under reduced pressure. The crude product was purified by flash column chromatography on silica gel (PE:EA, 8:2, R_{f}=0.3) to obtain (2,2′-dimethyl-3′-(prop-2-yn-1-yloxy)-[1,1′-biphenyl]-3-yl)methanol (24) (2.20 g, 8.26 mmol, 91%) as a colorless oil. ¹H NMR (600 MHz, CDCl₃) δ=7.39 (d, 3J=7.5 Hz, 1H), 7.25 (t, ³J=7.5 Hz, 1H), 7.20 (t, ³J=8.0 Hz, 1H), 7.08 (d, ³J=7.5 Hz, 1H), 6.98 (d, ³J=8.0 Hz, 1H), 6.79 (d, ³J=7.8 Hz, 1H), 4.76-4.77 (m, 4H), 2.54 (t, 4J=2.5 Hz, 1H), 2.05 (s, 3H), 1.93 ppm (s, 3H). ¹³C NMR (151 MHz, CDCl₃) δ=156.0, 143.3, 142.2, 139.0, 134.2, 129.2, 126.7, 126.0, 125.7, 125.6, 122.9, 110.5, 79.2, 75.4, 64.2, 56.3, 15.4, 13.1 ppm. IR (ATR): \tilde{ν}=3286 (m), 2920 (m), 1676 (w), 1576 (m), 1445 (m), 1372 (m), 1310 (w), 1256 (m), 1233 (w), 1180 (m), 1143 (m), 1085 (s), 1009 (s), 925 (w), 892 (w), 784 (s), 720 (m), 650 (m), 541 cm⁻¹ (w). MS (HR-ESI⁺): Exact mass calculated for [M+Na]⁺: m/z=289.1204, measured: m/z=289.1204.

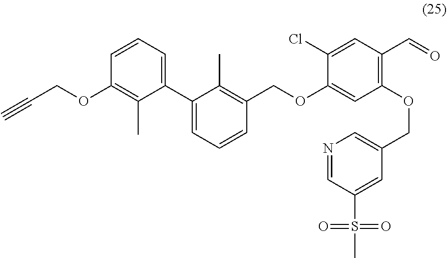 [SMILES: C#CCOc1cccc(-c2cccc(COc3cc(OCc4cncc(S(C)(=O)=O)c4)c(C=O)cc3Cl)c2C)c1C]

5-Chloro-4-((2,2′-dimethyl-3′-(prop-2-yn-1-yloxy)-[1,1′-biphenyl]-3-yl)methoxy)-2-((5-(methylsulfonyl)pyridin-3-yl)me-thoxy)benzaldehyde (25). (2,2′-Dimethyl-3′-(prop-2-yn-1-yloxy)-[1,1′-biphenyl]-3-yl)methanol (24) (300 mg, 878 μmol, 1.00 equiv.), phenol 6 (257 mg, 966 μmol, 1.10 equiv.) and triphenylphosphine (299 mg, 1.14 mmol, 1.30 equiv.) were dissolved in abs. DMF (3 mL) and reacted with DEAD (179 μL, 1.14 mmol, 1.30 equiv.) according to GP-2. Purification by flash column chromatography on silica gel (PE:EA, 4:6, R_{f}=0.2) gave compound 25 (444 mg, 752 μmol, 86%) as a colorless solid. mp=188° C. ¹H NMR (600 MHz, CDCl₃) δ=10.26 (s, 1H), 9.18 (d, ⁴J=2.1 Hz), 9.98 (d, ⁴J=2.1 Hz), 8.38 (d, ⁴J=2.1 Hz), 7.91 (s, 1H), 7.46 (d, ³J=7.0 Hz, 1H), 7.28 (t, ³J=7.7 Hz, 1H), 7.22 (t, ³J=7.7 Hz, 1H), 7.16-7.17 (m, 1H), 7.00 (d, ³J=7.7 Hz, 1H), 6.80 (d, ³J=7.0 Hz, 1H), 6.69 (s, 1H), 5.26 (s, 2H), 5.26 (s, 2H), 4.77 (d, ⁴J=2.4 Hz, 2H), 3.16 (s, 3H), 2.54 (t, ⁴J=2.4 Hz, 1H), 2.09 ppm (s, 3H). ¹³C NMR (151 MHz, CDCl₃) δ=186.5, 160.2, 160.1, 156.0, 153.3, 148.7, 142.8, 142.6, 137.4, 134.8, 134.5, 133.3, 132.3, 130.9, 130.3, 127.6, 126.1, 125.9, 125.6, 122.8, 119.5, 117.6, 110.7, 98.8, 79.1, 75.5, 70.7, 68.0, 56.3, 45.0, 15.9, 13.1 ppm. IR (ATR): G=3270 (m), 2924 (w), 1662 (m), 1592 (s), 1498 (w), 1457 (m), 1436 (w), 1413 (m), 1378 (m), 1306 (s), 1274 (s), 1249 (s), 1233 (m), 1214 (w), 1177 (m), 1136 (s), 1107 (m), 1093 (w), 1025 (s), 959 (m), 906 (w), 871 (m), 817 (w), 801 (w), 768 (m), 752 (w), 717 (m), 698 (s), 698 (m), 650 (m), 592 (w), 550 (m), 529 (s), 505 (w), 432 cm⁻¹ (w). MS (HR-ESI⁺): Exact mass calculated for [M+H]⁺: m/z=590.1404, measured: m/z=590.1399.

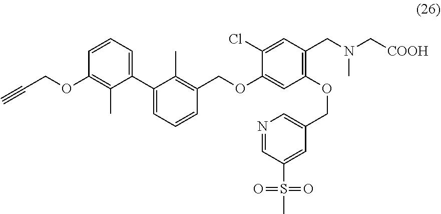 [SMILES: C#CCOc1cccc(-c2cccc(COc3cc(OCc4cncc(S(C)(=O)=O)c4)c(CN(C)CC(=O)O)cc3Cl)c2C)c1C]

N-(5-Chloro-4-((2,2′-dimethyl-3′-(prop-2-yn-1-yloxy)-[1,1′-biphenyl]-3-yl)methoxy)-2-((5-methylsulfonyl)pyridin-3-yl)methoxy)benzyl)-N-methylglycine (26). Aldeyhde (25) (50.0 mg, 84.7 mmol, 1.00 equiv.), sarcosine (14.6 mg, 339 μmol, 4.00 equiv.) and sodium cyanoborohydride (8.0 mg, 127 μmol, 1.50 equiv.) were reacted in an 1:1 mixture of abs. DMF/MeOH (2 mL) and subsequently worked up according to GP-3. After flash column chromatography on silica gel (MeCN:MeOH:Et₃N, 100:20:1, R_{f}=0.2) compound (26) (21.2 mg, 32.3 μmol, 38%) was obtained as a colorless solid. mp=103° C. Rₜ=12.10 min (System A), purity: 99.6%. ¹H NMR (400 MHz, methanol-d₄) δ=9.11 (s, 1H), 9.03 (s, 1H), 8.56 (s, 1H), 7.56 (s, 1H), 7.47 (d, ³J=7.6 Hz, 1H), 7.27-7.19 (m, 2H), 7.13-7.09 (m, 2H), 7.04 (d, ³J=8.3 Hz, 1H), 6.74 (d, 3J=7.6 Hz, 1H), 5.45 (s, 2H), 5.32 (s, 2H), 4.80 (s, 2H), 4.41 (s, 2H), 3.83 (s, 2H), 3.27 (s, 3H), 2.94 (s, 1H), 2.84 (s, 3H), 2.09 (s, 3H), 1.89 ppm (s, 3H). ¹³C NMR (101 MHz, methanol-d₄) δ=168.9, 158.3, 158.3, 157.3, 154.5, 149.0, 144.2, 143.7, 139.0, 136.6, 136.3, 135.7, 135.0, 130.9, 129.1, 127.1, 126.6, 126.3, 123.5, 116.7, 112.1, 112.0, 11.4, 101.1, 80.1, 76.5, 71.6, 68.9, 57.7, 57.0, 55.4, 44.5, 41.8, 15.9, 13.1 ppm. IR (ATR): \tilde{ν}=3285 (w), 3012 (w), 2922 (w), 1732 (w), 1673 (w), 1634 (w), 1605 (m), 1575 (m), 1505 (w), 1455 (w), 1406 (w), 1380 (w), 1303 (m), 1269 (w), 1255 (w), 1234 (m), 1198 (m), 1170 (m), 1143 (s), 1089 (w), 1077 (w), 1014 (m), 965 (w), 891 (w), 798 (w), 784 (w), 766 (m), 719 (m), 694 cm⁻¹ (w). MS (HR-ESI⁻): Exact mass calculated for [M−H]⁻: m/z=661.1781, measured: m/z=661.1783.

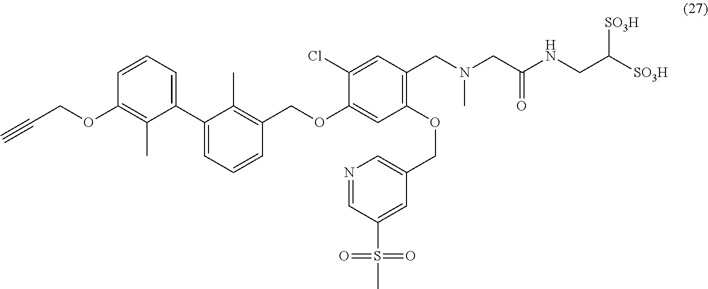 [SMILES: C#CCOc1cccc(-c2cccc(COc3cc(OCc4cncc(S(C)(=O)=O)c4)c(CN(C)CC(=O)NCC(S(=O)(=O)O)S(=O)(=O)O)cc3Cl)c2C)c1C]

Bis(sulfonic acid) (27). Carboxylic acid (26) (120 mg, 185 μmol, 1.00 equiv.) reacted with 2-aminoethane-1,1-disulfonic acid (TBA salt) (18) (361 mg, 924 μmol, 5.00 equiv.) in presence of DIPEA (64.5 μL, 370 μmol, 2.00 equiv.) and HTBU (154 mg, 407 μmol, 2.20 equiv.) in abs. DMF (2 mL) according to GP-6. Purification was performed by preparative RP-HPLC (System D, Rₜ=25 min) and after cation exchange and lyophilization, bis(sulfonated) product 27 (58.0 mg, 68.2 μmol, 37%) was obtained as a pale-brownish solid. mp=180-185° C. (decomposition). Rₜ=10.79 min (System A), purity: 90.3%. ¹H NMR (400 MHz, DMF-d₇) δ=9.66 (bs, 1H), 9.49 (s, 1H), 9.27 (s, 1H), 8.86 (s, 1H), 8.26 (s, 1H), 7.81 (s, 1H), 7.66 (d, ³J=7.5 Hz, 1H), 7.47 (s, 1H), 7.36-7.27 (m, 2H), 7.16-7.14 (m, 2H), 6.80 (d, ³J=7.5 Hz, 1H), 5.70 (s, 2H), 5.47 (s, 2H), 4.96 (s, 2H), 4.67-4.62 (m, 2H), 4.05-4.04 (m, 2H), 3.90 (s, 1H), 3.58 (t, 4J=2.4 Hz, 1H), 3.53 (s, 3H), 3.03 (s, 3H), 2.14 (s, 3H), 1.92 ppm (s, 3H). ¹³C NMR (101 MHz, DMF-d₇) δ=164.6, 157.6, 156.7, 156.2, 151.2, 143.2, 142.3, 138.6, 138.2, 135.3, 134.6, 129.8, 128.4, 126.5, 125.9, 124.9, 122.6, 114.4, 112.0, 111.1, 109.8, 100.6, 79.8, 77.5, 7.46, 70.4, 67.9, 57.0, 56.3, 53.8, 43.8, 41.1, 39.5, 15.4, 12.7 ppm. IR (ATR): \tilde{ν}=3289 (m), 3055 (w), 2924 (w), 1682 (m), 1606 (w), 1576 (w), 1506 (w), 1456 (m), 1409 (w), 1307 (m), 1234 (s), 1177 (s), 1154 (s), 1090 (w), 1063 (m), 1016 (m), 768 (w), 722 (w), 674 (w), 590 cm⁻¹ (w). MS (HR-ESI⁻): Exact mass calculated for [M−H]⁻: m/z=848.1390, measured: m/z=848.1397.

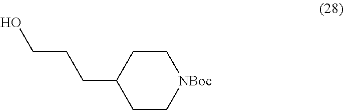 [SMILES: CC(C)(C)OC(=O)N1CCC(CCCO)CC1]

was synthesized according to literature [11].

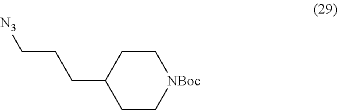 [SMILES: CC(C)(C)OC(=O)N1CCC(CCCN=[N+]=[N-])CC1]

tert-Butyl 4-(3-azidopropyl)piperidine-1-carboxylate (29). tert-Butyl 4-(3-hydroxypropyl)piperidine-1-carboxylate (28) (320 mg, 1.32 mmol, 1.00 equiv.) was dissolved in abs. THF (3 mL) and ADMP (450 mg, 1.58 mmol, 1.20 equiv.) and DBU (255 μL, 1.71 mmol, 1.30 equiv.) were added. The reaction mixture was stirred at room temperature for 16 h and after complete conversion, DCM (10 mL) and conc. NH₄Cl solution (10 mL) were added, phases were separated and the aqueous phase was extracted with DCM (3×10 mL). The combined organic fractions were dried over sodium sulfate, filtrated and concentrated under reduced pressure. Purification by flash column chromatography on silica gel (DCM:EA, 90:10, R_{f}=0.3) lead to tert-Butyl 4-(3-azidopropyl)piperidine-1-carboxylate (29) (300 mg, 1.18 mmol, 85%) as a colorless oil. ¹H NMR (400 MHz, CDCl₃) δ=4.10-4.06 (m, 2H), 3.26 (t, ³J=6.9 Hz, 2H), 2.71-2.64 (m, 2H), 1.64-1.60 (m, 4H), 1.45 (s, 9H), 1.40-1.39 (m, 1H), 1.34-1.28 (m, 2H), 1.14-1.04 ppm (m, 2H). ¹³C NMR (101 MHz, CDCl₃) δ=155.0, 79.4, 51.8, 44.1, 35.9, 33.7, 32.2, 28.6, 26.2 ppm. IR (ATR): G=2975 (w), 2928 (m), 2851 (w), 2092 (s), 1688 (s), 1452 (w), 1419 (m), 1364 (m), 1276 (m), 1243 (m), 1161 (s), 1134 (m), 1089 (w), 971 (w), 934 (w), 868 (w), 768 cm⁻¹ (w). MS (HR-ESI⁺): Exact mass calculated for [M+Na]⁺: m/z=291.1791, measured: m/z=291.1790.

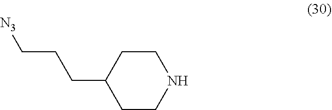 [SMILES: [N-]=[N+]=NCCCC1CCNCC1]

4-(3-Azidopropyl)piperidine (30). tert-Butyl 4-(3-azidopropyl)piperidine-1-carboxylate (29) (300 mg, 1.12 mmol, 1.00 equiv.) was dissolved at 0° C. in an 1:1 mixture of DCM/TFA (3 mL) and the mixture was stirred at room temperature for 16 h. The solvents were removed in vacuo to yield 4-(3-azidopropyl)piperidine (30) (187 mg, 1.12 mmol, quant.) as a yellowish oil, which was used without further purification. ¹H NMR (400 MHz, CDCl₃) δ=5.99 (bs, 1H), 3.28-3.20 (m, 4H), 2.74-2.67 (m, 2H), 1.78-1.75 (m, 2H), 1.64-1.57 (m, 2H), 1.45-1.40 (m, 1H), 1.36-1.29 ppm (m, 4H). ¹³C NMR (400 MHz, CDCl₃) δ=51.7, 45.5, 35.1, 33.7, 31.3, 26.0 ppm. IR (ATR): G=2927 (m), 2854 (m), 2510 (w), 2360 (w), 2092 (s), 1673 (s), 1454 (w), 1256 (w), 1199 (m), 1174 (m), 1127 (m), 831 (w), 798 (w), 720 cm⁻¹ (m). MS (HR-EI⁺): Exact mass calculated for [M]⁺: m/z=168.1375, measured: m/z=168.1359.

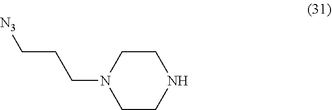 [SMILES: [N-]=[N+]=NCCCN1CCNCC1]

was synthesized according to literature [12].

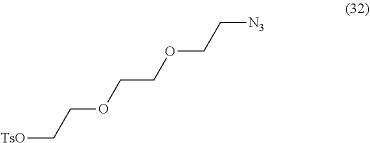 [SMILES: Cc1ccc(S(=O)(=O)OCCOCCOCCN=[N+]=[N-])cc1]

2-(2-(2-Azidoethoxy)ethoxy)ethyl 4-methylbenzenesulfonate (32). 2-(2-(2-Azidoethoxy)ethoxy)ethan-1-ol (230 mg, 1.31 mmol, 1.00 equiv.) was dissolved in abs. DCM (5 mL) under argon. After the addition of tosylchlorid (376 mg, 1.97 mmol, 1.50 equiv.) and pyridine (212 μL, 2.63 mmol, 2.00 equiv.) at 0° C., the reaction mixture was stirred at room temperature for 16 h. The mixture was diluted with ethyl acetate (50 mL), 1M HCl (50 mL) was added, the phases were separated and the aqueous phase was extracted with ethyl acetate (3×40 mL). The combined organic fractions were dried over sodium sulfate, filtrated and concentrated under reduced pressure. Purification by flash column chromatography on silica gel (PE:EA, 7:3, R_{f}=0.3) yielded 2-(2-(2-azidoethoxy)ethoxy)ethyl 4-methylbenzenesulfonate (32) (404 mg, 1.23 mmol, 93%) as a colorless oil. ¹H NMR (400 MHz, CDCl₃) (=7.80 (d, ³J=8.3 Hz, 2H), 7.34 (d, ³J=8.3 Hz, 2H), 4.18-4.15 (m, 2H), 3.71-3.69 (m, 2H), 3.65-3.63 (m, 2H), 3.60 (s, 4H), 3.38-3.35 (m, 2H), 2.45 ppm (s, 3H). ¹³C NMR (101 MHz, CDCl₃) δ=144.9, 133.2, 130.0, 128.1, 70.9, 70.8, 70.2, 69.4, 68.9, 50.8, 21.8 ppm. IR (ATR): \tilde{ν}=2952 (m), 2921 (m), 2854 (w), 2100 (m), 1598 (w), 1454 (w), 1354 (m), 1290 (w), 1189 (m), 1175 (s), 11230 (m), 1096 (m), 1017 (w), 919 (m), 816 (m), 774 (w), 690 (w), 662 (m), 584 (w), 553 cm⁻¹ (m). MS (HR-ESI⁺): Exact mass calculated for [M+H]⁺: m/z=352.0943, measured: m/z=352.0936.

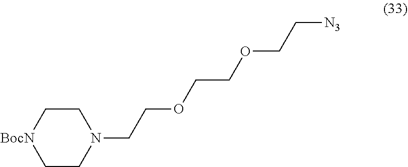 [SMILES: CC(C)(C)OC(=O)N1CCN(CCOCCOCCN=[N+]=[N-])CC1]

tert-Butyl 4-(2-(2-(2-azidoethoxy)ethoxy)ethyl)piperazine-1-carboxylate (33). tert-Butyl piperazine-1-carboxylate (190 mg, 1.02 mmol, 1.00 equiv.) reacted with 2-(2-(2-azidoethoxy)ethoxy)ethyl 4-methylbenzenesulfonate (32) (386 mg, 1.17 mmol, 1.15 equiv.) and potassium carbonate (282 mg, 2.04 mmol, 2.00 equiv.) as a base in abs. DMF (5 mL) according to GP-1. Purification by flash column chromatography on silica gel (DCM:MeOH, 94:6, R_{f}=0.2) provided tert-butyl 4-(2-(2-(2-azidoethoxy)ethoxy)ethyl)piperazine-1-carboxylate (33) (255 mg, 724 μmol, 73%) as a colorless oil. ¹H NMR (400 MHz, methanol-d₄) δ=3.71-3.66 (m, 8H), 3.46-3.39 (m, 6H), 2.66-2.63 (m, 2H), 2.54-2.51 (m, 4H), 1.48 ppm (s, 9H). ¹³C NMR (101 MHz, methanol-d₄) δ=156.4, 81.2, 71.6, 71.5, 71.1, 69.7, 62.2, 58.7, 54.3, 51.8, 286 ppm. IR (ATR): G=2974 (w), 2927 (m), 2865 (m), 2810 (w), 2100 (m), 1690 (s), 1457 (w), 1418 (m), 1392 (w), 1365 (m), 1289 (m), 1244 (m), 1169 (m), 1119 (s), 1004 (m), 937 (w), 865 (w), 831 (w), 796 cm⁻¹ (w). MS (HR-ESI⁺): Exact mass calculated for [M+Na]⁺: m/z=344.2298, measured: m/z=344.2293.

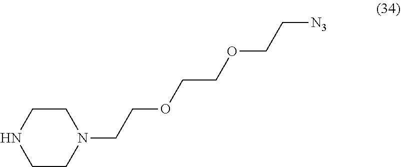 [SMILES: [N-]=[N+]=NCCOCCOCCN1CCNCC1]

1-(2-(2-(2-Azidoethoxy)ethoxy)ethyl)piperazine (34). tert-Butyl 4-(2-(2-(2-azidoethoxy)ethoxy)ethyl)piperazine-1-carboxylate (33) (90.0 mg, 262 μmol, 1.00 equiv.) was dissolved at 0° C. in an 1:1 mixture of DCM/TFA (3 mL) and was stirred at room temperature for 16 h. The solvents were removed in vacuo to yield 1-(2-(2-(2-azidoethoxy)ethoxy)ethyl)piperazine (34) (63.8 mg, 261 μmol, quant.) as a yellow oil, which was used without further purification. ¹H NMR (400 MHz, methanol-d₄) δ=3.85-3.82 (m, 2H), 3.70-3.68 (m, 6H), 3.49-3.46 (m, 4H), 3.43-3.40 (m, 2H), 3.38-3.37 (m, 4H), 3.24-3.21 ppm (m, 2H). ¹³C NMR (101 MHz, methanol-d₄) δ=71.5, 71.4, 71.0, 67.2, 57.9, 51.8, 50.6, 43.0 ppm. IR (ATR): G=3445 (w), 3015 (w), 2873 (w), 2747 (w), 2487 (w), 2360 (m), 2342 (w), 2110 (m), 1668 (s), 1456 (m), 1423 (m), 1302 (w), 1176 (s), 1121 (s), 1009 (w), 916 (w), 832 (m), 798 (m), 720 (m), 595 cm⁻¹ (w). MS (HR-ESI⁺): Exact mass calculated for [M+H]⁺: m/z=244.1773, measured: m/z=244.1767.

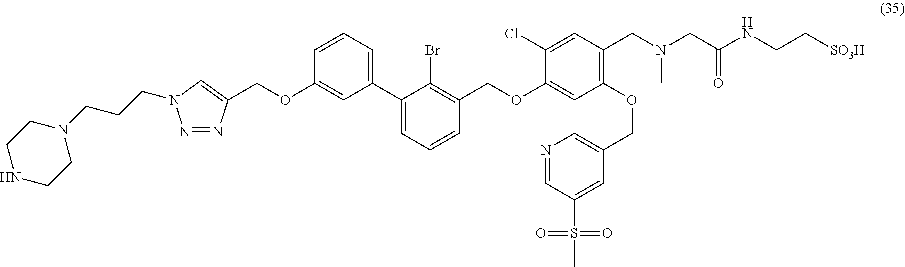 [SMILES: CN(CC(=O)NCCS(=O)(=O)O)Cc1cc(Cl)c(OCc2cccc(-c3cccc(OCc4cn(CCCN5CCNCC5)nn4)c3)c2Br)cc1OCc1cncc(S(C)(=O)=O)c1]

2-(2-((4-((2-Bromo-3′-((1-(3-(piperidin-4-yl)propyl)-1H-1,2,3-triazol-4-yl)methoxy)[1,1′-biphenyl]-3-yl)methoxy)-5-chloro-2-((5-(methylsulfonyl)pyridin-3-yl)methoxy)benzyl)(methyl)amino)acetamido)ethane-1,1-disulfonic acid (35). Alkyne 19 (15.0 mg, 16.4 μmol, 1.00 equiv.) and azide linker 30 (4.1 mg, 24.6 μmol, 1.50 equiv.) were dissolved in DMF (1 mL), reacted in presence of TBTA (0.1 mg, 0.2 μmol, 0.01 equiv.) and [Cu(MeCN)₄]PF₆ (2.5 mg, 6.8 μmol, 0.50 equiv.) according to GP-4. Purification by semi-preparative RP-HPLC (System C, Rₜ=21 min) yielded compound 35 (11.5 mg, 10.8 μmol, 66%) as a colorless powder. mp=140° C. (decomposition). Rₜ=9.42 min (System A), purity: 97.0%. ¹H NMR (400 MHz, DMSO-d₆) δ=9.55 (bs, 1H), 9.12 (bs, 2H), 8.48 (s, 1H), 8.29 (s, 1H), 8.22 (s, 1H), 8.06 (bs, 2H), 7.68-7.64 (m, 2H), 7.53-7.49 (m, 1H), 7.41-7.37 (m, 2H), 7.20 (s, 1H), 7.09 (d, ³J=8.1 Hz, 1H), 7.04 (s, 1H), 6.96 (d, ³J=7.5 Hz, 1H), 5.49-5.48 (m, 2H), 5.38 (s, 2H), 4.37-4.33 (m, 4H), 3.82 (s, 2H), 3.61-3.54 (m), 3.22-3.19 (m, 2H), 2.73 (s, 4H), 1.80-1.79 (m, 2H), 1.69-1.66 (m, 2H), 1.53-1.45 (m, 2H), 1.28-1.23 (m, 2H), 1.14-1.12 ppm. (m, 2H). ¹³C NMR (101 MHz, DMSO-d₆) δ=163.4, 157.6, 156.9, 155.6, 153.3, 151.2, 147.3, 142.8, 142.6, 142.1, 136.0, 134.4, 134.0, 131.3, 129.6, 129.4, 128.0, 127.7, 125.5, 124.4, 123.3, 121.8, 115.9, 114.3, 113.4, 109.8, 100.2, 73.8, 71.2, 67.5, 61.3, 53.8, 50.6, 49.4, 43.7, 43.3, 41.0, 32.5, 32.1, 28.3, 28.1, 26.7, 25.2 ppm. IR (ATR): \tilde{ν}=3445 (m), 3055 (m), 2928 (m), 2857 (w), 1682 (m), 1605 (w), 1580 (w), 1506 (w), 1456 (w), 1406 (w), 1304 (m), 1200 (s), 1146 (s), 1064 (m), 1021 (m), 799 (w), 779 (w), 719 (w), 700 (w), 652 (w), 596 cm⁻¹ (w). MS (HR-ESI⁺): Exact mass calculated for [M+H]⁺: m/z=1068.1703, measured: m/z=1068.1697.

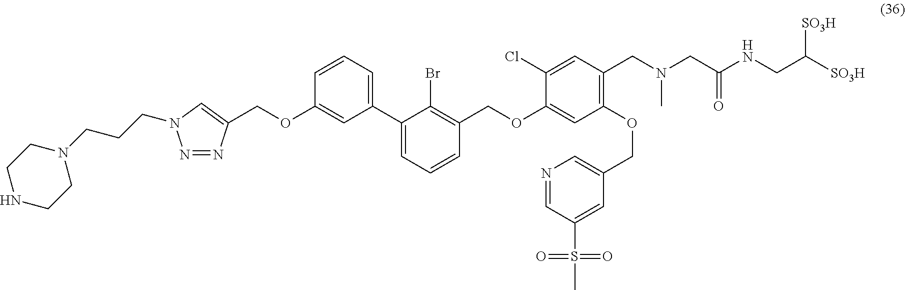 [SMILES: CN(CC(=O)NCC(S(=O)(=O)O)S(=O)(=O)O)Cc1cc(Cl)c(OCc2cccc(-c3cccc(OCc4cn(CCCN5CCNCC5)nn4)c3)c2Br)cc1OCc1cncc(S(C)(=O)=O)c1]

2-(2-((4-((2-Bromo-3′-((1-(3-(piperazin-1-yl)propyl)-1H-1,2,3-triazol-4-yl)methoxy)[1,1′-biphenyl]-3-yl)methoxy)-5-chloro-2-((5-(methylsulfonyl)pyridin-3-yl)methoxy)benzyl)(methyl)amino)acetamido)ethane-1,1-disulfonic acid (36). Alkyne 19 (15.0 mg, 16.4 μmol, 1.00 equiv.) and azide linker 31 (3.5 mg, 24.6 μmol, 1.50 equiv.) were dissolved in DMF (1 mL), reacted in presence of TBTA (0.1 mg, 0.2 μmol, 0.01 equiv.) and [Cu(MeCN)₄]PF₆ (2.5 mg, 6.8 μmol, 0.50 equiv.) according to GP-4. Purification by semi-preparative RP-HPLC (System C, Rₜ=20 min) yielded compound 36 (5.1 mg, 4.8 μmol, 29%) as a colorless powder. mp=150-153° C. Rₜ=10.06 min (System A), purity: 100%. ¹H NMR (400 MHz, DMSO-d₆) δ=9.69 (bs, 1H), 9.06-9.05 (m, 2H), 8.45 (s, 1H), 8.25 (s, 1H), 8.06 (s, 1H), 7.67-7.63 (m, 2H), 7.50 (t, 3J=7.5 Hz, 2H), 7.41-7.37 (m, 2H), 7.15 (s, 1H), 7.09 (d, ³J=8.1 Hz, 1H), 7.04 (s, 1H), 6.95 (d, ³J=7.6 Hz, 1H), 5.43 (s, 4H), 5.19 (s, 2H), 4.45 (bs, 2H), 4.27 (bs, 2H), 3.75-3.37 (m), 2.73 (s, 2H), 2.24 ppm (bs, 2H). ¹³C NMR (101 MHz, DMSO-d₆) δ=163.6, 158.1, 157.8, 157.5, 156.9, 155.5, 153.2, 147.3, 142.9, 142.6, 142.0, 136.9, 136.1, 134.4, 134.0, 132.8, 131.3, 129.7, 129.5, 127.8, 124.7, 123.4, 121.7, 116.0, 114.2, 113.5, 109.6, 100.3, 73.9, 71.2, 67.4, 61.2, 55.3, 53.8, 52.7, 48.5, 46.9, 43.7, 41.2, 38.7 ppm.

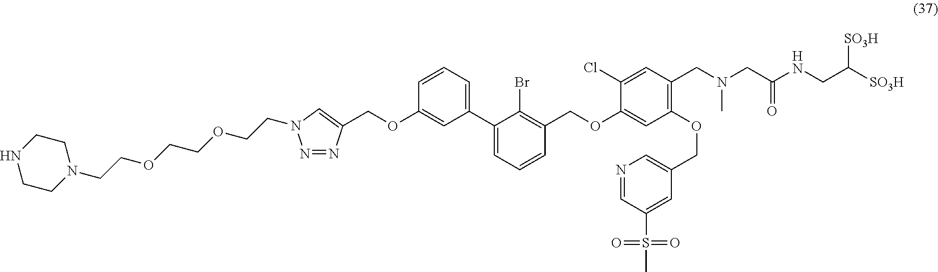 [SMILES: CN(CC(=O)NCC(S(=O)(=O)O)S(=O)(=O)O)Cc1cc(Cl)c(OCc2cccc(-c3cccc(OCc4cn(CCOCCOCCN5CCNCC5)nn4)c3)c2Br)cc1OCc1cncc(S(C)(=O)=O)c1]

2-(2-((4-((2-Bromo-3′-((1-(2-(2-(2-(piperazin-1-yl)ethoxy)ethoxy)ethyl)-1H-1,2,3-triazol-4-yl)methoxy)-[1,1′-biphenyl]-3-yl)methoxy)-5-chloro-2-((5-(methylsulfonyl)pyridin-3-yl)methoxy)benzyl)(methyl)amino)acetamido)ethane-1,1-disulfonic acid (37). Alkyne 19 (20.0 mg, 21.9 μmol, 1.00 equiv.) and azide linker 34 (8.0 mg, 32.8 μmol, 1.50 equiv.) were dissolved in DMF (1 mL), reacted in presence of TBTA (0.1 mg, 0.2 μmol, 0.01 equiv.) and [Cu(MeCN)₄]PF6 (3.5 mg, 9.4 μmol, 0.50 equiv.) according to GP-4. Purification by semi-preparative RP-HPLC (System C, Rₜ=25 min) yielded compound (37) (17.0 mg, 14.9 μmol, 68%) as a colorless powder. mp=165-168° C. Rₜ=9.06 min (System A), purity: 95.6%. ¹H NMR (400 MHz, DMSO-d₆) δ=9.67 (bs, 1H), 9.07 (s, 2H), 8.47 (s, 1H), 8.22 (s, 1H), 8.07 (bs, 1H), 7.67-7.63 (m, 2H), 7.51 (t, 3J=7.5 Hz, 1H), 7.42-7.38 (m, 2H), 7.18 (bs, 1H), 7.11-7.09 (m, 1H), 7.06 (bs, 1H), 6.95 (d, ³J=7.5 Hz, 1H), 5.47 (s, 2H), 5.39 (s, 2H), 5.20 (s, 2H), 4.55-4.53 (m, 2H), 4.28 (bs, 2H), 3.84-3.37 (m), 2.75 ppm (s, 3H). ¹³C NMR (101 MHz, DMSO-d₆) δ=157.5, 156.9, 155.6, 153.3, 147.3, 142.9, 142.5, 142.1, 136.9, 136.0, 134.5, 134.0, 132.8, 131.3, 129.6, 129.4, 127.8, 124.9, 132.4, 121.8, 115.8, 114.3, 113.5, 109.6, 100.2, 73.9, 71.3, 69.3, 69.2, 68.6, 67.5, 64.7, 61.2, 55.7, 52.9, 49.5, 48.6, 43.7, 41.6, 41.5 ppm. IR (ATR): \tilde{ν}=3444 (w), 3023 (w), 2874 (w), 1679 (m), 1605 (w), 1580 (w), 1506 (w), 1461 (w), 1406 (w), 1304 (m), 1199 (s), 1175 (s), 1145 (s), 1063 (m), 1019 (m), 916 (w), 829 (w), 798 (w), 778 (w), 720 (w), 700 (w), 650 (w), 593 cm⁻¹ (w). MS (HR-ESI⁺): Exact mass calculated for [M+H]⁺: m/z=1143.2023, measured: m/z=1143.2010.

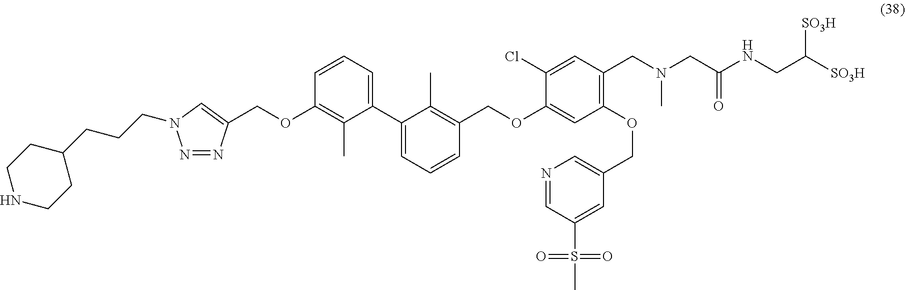 [SMILES: Cc1c(COc2cc(OCc3cncc(S(C)(=O)=O)c3)c(CN(C)CC(=O)NCC(S(=O)(=O)O)S(=O)(=O)O)cc2Cl)cccc1-c1cccc(OCc2cn(CCCC3CCNCC3)nn2)c1C]

2-(2-((5-Chloro-4-((2,2′-dimethyl-3′-((1-(3-(piperidin-4-yl)propyl)-1H-1,2,3-triazol-4-yl)methoxy)-[1,1′-biphenyl]-3-yl)methoxy)-2-((5-(methylsulfonyl)pyridin-3-yl)methoxy)benzyl)(methyl)amino)acetamido)ethane-1,1-disulfonic acid (38). Alkyne 27 (26.0 mg, 30.6 μmol, 1.00 equiv.) and azide linker 30 (7.7 mg, 45.9 μmol, 1.50 equiv.) were dissolved in DMF (1 mL), reacted in presence of TBTA (0.1 mg, 0.2 μmol, 0.01 equiv.) and [Cu(MeCN)₄]PF6 (4.3 mg, 11.5 μmol, 0.50 equiv.) according to GP-4. Purification by semi-preparative RP-HPLC (System C, Rₜ=20 min) yielded compound 38 (19.4 mg, 18.6 μmol, 61%) as a colorless powder. mp=197-200° C. (decomposition). Rₜ=9.24 min (System A), purity: 96.2%. ¹H NMR (400 MHz, DMSO-d₆) δ=9.61 (bs, 1H), 9.10 (bs, 2H), 8.49 (s, 1H), 8.26-8.22 (m, 2H), 8.05 (bs, 2H), 7.61 (s, 1H), 7.50 (d, ³J=7.6 Hz, 1H), 7.29-7.21 (m, 2H), 7.15 (d, ³J=8.1 Hz, 1H), 7.08 (d, 3J=7.7 Hz, 1H), 6.73 (d, ³J=7.5 Hz, 1H), 5.49-5.45 (m, 2H), 5.35-5.34 (m, 2H), 5.25-5.16 (m, 2H), 4.38-4.27 (m, 3H), 3.83 (bs, 2H), 3.61-3.38 (m), 3.21-3.18 (m, 2H), 2.74-2.73 (m, 4H), 2.02 (s, 3H), 1.83-1.77 (m, 4H), 1.68-1.64 (m, 2H), 1.44 (bs, 2H), 1.29-1.23 (m, 3H), 1.12-1.10 ppm (m, 2H). ¹³C NMR (101 MHz, DMSO-d₆) δ=163.4, 156.0, 154.2, 153.3, 151.9, 147.3, 143.1, 142.2, 141.6, 134.5, 134.4, 133.9, 129.3, 128.0, 126.3, 125.5, 124.3, 124.1, 121.7, 113.4, 109.6, 100.3, 73.9, 69.9, 67.4, 61.9, 55.7, 52.9, 49.3, 43.7, 43.3, 41.3, 32.4, 32.0, 28.1, 26.7, 15.3, 12.8 ppm. IR (ATR): \tilde{ν}=3451 (w), 3015 (w), 2929 (w),1682 (m), 1606 (w), 1576 (w), 1506 (w), 1456 (m), 1409 (w), 1306 (m), 1199 (s), 1179 (s), 1146 (s), 1091 (w), 1064 (w), 1019 (m), 798 (w), 769 (w), 720 (w), 592 cm⁻¹ (w). MS (HR-ESI⁺): Exact mass calculated for [M+H]⁺: m/z=1018.2911, measured: m/z=1018.2910.

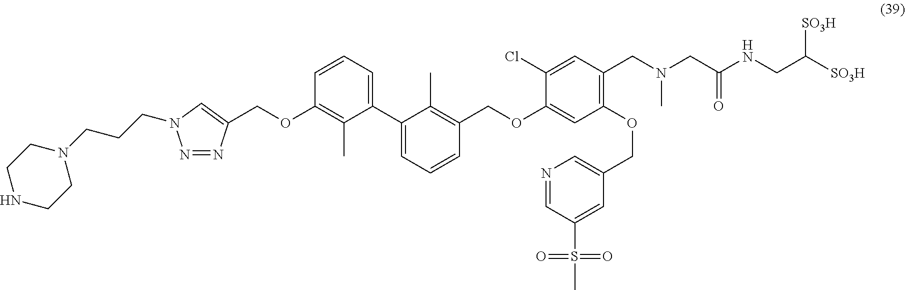 [SMILES: Cc1c(COc2cc(OCc3cncc(S(C)(=O)=O)c3)c(CN(C)CC(=O)NCC(S(=O)(=O)O)S(=O)(=O)O)cc2Cl)cccc1-c1cccc(OCc2cn(CCCN3CCNCC3)nn2)c1C]

2-(2-((5-Chloro-4-((2,2′-dimethyl-3′-((1-(3-(piperazin-1-yl)propyl)-1H-1,2,3-triazol-4-yl)methoxy)-[1,1′-biphenyl]-3-yl)methoxy)-2-((5-(methylsulfonyl)pyridin-3-yl)methoxy)benzyl)(methyl)amino)acetamido)ethane-1,1-disulfonic acid (39). Alkyne 27 (15.0 mg, 17.6 μmol, 1.50 equiv.) and azide linker 31 (3.7 mg, 26.5 μmol, 1.50 equiv.) were dissolved in DMF (1 mL), reacted in presence of TBTA (0.1 mg, 0.2 μmol, 0.01 equiv.) and [Cu(MeCN)₄]PF₆ (2.6 mg, 6.9 μmol, 0.50 equiv.) according to GP-4. Purification by semi-preparative RP-HPLC (System C, Rₜ=20 min) yielded compound 39 (8.1 mg, 7.8 μmol, 45%) as a colorless powder. mp=155-159° C. Rₜ=9.09 min (System A), purity: 98.8%. ¹H NMR (400 MHz, DMSO-d₆) δ=9.66 (bs, 1H), 9.09 (bs, 2H), 8.47 (s, 1H), 8.27 (s, 1H), 8.12 (s, 1H), 7.59 (s, 1H), 7.50 (d, ³J=7.3 Hz, 1H), 7.25-7.15 (m, 3H), 7.07 (d, ³J=7.4 Hz, 1H), 6.72 (d, ³J=7.4 Hz, 1H), 5.42-5.38 (m, 4H), 5.23-5.16 (m, 2H), 4.48-4.45 (m, 2H), 4.26 (bs, 2H), 3.78-3.37 (m), 3.04 (bs, 2H), 2.70 (s, 3H), 2.26 (bs, 2H), 2.01 (s, 3H), 1.73 ppm (s, 3H). ¹³C NMR (101 MHz, DMSO-d₆) (=163.6, 156.8, 156.1, 155.5, 153.2, 147.4, 143.2, 142.2, 141.7, 137.0, 135.0, 134.4, 134.4, 133.8, 132.8, 129.3, 128.3, 126.4, 125.5, 124.3, 124.2, 121.6, 113.6, 110.9, 109.6, 100.5, 73.9, 70.0, 67.4, 61.9, 55.5, 53.7, 52.9, 48.4, 46.9, 43.7, 41.1, 24.3, 15.5, 12.7 ppm.

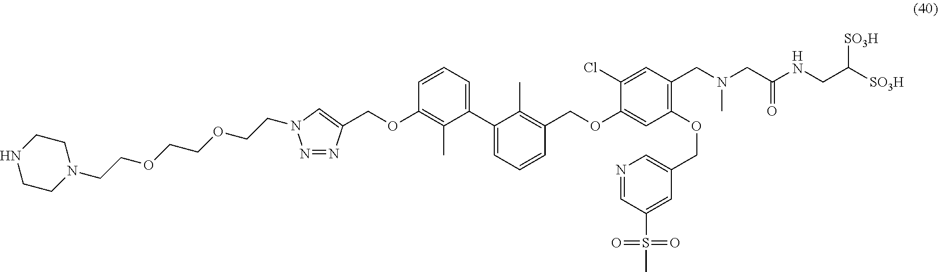 [SMILES: Cc1c(COc2cc(OCc3cncc(S(C)(=O)=O)c3)c(CN(C)CC(=O)NCC(S(=O)(=O)O)S(=O)(=O)O)cc2Cl)cccc1-c1cccc(OCc2cn(CCOCCOCCN3CCNCC3)nn2)c1C]

2-(2-((5-Chloro-4-((2,2′-dimethyl-3′-((1-(2-(2-(2-(piperazin-1-yl)ethoxy)ethoxy)ethyl)-1H-1,2,3-triazol-4-yl)methoxy)-[,1,1′-biphenyl]-3-yl)methoxy)-2-((5-(methylsulfonyl)pyridin-3-yl)methoxy)benzyl)(methyl)amino)acetamido)ethane-1,1-disulfonic acid (40). Alkyne 27 (20.0 mg, 23.5 μmol, 1.00 equiv.) and azide linker 34 (8.6 mg, 35.3 μmol, 1.50 equiv.) were dissolved in DMF (1 mL), reacted in presence of TBTA (0.1 mg, 0.2 μmol, 0.01 equiv.) and [Cu(MeCN)₄]PF₆ (3.6 mg, 9.5 μmol, 0.50 equiv.) according to GP-4. Purification by semi-preparative RP-HPLC (System C, Rₜ=22 min) yielded compound 40 (14.0 mg, 12.8 μmol, 54%) as a colorless powder. mp=173-176° C. Rₜ=9.17 min (System A), purity: 89.8%. ¹H NMR (400 MHz, DMSO-d₆) δ=9.64 (bs, 1H), 9.09 (s, 2H), 8.49 (s, 1H), 8.21 (s, 1H), 8.10 (bs, 1H), 7.59 (s, 1H), 7.50-7.48 (m, 1H), 7.29-7.17 (m, 3H), 7.09 (d, ³J=7.5 Hz, 1H), 6.73 (d, ³J=7.2 Hz, 1H), 5.46 (bs, 2H), 5.35 (s, 2H), 5.25-5.17 (m, 2H), 4.56-4.54 (m, 2H), 4.26 (bs, 2H), 3.85-3.22 (m), 2.74 (s, 3H), 2.02 (s, 3H), 1.77 (s, 3H). ¹³C NMR (101 MHz, DMSO-d₆) δ=156.1, 153.3, 147.3, 143.1, 142.2, 141.67, 136.9, 134.5, 132.8, 128.2, 126.4, 125.5, 124.7, 124.2, 121.7, 113.5, 111.0, 109.6, 100.4, 73.9, 69.4, 69.2, 68.7, 67.4, 61.9, 55.8, 53.0, 49.4, 48.6, 43.6, 41.6, 15.4, 12.8 ppm. IR (ATR): \tilde{ν}=3445 (w), 3022 (w), 2874 (w), 1682 (m), 1606 (w), 1576 (w), 1506 (w),1456 (m), 1409 (w), 1305 (m), 1235 (m), 1199 (s), 1178 (s), 1145 (s), 1062 (m), 1016 (m), 916 (w), 828 (w), 799 (w), 767 (w), 720 (m), 649 (w), 594 cm⁻¹ (w). MS (HR-ESI⁺): Exact mass calculated for [M+H]⁺: m/z=1093.3231, measured: m/z=1093.3225.

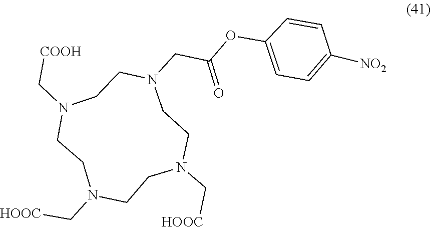 [SMILES: O=C(O)CN1CCN(CC(=O)O)CCN(CC(=O)Oc2ccc([N+](=O)[O-])cc2)CCN(CC(=O)O)CC1]

was synthesized according to literature[13].

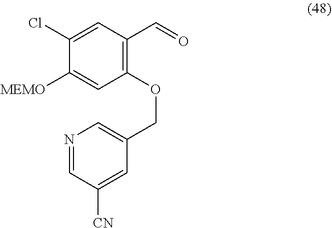

5-((4-Chloro-2-formyl-5-((2-methoxyethoxy)methoxy)phenoxy)methyl)nicotinonitrile (48). 5-chloro-2-hydroxy-4-((2-methoxyethoxy)methoxy)benzaldehyde (2) (644 mg, 2.47 mmol, 1.00 equiv.) reacted with 5-(bromomethyl)nicotinonitrile (730 mg, 3.71 mmol, 1.50 equiv.) and potassium carbonate (683 mg, 4.94 mmol, 2.00 equiv.) as a base in abs. DMF (10 mL) according to GP-1. Purification by flash column chromatography on silica gel (EA, R_{f}=0.3) lead to 5-((4-chloro-2-formyl-5-((2-methoxyethoxy)methoxy)phenoxy)methyl)nicotinonitrile (48) (897 mg, 2.38 mmol, 96%) as a yellowish solid. mp=97° C. ¹H NMR (600 MHz, CDCl₃) δ=10.29 (s, 1H), 8.91 (d, 4J=1.9 Hz, 1H), 8.90 (d, ⁴J=1.9 Hz, 1H), 8.11 (s, 1H), 7.87 (s, 1H), 6.98 (s, 1H), 5.42 (s, 2H), 5.23 (s, 2H), 3.88-3.89 (m, 2H), 3.57-3.58 (m, 2H), 3.37 ppm (s, 3H). ¹³C NMR (151 MHz, CDCl₃) δ=186.6, 159.9, 158.9, 152.4, 152.1, 138.2, 132.1, 130.6, 120.2, 117.5, 116.2, 110.5, 100.9, 94.3, 71.5, 68.5, 67.5, 59.1 ppm. IR (ATR): G=3059 (w), 2920 (w), 2870 (w), 2235 (w), 1676 (s), 1598 (m), 1563 (m), 1493 (m), 1444 (m), 1422 (m), 1373 (m), 1318 (w), 1293 (m), 1185 (s), 1112 (s), 1082 (s), 1054 (m), 1009 (s), 951 (s), 892 (m), 840 (s), 753 (w), 702 (s), 652 (m), 572 (w), 541 (m), 511 (w), 464 (w), 421 cm⁻¹ (w). MS (HR-ESI⁺): Exact mass calculated for [M+H]⁺: m/z=377.0904, measured: m/z=377.0899.

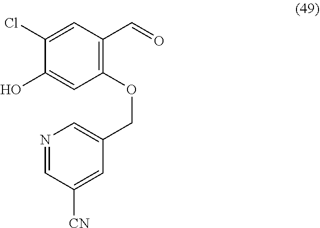 [SMILES: N#Cc1cncc(COc2cc(O)c(Cl)cc2C=O)c1]

5-((4-Chloro-2-formyl-5-hydroxyphenoxy)methyl)nicotinonitrile (49). MEM-Protected phenol 48 (870 mg, 2.31 mmol, 1.00 equiv.) was dissolved in abs. dichloromethane (12 mL) under argon atmosphere and the solution was cooled to 0° C. After addition of trifluoroacetic acid (3 mL), the reaction mixture was stirred at room temperature for 16 h. Ethyl acetate (100 mL) was added to the solution and a solid precipitated, which was isolated by filtration. The pale yellowish precipitate turned out to be 5-((4-chloro-2-formyl-5-hydroxyphenoxy)methyl)nicotinonitrile (49) (498 mg, 1.61 mmol, 70%), which was used in the next step without further purification. mp=180° C. (decomposition). ¹H NMR (600 MHz, DMSO-d₆) δ=11.58 (s, 1H), 10.16 (s, 1H), 9.03 (d, 4J=1.9 Hz, 1H), 9.01 (d, ⁴J=1.9 Hz, 1H), 8.51 (s, 1H), 7.66 (s, 1H), 6.77 (s, 1H), 5.35 ppm (s, 2H). ¹³C NMR (151 MHz, DMSO-d₆) δ=186.7, 160.2, 159.8, 152.4, 152.0, 138.9, 132.5, 129.4, 117.9, 116.8, 113.5, 109.1, 101.4, 66.8 ppm. IR (ATR): G=3012 (w), 2241 (w), 1655 (m), 1593 (m), 1568 (m), 1493 (m), 1452 (w), 1433 (m), 1389 (m), 1294 (s), 1198 (m), 1174 (m), 1133 (w), 1057 (m), 1022 (s), 947 (w), 887 (m), 843 (m), 718 (w), 698 (m), 682 (s), 636 (m), 597 (m), 552 (m), 466 (m), 417 cm⁻¹ (w). MS (HR-EI⁺): Exact mass calculated for [M]′⁺: m/z=288.0302, measured: m/z=288.0286.

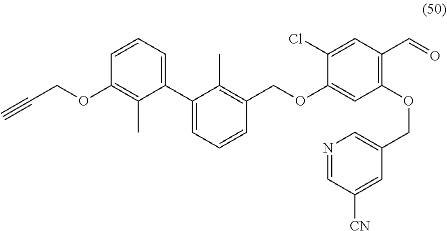 [SMILES: C#CCOc1cccc(-c2cccc(COc3cc(OCc4cncc(C#N)c4)c(C=O)cc3Cl)c2C)c1C]

5-((4-Chloro-5-((2,2′-dimethyl-3′-(prop-2-yn-1-yloxy)-[1,1′-biphenyl]-3-yl)-methoxy)-2-formylphenoxy)methyl)nicotinonitrile (50). (2,2′-Dimethyl-3′-(prop-2-yn-1-yloxy)-[1,1′-biphenyl]-3-yl)methanol (24) (110 mg, 381 μmol, 1.00 equiv.), phenol 49 (112 mg, 419 μmol, 1.10 equiv.), triphenylphosphine (130 mg, 495 μmol, 1.30 equiv.) were dissolved in abs. DMF (1.5 mL) and reacted with DEAD (77.7 μL, 495 μmol, 1.30 equiv.) according to GP-2. Purification by flash column chromatography on silica gel (PE:EA, 6:4, R_{f}=0.2) gave compound 50 (149 mg, 278 μmol, 73%) as a colorless solid. mp=157° C. ¹H NMR (600 MHz, CDCl₃) δ=10.27 (s, 1H), 8.91-8.92 (m, 2H), 8.10 (s, 1H), 7.92 (s, 1H), 7.45 (d, ³J=7.6 Hz, 1H), 7.28 (t, ³J=7.6 Hz, 1H), 7.22 (t, 3J=7.3 Hz, 1H), 7.17 (d, ³J=7.5 Hz, 1H), 7.00 (d, ³J=8.0 Hz, 1H), 6.80 (d, 3J=7.6 Hz, 1H), 6.65 (s, 1H), 5.25 (s, 2H), 5.22 (s, 2H), 4.77 (d, ⁴J=2.3 Hz, 2H), 2.55 (t, ⁴J=2.3 Hz, 1H), 2.09 (s, 3H), 1.93 ppm (s, 3H). ¹³C NMR (151 MHz, CDCl₃) δ=186.4, 160.1, 160.1, 156.0, 152.5, 151.9, 142.8, 142.6, 138.1, 134.8, 133.3, 132.0, 130.9, 130.3, 127.5, 126.2, 125.9, 125.6, 122.8, 119.5, 117.7, 116.2, 110.7, 110.5, 98.7, 79.1, 75.5, 70.7, 67.7, 56.3, 15.9, 13.1 ppm. IR (ATR): \tilde{ν}=3277 (w), 2918 (w), 2234 (w), 1672 (m), 1597 (s), 1501 (m), 1448 (m), 1410 (m), 1382 (m), 1312 (m), 1271 (s), 1171 (s), 1144 (s), 1076 (m), 1016 (s), 924 (w), 873 (m), 809 (m), 787 (m), 722 (m), 690 (s), 640 (m), 545 (w), 503 (w), 460 cm⁻¹ (w). MS (HR-MALDI): Exact mass calculated for [M+H]⁺: m/z=537.1576, measured 2 m/z=537.1577.

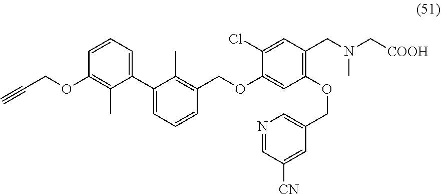 [SMILES: C#CCOc1cccc(-c2cccc(COc3cc(OCc4cncc(C#N)c4)c(CN(C)CC(=O)O)cc3Cl)c2C)c1C]

N-(5-Chloro-2-((5-cyanopyridin-3-yl)methoxy)-4-((2,2′-dimethyl-3′-(prop-2-yn-1-yloxy)-[1,1′-biphenyl]-3-yl)methoxy)benzyl)-N-methylglycine (51). Aldehyde 50 (160 mg, 298 mmol, 1.00 equiv.), sarcosine (133 mg, 1.49 mmol, 5.00 equiv.) and sodium cyanoborohydride (28.1 mg, 447 μmol, 1.50 equiv.) were reacted in an 1:1 mixture of abs. DMF/MeOH (4 mL) and subsequently worked up according to GP-3. After flash column chromatography on silica gel (MeCN:MeOH:Et3N, 100:20:1, R_{f}=0.2) compound 51 (107 mg, 175 μmol, 59%) was obtained as a colorless solid. mp=91° C. Rₜ=12.46 min (System A), purity: 98.6%. ¹H NMR (400 MHz, methanol-d₄) δ=8.97 (s, 1H), 8.92 (s, 1H), 8.41 (s, 1H), 7.57 (s, 1H), 7.46 (d, ³J=7.6 Hz, 1H), 7.26-7.18 (m, 2H), 7.03-7.10 (m, 3H), 6.74 (d, ³J=7.5 Hz, 1H), 5.38 (s, 2H), 5.30 (s, 2H), 4.76 (s, 2H), 4.34 (s, 2H), 3.57 (s, 2H), 2.94 (t, ⁴J=2.3 Hz, 1H), 2.78 (s, 3H), 2.08 (s, 3H), 1.88 ppm (s, 3H). ¹³C NMR (101 MHz, methanol-d₄) δ=170.0, 158.1, 157.9, 157.3, 153.4, 153.1, 144.2, 143.7, 140.7, 137.0, 136.2, 135.7, 135.3, 135.2, 134.7, 134.4, 131.7, 131.5, 130.8, 129.0, 127.1, 126.5, 126.3, 123.5, 117.3, 116.6, 113.0, 112.0, 111.6, 101.1, 80.1, 76.5, 71.5, 68.7, 59.2, 57.0, 55.2, 41.8, 15.8, 13.1 ppm. MS (HR-ESI⁺): Exact mass calculated for [M+H]⁺: m/z=610.2103, measured: m/z=610.2105.

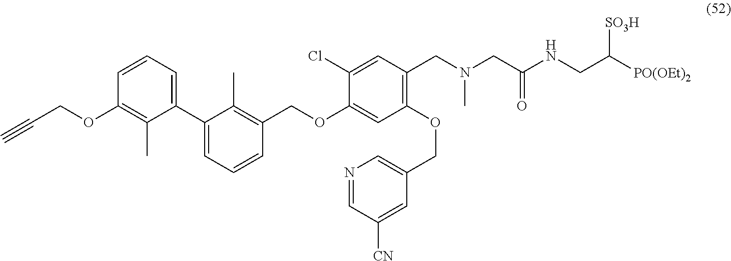

2-(2-((5-Chloro-2-((5-cyanopyridin-3-yl)methoxy)-4-((2,2′-dimethyl-3′-(prop-2-yn-1-yloxy)-[1,1′-biphenyl]-3-yl)methoxy)benzyl)(methyl)amino)acetamido)ethane-1,1-disulfonic acid (52). Carboxylic acid 51 (100 mg, 164 μmol, 1.00 equiv.) reacted with 2-aminoethane-1,1-disulfonic acid (TBA salt) (18) (320 mg, 820 μmol, 5.00 equiv.) in presence of DIPEA (57.3 μL, 328 μmol, 2.00 equiv.) and HTBU (137 mg, 361 μmol, 2.20 equiv.) in abs. DMF (3 mL) according to GP-6. Purification was performed by semi-preparative RP-HPLC (System B, Rₜ=9 min) and after cation exchange and lyophilization, compound 52 (106 mg, 133 μmol, 81%) was obtained as a colorless solid. mp=170° C. (decomposition). Rₜ=10.92 min (System A), purity: 96.8%. ¹H NMR (400 MHz, DMSO-d₆) δ=9.56 (bs, 1H), 9.07 (s, 2H), 8.55 (s, 1H), 8.14 (bs, 1H), 7.63 (s, 1H), 7.52 (d, ³J=7.7 Hz, 1H), 7.22-7.28 (m, 2H), 7.05-7.10 (m, 2H), 6.75 (d, 3J=7.5 Hz, 1H), 5.34-5.43 (m, 4H), 4.86 (s, 2H), 4.27-4.36 (m), 3.87-3.91 (m, 2H), 3.70-3.71 (m, 2H), 3.54-3.59 (m, 2H), 2.71 (s, 3H), 2.04 (s, 3H), 1.83 ppm (s, 3H). ¹³C NMR (101 MHz, DMSO-d₆) δ=163.4, 156.8, 155.7, 155.4, 152.2, 151.6, 142.3, 141.5, 139.6, 134.6, 134.5, 133.9, 132.8, 129.3, 127.9, 126.2, 125.5, 124.1, 122.1, 116.7, 113.4, 111.1, 110.9, 109.2, 100.2, 79.5, 78.2, 74.0, 69.7, 67.2, 56.2, 55.8, 52.9, 40.7, 15.3, 12.8 ppm. MS (HR-ESI⁺): Exact mass calculated for [M+H]⁺: m/z=797.1713, measured: m/z=797.1712.

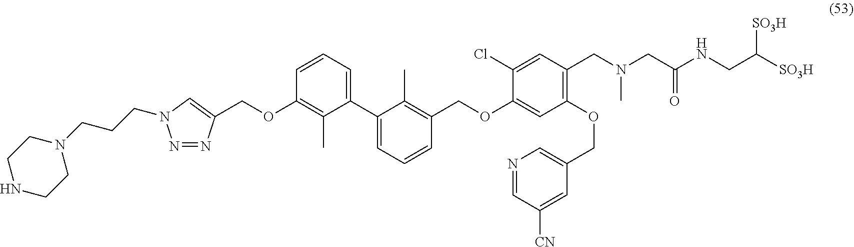 [SMILES: Cc1c(COc2cc(OCc3cncc(C#N)c3)c(CN(C)CC(=O)NCC(S(=O)(=O)O)S(=O)(=O)O)cc2Cl)cccc1-c1cccc(OCc2cn(CCCN3CCNCC3)nn2)c1C]

2-(2-((5-Chloro-2-((5-cyanopyridin-3-yl)methoxy)-4-((2,2′-dimethyl-3′-((1-(3-(piperazin-1-yl)propyl)-1H-1,2,3-triazol-4-yl)methoxy)-[1,1′-biphenyl]-3-yl)methoxy)benzyl)(methyl)amino)acetamido)ethane-1,1-disulfonic acid (53). Alkyne 52 (25.0 mg, 31.4 μmol, 1.00 equiv.) and 1-(3-azidopropyl)piperazine (31) (10.6 mg, 62.7 μmol, 2.00 equiv.) were dissolved in DMF (1 mL), reacted in presence of TBTA (0.2 mg, 0.3 μmol, 0.01 equiv.) and [Cu(MeCN)₄]PF₆ (5.8 mg, 15.7 μmol, 0.50 equiv.) according to GP-4. Purification by semi-preparative RP-HPLC (System C, Rₜ=18 min) yielded compound 53 (14.0 mg, 14.5 μmol, 46%) as a colorless powder. mp=150° C. (decomposition). Rₜ=9.26 min (System A), purity: 95.2%. ¹H NMR (400 MHz, DMSO-d₆) δ=9.62 (bs, 2H), 9.02 (dd, ⁴J=1.8, ³J=11.0 Hz, 2H), 8.48 (s, 1H), 8.27 (s, 1H), 8.15 (bs, 1H), 7.58 (s, 1H), 7.50 (d, ³J=7.3 Hz, 1H), 7.21-7.27 (m, 2H), 7.14-7.16 (m, 2H), 7.07 (d, ³J=7.4 Hz, 1H), 6.72 (d, ³J=7.3 Hz, 1H), 5.30-5.39 (m, 4H), 5.16-5.23 (m, 2H), 4.46 (t, ³J=6.9 Hz, 2H), 1.92 (bs, 2H), 3.78-3.41 (m), 2.70 (s, 3H), 2.24 (bs, 2H), 1.99 (s, 3H), 1.71 ppm (bs, 3H). ¹³C NMR (101 MHz, DMSO-d₆) δ=163.6, 156.6, 156.1, 152.5, 152.1, 146.6, 143.2, 142.2, 141.7, 139.1, 135.1, 134.7, 133.8, 132.6, 129.3, 126.4, 125.4, 124.4, 124.3, 121.6, 116.9, 113.6, 111.0, 109.1, 100.5, 73.9, 70.0, 67.2, 61.9, 53.7, 52.8, 48.5, 46.9, 41.2, 15.5, 12.7 ppm. MS (HR-ESI⁺): Exact mass calculated for [M+H]⁺: m/z=966.3040, measured: m/z=966.3045.

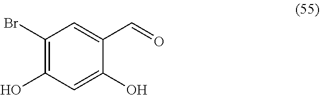 [SMILES: O=Cc1cc(Br)c(O)cc1O]

was synthesized according to literature[14].

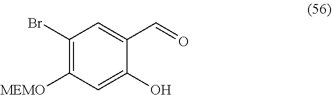

5-Bromo-2-hydroxy-4-((2-methoxyethoxy)methoxy)benzaldehyde (56). 5-Bromo-2,4-dihydroxybenzaldehyde (55) (5.50 g, 25.3 mmol, 1.00 equiv.) was dissolved in abs. THF (100 mL) under argon and abs. DIPEA (8.83 mL, 50.7 mmol, 2.00 equiv.) was added in one portion. After cooling to 0° C., MEM chloride (4.34 mL, 38.0 mmol, 1.50 equiv.) was added via syringe in small portions and the reaction mixture was stirred at room temperature for 16 h under argon. Water (100 mL) was added and the phases were separated. The aqueous phase was extracted with DCM (3×100 mL) and the combined organic extracts were dried over sodium sulfate, filtrated and concentrated under reduced pressure. The residue was purified by flash column chromatography on silica gel (PE:EA, 7:3, R_{f}=0.25) to afford 5-bromo-2-hydroxy-4-((2-methoxyethoxy)methoxy)benzaldehyde (56) (3.78 g, 12.4 mmol, 60%) as a yellowish oil. mp=56° C. ¹H NMR (400 MHz, CDCl₃) δ=11.27 (s, 1H), 9.69 (s, 1H), 7.67 (s, 1H), 6.76 (s, 1H), 5.38 (s, 2H), 3.84-3.86 (m, 2H), 3.57-3.57 ppm (m, 3H). ¹³C NMR (101 MHz, CDCl₃) δ=193.9, 163.3, 160.4, 137.5, 116.7, 103.6, 102.8, 94.0, 71.5, 68.8, 59.2 ppm. MS (HR-ESI⁺): Exact mass calculated for [M+Na]⁺: m/z=326.9844, measured: m/z=326.9838.

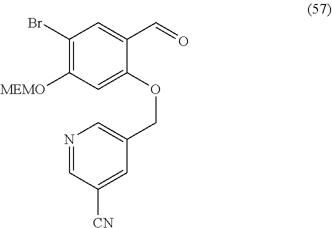

5-((4-Bromo-2-formyl-5-((2-methoxyethoxy)methoxy)phenoxy)methyl)nicotinonitrile (57). 5-Bromo-2-hydroxy-4-((2-methoxyethoxy)methoxy)benzaldehyde (56) (1.28 g, 4.20 mmol, 1.00 equiv.) reacted with 5-(bromomethyl)nicotinonitrile (992 mg, 5.03 mmol, 1.20 equiv.) and potassium carbonate (1.16 g, 8.39 mmol, 2.00 equiv.) as a base in abs. DMF (20 mL) according to GP-1. Purification by flash column chromatography on silica gel (EE, R_{f}=0.3) lead to 5-((4-bromo-2-formyl-5-((2-methoxyethoxy)methoxy)phenoxy)methyl)nicotinonitrile (57) (1.53 g, 3.63 mmol, 87%) as a yellowish solid. mp=110° C. ¹H NMR (400 MHz, CDCl₃) δ=10.21 (s, 1H), 8.89 (d, 4J=1.9 Hz, 1H), 8.85 (d, ⁴J=1.6 Hz, 1H), 8.08-8.09 (s, 1H), 7.94 (d, ⁴J=1.0 Hz, 1H), 6.91 (s, 1H), 5.38 (s, 2H), 5.20 (s, 2H), 3.83-3.86 (m, 2H), 3.53-3.55 (m, 2H), 3.33 ppm (s, 3H)¹³C NMR (101 MHz, CDCl₃) δ=186.4, 160.4, 159.7, 152.3, 151.9, 138.2, 133.5, 132.0, 120.5, 116.2, 110.2, 105.4, 100.6, 94.2, 71.3, 68.4, 67.3, 59.0 ppm. MS (HR-ESI⁺): Exact mass calculated for [M+Na]⁺: m/z=443.0219, measured: m/z=443.0215.

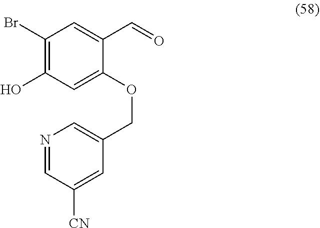 [SMILES: N#Cc1cncc(COc2cc(O)c(Br)cc2C=O)c1]

5-((4-Bromo-2-formyl-5-hydroxyphenoxy)methyl)nicotinonitrile (58). MEM-Protected phenol 57 (760 mg, 1.80 mmol, 1.00 equiv.) was dissolved in abs. dichloromethane (10 mL) under argon atmosphere and the solution was cooled to 0° C. After addition of trifluoroacetic acid (5 mL), the reaction mixture was stirred at room temperature for 16 h. Ethyl acetate (100 mL) was added to the solution and a solid precipitated, which was isolated by filtration. The pale yellowish precipitate turned out to be 5-((4-bromo-2-formyl-5-hydroxyphenoxy)methyl)nicotinonitrile (58) (495 mg, 1.49 mmol, 82%), which was used in the next step without further purification. mp=200° C. (decomposition). ¹H NMR (400 MHz, DMSO-d₆) δ=11.62 (s, 1H), 10.14 (s, 1H), 9.02 (s, 2H), 8.51 (s, 1H), 7.80 (s, 1H), 6.75 (s, 1H), 5.32 ppm (s, 2H). ¹³C NMR (101 MHz, DMSO-d₆) δ=186.6, 160.8, 160.8, 152.3, 152.0, 138.9, 132.6, 132.5, 118.5, 116.8, 109.0, 102.1, 101.1, 66.7 ppm. MS (HR-ESI⁺): Exact mass calculated for [M+Na]⁺: m/z=331.9897, measured: m/z=331.9782.

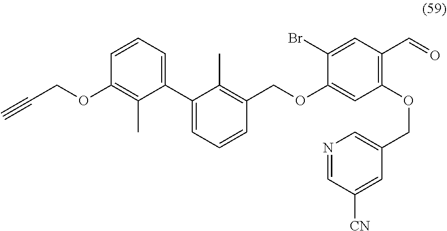 [SMILES: C#CCOc1cccc(-c2cccc(COc3cc(OCc4cncc(C#N)c4)c(C=O)cc3Br)c2C)c1C]

5-((4-Bromo-5-((2,2′-dimethyl-3′-(prop-2-yn-1-yloxy)-[1,1′-biphenyl]-3-yl)-methoxy)-2-formylphenoxy)methyl)nicotinonitrile (59). (2,2′-Dimethyl-3′-(prop-2-yn-1-yloxy)-[1,1′-biphenyl]-3-yl)methanol (24) (150 mg, 563 μmol, 1.00 equiv.), phenol 58 (216 mg, 648 μmol, 1.15 equiv.) and triphenylphosphine (192 mg, 732 μmol, 1.30 equiv.) were dissolved in abs. DMF (3 mL) and reacted with DMEAD (172 mg, 732 μmol, 1.30 equiv.) according to GP-2. Purification by flash column chromatography on silica gel (DCM:EA, 95:5, R_{f}=0.2) gave title compound 59 (185 mg, 318 μmol, 57%) as a colorless solid. mp=95° C. (decomposition). ¹H NMR (400 MHz, CDCl₃) δ=10.25 (s, 1H), 9.02 (bs, 2H), 8.09-8.11 (m, 2H), 7.46 (d, ³J=7.5 Hz, 1H), 7.16-7.28 (m, 3H), 6.99 (d, ³J=8.2 Hz, 1H), 6.79 (d, ³J=7.6 Hz, 1H), 6.62 (s, 1H), 5.23-5.25 (m, 4H), 4.77 (s, 2H), 2.54 (s, 1H), 2.09 (s, 3H), 1.93 ppm (s, 3H). ¹³C NMR (101 MHz, DMSO-d₆) δ=186.3, 161.0, 160.8, 156.0, 152.5, 151.8, 142.8, 142.6, 138.1, 134.7, 134.2, 133.3, 130.3, 127.5, 126.2, 125.9, 125.6, 122.8, 120.1, 116.2, 110.7, 110.2, 105.8, 98.5, 79.1, 75.5, 70.7, 67.7, 56.3, 15.9, 13.1 ppm. MS (HR-ESI⁺): Exact mass calculated for [M+Na]⁺: m/z=603.0895, measured: m/z=603.0896.

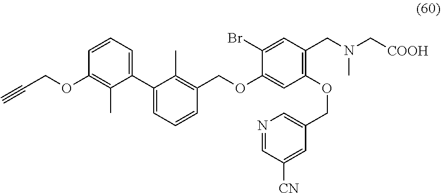 [SMILES: C#CCOc1cccc(-c2cccc(COc3cc(OCc4cncc(C#N)c4)c(CN(C)CC(=O)O)cc3Br)c2C)c1C]

N-(5-Bromo-2-((5-cyanopyridin-3-yl)methoxy)-4-((2,2′-dimethyl-3′-(prop-2-yn-1-yloxy)-[1,1′-biphenyl]-3-yl)methoxy)benzyl)-N-methylglycine (60). Aldeyhde 59 (180 mg, 310 mmol, 1.00 equiv.), sarcosine (138 mg, 1.55 mmol, 5.00 equiv.) and sodium cyanoborohydride (29.2 mg, 464 μmol, 1.50 equiv.) were reacted in an 1:1 mixture of abs. DMF/MeOH (4 mL) and subsequently worked up according to GP-3. After flash column chromatography on silica gel (MeCN:MeOH:Et₃N, 100:20:1, R_{f}=0.2) compound 60 (131 mg, 200 μmol, 65%) was obtained as a colorless solid. mp=97-99° C. Rₜ=12.52 min (System A), purity: 96.2%. ¹H NMR (400 MHz, methanol-d₄) δ=8.97 (s, 1H), 8.91 (s, 1H), 8.41 (s, 1H), 7.73 (s, 1H), 7.47 (d, ³J=7.6 Hz, 1H), 7.18-7.26 (m, 2H), 7.09 (d, ³J=7.6 Hz, 2H), 7.02-7.04 (m, 2H), 6.74 (d, ³J=7.5 Hz, 1H), 5.38 (s, 2H), 5.29 (s, 2H), 4.79 (d, ⁴J=1.8 Hz, 2H), 4.36 (s, 2H), 3.59 (s, 2H), 2.94 (t, 4J=1.8 Hz, 1H), 2.79 (s, 3H), 2.08 (s, 3H), 1.88 ppm (s, 3H). ¹³C NMR (101 MHz, methanol-d₄) δ=169.8, 159.0, 158.8, 157.3, 153.4, 153.1, 144.2, 143.6, 140.7, 137.8, 136.2, 135.7, 134.4, 130.8, 128.9, 127.1, 126.5, 126.3, 123.5, 117.3, 133.4, 122.0, 111.6, 104.7, 100.8, 80.1, 76.5, 71.5, 68.7, 59.1, 57.0, 55.2, 41.8, 15.9, 13.2 ppm. MS (HR-ESI⁺): Exact mass calculated for [M+H]⁺: m/z=654.1598, measured: m/z=654.1610.

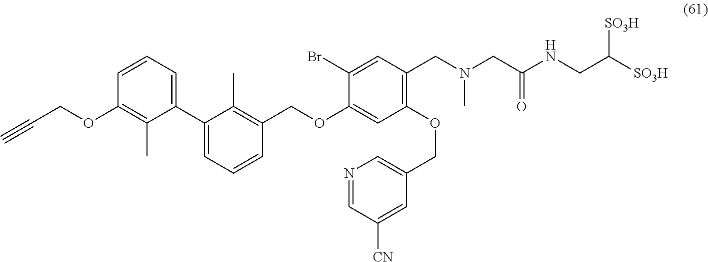 [SMILES: C#CCOc1cccc(-c2cccc(COc3cc(OCc4cncc(C#N)c4)c(CN(C)CC(=O)NCC(S(=O)(=O)O)S(=O)(=O)O)cc3Br)c2C)c1C]

2-(2-((5-Bromo-2-((5-cyanopyridin-3-yl)methoxy)-4-((2,2′-dimethyl-3′-(prop-2-yn-1-yloxy)-[1,1′-biphenyl]-3-yl)methoxy)benzyl)(methyl)amino)acetamido)ethane-1,1-disulfonic acid (61). Carboxylic acid 60 (120 mg, 209 μmol, 1.00 equiv.) reacted with 2-aminoethane-1,1-disulfonic acid (TBA salt) (18) (408 mg, 1.04 mol, 5.00 equiv.) in presence of DIPEA (72.8 μL, 418 μmol, 2.00 equiv.) and HTBU (174 mg, 459 μmol, 2.20 equiv.) in abs. DMF (3 mL) according to GP-6. Purification was performed by semi-preparative RP-HPLC (System B, Rₜ=10 min) and after cation exchange and lyophilization, compound 61 (110 mg, 131 μmol, 63%) was obtained as a colorless solid. mp=170° C. (decomposition). Rₜ=11.06 min (System A), purity: 99.8%. ¹H NMR (400 MHz, DMSO-d₆) δ=9.56 (bs, 1H), 9.07 (s, 2H), 8.55 (s, 1H), 8.15 (bs, 1H), 7.76 (s, 1H), 7.54 (d, ³J=7.4 Hz, 1H), 7.28-7.24 (m, 2H), 7.18 (s, 1H), 7.04-7.10 (m, 2H), 6.75 (d, ³J=7.5 Hz, 1H), 5.33-5.47 (m, 4H), 4.82-4.85 (m), 4.26-4.35 (m, 2H), 3.88-3.91 (m, 2H), 3.69-3.72 (m, 2H), 3.54-3.59 (m, 2H), 2.70 (s, 3H), 2.05 (s, 3H), 1.84 ppm (s, 3H). ¹³C NMR (101 MHz, DMSO-d₆) δ=163.4, 157.5, 156.6, 155.4, 152.1, 151.6, 142.3, 141.5, 139.6, 136.8, 134.6, 134.5, 132.9, 129.3, 127.8, 126.2, 125.5, 124.1, 122.1, 116.7, 111.8, 110.9, 109.2, 102.1, 100.0, 76.5, 78.2, 74.0, 69.8, 67.1, 56.2, 55.8, 52.9, 40.7, 15.4, 12.8 ppm. MS (HR-ESI⁺): Exact mass calculated for [M+H]⁺: m/z=841.1207, measured: m/z=841.1209.

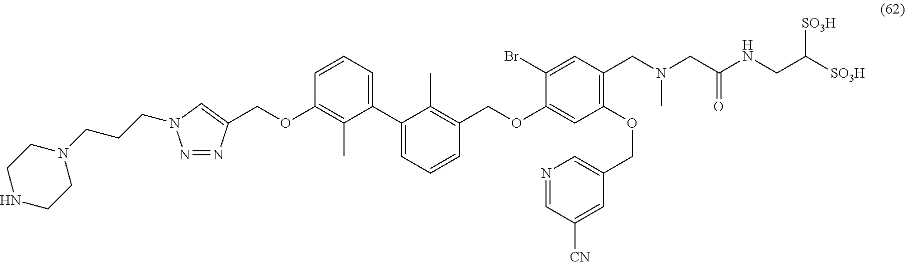 [SMILES: Cc1c(COc2cc(OCc3cncc(C#N)c3)c(CN(C)CC(=O)NCC(S(=O)(=O)O)S(=O)(=O)O)cc2Br)cccc1-c1cccc(OCc2cn(CCCN3CCNCC3)nn2)c1C]

2-(2-((5-Bromo-2-((5-cyanopyridin-3-yl)methoxy)-4-((2,2′-dimethyl-3′-((1-(3-(piperazin-1-yl)propyl)-1H-1,2,3-triazol-4-yl)methoxy)-[1,1′-biphenyl]-3-yl)methoxy)benzyl)(methyl)amino)acetamido)ethane-1,1-disulfonic acid (62). Alkyne 61 (25.0 mg, 29.7 μmol, 1.00 equiv.) reacted with 1-(3-azidopropyl)piperazine (7.61 mg, 45.0 μmol, 2.00 equiv.) with a premixed catalyst consisting of CuSO₄ (1.42 mg, 8.91 μmol, 0.30 equiv.), THPTA (1.68 mg, 3.86 μmol, 0.13 equiv.) and sodium ascorbate (29.4 mg, 149 μmol, 5.00 equiv.) in an 1:1 mixture of H₂O/t-BuOH (2 mL) at room temperature for 5 h according to GP-5. Purification by semi-preparative RP-HPLC (System C, Rₜ=21 min) and subsequent lyophilization yielded compound (62) (19.0 mg, 18.8 μmol, 63%) as a colorless solid. mp=200° C. (decomposition). Rₜ=9.37 min (System A), purity: 97.9%. ¹H NMR (400 MHz, DMSO-d₆) δ=9.81 (bs, 2H), 9.26 (s, 1H), 9.23 (s, 1H), 8.70 (s, 1H), 8.49 (s, 1H), 8.37 (bs, 1H), 7.94 (s, 1H), 7.73 (bs, 1H), 7.43-7.50 (m, 2H), 7.28-7.39 (m, 3H), 6.95 (d, ³J=7.3 Hz, 1H), 5.51-5.63 (m, 4H), 5.39-5.46 (m, 2H), 4.69 (t, ³J=6.9 Hz, 2H), 4.47 (bs), 3.68-3.99 (m), 3.22 (bs), 2.93 (s, 3H), 2.73 (s, 3H), 2.22 (s, 3H), 1.90-1.95 ppm (m, 2H). ¹³C NMR (101 MHz, DMSO-d₆) δ=163.6, 156.0, 152.4, 152.0, 143.3, 142.2, 141.8, 139.0, 136.7, 135.1, 134.4, 132.5, 129.2, 126.4, 125.4, 124.3, 121.6, 116.9, 111.1, 109.6, 109.1, 102.3, 73.9, 67.1, 61.9, 53.7, 48.4, 46.8, 41.3, 24.1, 15.6, 12.8 ppm. MS (HR-ESI⁺): Exact mass calculated for [M+H]⁺: m/z=1010.2540, measured: m/z=1010.2533.

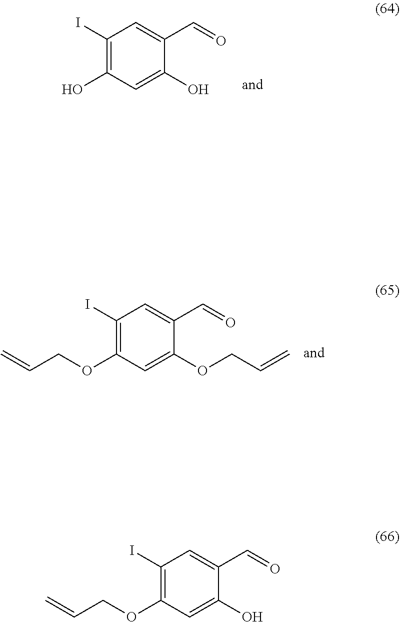

were synthesized according to literature [15].

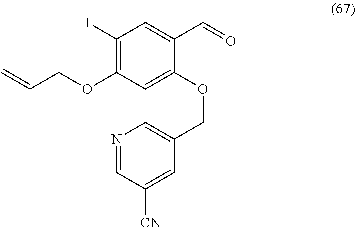 [SMILES: C=CCOc1cc(OCc2cncc(C#N)c2)c(C=O)cc1I]

5-((5-(Allyloxy)-2-formyl-4-iodophenoxy)methyl)nicotinonitrile (67). 4-(Allyloxy)-2-hydroxy-5-iodobenzaldehyde (66) (1.28 g, 4.21 mmol, 1.00 equiv.) reacted with 5-(bromomethyl)nicotinonitrile (995 mg, 5.05 mmol, 1.20 equiv.) and potassium carbonate (3.54 g, 8.42 mmol, 2.00 equiv.) as a base in abs. DMF (5 mL) according to GP-1. Purification by flash column chromatography on silica gel (DCM:EtOH, 90:10, R_{f}=0.3) lead to 5-((5-(allyloxy)-2-formyl-4-iodophenoxy)methyl)nicotinonitrile (67) (1.30 g, 3.09 mmol, 74%) as a beige solid. mp=153° C. ¹H NMR (400 MHz, CDCl₃) δ=10.02 (s, 1H), 8.90-8.92 (m, 2H), 8.25 (s, 1H), 8.10 (s, 1H), 6.42 (s, 1H), 6.00-6.05 (m, 1H), 5.49-5.54 (m, 1H), 5.36-5.39 (m, 1H), 5.22 (s, 2H), 4.68-4.69 ppm (m, 2H). ¹³C NMR (101 MHz, CDCl₃) δ=186.3, 162.9, 161.8, 152.4, 151.8, 140.3, 138.2, 132.0, 131.5, 120.8, 118.7, 116.2, 110.5, 97.3, 77.8, 70.3, 67.4 ppm. MS (HR-ESI⁺): Exact mass calculated for [M+H]⁺: m/z=421.0044, measured: m/z=421.0049.

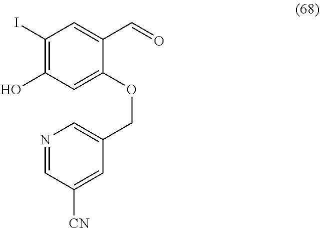 [SMILES: N#Cc1cncc(COc2cc(O)c(I)cc2C=O)c1]

5-((2-Formyl-5-hydroxy-4-iodophenoxy)methyl)nicotinonitrile (68). 5-((5-(Allyloxy)-2-formyl-4-iodophenoxy)methyl)nicotinonitrile (67) (1.21 g, 2.87 mmol, 1.00 equiv.) was suspended in MeOH (50 mL) and this solution was degassed with argon for 30 min. Tetrakis(triphenylphosphine)palladium(0) (33.2 mg, 28.7 μmol, 0.01 equiv.) and potassium carbonate (1.19 g, 8.62 mmol, 3.00 equiv.) were added to initialize the reaction, which was stirred under argon at room temperature for 3 h. The solvent was removed under reduced pressure, ethyl acetate (100 mL) and water (100 mL) were added, the phases were separated and the aqueous phase was extracted with ethyl acetate (3×50 mL). The combined organic fractions were dried over sodium sulfate, filtrated and the solvent was removed in vacuo. The crude product was purified on silica gel (EA, R_{f}=0.3) to yield 5-((2-formyl-5-hydroxy-4-iodophenoxy)methyl)nicotinonitrile (68) (360 mg, 947 μmol, 33%) as a brownish solid. mp=160° C. (decomposition). ¹H NMR (400 MHz, DMSO-d₆) δ=11.64 (s, 1H), 10.10 (s, 1H), 9.01-9.02 (m, 2H), 8.51 (s, 1H), 7.98-7.99 (s, 1H), 6.69 (s, 1H), 5.31 ppm (s, 1H). ¹³C NMR (101 MHz, DMSO-d₆) δ=186.4, 163.3, 161.7, 152.3, 152.0, 138.9, 138.8, 132.5, 119.3, 116.8, 109.0, 99.9, 75.8, 66.6 ppm. MS (HR-ESI⁺): Exact mass calculated for [M+H]⁺: m/z=380.9736, measured: m/z=380.9732.

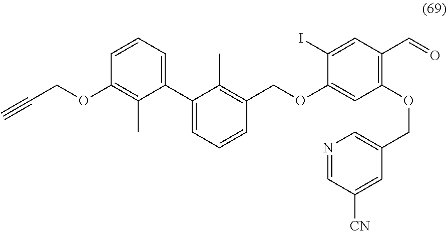 [SMILES: C#CCOc1cccc(-c2cccc(COc3cc(OCc4cncc(C#N)c4)c(C=O)cc3I)c2C)c1C]

5-((5-((2,2′-Dimethyl-3′-(prop-2-yn-1-yloxy)-[1,1′-biphenyl]-3-yl)methoxy)-2-formyl-4-iodophenoxy)methyl)nicotinonitrile (69). (2,2′-Dimethyl-3′-(prop-2-yn-1-yloxy)-[1,1′-biphenyl]-3-yl)methanol (24) (135 mg, 355 μmol, 1.00 equiv.), phenol (68) (109 mg, 408 μmol, 1.15 equiv.) and triphenylphosphine (121 mg, 462 μmol, 1.30 equiv.) were dissolved in abs. DMF (3 mL) and reacted with DMEAD (108 mg, 462 μmol, 1.30 equiv.) according to GP-2. Purification by flash column chromatography on silica gel (DCM:EA, 95:5, R_{f}=0.2) gave title compound (69) (126 mg, 201 μmol, 57%) as a colorless solid. mp=142° C. ¹H NMR (400 MHz, CDCl₃) δ=10.21 (s, 1H), 8.91 (s, 2H), 8.29 (s, 1H), 8.12 (s, 1H), 7.48 (d, ³J=7.6 Hz, 1H), 7.00-7.27 (m, 3H), 6.99 (d, ³J=8.2 Hz, 1H), 6.79 (d, ³J=7.6 Hz, 1H), 6.55 (s, 1H), 5.23 (s, 4H), 4.76-4.77 (m, 2H), 2.53-2.55 (m, 1H), 2.09 (s, 3H), 1.94 ppm (s, 3H). ¹³C NMR (101 MHz, CDCl₃) δ=186.3, 163.1, 161.8, 156.0, 152.5, 151.8, 142.8, 142.6, 140.5, 138.2, 134.7, 133.3, 132.0, 130.2, 127.6, 126.2, 125.8, 125.6, 122.8, 120.9, 116.1, 110.7, 110.6, 97.5, 79.1, 78.0, 75.5, 70.9, 67.5, 56.3, 16.1, 13.1 ppm. MS (HR-ESI⁺): Exact mass calculated for [M+H]⁺: m/z=629.0932, measured: m/z=629.0942.

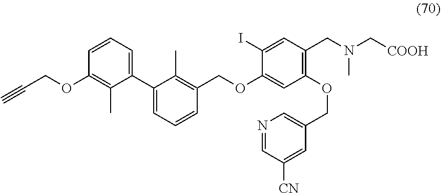 [SMILES: C#CCOc1cccc(-c2cccc(COc3cc(OCc4cncc(C#N)c4)c(CN(C)CC(=O)O)cc3I)c2C)c1C]

N-(5-Iodo-2-((5-cyanopyridin-3-yl)methoxy)-4-((2,2′-dimethyl-3′-(prop-2-yn-1-yloxy)-[1,1′-biphenyl]-3-yl)methoxy)benzyl)-N-methylglycine (70). Aldeyhde 69 (120 mg, 191 mmol, 1.00 equiv.), sarcosine (85.1 mg, 955 mmol, 5.00 equiv.) and sodium cyanoborohydride (18.0 mg, 287 μmol, 1.50 equiv.) were reacted in an 1:1 mixture of abs. DMF/MeOH (3 mL) and subsequently worked up according to GP-3. After flash column chromatography on silica gel (MeCN:MeOH:Et3N, 100:20:1, R_{f}=0.2) compound 70 (91.0 mg, 130 μmol, 68%) was obtained as a colorless solid. mp=75° C. Rₜ=14.25 min (System A), purity: 97.7%. ¹H NMR (400 MHz, methanol-d₄) δ=8.97 (d, ⁴J=2.1 Hz, 1H), 8.90 (d, ⁴J=2.0 Hz, 1H), 8.40 (t, ⁴J=2.0 Hz, 1H), 7.91 (s, 1H), 7.50 (dd, ⁴J=1.0 Hz, ³J=7.6 Hz, 1H), 7.16-7.23 (m, 2H), 7.08 (dd, ⁴J=1.2 Hz, 3J=7.6 Hz, 1H), 7.01-7.03 (m, 1H), 6.95 (s, 1H), 6.73 (dd, ⁴J=0.8 Hz, ³J=7.5 Hz, 1H), 5.38 (s, 2H), 5.26 (s, 2H), 4.76 (dd, ⁴J=2.4 Hz, ³J=9.3 Hz, 2H), 4.34 (s, 2H), 3.59 (s, 2H), 2.94 (t, ⁴J=2.4 Hz, 1H), 2.79 (s, 3H), 2.08 (s, 3H), 1.88 ppm (s, 3H). ¹³C NMR (101 MHz, methanol-d₄) δ=170.0, 161.3, 159.9, 157.3, 153.4, 153.1, 144.1, 143.7, 143.5, 140.6, 136.1, 135.7, 134.3, 130.7, 128.9, 127.1, 126.5, 126.3, 123.5, 117.4, 114.4, 111.9, 111.5, 99.7, 80.1, 76.8, 76.6, 71.6, 68.5, 59.1, 57.0, 55.0, 49.9, 41.8, 16.2, 13.3 ppm. MS (HR-ESI⁺): Exact mass calculated for [M+H]⁺: m/z=702.1460, measured: m/z=702.1462.

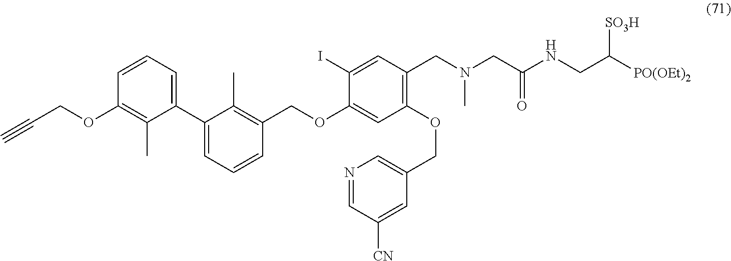

2-(2-((2-((5-Cyanopyridin-3-yl)methoxy)-4-((2,2′-dimethyl-3′-(prop-2-yn-1-yloxy)-[1,1′-biphenyl]-3-yl)methoxy)-5-iodobenzyl)(methyl)amino)acetamido)ethane-1,1-disulfonic acid (71). Carboxylic acid 70 (85.0 mg, 121 μmol, 1.00 equiv.) reacted with 2-aminoethane-1,1-disulfonic acid (TBA salt) (18) (237 mg, 606 mol, 5.00 equiv.) in presence of DIPEA (42.1 μL, 242 μmol, 2.00 equiv.) and HTBU (101 mg, 267 μmol, 2.20 equiv.) in abs. DMF (2 mL) according to GP-5. Purification was performed by semi-preparative RP-HPLC (Agilent Zorbax SB C-18 5 μm 80 Å, 9.4×250 mm with 30-90% acetonitrile (0.1% TFA) in water (0.1% TFA) in a linear gradient over 45 min, 6 mL/min, Rₜ=12 min) and after cation exchange and lyophilization, compound 71 (60.0 mg, 67.5 μmol, 56%) was obtained as a colorless solid. mp=150° C. (decomposition). Rₜ=11.58 min (System A), purity: 96.1%. ¹H NMR (600 MHz, DMSO-d₆) (=9.54 (bs, 1H), 9.09 (s, 1H), 9.07 (s, 1H), 8.57 (s, 1H), 8.16 (t, ³J=5.1 Hz, 1H), 7.92 (s, 1H), 7.58 (d, ³J=7.5 Hz, 1H), 7.28 (t, ³J=7.6 Hz, 1H), 7.24 (t, ³J=7.9 Hz, 1H), 7.09-7.10 (m, 2H), 7.06 (d, ³J=8.3 Hz, 1H), 6.75 (d, ³J=7.5 Hz, 1H), 5.39-5.44 (m, 2H), 5.25-5.30 (m), 4.85 (d, ⁴J=2.2 Hz, 2H), 4.23-4.32 (m, 2H), 3.86-3.94 (m, 2H), 3.67-3.76 (m, 2H), 3.58-3.60 (m, 2H), 2.69 (s, 3H), 2.06 (s, 3H), 1.85 ppm (s, 3H). ¹³C NMR (151 MHz, DMSO-d₆) δ=163.5, 159.1, 158.4, 155.4, 151.9, 151.4, 142.5, 142.4, 141.4, 139.8, 134.6, 134.5, 133.0, 129.2, 127.7, 126.2, 125.4, 124.2, 122.0, 116.6, 112.6, 110.9, 109.3, 99.1, 79.5, 78.1, 76.0, 74.0, 69.8, 67.0, 56.1, 55.9, 52.7, 40.6, 15.6, 12.8 ppm. MS (HR-ESI⁺): Exact mass calculated for [M+H]⁺: m/z=889.1069, measured: m/z=889.1067.

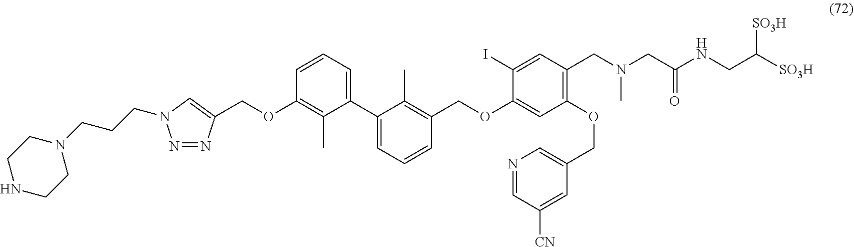 [SMILES: Cc1c(COc2cc(OCc3cncc(C#N)c3)c(CN(C)CC(=O)NCC(S(=O)(=O)O)S(=O)(=O)O)cc2I)cccc1-c1cccc(OCc2cn(CCCN3CCNCC3)nn2)c1C]

2-(2-((2-((5-Cyanopyridin-3-yl)methoxy)-4-((2,2′-dimethyl-3′-((1-(3-(piperazin-1-yl)propyl)-1H-1,2,3-triazol-4-yl)methoxy)-[1,1′-biphenyl]-3-yl)methoxy)-5-iodobenzyl)(methyl)amino)acetamido)ethane-1,1-disulfonic acid (72). Alkyne 71 (20.0 mg, 22.5 μmol, 1.00 equiv.) reacted with 1-(3-azidopropyl)piperazine (31) (7.62 mg, 45.0 μmol, 2.00 equiv.) with a premixed catalyst consisting of CuSO₄ (1.08 mg, 6.75 μmol, 0.30 equiv.), THPTA (1.27 mg, 2.93 μmol, 0.13 equiv.) and sodium ascorbate (22.3 mg, 113 μmol, 5.00 equiv.) in an 1:1 mixture of H₂O/t-BuOH (2 mL) at room temperature for 5 h according to GP-5. Purification by semi-preparative RP-HPLC (System C, Rₜ=22 min) and subsequent lyophilization yielded compound 72 (15.9 mg, 15.0 μmol, 67%) as a colorless solid. mp=205° C. (decomposition). Rₜ=9.83 min (System A), purity: 95.8%. ¹H NMR (600 MHz, DMSO-d₆) δ=9.57 (bs, 2H), 9.02 (dd, 4J=1.9 Hz, ³J=7.5 Hz, 2H), 8.48 (s, 1H), 8.28 (s, 1H), 8.13 (bs, 1H), 7.88 (s, 1H), 7.54 (d, ³J=7.5 Hz, 1H), 7.22-7.27 (m, 2H), 7.16 (d, ³J=8.3 Hz, 1H), 7.04-7.07 (m, 2H), 6.72 (d, ³J=7.4 Hz, 1H), 5.33-5.35 (m, 4H), 5.17-5.22 (m, 2H), 4.46 (t, ³J=7.0 Hz, 2H), 4.26 (bs, 2H), 3.42-3.80 (m), 2.69 (s, 3H), 2.26 (bs, 2H), 2.02 (s, 3H), 1.76 ppm (s, 3H). ¹³C NMR (151 MHz, DMSO-d₆) δ=163.6, 159.0, 158.3, 158.0, 156.1, 152.4, 152.0, 143.1, 142.4, 142.2, 141.7, 139.0, 134.8, 134.5, 132.5, 129.2, 128.0, 126.3, 125.4, 124.4, 124.2, 121.6, 116.9, 112.4, 110.9, 99.2, 76.0, 73.9, 70.0, 67.0, 61.8, 55.8, 53.7, 52.8, 48.4, 46.9, 41.0, 40.6, 38.9, 24.4, 15.7, 12.8 ppm. MS (HR-ESI⁺): Exact mass calculated for [M+H]⁺: m/z=1058.2396, measured: m/z=1058.2400.

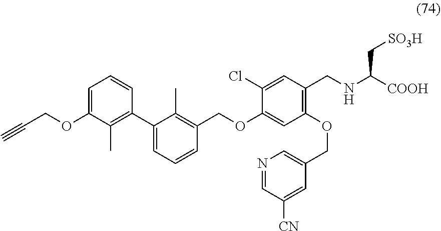 [SMILES: C#CCOc1cccc(-c2cccc(COc3cc(OCc4cncc(C#N)c4)c(CN[C@@H](CS(=O)(=O)O)C(=O)O)cc3Cl)c2C)c1C]

(5-Chloro-2-((5-cyanopyridin-3-yl)methoxy)-4-((2,2′-dimethyl-3′-(prop-2-yn-1-yloxy)-[1,1′-biphenyl]-3-yl)methoxy)benzyl)(sulfo)-L-alanine (74). Aldehyde 50 (700 mg, 130 μmol, 1.00 equiv.), L-cysteic acid (231 mg, 652 μmol, 5.00 equiv.) and sodium cyanoborohydride (12.3 mg, 196 μmol, 1.50 equiv.) were reacted in a mixture of absolute MeOH (1 mL) and DMF (1 mL) according to GP-3. After reaction completion monitored by RP-HPLC (system A), the solvent was removed in vacuo and the residue was purified by semi-preparative RP-HPLC (Agilent Zorbax SB C-18 5 μm 80 Å, 9.4×250 mm with 37-90% acetonitrile (0.1% TFA) in water (0.1% TFA) in a linear gradient over 45 min, 6 mL/min, Rₜ=12 min). Subsequent lyophilization gave carboxylic acid 74 (80.0 mg, 116 μmol, 89%) as a colorless powder. mp=180° C. Rₜ=12.53 min (System A), purity: 99.1%. ¹H NMR (400 MHz, DMSO-d₆) δ=9.34 (bs, 1H), 9.12 (bs, 1H), 9.02-9.03 (m, 2H), 8.49 (s, 1H), 7.55 (s, 1H), 7.46 (d, ³J=7.7 Hz, 1H), 7.22-7.28 (m, 2H), 7.18 (s, 1H), 7.04-7.09 (m, 2H), 6.74 (d, ³J=7.6 Hz, 1H), 5.39-5.40 (m, 2H), 5.30-5.32 (m, 2H), 4.85 (d, ⁴J=2.3 Hz, 2H), 4.32-4.35 (m, 1H), 4.20-4.24 (m, 2H), 3.59 (t, ⁴J=2.0 Hz, 1H), 3.05-3.07 (m, 1H), 2.88-2.94 (m, 1H), 2.03 (s, 3H), 1.83 ppm (s, 3H). ¹³C NMR (101 MHz, DMSO-d₆) δ=168.4, 156.3, 155.4, 155.4, 152.4, 151.9, 142.3, 141.5, 139.0, 134.5, 134.4, 132.5, 132.4, 129.3, 127.7, 126.2, 125.4, 124.1, 122.0, 116.9, 113.1, 113.0, 110.9, 109.1, 100.2, 79.5, 78.1, 69.7, 67.0, 56.2, 55.8, 48.6, 44.6, 15.3, 12.8 ppm. MS (HR-ESI⁺): Exact mass calculated for [M+H]⁺: m/z=690.1672, measured: m/z=699.1674.

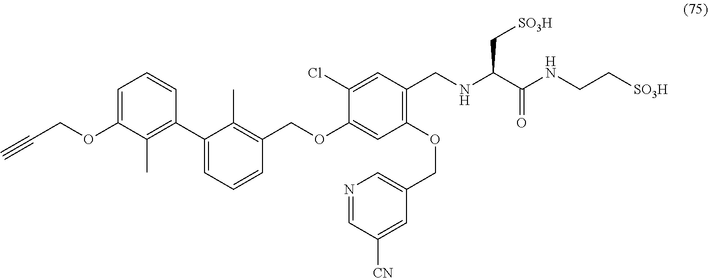 [SMILES: C#CCOc1cccc(-c2cccc(COc3cc(OCc4cncc(C#N)c4)c(CN[C@@H](CS(=O)(=O)O)C(=O)NCCS(=O)(=O)O)cc3Cl)c2C)c1C]

(R)-2-((5-Chloro-2-((5-cyanopyridin-3-yl)methoxy)-4-((2,2′-dimethyl-3′-(prop-2-yn-1-yloxy)-[1,1′-biphenyl]-3-yl)methoxy)benzyl)amino)-3-oxo-3-((2-sulfoethyl)amino)propane-1-sulfonic acid (75). The carboxylic acid 74 (80.0 mg, 116 μmol, 1.00 equiv.) was reacted with 2-aminoethan-1-sulfonic acid (TBA salt) (21.8 mg, 174 μmol, 1.50 equiv.) in presence of DIPEA (5.0 μL, 232 mol, 2.00 equiv.) and HBTU (52.8 mg, 139 mmol, 1.20 equiv.) in abs. DMF (5 mL) and subsequently worked up according to GP-6. After purification by semi-preparative RP-HPLC (Agilent Zorbax SB C-18 5 μm 80 Å, 9.4×250 mm with 30-90% acetonitrile (0.1% TFA) in water (0.1% TFA) in a linear gradient over 45 min, 6 mL/min, Rₜ=11 min) and lyophilization, compound 75 (80.0 mg, 100 μmol, 87%) was obtained as a colorless powder. mp=180-190° C. (decomposition). Rₜ=11.07 min (System A), purity: 92.0%. ¹H NMR (400 MHz, DMSO-d₆) δ=9.36 (bs, 1H), 9.02 (bs, 2H), 8.80 (bs, 1H), 8.51 (s, 1H), 7.53 (s, 1H), 7.46 (bs, 1H), 7.22-7.28 (m, 2H), 7.16 (s, 1H), 7.04-7.09 (m, 2H), 6.74 (d, ³J=7.5 Hz, 1H), 5.25-5.45 (m, 4H), 4.85 (s, 1H), 4.74 (bs, 1H), 4.21-4.25 (m, 2H), 3.95-4.05 (m, 1H), 3.58-3.59 (m, 2H), 3.37-3.39 (m, 1H), 2.98-3.01, (m, 1H), 2.71-2.77 (m, 1H), 2.64 (t, 3J=6.8 Hz, 1H), 2.03 (s, 3H), 1.83 ppm (s, 3H). ¹³C NMR (101 MHz, DMSO-d₆) δ=165.3, 156.2, 155.4, 155.4, 152.3, 151.8, 142.3, 141.5, 139.1, 134.5, 134.5, 132.7, 129.3, 127.7, 126.2, 125.5, 124.1, 122.1, 116.9, 113.0, 112.7, 110.9, 109.1, 100.0, 79.5, 78.2, 69.7, 66.9, 56.4, 55.8, 49.8, 49.7, 44.4, 36.3, 15.3, 12.8 ppm. MS (HR-ESI⁺): Exact mass calculated for [M+H]⁺: m/z=797.1713, measured: m/z=797.1712.

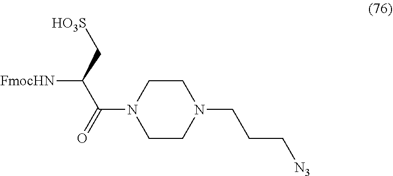

(R)-2-((((9H-Fluoren-9-yl)methoxy)carbonyl)amino)-3-(4-(3-azidopropyl)piperazin-1-yl)-3-oxopropane-1-sulfonic acid (76). Fmoc-L-cysteic acid (120 mg, 307 μmol, 1.00 equiv.), 1-(3-azidopropyl)piperazine (31) (77.8 mg, 460 μmol, 1.50 equiv.) and pyridine (79 μL, 921 μmol, 3.00 equiv.) were dissolved in abs. DMF (3 mL) and cooled down to 0° C. HBTU (233 mg, 613 μmol, 2.00 equiv.) was added, the reaction was allowed to warm to room temperature and was then stirred for 16 h. After completion of the reaction (monitored by RP-HPLC, system A), the solvent was removed and the residue was purified by semi-preparative RP-HPLC (System C, Rₜ=13 min). Subsequent lyophilization yielded compound 76 (116 mg, 214 μmol, 70%) as a colorless powder. m.p.=155° C. Rₜ=9.12 min (System A), purity: 98.1%. ¹H NMR (400 MHz, DMSO-d₆) δ=7.89 (d, ³J=7.5 Hz, 2H), 7.71 (d, ³J=7.4 Hz, 2H), 7.41 (t, ³J=7.4 Hz, 2H), 7.31-7.34 (m, 2H), 4.77-4.82 (m, 1H), 4.46-4.50 (m, 1H), 4.18-4.27 (m, 3H), 3.45-3.47 (m, 4H), 3.10-3.17 (m, 2H), 2.92-3.03 (m, 2H), 2.72 (m, 1H), 1.88-1.92 ppm (m, 2H). ¹³C NMR (101 MHz, DMSO-d₆) δ=170.3, 155.6, 143.8, 143.7, 140.7, 127.6, 127.1, 127.0, 125.4, 125.3, 120.1, 65.8, 53.6, 53.4, 50.5, 4.78, 47.1, 46.6, 43.6, 42.5, 42.1, 23.8, 22.9 ppm. MS (HR-ESI⁺): Exact mass calculated for [M+H]⁺: m/z=543.2021, measured: m/z=543.2025.

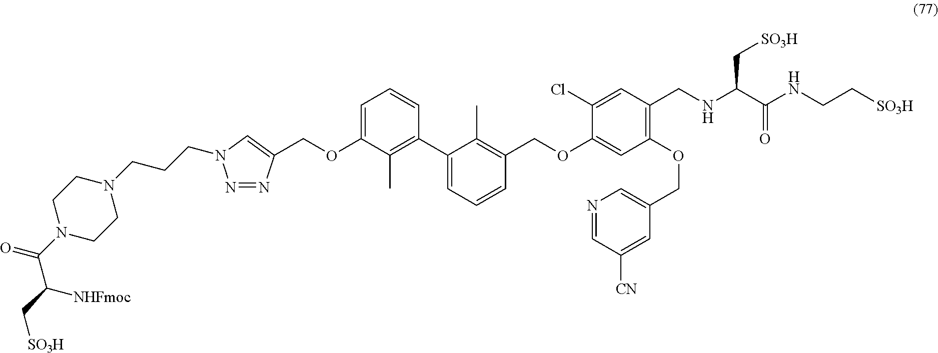

(R)-2-((4-((3′-((1-(3-(4-((((9H-Fluoren-9-yl)methoxy)carbonyl)(sulfo)-D-alanyl)piperazin-1-yl)propyl)-1H-1,2,3-triazol-4-yl)methoxy)-2,2′-dimethyl-[1,1′-biphenyl]-3-yl)methoxy)-5-chloro-2-((5-cyanopyridin-3-yl)methoxy)benzyl)amino)-3-oxo-3-((2-sulfoethyl)amino)propane-1-sulfonic acid (77). Alkyne 75 (80.0 mg, 100 μmol, 1.00 equiv.) reacted with the linker structure 76 (65.3 mg, 120 μmol, 1.20 equiv.) with a premixed catalyst consisting of CuSO₄ (4.80 mg, 30.1 μmol, 0.30 equiv.), THPTA (6.54 mg, 15.1 μmol, 0.15 equiv.) and sodium ascorbate (99.3 mg, 502 μmol, 5.00 equiv.) in an 1:1 mixture of H₂O/t-BuOH (3 mL) at room temperature for 16 h according to GP-5. The solvent was removed in vacuo and the residue was used in the next step without further purification.

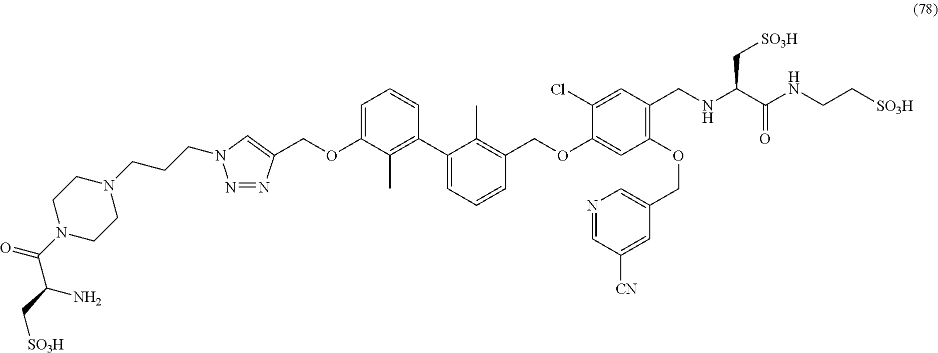 [SMILES: Cc1c(COc2cc(OCc3cncc(C#N)c3)c(CN[C@@H](CS(=O)(=O)O)C(=O)NCCS(=O)(=O)O)cc2Cl)cccc1-c1cccc(OCc2cn(CCCN3CCN(C(=O)[C@@H](N)CS(=O)(=O)O)CC3)nn2)c1C]

(R)-2-Amino-3-(4-(3-(4-(((3′-((2-chloro-5-((5-cyanopyridin-3-yl)methoxy)-4-((((R)-1-oxo-3-sulfo-1-((2-sulfoethyl)amino)propan-2-yl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-3-oxopropane-1-sulfonic acid (78). The residue from the previous reaction was dissolved in abs. DMF (3 mL) and sodium azide (13.0 mg, 200 μmol, 2.00 equiv.) was added. After heating at 60° C. for 3 h, the reaction was stopped and the solvent was removed under reduced pressure. The residue was purified by semi-preparative RP-HPLC (Agilent Zorbax SB C-18 5 μm 80 Å, 9.4×250 mm with 30-70% acetonitrile (0.1% TFA) in water (0.1% TFA) in a linear gradient over 45 min, 6 mL/min, Rₜ=7 min) and subsequent lyophilization yielded primary amine 78 (44.0 mg, 39.4 μmol, 39% over two steps) as a colorless solid. m.p.=240-250° C. Rₜ=8.97 min (System A), purity: 98.5%. ¹H NMR (400 MHz, DMSO-d₆) δ=9.73 (bs, 1H), 9.36 (bs, 1H), 9.00-9.02 (m, 2H), 8.78-8.81 (m, 1H), 8.50 (s, 1H), 8.30 (s, 1H), 8.10-8.10 (m, 2H), 7.52 (d, ³J=7.5 Hz, 1H), 7.45 (t, ³J=7.6 Hz, 1H), 7.16-7.26 (m, 3H), 7.07 (d, 3J=7.6 Hz, 1H), 6.73 (d, ³J=7.5 Hz, 1H), 5.34-5.45 (m, 2H), 5.17-5.31 (m, 3H), 4.51 (t, ³J=6.7 Hz, 2H), 4.26 (bs), 4.00 (bs), 3.55 (bs), 3.38-3.39 (m), 3.19 (bs), 2.96-3.03 (m), 2.73-2.80 (m), 2.65-2.67 (m, 1H), 2.29 (bs, 2H), 2.02-2.03 (m, 3H), 1.79 ppm (m, 3H). ¹³C NMR (101 MHz, DMSO-d₆) δ=166.4, 165.4, 158.6, 158.2, 156.3, 156.2, 156.2, 155.4, 155.4, 152.4, 151.9, 143.3, 142.3, 141.6, 139.0, 134.8, 134.7, 134.5, 132.7, 129.3, 128.0, 126.4, 125.5, 124.5, 124.0, 121.8, 117.0, 113.1, 113.1, 112.7, 110.8, 109.1, 100.1, 69.8, 66.9, 61.8, 56.4, 53.2, 50.8, 49.8, 49.7, 47.6, 46.7, 44.4, 42.0, 36.2, 24.3, 15.4, 12.8 ppm. MS (HR-ESI⁺): Exact mass calculated for [M+H]⁺: m/z=1117.2979, measured: m/z=1117.2975.

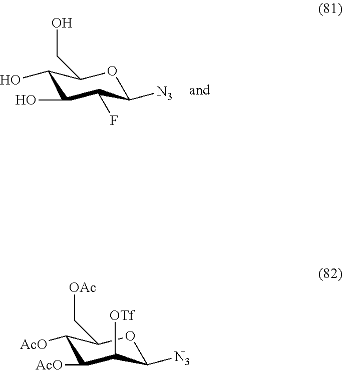

were synthesized according to the literature [16].

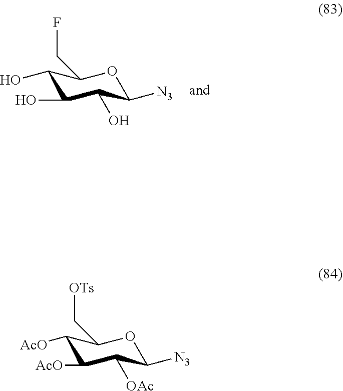

were synthesized according to the literature[17].

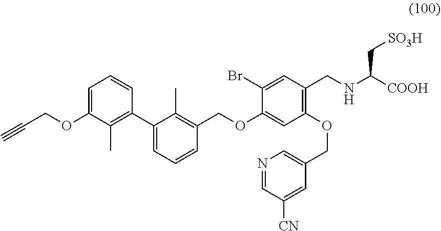 [SMILES: C#CCOc1cccc(-c2cccc(COc3cc(OCc4cncc(C#N)c4)c(CN[C@@H](CS(=O)(=O)O)C(=O)O)cc3Br)c2C)c1C]

(5-Bromo-2-((5-cyanopyridin-3-yl)methoxy)-4-((2,2′-dimethyl-3′-(prop-2-yn-1-yloxy)-[1,1′-biphenyl]-3-yl)methoxy)benzyl)(sulfo)-L-alanine (100). Aldehyde 59 (100 mg, 172 μmol, 1.00 equiv.), L-cysteic acid (305 mg, 860 μmol, 5.00 equiv.) and sodium cyanoborohydride (16.2 mg, 258 μmol, 1.50 equiv.) were reacted in a mixture of absolute MeOH/DMF (2 mL) according to GP-3. After reaction completion monitored by RP-HPLC (system A), the solvent was removed in vacuo and the residue was purified by semi-preparative RP-HPLC (Agilent Zorbax SB C-18 5 μm 80 Å, 9.4×250 mm with 40-90% acetonitrile (0.1% TFA) in water (0.1% TFA) in a linear gradient over 45 min, 6 mL/min, Rₜ=10 min). Subsequent lyophilization gave to carboxylic acid (100) (113 mg, 154 μmol, 89%) as a colorless powder. m.p.=185-190° C. Rₜ=13.09 min (System A), purity: 98.5%. ¹H NMR (400 MHz, DMSO-d₆) δ=9.21 (bs, 1H), 9.02 (dd, ⁴J=1.9 Hz, ³J=4.5 Hz, 2H), 8.49 (t, ⁴J=2.0 Hz, 1H), 7.67 (s, 1H), 7.48 (dd, ⁴J=1.8, 3J=7.6 Hz, 1H), 7.22-7.26 (bs, 2H), 7.15 (s, 1H), 7.04-7.09 (m, 2H), 6.74 (d, ³J=7.6 Hz, 1H), 5.36-5.44 (m, 2H), 5.26-5.34 (m, 2H), 4.85-4.86 (m, 2H), 4.31-4.34 (m, 1H), 4.16-4.24 (m, 2H), 3.59 (t, ⁴J=2.4 Hz, 1H), 3.02-3.07 (m, 1H), 2.87-2.93 (m, 1H), 2.03 (s, 3H), 1.83 ppm (s, 3H). ¹³C NMR (101 MHz, DMSO-d₆) δ=168.4, 156.9, 156.4, 155.4, 152.5, 151.9, 142.3, 141.5, 139.0, 135.3, 134.5, 133.3, 132.5, 129.2, 127.6, 126.2, 125.4, 124.1, 122.0, 116.9, 113.7, 110.9, 109.1, 101.8, 100.0, 79.5, 78.1, 69.7, 66.9, 56.3, 55.8, 48.6, 44.6, 15.4, 12.8 ppm. MS (HR-ESI⁺): Exact mass calculated for [M+H]⁺: m/z=734.1166, measured: m/z=734.1165.

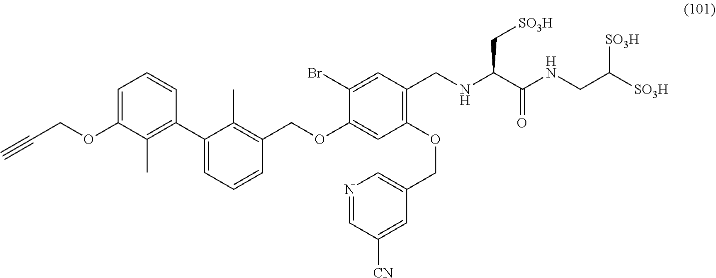 [SMILES: C#CCOc1cccc(-c2cccc(COc3cc(OCc4cncc(C#N)c4)c(CN[C@@H](CS(=O)(=O)O)C(=O)NCC(S(=O)(=O)O)S(=O)(=O)O)cc3Br)c2C)c1C]

(S)-2-(2-((5-Bromo-2-((5-cyanopyridin-3-yl)methoxy)-4-((2,2′-dimethyl-3′-(prop-2-yn-1-yloxy)-[1,1′-biphenyl]-3-yl)methoxy)benzyl)amino)-3-sulfopropanamido)ethane-1,1-disulfonic acid (101). Carboxylic acid 100 (100 mg, 136 μmol, 1.00 equiv.) was reacted with 2-aminoethan-1,1-disulfonic acid (TBA salt) (18) (160 mg, 408 μmol, 3.00 equiv.) in presence of DIPEA (71.3 μL, 408 μmol, 3.00 equiv.) and HBTU (103 mg, 272 μmol, 2.00 equiv.) in abs. DMF (2 mL) and subsequently worked up according to GP-6. After purification by semi-preparative RP-HPLC (Agilent Zorbax SB C-18 5 μm 80 Å, 9.4×250 mm with 30-80% acetonitrile (0.1% TFA) in water (0.1% TFA) in a linear gradient over 45 min, 6 mL/min, Rₜ=9 min) and subsequent desalting and lyophilization, compound 101 (100 mg, 109 μmol, 80%) was obtained as a colorless powder. m.p.=180° C. (decomposition). Rₜ=10.72 min (System A), purity: 100%. ¹H NMR (600 MHz, DMSO-d₆) δ=9.35 (bs, 1H), 9.03 (d, ⁴J=1.8 Hz, 1H), 8.99 (s, 1H), 8.51 (s, 1H), 8.43-8.44 (m, 1H), 7.72 (bs, 1H), 7.59 (bs, 1H), 7.50 (bs, 1H), 7.22-7.28 (m, 2H), 7.05-7.08 (m, 3H), 6.75 (d, ³J=7.5 Hz, 1H), 5.23-5.35 (bs, 4H), 4.86 (d, 3J=4.9 Hz, 2H), 3.58-3.61 (m, 6H), 3.13 (bs, 5H), 2.03 (s, 3H), 1.84 ppm (s, 3H). ¹³C NMR (101 MHz, DMSO-d₆) δ=155.4, 152.5, 151.8, 142.4, 141.4, 139.1, 134.4, 134.4, 129.1, 127.5, 126.2, 125.4, 124.2, 122.1, 116.9, 110.9, 109.0, 79.5, 78.1, 75.5, 73.6, 69.6, 67.2, 66.9, 60.9, 55.8, 53.6, 49.5, 45.7, 44.4, 41.8, 15.3, 12.8 ppm. MS (HR-ESI): Exact mass calculated for [M−H]⁺: m/z=919.0630, measured: m/z=919.0622.

### Preparation of the fluorine Compounds according to the invention

### Example 1—Compound 85

### 2-(2-((4-((2-Bromo-3′-((1-(3-fluoropropyl)-1H-1,2,3-triazol-4-yl)methoxy)-[1,1′-biphenyl]-3-yl)methoxy)-5-chloro-2-((5-(methylsulfonyl)pyridin-3-yl)methoxy)benzyl)(methyl)amino)acetamido)ethane-1,1-disulfonic acid (85)

Compound 19 (25.0 mg, 23.0 μmol, 1.00 equiv.) and 3-fluoro-azidopropane (1 M in THF, 232 μL, 115 μmol, 5.00 equiv.) were dissolved in DMF (2 mL), reacted in presence of TBTA (0.1 mg, 0.2 mol, 0.01 equiv.) and [Cu(MeCN)₄]PF₆ (4.3 mg, 11.5 μmol, 0.50 equiv.) according to GP-4 and purified by semi-preparative RP-HPLC (System C, Rt=22 min) to yield fluoropropyl compound (85) (16.3 mg, 16.0 μmol, 69%) as a colorless powder.

Rt=9.86 min (System A), purity: 95.0%. ¹H NMR (400 MHz, DMSO-d₆) δ=9.61 (bs, 1H), 8.59-8.57 (m, 1H), 8.28 (s, 1H), 8.13 (bs, 1H), 7.70-7.65 (m, 2H), 7.56 (t, 3J=7.6 Hz, 1H), 7.42-7.39 (m, 2H), 7.22 (s, 1H), 7.10 (d, 3J=8.5 Hz, 1H), 7.04 (s, 1H), 6.98 (d, 3J=7.5 Hz, 1H), 5.53 (s, 2H), 5.80 (s, 2H), 5.19 (s, 2H), 4.74 (bs, 2H), 4.54-4.31 (m, 4H), 3.92 (bs, 2H), 3.68 (bs, 2H), 3.38 (s, 3H), 2.72 (s, 3H), 2.29-2.18 ppm (m, 2H). ¹³C NMR (101 MHz, DMSO-d₆) δ=163.4, 157.7, 156.9, 155.6, 142.9, 142.6, 142.1, 136.0, 134.0, 131.2, 129.4, 129.3, 127.8, 124.7, 123.1, 121.9, 115.7, 114.0, 113.4, 111.3, 109.6, 100.2, 81.8, 80.2, 71.2, 6.12, 56.0, 52.8, 45.9, 45.9, 43.7, 40.6, 30.7, 30.5 ppm. ¹⁹F NMR (376 MHz, DMSO-d6) δ=−220.2-(−220.6) ppm (m). IR (ATR): \tilde{v}=3365 (m), 3054 (m), 2925 (w), 1682 (m), 1605 (m), 1580 (w), 1506 (w), 1461 (w), 1406 (w), 1536 (m), 1202 (s), 1175 (s), 1154 (s), 1062 (m), 1018 (s), 931 (w), 778 (w), 700 (w), 651 (w), 590 cm⁻¹ (w).

### Example 2—Compound (86)

### 2-(2-((4-((2-Bromo-3′-((1-((2R,3R,4S,5S,6R)-3-fluoro-4,5-dihydroxy-6-(hydroxymethyl)tetrahydro-2H-pyran-2-yl)-1H-1,2,3-triazol-4-yl)methoxy)-[1,1′-biphenyl]-3-yl)methoxy)-5-chloro-2-((5-(methylsulfonyl)pyridin-3-yl)methoxy)benzyl)(methyl)amino)acetamido)ethane-1,1-disulfonic acid (86)

Compound 19 (25.0 mg, 23.0 μmol, 1.00 equiv.) and azido sugar 81 (9.5 mg, 46.0 μmol, 2.00 equiv.) were dissolved in DMF (2 mL), reacted in presence of TBTA (0.1 mg, 0.2 mol, 0.01 equiv.) and [Cu(MeCN)4]PF6 (4.3 mg, 11.5 μmol, 0.50 equiv.) according to GP-4 and purified by semi-preparative RP-HPLC (System C, Rt=18 min) to yield 2-F-sugar compound (86) (10.1 mg, 10.8 μmol, 47%) as a colorless powder.

Rt=8.94 min (System A), purity: 95.5%. 1H NMR (400 MHz, DMSO-d6) δ=9.62 (bs, 1H), 9.16-9.09 (m, 2H), 8.65 (s, 1H), 8.56-8.52 (m, 1H), 8.13-8.12 (m, 1H), 7.71-7.69 (m, 1H), 7.66 (s, 1H), 7.56 (t, 3J=7.6 Hz, 1H), 7.43-7.39 (m, 2H), 7.22 (s, 1H), 7.12 (d, 3J=8.4 Hz, 1H), 7.06 (s, 1H), 6.99 (d, 3J=7.5 Hz, 1H), 6.07 (d, 3J=7.4 Hz, 1H), 5.50 (s, 2H), 5.38 (s, 2H), 5.23 (s, 2H), 4.87 (dt, 2J=51.0, 3J=9.11 Hz, 2H), 3.92-3.30 (m), 2.72 ppm (s, 3). 13C NMR (101 MHz, DMSO-d₆) δ=163.4, 157.7, 156.9, 155.6, 152.7, 146.8, 143.2, 142.8, 142.2, 137.1, 136.0, 135.1, 134.0, 133.2, 131.2, 129.4, 129.3, 127.8, 124.2, 123.1, 122.0, 115.6, 114.0, 113.4, 111.3, 109.5, 100.2, 91.8, 89.9, 84.2, 83.9, 79.9, 74.4, 74.2, 74.1, 71.2, 69.4, 69.3, 67.4, 61.0, 60.4, 56.1, 52.8, 43.7, 40.7 ppm. 19F NMR (376 MHz, DMSO-d6) δ=−197.4-(−197.6) ppm. IR (ATR): \tilde{v}=3364 (m), 3086 (w), 2926 (w), 1682 (m), 1606 (w), 1580 (w), 1506 (w), 1461 (w), 1407 (w), 1306 (m), 1203 (s), 1175 (s), 1155 (s), 1064 (m), 1021 (s), 899 (w), 777 (w), 699 (w), 659 (w), 591 cm⁻¹ (w).

### Example 3

### 2-(2-((4-((2-Bromo-3′-((1-((2R,3R,4S,5S,6S)-6-(fluoromethyl)-3,4, 5-trihydroxytetrahydro-2H-pyran-2-yl)-1H-1,2,3-triazol-4-yl)methoxy)-[1,1′-biphenyl]-3-yl)methoxy)-5-chloro-2-((5-(methylsulfonyl)pyridin-3-yl)methoxy)benzyl)(methyl)amino)acetamido)ethane-1,1-disulfonic acid (87)

Compound 19 (25.0 mg, 23.0 μmol, 1.00 equiv.) and azido sugar 83 (9.5 mg, 46.0 μmol, 2.00 equiv.) were dissolved in DMF (2 mL), reacted in presence of TBTA (0.1 mg, 0.2 mol, 0.01 equiv.) and [Cu(MeCN)4]PF6 (4.3 mg, 11.5 μmol, 0.50 equiv.) according to GP-4 and purified by semi-preparative RP-HPLC (System C, Rt=17 min) to yield 6-F-sugar compound (87) (4.9 mg, 4.5 μmol, 20%) as a colorless solid.

Rt=8.97 min (System A), purity: 98.6%. 1H NMR (400 MHz, DMF-d₇) δ=9.68 (bs, 1H), 9.47 (s, 1H), 9.25 (s, 1H), 8.83 (s, 1H), 8.58 (s, 1H), 8.26 (bs, 1H), 7.84-7.80 (m, 2H), 7.58 (t, 1H), 7.46-7.42 (m, 3H), 7.18-7.17 (m, 2H), 7.03 (d, 3J=7.6 Hz, 1H), 5.81 (d, 3J=9.3 Hz, 1H), 5.71 (s, 2H), 5.50 (s, 2H), 5.31 (s, 2H), 4.74-4.58 (m), 4.07-3.83 (m), 3.69 (t, 3J=8.9 Hz, 1H), 3.52-3.47 (m, 4H), 3.03 ppm (s, 3H). ¹³C NMR (101 MHz, DMF-d₇) δ=164.6, 158.5, 157.7, 156.5, 151.3, 145.5, 143.7, 143.5, 143.0, 138.8, 138.0, 136.9, 135.2, 134.7, 131.6, 129.7, 129.6, 128.1, 124.2, 123.4, 122.4, 116.1, 114.7, 114.4, 112.3, 110.3, 100.6, 88.3, 83.8, 82.1, 78.3, 78.1, 77.8, 74.6, 73.0, 71.8, 69.4, 69.3, 68.0, 61.8, 57.0, 53.8, 43.9, 41.2, 39.5 ppm. ¹⁹F NMR (376 MHz, DMF-d7) δ=−233.3-(−233.6) ppm. IR (ATR): \tilde{v}=3364 (m), 3087 (w), 2924 (w), 1682 (m), 1606 (m), 1580 (w), 1506 (w), 1461 (w), 1407 (w), 1305 (m), 1203 (s), 1155 (m), 1064 (m), 1019 (s), 903 (w), 778 (s), 699 (w), 659 (w), 590 cm⁻¹ (m).

### Example 4—Compound 88

### 2-(2-((5-Chloro-4-((3′-((1-(3-fluoropropyl)-1H-1,2,3-triazol-4-yl)methoxy)-2,2′-dimethyl-[1,1′-biphenyl]-3-yl)methoxy)-2-((5-(methylsulfonyl)pyridin-3-yl)methoxy)benzyl)(methyl)amino)acetamido)ethane-1,1-disulfonic acid (88)

Compound 27 (25.0 mg, 24.1 μmol, 1.00 equiv.) and 3-fluoro-azidopropane (1 M in THF, 297 μL, 147 μmol, 5.00 equiv.) were dissolved in DMF (2 mL), reacted in presence of TBTA (0.1 mg, 0.2 mol, 0.01 equiv.) and [Cu(MeCN)4]PF6 (4.4 mg, 11.8 μmol, 0.50 equiv.) according to GP-4 and purified by semi-preparative RP-HPLC (System C, Rt=23 min) to yield fluoropropyl compound (88) (13.5 mg, 13.8 μmol, 46%) as a colorless powder.

Rt=10.11 min (System A), purity: 99.3%. 1H NMR (400 MHz, DMF-d₇) δ=9.68 (bs, 1H), 9.52 (s, 1H), 9.28 (s, 1H), 8.87 (s, 1H), 8.39 (s, 1H), 8.28 (s, 1H), 7.81 (s, 1H), 7.65 (d, 3J=7.6 Hz, 1H), 7.47 (s, 1H), 7.36-7.32 (m, 1H), 7.29-7.26 (m, 2H), 7.15 (d, 3J=7.6 Hz, 1H), 6.78-6.77 (m, 1H), 5.71 (s, 2H), 5.47 (s, 2H), 5.30 (s, 2H), 4.67-4.48 (m, 6H), 3.53 (s, 3H), 3.03 (s, 3H), 2.41-2.34 (m, 2H), 2.13 (s, 3H), 1.88 ppm (s, 3H). ¹³C NMR (400 MHz, DMF-d₇) δ=164.6, 157.6, 157.1, 156.7, 150.9, 145.2, 144.0, 143.1, 142.4, 138.6, 135.4, 135.3, 135.3, 134.6, 129.9, 128.4, 126.7, 125.9, 124.8, 124.7, 122.3, 114.4, 112.0, 111.1, 100.6, 82.3, 80.7, 74.7, 70.5, 67.9, 62.4, 57.0, 53.7, 46.5, 46.5, 43.8, 41.1, 39.5, 31.5, 31.3, 15.4, 12.8 ppm. ¹⁹F NMR (376 MHz, DMF-d7) δ=−220.0-(−222.4) ppm. IR (ATR): \tilde{v}=3445 (m), 3010 (m), 2925 (m), 1682 (m), 1607 (w), 1576 (w), 1509 (w), 1455 (m), 1408 (w), 1308 (m), 1240 (s), 1202 (s), 1172 (s), 1146 (s), 1063 (m), 1017 (m), 770 (w), 719 (w), 675 (w), 590 cm⁻¹ (w).

### Example 5—Compound 89

### 2-(2-((5-Chloro-4-((3′-((1-((2R,3R,4S,5S,6R)-3-fluoro-4,5-dihydroxy-6-(hydroxymethyl)tetrahydro-2H-pyran-2-yl)-1H-1,2,3-triazol-4-yl)methoxy)-2,2′-dimethyl-[1,1′-biphenyl]-3-yl)methoxy)-2-((5-(methylsulfonyl)pyridin-3-yl)methoxy)benzyl)(methyl)amino)acetamido)ethane-1,1-disulfonic acid (89)

Compound 27 (25.0 mg, 24.1 μmol, 1.00 equiv.) and azido sugar 81 (10.0 mg, 48.3 μmol, 2.00 equiv.) were dissolved in DMF (2 mL), reacted in presence of TBTA (0.1 mg, 0.2 mol, 0.01 equiv.) and [Cu(MeCN)₄]PF₆ (4.4 mg, 11.8 μmol, 0.50 equiv.) according to GP-4 and purified by semi-preparative RP-HPLC (System C, Rt=18 min) to yield 2-F-sugar compound 89 (15.2 mg, 14.2 μmol, 59%) as a colorless powder.

Rt=9.04 min (System A), purity: 97.4%. ¹H NMR (400 MHz, DMSO-d₆) δ=9.60 (bs, 1H), 9.18-9.12 (m, 2H), 8.65 (s, 1H), 8.58-8.53 (m, 1H), 8.13-8.11 (m, 1H), 7.63 (d, 3J=4.0 Hz, 1H), 7.52 (d, 3J=7.6 Hz, 1H), 7.29-7.21 (m, 4H), 7.09 (d, 3J=7.3 Hz, 1H), 6.74 (d, 3J=7.3 Hz, 1H), 6.07 (d, 3J=9.1, 2.2 Hz, 1H), 5.50 (s, 2H), 5.37-5.30 (m, 2H), 5.24 (s, 2H), 4.91 (dt, 2J=50.9 Hz, 3J=9.0 Hz, 2H), 3.79-3.35 (m), 3.32 (t, 3J=9.3 Hz, 1H), 2.71 (s, 3H), 2.04 (s, 3H), 1.83 ppm (s, 3H). 13C NMR (101 MHz, DMSO-d6) δ=163.4, 156.9, 156.1, 155.7, 152.7, 146.8, 143.6, 142.3, 141.6, 137.2, 135.2, 134.7, 134.4, 133.9, 133.3, 129.4, 127.9, 126.4, 125.5, 124.0, 123.9, 121.8, 113.4, 111.0, 110.6, 109.6, 100.3, 91.7, 89.9, 84.2, 84.0, 79.9, 74.4, 74.3, 74.0, 69.8, 69.4, 69.3, 67.4, 61.5, 60.4, 56.0, 52.8, 43.7, 40.6, 15.3, 12.9 ppm. 19F NMR (376 MHz, DMSO-d6) δ=−197.4-(−197.55) ppm. IR (ATR): \tilde{v}=3366 (m), 3090 (w), 2926 (w), 1682 (m), 1607 (w), 1576 (w), 1509 (w), 1455 (m), 1576 (w), 1509 (w), 1455 (m), 1409 (w), 1308 (m), 1240 (s), 1203 (s), 1179 (s), 1152 (s), 1065 (m), 1044 (m), 1020 (s), 898 (w), 744 (w), 719 (w), 674 (w), 590 cm⁻¹ (w).

### Example 6—Compound 90

### 2-(2-((5-Chloro-4-((3′-((1-((2R,3R,4S,5S,6S)-6-(fluoromethyl)-3,4, 5-trihydroxytetrahydro-2H-pyran-2-yl)-1H-1,2,3-triazol-4-yl)methoxy)-2,2′-dimethyl-[1,1′-biphenyl]-3-yl)methoxy)-2-((5-(methylsulfonyl)pyridin-3-yl)methoxy)benzyl)(methyl)amino)acetamido)ethane-1,1-disulfonic acid (90)

Compound 27 (25.0 mg, 24.1 μmol, 1.00 equiv.) and azido sugar 83 (10.0 mg, 48.3 μmol, 2.00 equiv.) were dissolved in DMF (2 mL), reacted in presence of TBTA (0.1 mg, 0.2 mol, 0.01 equiv.) and [Cu(MeCN)₄]PF₆ (4.3 mg, 11.5 μmol, 0.50 equiv.) according to GP-4 and purified by semi-preparative RP-HPLC (System C, Rt=17 min) to yield 6-F-sugar compound (90) (17.9 mg, 17.0 μmol, 71%) as a colorless powder.

Rt=9.07 min (System A), purity: 98.4%. ¹H NMR (400 MHz, DMF-d₇) δ=9.70 (bs, 1H), 9.53 (s, 1H), 9.29 (s, 1H), 8.88 (s, 1H), 8.59 (s, 1H), 8.29 (s, 1H), 7.81 (s, 1H), 7.65 (d, 3J=7.4 Hz, 1H), 7.46 (s, 1H), 7.36-7.29 (m, 3H), 7.15 (d, 3J=7.3 Hz, 1H), 6.79 (m, 1H), 5.82 (d, 3J=9.3 Hz, 1H), 5.70 (s, 2H), 5.47 (s, 2H), 5.31 (s, 2H), 4.75-4.57 (m, 4H), 3.69 (t, 3J=8.9 Hz, 2H), 3.53-3.47 (m, 4H), 3.03 (s, 3H), 2.13 (s, 3H), 1.88 ppm (s, 3H). 13C NMR (101 MHz, DMF-d7) δ=164.6, 157.6, 157.1, 156.7, 150.9, 145.1, 143.9, 143.9, 143.1, 142.4, 138.7, 138.4, 135.4, 135.3, 135.2, 134.6, 129.9, 128.4, 126.7, 125.9, 124.8, 123.9, 122.3, 114.4, 111.9, 111.0, 110.3, 100.6, 88.3, 83.8, 82.1, 78.3, 78.1, 77.9, 74.7, 72.9, 70.5, 69.4, 69.3, 67.9, 62.3, 56.9, 53.7, 43.8, 41.1, 39.5, 15.4, 12.8 ppm. ¹⁹F NMR (376 MHz, DMF-d7) δ=−233.4-(−233.6) ppm. IR (ATR): \tilde{v}=3367 (m), 3055 (w), 2924 (w), 1682 (m), 1607 (w), 1578 (w), 1510 (w), 1555 (m), 1408 (w), 1309 (m), 1239 (s), 1202 (s), 1172 (s), 1153 (s), 1092 (m), 1063 (m), 1016 (s), 903 (w), 775 (w), 674 (w), 590 cm⁻¹ (m).

### Example 7—Compound 91

### 2-(2-((5-Chloro-2-((5-cyanopyridin-3-yl)methoxy)-4-((3′-((1-(3-fluoropropyl)-1H1,2,3-triazol-4-yl)methoxy)-2,2′-dimethyl-[1,1′-biphenyl]-3-yl)methoxy)benzyl)(methyl)amino)acetamido)ethane-1,1-disulfonic acid (91)

Alkyne 52 (2.51 μmol, 1.00 equiv.) was reacted with 3-fluoro-azidopropane (1 M in THF, 12.5 μL, 12.5 μmol, 5.00 equiv.) with a premixed catalyst consisting of CuSO₄ (0.12 mg, 0.75 μmol, 0.30 equiv.), THPTA (0.14 mg, 0.33 μmol, 0.15 equiv.) and sodium ascorbate (2.49 mg, 12.5 μmol, 5.00 equiv.) in an 1:1 mixture of H₂O/t-BuOH (200 μL) at room temperature for 16 h according to GP-5. The reaction mixture was directly injected in semi-preparative RP-HPLC (System C, Rt=15 min) and after lyophilization, compound 91 (1.58 mg, 1.75 μmol, 70%) was obtained as colorless solid. Rₜ=10.39 min (System A), purity: 98.1%.

### Example 8—Compound 92

### 2-(2-((5-bromo-2-((5-cyanopyridin-3-yl)methoxy)-4-((3′-((1-(3-fluoropropyl)-1H-1,2,3-triazol-4-yl)methoxy)-2,2′-dimethyl-[1,1′-biphenyl]-3-yl)methoxy)benzyl)(methyl)amino)acetamido)ethane-1,1-disulfonic acid (92)

Alkyne 61 (2.51 μmol, 1.00 equiv.) was reacted with 3-fluoro-azidopropane (1 M in THF, 12.5 μL, 12.5 μmol, 5.00 equiv.) with a premixed catalyst consisting of CuSO₄ (0.12 mg, 0.75 μmol, 0.30 equiv.), THPTA (0.14 mg, 0.33 μmol, 0.15 equiv.) and sodium ascorbate (2.49 mg, 12.5 μmol, 5.00 equiv.) in an 1:1 mixture of H₂O/t-BuOH (200 μL) at room temperature for 16 h according to GP-5. The reaction mixture was directly injected in semi-preparative RP-HPLC (System C, Rt=18 min) and after lyophilization, compound 92 (1.70 mg, 1.83 μmol, 77%) was obtained as colorless solids. Rₜ=10.46 min (System A), purity: 100%.

### Example 9—Compound 93

### 2-(2-((5-iodo-2-((5-cyanopyridin-3-yl)methoxy)-4-((3′-((1-(3-fluoropropyl)-1H-1,2,3-triazol-4-yl)methoxy)-2,2′-dimethyl-[1,1′-biphenyl]-3-yl)methoxy)benzyl)(methyl)amino)acetamido)ethane-1,1-disulfonic acid (93)

Alkyne 71 (2.51 μmol, 1.00 equiv.) was reacted with 3-fluoro-azidopropane (1 M in THF, 12.5 μL, 12.5 μmol, 5.00 equiv.) with a premixed catalyst consisting of CuSO₄ (0.12 mg, 0.75 μmol, 0.30 equiv.), THPTA (0.14 mg, 0.33 μmol, 0.15 equiv.) and sodium ascorbate (2.49 mg, 12.5 μmol, 5.00 equiv.) in an 1:1 mixture of H₂O/t-BuOH (200 μL) at room temperature for 16 h according to GP-5. The reaction mixture was directly injected in semi-preparative RP-HPLC (System C, Rₜ=20 min) and after lyophilization, compound 93 (1.87 mg, 1.89 μmol, 84%) was obtained as colorless solid. Rₜ=10.49 min (System A), purity: 96.0%.

### Example 10—Compound 94

### 2-(2-((5-Chloro-2-((5-cyanopyridin-3-yl)methoxy)-4-((3′-((1-((2R,3R,4S,5S,6R)-3-fluoro-4,5-dihydroxy-6-(hydroxymethyl)tetrahydro-2H-pyran-2-yl)-1H-1,2,3-triazol-4-yl)methoxy)-2,2′-dimethyl-[1,1′-biphenyl]-3-yl)methoxy)benzyl)(methyl)amino)acetamido)ethane-1,1-disulfonic acid (94)

Alkyne 52 (2.51 μmol, 1.00 equiv.) was reacted with 2-F-sugar 81 (1.04 mg, 5.02 μmol, 2.00 equiv.) with a premixed catalyst consisting of CuSO₄ (0.12 mg g, 0.75 μmol, 0.30 equiv.), THPTA (0.14 mg, 0.33 μmol, 0.15 equiv.) and sodium ascorbate (2.49 mg, 12.5 μmol, 5.00 equiv.) in an 1:1 mixture of H₂O/t-BuOH (200 μL) at room temperature for 16 h according to GP-5. The reaction mixture was directly injected in semi-preparative RP-HPLC (System C, Rₜ=13) and after lyophilization, compound 94 (0.91 mg, 0.91 μmol, 36%) was obtained as colorless solid. Rₜ=9.37 min (System A), purity: 97.5%.

### Example 11—Compound 95

### 2-(2-((5-bromo-2-((5-cyanopyridin-3-yl)methoxy)-4-((3′-((1-((2R,3R,4S,5S,6R)-3-fluoro-4,5-dihydroxy-6-(hydroxymethyl)tetrahydro-2H-pyran-2-yl)-1H-1,2,3-triazol-4-yl)methoxy)-2,2′-dimethyl-[1,1′-biphenyl]-3-yl)methoxy)benzyl)(methyl)amino)acetamido)ethane-1,1-disulfonic acid (95)

Alkyne 61 (2.51 μmol, 1.00 equiv.) was reacted with 2-F-sugar 81 (1.04 mg, 5.02 μmol, 2.00 equiv.) with a premixed catalyst consisting of CuSO₄ (0.12 mg g, 0.75 μmol, 0.30 equiv.), THPTA (0.14 mg, 0.33 μmol, 0.15 equiv.) and sodium ascorbate (2.49 mg, 12.5 μmol, 5.00 equiv.) in an 1:1 mixture of H₂O/t-BuOH (200 μL) at room temperature for 16 h according to GP-5. The reaction mixture was directly injected in semi-preparative RP-HPLC (System C, Rₜ=13) and after lyophilization, compound 95 (1.71 mg, 1.63 μmol, 69%) was obtained as colorless solid. Rₜ=9.42 min (System A), purity: 100%.

### Example 12—Compound 96

### 2-(2-((5-iodo-2-((5-cyanopyridin-3-yl)methoxy)-4-((3′-((1-((2R,3R,4S,5S,6R)-3-fluoro-4,5-dihydroxy-6-(hydroxymethyl)tetrahydro-2H-pyran-2-yl)-1H-1,2,3-triazol-4-yl)methoxy)-2,2′-dimethyl-[1,1′-biphenyl]-3-yl)methoxy)benzyl)(methyl)amino)acetamido)ethane-1,1-disulfonic acid (96)

Alkyne 71 (2.51 μmol, 1.00 equiv.) was reacted with 2-F-sugar 81 (1.04 mg, 5.02 μmol, 2.00 equiv.) with a premixed catalyst consisting of CuSO₄ (0.12 mg g, 0.75 μmol, 0.30 equiv.), THPTA (0.14 mg, 0.33 μmol, 0.15 equiv.) and sodium ascorbate (2.49 mg, 12.5 μmol, 5.00 equiv.) in an 1:1 mixture of H₂O/t-BuOH (200 μL) at room temperature for 16 h according to GP-5. The reaction mixture was directly injected in semi-preparative RP-HPLC (System C, Rₜ=15 min) and after lyophilization, compound 96 (1.97, 1.80 μmol, 80%) was obtained as colorless solid. Rₜ=8.52 min (System A), purity: 99.1%.

### Example 13—Compound 97

### 2-(2-((5-Chloro-2-((5-cyanopyridin-3-yl)methoxy)-4-((3′-((1-((2R,3R,4S,5S,6S)-6-(fluoromethyl)-3,4,5-trihydroxytetrahydro-2H-pyran-2-yl)-1H-1,2,3-triazol-4-yl)methoxy)-2,2′-dimethyl-[1,1′-biphenyl]-3-yl)methoxy)benzyl)(methyl)amino)acetamido)ethane-1,1-disulfonic acid (97)

Alkyne 52 (2.51 μmol, 1.00 equiv.) was reacted with 6-F-sugar 83 (1.04 mg, 5.02 μmol, 2.00 equiv.) with a premixed catalyst consisting of CuSO₄ (0.12 mg g, 0.75 μmol, 0.30 equiv.), THPTA (0.14 mg, 0.33 μmol, 0.15 equiv.) and sodium ascorbate (2.49 mg, 12.5 μmol, 5.00 equiv.) in an 1:1 mixture of H₂O/t-BuOH (200 μL) at room temperature for 16 h according to GP-5. The reaction mixture was directly injected in semi-preparative RP-HPLC (System C, Rₜ=13) and after lyophilization, compound 97 (0.89 mg, 0.89 μmol, 35%) were obtained as colorless solid. Rₜ=9.40 min (System A), purity: 96.9%.

### Example 14—Compound 98

### 2-(2-((5-bromo-2-((5-cyanopyridin-3-yl)methoxy)-4-((3′-((1-((2R,3R,4S,5S,6S)-6-(fluoromethyl)-3,4,5-trihydroxytetrahydro-2H-pyran-2-yl)-1H-1,2,3-triazol-4-yl)methoxy)-2,2′-dimethyl-[1,1′-biphenyl]-3-yl)methoxy)benzyl)(methyl)amino)acetamido)ethane-1,1-disulfonic acid (98)

Alkyne 61 (2.51 μmol, 1.00 equiv.) was reacted with 6-F-sugar 83 (1.04 mg, 5.02 μmol, 2.00 equiv.) with a premixed catalyst consisting of CuSO₄ (0.12 mg g, 0.75 μmol, 0.30 equiv.), THPTA (0.14 mg, 0.33 μmol, 0.15 equiv.) and sodium ascorbate (2.49 mg, 12.5 μmol, 5.00 equiv.) in an 1:1 mixture of H₂O/t-BuOH (200 μL) at room temperature for 16 h according to GP-5. The reaction mixture was directly injected in semi-preparative RP-HPLC (System C, Rₜ=13) and after lyophilization, compound 98 (1.19 mg, 1.13 μmol, 48%) was obtained as colorless solid. Rₜ=9.45 min (System A), purity: 99.0%.

### Example 15—Compound 99

### 2-(2-((5-iodo-2-((5-cyanopyridin-3-yl)methoxy)-4-((3′-((1-((2R,3R,4S,5S,6S)-6-(fluoromethyl)-3,4,5-trihydroxytetrahydro-2H-pyran-2-yl)-1H-1,2,3-triazol-4-yl)methoxy)-2,2′-dimethyl-[1,1′-biphenyl]-3-yl)methoxy)benzyl)(methyl)amino)acetamido)ethane-1,1-disulfonic acid (99)

Alkyne 71 (2.51 μmol, 1.00 equiv.) was reacted with 6-F-sugar 83 (1.04 mg, 5.02 μmol, 2.00 equiv.) with a premixed catalyst consisting of CuSO₄ (0.12 mg g, 0.75 μmol, 0.30 equiv.), THPTA (0.14 mg, 0.33 μmol, 0.15 equiv.) and sodium ascorbate (2.49 mg, 12.5 μmol, 5.00 equiv.) in an 1:1 mixture of H₂O/t-BuOH (200 μL) at room temperature for 16 h according to GP-5. The reaction mixture was directly injected in semi-preparative RP-HPLC (System C, Rₜ=15 min) and after lyophilization, compound 99 (1.05, 0.96 μmol, 43%) was obtained as colorless solid. Rₜ=9.55 min (System A), purity: 98.8%.

### Example 16—Compound 102

### (S)-2-(2-((5-Bromo-2-((5-cyanopyridin-3-yl)methoxy)-4-((3′-((1-(3-fluoropropyl)-1H-1,2,3-triazol-4-yl)methoxy)-2,2′-dimethyl-[1,1′-biphenyl]-3-yl)methoxy)benzyl)amino)-3-sulfopropanamido)ethane-1,1-disulfonic acid (102)

Alkyne 101 (2.00 mg, 2.17 μmol, 1.00 equiv.) reacted with 3-fluoro-azidopropane (1 M in THF, 10.8 μL, 12.5 μmol, 5.00 equiv.) with a premixed catalyst consisting of CuSO₄ (0.10 mg, 0.65 μmol, 0.30 equiv.), THPTA (0.12 mg, 0.28 μmol, 0.15 equiv.) and sodium ascorbate (2.15 mg, 10.8 μmol, 5.00 equiv.) in an 1:1 mixture of H₂O/t-BuOH (200 μL) at room temperature for 16 h according to GP-5. The reaction mixture was directly injected in semi-preparative RP-HPLC (System C, Rₜ=15 min) and after lyophilization, compound 102 (1.65 mg, 1.61 μmol, 74%) was obtained as colorless solid. Rₜ=9.83 min (System A), purity: 98.8%.

### Example 17—Compound 103

### 2-((S)-2-((5-bromo-2-((5-cyanopyridin-3-yl)methoxy)-4-((3′-((1-((2R,3R,4S,5S,6R)-3-fluoro-4,5-dihydroxy-6-(hydroxymethyl)tetrahydro-2H-pyran-2-yl)-1H-1,2,3-triazol-4-yl)methoxy)-2,2′-dimethyl-[1,1′-biphenyl]-3-yl)methoxy)benzyl)amino)-3-sulfopropanamido)ethane-1,1-disulfonic acid (103)

Alkyne 101 (2.00 μmol, 2.17 μmol, 1.00 equiv.) reacted with 2-F-sugar 81 (0.90 mg, 4.34 μmol, 2.00 equiv.) with a premixed catalyst consisting of CuSO₄ (0.10 mg g, 0.65 μmol, 0.30 equiv.), THPTA (0.12 mg, 0.33 μmol, 0.15 equiv.) and sodium ascorbate (2.15 mg, 10.8 μmol, 5.00 equiv.) in an 1:1 mixture of H₂O/t-BuOH (200 μL) at room temperature for 16 h according to GP-5. The reaction mixture was directly injected in semi-preparative RP-HPLC (System C, Rₜ=10 min) and after lyophilization, compound (103) (1.95 mg, 1.73 μmol, 80%) was obtained as colorless solid. Rₜ=8.98 min (System A), purity: 98.2%.

### Example 18—Compound 104

### 2-((S)-2-((5-Bromo-2-((5-cyanopyridin-3-yl)methoxy)-4-((3′-((1-((2R,3R,4S,5S,6S)-6-(fluoromethyl)-3, 4,5-trihydroxytetrahydro-2H-pyran-2-yl)-1H-1,2,3-triazol-4-yl)methoxy)-2,2′-dimethyl-[1,1′-biphenyl]-3-yl)methoxy)benzyl)amino)-3-sulfopropanamido)ethane-1,1-disulfonic acid (104)

Alkyne 101 (2.00 μmol, 2.17 μmol, 1.00 equiv.) reacted with 6-F-sugar 83 (0.90 mg, 4.34 μmol, 2.00 equiv.) with a premixed catalyst consisting of CuSO₄ (0.10 mg g, 0.65 μmol, 0.30 equiv.), THPTA (0.12 mg, 0.33 μmol, 0.15 equiv.) and sodium ascorbate (2.15 mg, 10.8 μmol, 5.00 equiv.) in an 1:1 mixture of H₂O/t-BuOH (200 μL) at room temperature for 16 h according to GP-5. The reaction mixture was directly injected in semi-preparative RP-HPLC (System C, Rₜ=10 min) and after lyophilization, compound 104 (1.50 mg, 1.33 μmol, 61%) was obtained as colorless solid. Rₜ=9.01 min (System A), purity: 98.0%.

### Preparation of the Chelate Compounds According to the Invention

### Example 19—Compound 42

### 2,2′, 2″-(10-(2-(4-(3-(4-(((2′-Bromo-3′-((2-chloro-4-(((2-((2,2-disulfoethyl)amino)-2-oxoethyl)(methyl)amino)methyl)-5-((5-(methylsulfonyl)pyridin-3-yl)methoxy)phenoxy)methyl)-[1,1′-biphenyl]-3-yl)oxy)methyl)-1H-1,2,3-triazol-1-yl)propyl)piperidin-1-yl)-2-oxoethyl)-1,4,7,10-tetraazacyclododecane-1,4,7-triyl)triacetic acid (42)

Propyl-piperidinyl linker structure 35 (7.0 mg, 6.5 μmol, 1.00 equiv.), DOTA-p-nitrophenylester (41) (6.9 mg, 13.1 μmol, 2.00 equiv.) and abs. DIPEA (5.7 μL, 32.7 μmol, 5.00 equiv.) were dissolved in abs. DMF (1 mL) and reacted according to GP-7. Purification by semi-preparative RP-HPLC (System C, Rₜ=21 min) and subsequent lyophilization yielded DOTA-conjugate (42) (3.4 mg, 2.3 μmol, 36%) as a colorless powder. mp=185° C. (decomposition). Rₜ=9.41 min (System A), purity: 99.8%.

### Example 20—Compound 43

### 2,2′, 2″-(10-(2-(4-(3-(4-(((2′-Bromo-3′-((2-chloro-4-(((2-((2,2-disulfoethyl)amino)-2-oxoethyl)(methyl)amino)methyl)-5-((5-(methylsulfonyl)pyridin-3-yl)methoxy)phenoxy)methyl)-[1,1′-biphenyl]-3-yl)oxy)methyl)-1H-1,2,3-triazol-1-yl)propyl)piperazin-1-yl)-2-oxoethyl)-1,4,7,10-tetraazacyclododecane-1,4,7-triyl)triacetic acid (43)

Propyl-piperazinyl linker structure 36 (4.0 mg, 3.7 μmol, 1.00 equiv.), DOTAp-nitrophenylester (41) (3.9 mg, 7.5 μmol, 2.00 equiv.) and abs. DIPEA (3.3 μL, 18.7 μmol, 5.00 equiv.) were dissolved in abs. DMF (1 mL) and reacted according to GP-7. Purification by semi-preparative RP-HPLC (System C, Rₜ=19 min) and subsequent lyophilization yielded DOTA-conjugate 43 (4.1 mg, 2.8 μmol, 75%) as a colorless powder. mp=181-184° C. (decomposition). Rₜ=6.45 min (System A), purity: 91.0%. MS (HR-ESI⁺): Exact mass calculated for [M+H]⁺: m/z=1405.4665, measured: m/z=1405.465.

### Example 21—Compound 44

### 2,2′, 2″-(10-(2-(4-(2-(2-(2-(4-(((2′-Bromo-3′-((2-chloro-4-(((2-((2,2-disulfoethyl)amino)-2-oxoethyl)(methyl)amino)methyl)- 5-((5-(methylsulfonyl)pyridin-3-yl)methoxy)phenoxy)methyl)-[1,1′-biphenyl]-3-yl)oxy)methyl)-1H-1,2,3-triazol-1-yl)ethoxy)ethoxy)ethyl)piperazin-1-yl)-2-oxoethyl)-1,4,7,10-tetraazacyclododecane-1,4,7-triyl)triacetic acid (44)

PEG-2-Piperazinyl linker structure 37 (17.0 mg, 14.9 μmol, 1.00 equiv.), DOTA-nitrophenylester (41) (15.6 mg, 29.7 μmol, 2.00 equiv.) and abs. DIPEA (12.9 μL, 74.3 μmol, 5.00 equiv.) were dissolved in abs. DMF (1) and reacted according to GP-7. Purification by semi-preparative RP-HPLC (System C, Rₜ=17 min) and subsequent lyophilization yielded DOTA-conjugate 44 (9.6 mg, 6.3 μmol, 42%) as a colorless powder. mp=190° C. (decomposition). Rₜ=8.85 min (System A), purity: 100%. MS (HR-ESI⁺): Exact mass calculated for [M+H]⁺: m/z=1093.3231, measured: m/z=1093.3225.

### Example 22—Compound 45

### 2,2′, 2″-(10-(2-(4-(3-(4-(((3′-((2-Chloro-4-(((2-((2,2-disulfoethyl)amino)-2-oxoethyl)(methyl)amino)methyl)-5-((5- (methylsulfonyl)pyridin-3-yl)methoxy)phenoxy)methyl)-2,2′-dimethyl-[1,1′-biphenyl]-3-yl)oxy)methyl)-1H-1,2,3-triazol-1-yl)propyl)piperidin-1-yl)-2-oxoethyl)-1,4,7,10-tetraazacyclododecane-1,4,7-triyl)triacetic acid (45)

Propyl-piperidinyl linker structure 38 (8.0 mg, 7.8 μmol, 1.00 equiv.), DOTAp-nitrophenylester (41) (8.2 mg, 15.5 μmol, 2.00 equiv.) and abs. DIPEA (6.8 μL, 38.8 μL, 5.00 equiv.) were dissolved in abs. DMF (1 mL) and reacted according to GP-7. Purification by semi-preparative RP-HPLC (System C, Rₜ=23 min) and subsequent lyophilization yielded DOTA-conjugate 45 (4.0 mg, 2.8 mol, 37%) as a colorless powder. mp=195° C. (decomposition). Rₜ=9.57 min (System A), purity: 99.7%. MS (HR-ESI⁺): Exact mass calculated for [(M+2H/2]⁺: m/z=702.7392, measured: m/z=702.7403.

### Example 23—Compound 46

### 2,2′, 2″-(10-(2-(4-(3-(4-(((3′-((2-Chloro-4-(((2-((2,2-disulfoethyl)amino)-2-oxoethyl)(methyl)amino)methyl)-5-((5- (methylsulfonyl)pyridin-3-yl)methoxy)phenoxy)methyl)-2,2′-dimethyl-[1,1′-biphenyl]-3-yl)oxy)methyl)-1H-1,2,3-triazol-1-yl)propyl)piperazin-1-yl)-2-oxoethyl)-1,4,7,10-tetraazacyclododecane-1,4,7-triyl)triacetic acid (46)

Propyl-piperazinyl linker structure 39 (5.0 mg, 4.9 μmol, 1.00 equiv.), DOTAp-nitrophenylester (41) (5.2 mg, 9.8 μmol, 2.00 equiv.) and abs. DIPEA (4.3 μL, 24.5 μmol, 5.00 equiv.) were dissolved in abs. DMF (1 mL) and reacted according to GP-7. Purification by semi-preparative RP-HPLC (System C, Rₜ=18 min) and subsequent lyophilization yielded DOTA-conjugate 46 (5.2 mg, 3.7 μmol, 75%) as a colorless powder. mp=190-193° C. (decomposition). Rₜ=8.91 min (System A), purity: 99.0%. MS (HR-ESI⁺): Exact mass calculated for [M+H]⁺: m/z=1405.4665, measured: m/z=1405.4650.

### Example 24—Compound 47

### 2,2′, 2″-(10-(2-(4-(2-(2-(2-(4-(((3′-((2-Chloro-4-(((2-((2,2-disulfoethyl)amino)-2-oxoethyl)(methyl)amino)methyl)-5-((5- (methylsulfonyl)pyridin-3-yl)methoxy)phenoxy)methyl)-2,2′-dimethyl-[1,1′-biphenyl]-3-yl)oxy)methyl)-1H-1,2,3-triazol-1-yl)ethoxy)ethoxy)ethyl)piperazin-1-yl)-2-oxoethyl)-1,4,7,10-tetraazacyclododecane-1,4,7-triyl)triacetic acid (47)

PEG-2-Piperazinyl linker structure 40 (14.0 mg, 12.8 μmol, 1.00 equiv.), DOTA-p-nitrophenylester (41) (13.5 mg, 25.6 μmol, 2.00 equiv.) and abs. DIPEA (11.2 μL, 64.0 μmol, 5.00 equiv.) were dissolved in abs. DMF (1 mL) and reacted according to GP-7. Purification by semi-preparative RP-HPLC (System C, Rₜ=16 min) and subsequent lyophilization yielded DOTA-conjugate 47 (6.2 mg, 4.2 μmol, 33%) as a colorless powder. mp=195° C. (decomposition). Rₜ=9.09 min (System A), purity: 100%. MS (HR-ESI⁺): Exact mass calculated for [M+H]⁺: m/z=1479.5032, measured: m/z=1479.5018.

### Example 25—Compound 54

### 2,2′, 2″-(10-(2-(4-(3-(4-(((3′-((2-Chloro-5-((5-cyanopyridin-3-yl)methoxy)-4-(((2-((2,2-disulfoethyl)amino)-2-oxoethyl)(methyl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-2-oxoethyl)-1,4,7,10-tetraazacyclododecane-1,4,7-triyl)triacetic acid (54)

Compound 53 (7.0 mg, 7.2 μmol, 1.00 equiv.), DOTA-p-nitrophenylester (41) (5.7 mg, 10.9 μmol, 1.50 equiv.) and abs. DIPEA (6.3 μL, 36.2 μmol, 5.00 equiv.) were dissolved in abs. DMF (1 mL) and reacted according to GP-7. Purification by semipreparative RP-HPLC (System C, Rₜ=19 min) and subsequent lyophilization yielded DOTA-conjugate 54 (6.0 mg, 4.4 μmol, 61%) as a colorless powder. mp=190° C. (decomposition). Rₜ=9.42 min (System A), purity: 96.6%. MS (HR-ESI⁺): Exact mass calculated for [M+H]⁺: m/z=1352.4842, measured: m/z=1352.4831.

### Example 26—Compound 63

### 2,2′, 2″-(10-(2-(4-(3-(4-(((3′-((2-Bromo-5-((5-cyanopyridin-3-yl)methoxy)-4-(((2-((2,2-disulfoethyl)amino)-2-oxoethyl)(methyl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-2-oxoethyl)-1,4,7,10-tetraazacyclododecane-1,4,7-triyl)triacetic acid (63)

Propyl-piperazinyl linker structure 62 (9.2 mg, 9.1 μmol, 1.00 equiv.), DOTAp-nitrophenylester (41) (7.2 mg, 13.7 μmol, 1.50 equiv.) and abs. DIPEA (8.0 μL, 45.5 μmol, 5.00 equiv.) were dissolved in abs. DMF (1 mL) and reacted according to GP-7. Purification by semi-preparative RP-HPLC (System C, Rₜ=21 min) and subsequent lyophilization yielded DOTA-conjugate 63 (6.3 mg, 4.5 μmol, 50%) as a colorless powder. mp=190° C. (decomposition). Rₜ=9.88 min (System A), purity: 100%. MS (HR-ESI⁺): Exact mass calculated for [M+H]⁺: m/z=1396.4336, measured: m/z=1396.4327.

### Example 27—Compound 73

### 2,2′, 2″-(10-(2-(4-(3-(4-(((3′-((5-((5-Cyanopyridin-3-yl)methoxy)-4-(((2-((2,2-disulfoethyl)amino)-2-oxoethyl)(methyl)amino)methyl)- 2-iodophenoxy)methyl)-2,2′-dimethyl-[1,1′-biphenyl]-3-yl)oxy)methyl)-1H-1,2,3-triazol-1-yl)propyl)piperazin-1-yl)-2-oxoethyl)-1,4,7,10-tetraazacyclododecane-1,4,7-triyl)triacetic acid (73)

Propyl-piperazinyl linker structure 72 (15.0 mg, 14.2 μmol, 1.00 equiv.), DOTA-p-nitrophenylester (41) (11.2 mg, 21.3 μmol, 1.50 equiv.) and abs. DIPEA (12.4 μL, 70.9 μmol, 5.00 equiv.) were dissolved in abs. DMF (1 mL) and reacted according to GP-7. Purification by semi-preparative RP-HPLC (System C, Rₜ=19 min) and subsequent lyophilization yielded DOTA-conjugate 73 (10.7 mg, 7.4 μmol, 52%) as a colorless powder. mp=185° C. (decomposition). Rₜ=9.91 min (System A), purity: 97.5%. MS (HR-ESI⁺): Exact mass calculated for [M+H]⁺: m/z=1444.4198, measured: m/z=1444.4191.

### Example 28—Compound 79

### (R)-2-((S)-4-(4,7-Bis(2-(tert-butoxy)-2-oxoethyl)-1,4,7-triazonan-1-yl)-5-(tert-butoxy)-5-oxopentanamido)-3-(4-(3-(4-(((3′-((2-chloro-5-((5-cyanopyridin-3-yl)methoxy)-4-((((R)-1-oxo-3-sulfo-1-((2-sulfoethyl)amino)propan-2-yl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-3-oxopropane-1-sulfonic acid (79)

Primary amine 78 (44.0 mg, 39.4 μmol, 1.00 equiv.), (R)-NODA-GA-tris(t-Bu)₃ (32.1 mg, 59.1 μmol, 1.50 equiv.) and pyridine (13.7 μL, 78.7 μmol, 2.00 equiv.) were dissolved in abs. DMF (1 mL) and cooled down to 0° C. DCC (16.2 mg, 78.7 μmol, 2.00 equiv.) was added, the reaction was allowed to warm to room temperature and was then stirred for 16 h. After reaction completion (monitored by RP-HPLC, system A), the solvent was removed and the residue was used in the next step without further purification.

### Example 29—Compound 80

### 2,2′-(7-((S)-1-Carboxy-4-(((R)-1-(4-(3-(4-(((3′-((2-chloro-5-((5-cyanopyridin-3-yl)methoxy)-4-((((S)-1-oxo-3-sulfo-1-((2-sulfoethyl)amino)propan-2-yl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-1-oxo-3-sulfopropan-2-yl)amino)-4-oxobutyl)-1,4,7-triazonane-1,4-diyl)diacetic acid (80)

The crude product from the previous reaction was dissolved in a mixture of TFA/DCM/TES/H₂O (20:20:8:7 (% v/v)) and was stirred at room temperature for 16 h. The solvent mixture was removed at reduced pressure at room temperature and the crude product was purified by semi-preparative RP-HPLC (Agilent Zorbax SB C-18 5 μm 80 Å, 9.4×250 mm with 20-70% acetonitrile (0.1% TFA) in water (0.1% TFA) in a linear gradient over 45 min, 6 mL/min, Rₜ=10 min). After lyophilization, the final compound 80 was obtained as a colorless solid. MS (HR-ESI⁺): Exact mass calculated for [M+H]⁺: m/z=1474.4515, measured: m/z=1474.4484.

### Example 30—Compounds 105 to 114

Labeling of Compounds 42-44, 45-47, 54, 63 and 73 with Ga-68

The Ga-68 generator was eluted with approx. 1 M HCl (approx. 1500 MBq in 300 μL). 8 μL of 1 mM stock solution of the corresponding DOTA-compound was added to a Protein LoBind Eppendorf tube containing 200 μL of an 1 M HEPES solution. 25 μL of the Ga-68 was added and the solution was shaken with 300 rpm at 90° C. for 15 min. The solution was cooled down to temperature and radio-TLC was performed with iTLC-SG as stationary phase and 0.1 M citrate solution (adjusted to pH 4 with 1 M NaOH) as mobile phase. The conversion was >95% and yielded approx. 90 MBq of the corresponding Ga-68 labelled DOTA-compound.

### Example 31—Compounds 115 to 124 and 125 to 133

Labeling of Compounds 42-44, 45-47, 54, 63 and 73 with Cu-64 or Lu-177, respectively

8 μL of 1 mM stock solution of the corresponding DOTA-compound was added to a Protein LoBind Eppendorf tube containing 200 μL of an approx. 1 M HEPES solution, which was prior adjusted with 1 M HCl to pH 4. The corresponding metal solution (both 0.01-0.1 M HCl) was added and the solution was shaken with 300 rpm at 90° C. for 15 min. The solution was cooled down to temperature and radio-TLC was performed with iTLC-SG as stationary phase and 0.1 M citrate solution (adjusted to pH 4 with 1 M NaOH) as mobile phase. The conversion was >99% and yielded the corresponding Cu-64 or Lu-177 labelled DOTA-compound.

### Example 32—Compounds 151, 136, 139 to 141 and 148

For the preparation of radio labeled compounds 151, 136, 139 to 141 and 148, at first the precursor compounds, that correspond the F-Compounds 85, 88, 91, 94, 97, and 102, in which a O-Tf group instead of fluorine is present, were prepared according Example 1, 4, 7-9, or 16, respectively. Then, the obtained compounds were radiolabeled according to literature [18].

### Example 33—Compounds 134, 137, 142 to 144 and 149

For the preparation of radio labeled compounds 134, 137, 142 to 144 and 149, at first the precursor compounds, that correspond the 2-F-sugar compounds 86, 89, 92, 95, 98, and 103, in which a O-Tf group instead of fluorine is present, were prepared according Example 2, 5, 10-12, or 17, respectively. Then, the obtained compounds were radiolabeled according to literature [16].

### Example 34—Compounds 135, 138, 145 to 147 and 150

For the preparation of radio labeled compounds 135, 138, 145 to 147 and 150, at first the precursor compounds, that correspond the 6-F-sugar compounds 87, 90, 93, 96, 99, and 104, in which a O-Ts group instead of fluorine is present, were prepared according Example 3, 6, 13-15, or 18, respectively. Then, the obtained compounds were radiolabeled according to literature [17].

### Example 35—In Vitro Experiments—Determination of Binding Affinities of PD-L1 Targeting Radioligands with a Saturation Binding Assay

Experiments were performed on a PC3 hPSCA PD-L1 overexpressing cell line in triplicates and three experiments were performed per compound, resulting in a 3×3 data set per compound (see FIGS. 1 to 3). The saturated, upper curve represents the total binding of the compound whereas the lower, linear curve represents the unspecific binding. The specific binding is represented as the difference between total and unspecific binding.

In the following the procedure for saturation binding assay is explained: The respective Cu-64 labeled compound is prepared freshly for each experiment. The cells with PD-L1 expression were prepared in 48-well plates. Three rows in the plates were designated for determination of total binding and one row for determination of the unspecific binding. For the latter, the cells are pretreated for 15 min with a 300 fold excess of a literature known PD-L1 inhibitor with high binding to PD-L1. Then, in all rows concentrations of 500 to 3.9 nM (displayed on x-axis) are pipetted on the cells which get incubated at 4° C. for 90 min. After washing steps with medium (PBS+2.5% BSA), the radioactive counts (displayed on y-axis after calculation with protein concentration) of the cells is determined by a gamma-counter. Protein determination takes place at the next day and provides the Bₘₐₓ value (total density of PD-L1 receptors on cell surface). Plotting and graphical interpretation provides curves and K_{d}-values from graph fitting.

The following table shows the binding affinities (K_{d}) and receptor densities (Bₘₐₓ) of compounds 115 to 124.

| Compound | K_{d} [nM] (n = 3) | Bₘₐₓ [pmol/mg] (n = 3) |
|---|---|---|
| 115 | 584.8 ± 39.6 | 11.7 ± 0.54 |
| 116 | 487.0 ± 44.2 | 8.91 ± 0.45 |
| 117 | 356.7 ± 66.5 (n = 6) | 5.85 ± 1.14 (n = 6) |
| 118 | 122.8 ± 11.7 | 13.5 ± 0.98 |
| 119 | 59.9 ± 4.63 | 5.43 ± 0.46 |
| 120 | 71.3 ± 10.1 | 7.04 ± 0.42 |
| 121 | 128.0 ± 13.2 | 20.9 ± 2.17 |
| 122 | 134.8 ± 2.34 | 8.28 ± 0.13 |
| 123 | 300.4 ± 20.6 | 49.6 ± 1.27 |
| 124 | 80.5 ± 3.40 | 7.05 ± 0.74 |

### Example 36—In Vivo Experiments

Compound 119 was tested in a PD-L1 overexpressing mouse xenograft model. PET images were recorded 2 h, 15 h and 24 h post injection (see FIG. 4). The experiments were performed with compound 119 in duplicates (two mice for each timepoint). About 10 MBq of radiolabelled compound were injected intravenously in the tail vain of an alive mouse. At 2 hrs p.i. the first image was recorded. Moderate uptake in liver, high uptake in bladder and intestines and low uptake in PD-L1 tumor have been observed, but the compound did still circulate in the blood pool. At 5 hrs p.i. the second image was recorded. High uptake in liver; high uptake in colon and intestine and increased uptake in PD-L1 tumor have been observed. At 24 hrs p.i. the third image was recorded. Moderate uptake in liver, low uptake in intestine and moderate uptake in the PD-L1 positive tumor has been observed.

### Preparation of Further Starting Compounds

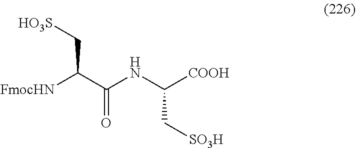

((((9H-Fluoren-9-yl)methoxy)carbonyl)(sulfo)-D-alanyl)(sulfo)-D-alanine (226). Fmoc-L-cysteic acid (100 mg, 256 μmol, 1.00 equiv.), N-hydroxy succinimide (30.9 mg, 268 μmol, 1.05 equiv.) and DCC (55.4 mg, 268 μmol, 1.05 equiv.) were dissolved in abs. DMF (2 mL) at 0° C. and were stirred at room temperature for 16 h. The reaction completion was observed by analytical RP-HPLC (System A) and then the reaction was solution was added dropwise to a solution of L-cysteic acid (43.2 mg, 256 μmol, 1.00 equiv.) in 6.8% sodium carbonate solution (1 mL) at 0° C. The reaction mixture was stirred at this temperature for 3 h until the complete conversion was verified by analytical RP-HPLC (System A). The mixture was acidified with 10% hydrochloric acid to pH 2, concentrated in vacuo and purified by semi-preparative RP-HPLC (Agilent Zorbax SB C-18 5 μm 80 Å, 9.4×250 mm with 10-70% acetonitrile (0.1% TFA) in water (0.1% TFA) in a linear gradient over 45 min, 6 mL/min, Rₜ=8 min). After lyophilization, the disulfonic acid (226) (74.2 mg, 136 μmol, 53% over two steps) was obtained as a colorless oil. Rₜ=6.82 min (System A), purity=100%. ¹H NMR (400 MHz, DMSO-d₆) δ=7.89 (d, ³J=7.5 Hz, 2H), 7.71 (d, ³J=7.4 Hz, 2H), 7.41 (t, 3J=7.4 Hz, 2H), 7.31-7.34 (m, 2H), 4.77-4.82 (m, 1H), 4.46-4.50 (m, 1H), 4.18-4.27 (m, 3H), 3.45-3.47 (m, 4H), 3.10-3.17 (m, 2H), 2.92-3.03 (m, 2H), 2.72 (m, 1H), 1.88-1.92 ppm (m, 2H). ¹³C NMR (101 MHz, DMSO-d₆) δ=8.33-8.34 (m, 1H), 7.88 (t, 3J=7.5 Hz, 2H), 7.72-7.73 (m, 2H), 7.41 (t, ³J=7.4 Hz, 2H), 7.31-7.39 (m, 2H), 4.36-4.38 (m, 1H), 4.22-4.23 (m, 4H), 2.81-2.99 ppm (m, 4H). ¹³C NMR (101 MHz, DMSO-d₆) δ=171.6, 170.5, 155.8, 143.9, 143.9, 140.7, 127.7, 127.3, 125.5, 120.1, 66.0, 52.5, 51.8, 50.9, 49.9, 46.6 ppm. MS (HR-ESI⁺): Exact mass calculated for [M+H]⁺: m/z=543.0743, measured: m/z=543.0731.

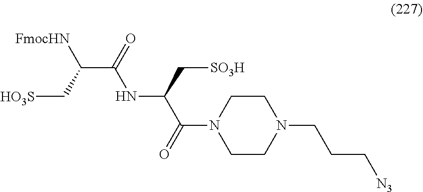

(R)-2-((((9H-Fluoren-9-yl)methoxy)carbonyl)amino)-3-(((R)-1-(4-(3-azidopropyl)piperazin-1-yl)-1-oxo-3-sulfopropan-2-yl)amino)-3-oxopropane-1-sulfonic acid (227). Disulfonic acid (226) (56.0 mg, 103 μmol, 1.00 equiv.), 1-(3-azidopropyl)piperazine (31) (35.0 mg, 206 μmol, 2.00 equiv.), HBTU (78.2 mg, 103.2 μmol, 2.00 equiv.) HOBt (13.9 mg, 103 μmol, 1.00 equiv.) and pyridine (18.6 μL, 310 μmol, 3.00 equiv.) reacted in abs. DMF (3 mL) according to GP-8. Semi-preparative RP-HPLC (Agilent Zorbax SB C-18 5 μm 80 Å, 9.4×250 mm with 15-60% acetonitrile (0.1% TFA) in water (0.1% TFA) in a linear gradient over 45 min, 6 mL/min, Rₜ=10 min) with subsequent lyophilization yielded linker structure (227) (37.2 mg, 53.6 μmol, 52%) as a yellowish oil. Rₜ=7.83 min (System A), purity=95.3%. ¹H NMR (400 MHz, DMSO-d₆) δ=9.48 (bs, 1H), 8.25-8.33 (m, 1H), 7.89 (d, ³J=7.5 Hz, 2H), 7.69-7.70 (m, 2H), 7.41 (t, ³J=7.4 Hz, 2H), 7.32-7.35 (m, 2H), 4.23-4.43 (m, 6H), 3.35-3.53 (m, 6H), 2.65-3.08 (m, 8H), 1.89 ppm (bs, 2H). ¹³C NMR (101 MHz, DMSO-d₆) δ=169.8, 155.6, 143.9, 143.8, 140.7, 127.6, 127.2 125.3, 120.1, 65.9, 53.6, 52.9, 52.1, 51.7, 50.4, 47.8, 46.6, 45.3, 42.6, 22.9 ppm. MS (HR-ESI⁺): Exact mass calculated for [M+H]⁺: m/z=694.1965, measured: m/z=694.1957.

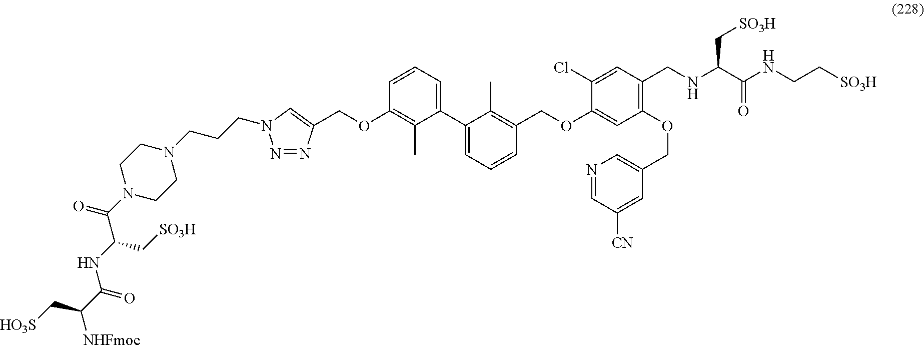

(R)-2-((4-((3′-((1-(3-(4-(((((9H-Fluoren-9-yl)methoxy)carbonyl)(sulfo)-D-alanyl)(sulfo)-D-alanyl)piperazin-1-yl)propyl)-1H-1,2,3-triazol-4-yl)methoxy)-2,2′-dimethyl-[1,1′-biphenyl]-3-yl)methoxy)-5-chloro-2-((5-cyanopyridin-3-yl)methoxy)benzyl)amino)-3-oxo-3-((2-sulfoethyl)amino)propane-1-sulfonic acid (228). Alkyne (75) (24.0 mg, 34.6 μmol, 1.00 equiv.) reacted with the linker structure (227) (30.3 mg, 38.1 μmol, 1.10 equiv.) with a premixed catalyst consisting of CuSO₄ (0.6 mg, 3.5 μmol, 0.10 equiv.), THPTA (2.8 mg, 5.2 μmol, 0.15 equiv.) and sodium ascorbate (34.3 mg, 173 μmol, 5.00 equiv.) in an 1:1 mixture of H₂O/t-BuOH (3 mL) at room temperature for 16 h. The solvent was removed in vacuo and the residue was used in the next step without further purification.

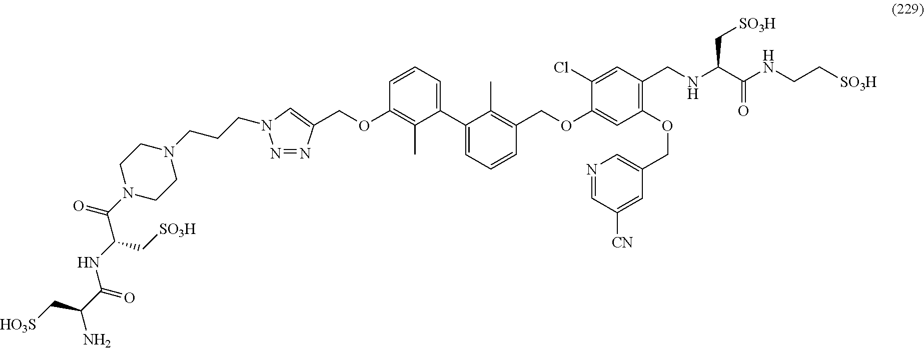 [SMILES: Cc1c(COc2cc(OCc3cncc(C#N)c3)c(CN[C@@H](CS(=O)(=O)O)C(=O)NCCS(=O)(=O)O)cc2Cl)cccc1-c1cccc(OCc2cn(CCCN3CCN(C(=O)[C@H](CS(=O)(=O)O)NC(=O)[C@@H](N)CS(=O)(=O)O)CC3)nn2)c1C]

(R)-2-Amino-3-(((R)-1-(4-(3-(4-(((3′-((2-chloro-5-((5-cyanopyridin-3-yl)methoxy)-4-((((R)-1-oxo-3-sulfo-1-((2-sulfoethyl)amino)propan-2-yl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-1-oxo-3-sulfopropan-2-yl)amino)-3-oxopropane-1-sulfonic acid (229). The residue from the previous reaction was dissolved in abs. DMF (2 mL) and the Fmoc group was removed with sodium azide (11.2 mg, 173 μmol, 5.00 equiv.) according to GP-9. Semi-preparative RP-HPLC purification (Agilent Zorbax SB C-18 5 μm 80 Å, 9.4×250 mm with 25-60% acetonitrile (0.1% TFA) in water (0.1% TFA) in a linear gradient over 45 min, 6 mL/min, Rₜ=8 min) with subsequent lyophilization, yielded primary amine (229) (27.3 mg, 21.5 μmol, 62% over two steps) as a colorless powder. mp=265° C. (decomposition). Rₜ=8.38 min (System A), purity=93.1%. ¹H NMR (400 MHz, DMSO-d₆) δ=9.61 (bs, 1H), 9.36 (bs, 1H), 9.08-9.09 (m, 1H), 9.01-9.03 (m, 3H), 8.80 (t, ³J=5.4 Hz, 1H), 8.51 (s, 1H), 8.30 (s, 1H), 8.08 (bs, 3H), 7.52-7.53 (m, 1H), 7.44-7.47 (m, 1H), 7.23-7.27 (m, 2H), 7.16-7.19 (m, 2H), 7.07 (d, ³J=7.5 Hz, 1H), 6.71 (d, ³J=7.5 Hz, 1H), 5.34-5.45 (m, 2H), 5.24-5.30 (m, 2H), 5.18-5.21 (m, 2H), 5.02-5.04 (m, 1H), 4.40-4.50 (m, 2H), 4.22-4.25 (m, 2H), 3.99-4.02 (m, 3H), 3.37-3.50 (m, 5H), 3.13 (bs, 3H), 2.92-3.02 (m, 4H), 2.72-2.78 (m, 2H), 2.66 (t, 3J=7.0, 2H), 2.28 (bs, 2H), 3.11 (s, 3H), 1.80-1.81 ppm (s, 3H). ¹³C NMR (101 MHz, DMSO-d₆) δ=169.3, 166.7, 166.5, 165.4, 158.6, 158.2, 157.8, 156.3 156.2, 155.5, 155.4, 152.3, 151.8, 143.2, 142.3, 141.6, 139.1, 134.7, 134.5, 132.7, 129.3, 127.8, 126.4, 125.5, 124.5, 124.0, 121.8, 116.9, 116.5, 113.6, 113.1, 113.1, 112.7, 110.8, 109.1, 100.1, 69.8, 66.9, 61.8, 56.4, 53.5, 50.5, 49.8, 46.7, 46.0, 44.4, 36.3, 24.3, 15.3, 12.9 ppm. \tilde{ν}=1651 (w), 1574 (w), 1505 (w), 1445 (w), 1167 (s), 1091 (w), 1036 (s), 723 cm⁻¹ (w). MS (HR-ESI⁺): Exact mass calculated for [M+H]⁺: m/z=1268.2924, measured: m/z=1268.2915.

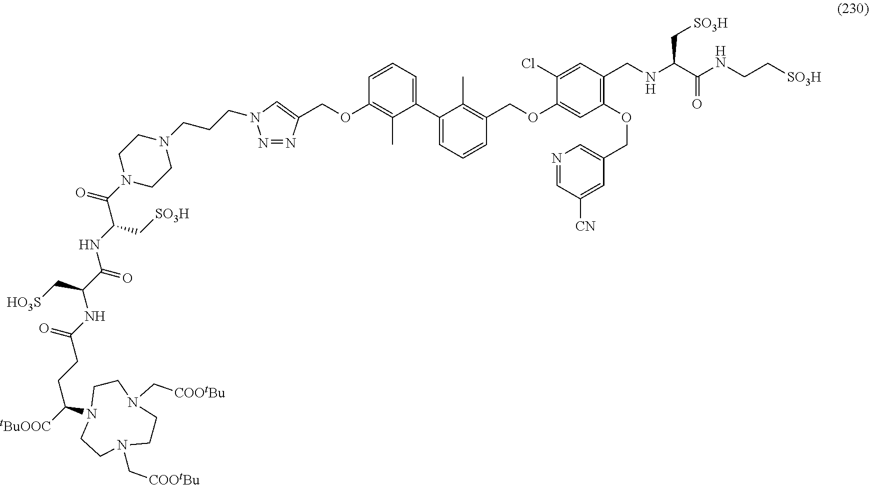

(R)-2-((R)-4-(4,7-Bis(2-(tert-butoxy)-2-oxoethyl)-1,4,7-triazonan-1-yl)-5-(tert-butoxy)-5-oxopentanamido)-3-(((R)-1-(4-(3-(4-(((3′-((2-chloro-5-((5-cyanopyridin-3-yl)methoxy)-4-((((R)-1-oxo-3-sulfo-1-((2-sulfoethyl)amino)propan-2-yl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-1-oxo-3-sulfopropan-2-yl)amino)-3-oxopropane-1-sulfonic acid (230). The amine (229) (17.0 mg, 13.4 μmol, 1.00 equiv.), (R)-NODAGA(t-Bu)₃ (14.6 mg, 26.8 μmol, 2.00 equiv.), DCC (5.5 mg, 26.8 μmol, 2.00 equiv.), pyridine (5.4 μL, 67.0 μmol, 5.00 equiv.) and HOBt (1.8 mg, 13.4 μmol, 1.00 equiv.) reacted in abs. DMF (1 mL) according to GP-8. Semi-preparative purification was omitted and the residue was subjected to tBu deprotection in the next step.

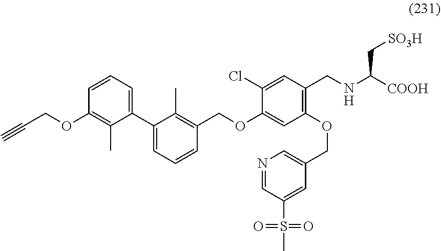 [SMILES: C#CCOc1cccc(-c2cccc(COc3cc(OCc4cncc(S(C)(=O)=O)c4)c(CN[C@@H](CS(=O)(=O)O)C(=O)O)cc3Cl)c2C)c1C]

(5-Chloro-4-((2,2′-dimethyl-3′-(prop-2-yn-1-yloxy)-[1,1′-biphenyl]-3-yl)methoxy)-2-((5-(methylsulfonyl)pyridin-3-yl)methoxy)benzyl)(sulfo)-D-alanine (231)- The aldehyde (25) (100 mg, 169 μmol, 1.00 equiv.), L-cysteic acid (180 mg, 508 μmol, 3.00 equiv.) and sodium cyanoborohydride (16.03 mg, 254 μmol, 1.50 equiv.) were reacted in a mixture of abs. MeOH (2 mL) and DMF (2 mL) according to GP-3. After reaction completion monitored by RP-HPLC (system A), the solvent was removed in vacuo and the residue was purified by semi-preparative RP-HPLC (Agilent Zorbax SB C-18 5 μm 80 Å, 9.4×250 mm with 45-90% acetonitrile (0.1% TFA) in water (0.1% TFA) in a linear gradient over 45 min, 6 mL/min, Rₜ=6 min). Subsequent lyophilization gave carboxylic acid (231) (108 mg, 145 μmol, 86%) as a colorless powder. mp=162-165° C. Rₜ=11.9 min (System A), purity=99.8%. ¹H NMR (400 MHz, DMSO-d₆) δ=9.93 (bs, 1H), 9.07-9.10 (m, 2H), 8.51 (s, 1H), 7.54 (s, 1H), 7.49 (d, 3J=7.5 Hz, 1H), 7.22-7.29 (m, 3H), 7.04-7.10 (m, 2H), 6.74 (d, ³J=7.5 Hz, 1H), 5.43-5.50 (m, 2H), 5.28-5.36 (m, 2H), 4.86 (d, ⁴J=1.9 Hz, 2H), 4.18-4.32 (m, 4H), 3.58 (s, 1H), 3.03-3.07 (m, 1H), 2.87-2.93 (m, 1H), 2.03 (s, 3H), 1.83 ppm (s, 3H). ¹³C NMR (101 MHz, DMSO-d₆) δ=168.4, 156.4, 155.5, 155.4, 153.5, 147.2, 142.3, 141.5, 136.9, 134.8, 134.6, 134.4, 132.7, 132.4, 129.3, 127.8, 126.2, 125.5, 124.1, 122.1, 113.1, 113.1, 110.9, 100.3, 79.5, 78.2, 69.7, 67.4, 56.2, 55.8, 48.6, 44.6, 43.6 ppm. IR (ATR): 1577 (w), 1505 (w), 1454 (w), 1409 (w), 1306 (m), 1232 (m), 1144 (s), 1089 (w), 1019 (m), 956 (w), 784 (w), 768 (w), 721 cm⁻¹ (w). MS (HR-ESI⁺): Exact mass calculated for [M+H]⁺: m/z=743.1500, measured: m/z=743.1491.

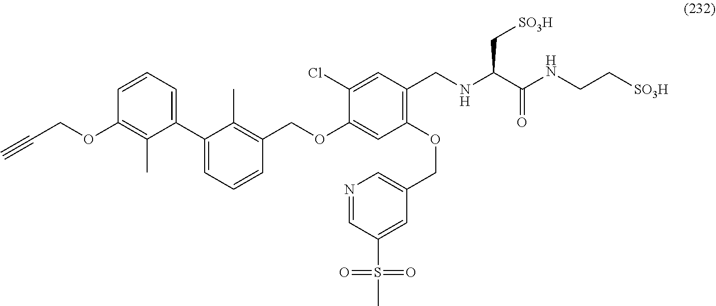 [SMILES: C#CCOc1cccc(-c2cccc(COc3cc(OCc4cncc(S(C)(=O)=O)c4)c(CN[C@@H](CS(=O)(=O)O)C(=O)NCCS(=O)(=O)O)cc3Cl)c2C)c1C]

(R)-2-((5-Chloro-4-((2,2′-dimethyl-3′-(prop-2-yn-1-yloxy)-[1,1′-biphenyl]-3-yl)methoxy)-2-((5-(methylsulfonyl)pyridin-3-yl)methoxy)benzyl)amino)-3-oxo-3-((2-sulfoethyl)amino)propane-1-sulfonic acid (232). The carboxylic acid (231) (40.0 mg, 56.2 μmol, 1.00 equiv.), taurine (14.1 mg, 113 μmol, 2.00 equiv.), HBTU (32.0 mg, 84.4 mmol, 1.50 equiv.), HOBt (7.6 mg, 56.2 mol, 1.00 equiv.) and abs. DIPEA (19.5 μL, 113 mmol, 2.00 equiv.) reacted in abs. DMF (3 mL) according to GP-8. Semipreparative RP-HPLC (Agilent Zorbax SB C-18 5 μm 80 Å, 9.4×250 mm with 35-90% acetonitrile (0.1% TFA) in water (0.1% TFA) in a linear gradient over 45 min, 6 mL/min, Rₜ=11 min) with subsequent lyophilization yielded alkyne (232) (41.2 mg, 64.7 μmol, 86%) as a colorless powder. mp=215° C. Rₜ=10.70 min (System A), purity=95.1%. ¹H NMR (400 MHz, DMSO-d₆) δ=9.35 (bs, 1H), 9.13 (s, 1H), 9.08 (s, 1H), 8.98 (bs, 1H), 8.76 (t, ³J=5.4 Hz, 1H), 8.54 (s, 1H), 7.53 (s, 1H), 7.49 (d, 3J=7.6 Hz, 1H), 7.22-7.29 (m, 3H), 7.04-7.10 (m, 2H), 6.74 (d, ³J=7.5 Hz, 1H), 5.42-5.52 (m, 2H), 5.26-5.35 (m, 2H), 4.85 (d, ⁴J=2.1 Hz, 2H), 4.18-4.21 (m, 1H), 4.00-4.02 (m, 2H), 3.58 (t, ⁴J=2.2 Hz, 1H), 3.34-3.36 (m, 1H), 2.98-3.01 (m, 1H), 2.71-2.78 (m, 1), 2.64 (t, ³J=7.1 Hz, 2H), 2.03 (s, 3H), 1.83 ppm (s, 3H). ¹³C NMR (101 MHz, DMSO-d₆) δ=165.3, 156.4, 155.5, 155.4, 153.2, 146.9, 142.3, 141.5, 137.0, 135.1, 134.6, 134.6, 134.5, 133.0, 132.7, 129.3, 127.8, 126.2, 125.5, 124.1, 122.1, 113.1, 112.7, 110.9, 100.1, 79.5, 78.2, 69.7, 67.3, 56.5, 55.9, 49.8, 49.7, 44.3, 43.6, 36.2, 15.3, 12.8 ppm. IR (ATR): \tilde{ν}=168 (w), 1607 (w), 1575 (w), 1506 (w), 1446 (w), 1409 (w), 1308 (m), 1154 (s), 1089 (w), 1036 (m), 769 (w), 722 (w), 673 cm⁻¹ (w). MS (HR-ESI⁺): Exact mass calculated for [M+H]⁺: m/z=850.1541, measured: m/z=850.1533.

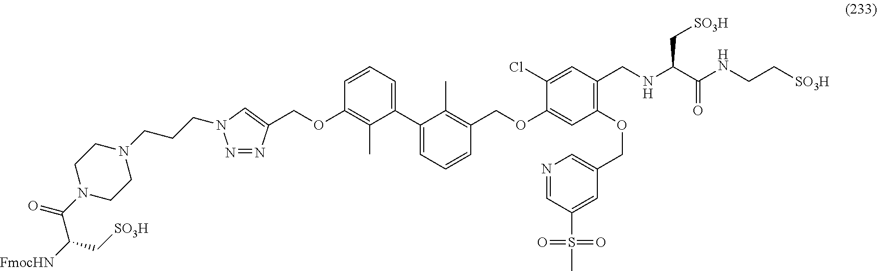

(R)-2-((4-((3′-((1-(3-(4-((((9H-Fluoren-9-yl)methoxy)carbonyl)(sulfo)-D-alanyl)piperazin-1-yl)propyl)-1H-1,2,3-triazol-4-yl)methoxy)-2,2′-dimethyl-[1,1′-biphenyl]-3-yl)methoxy)-5-chloro-2-((5-(methylsulfonyl)pyridin-3-yl)methoxy)benzyl)amino)-3-oxo-3-((2-sulfoethyl)amino)propane-1-sulfonic acid (233). Alkyne (232) (27.0 mg, 31.8 μmol, 1.00 equiv.) reacted with the linker structure 118 (16.4 mg, 30.2 μmol, 0.95 equiv.) with a premixed catalyst consisting of CuSO₄ (0.8 mg, 4.8 μmol, 0.15 equiv.), THPTA (1.4 mg, 3.2 μmol, 0.10 equiv.) and sodium ascorbate (31.5 mg, 159 μmol, 5.00 equiv.) in an 1:1 mixture of H₂O/t-BuOH (3 mL) at room temperature for 16 h. The solvent was removed in vacuo and the residue was used in the next step without further purification.

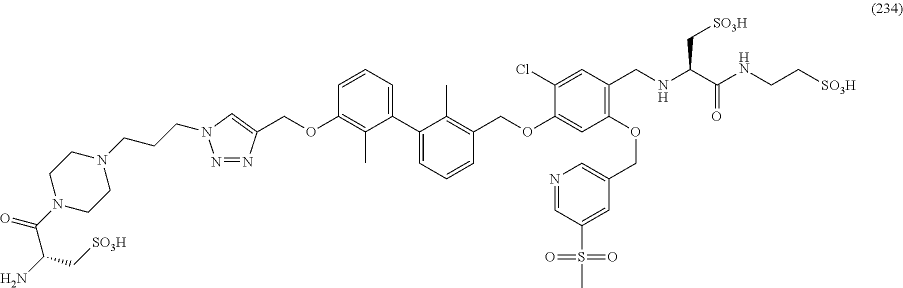 [SMILES: Cc1c(COc2cc(OCc3cncc(S(C)(=O)=O)c3)c(CN[C@@H](CS(=O)(=O)O)C(=O)NCCS(=O)(=O)O)cc2Cl)cccc1-c1cccc(OCc2cn(CCCN3CCN(C(=O)[C@@H](N)CS(=O)(=O)O)CC3)nn2)c1C]

(R)-2-Amino-3-(4-(3-(4-(((3′-((2-chloro-5-((5-(methylsulfonyl)pyridin-3-yl)methoxy)-4-((((R)-1-oxo-3-sulfo-1-((2-sulfoethyl)amino)propan-2-yl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-3-oxopropane-1-sulfonic acid (234). The residue from the previous reaction was dissolved in abs. DMF (3 mL) and the Fmoc group was removed with sodium azide (10.3 mg, 159 μmol, 5.00 equiv.) according to general procedure GP-9. Semi-preparative RP-HPLC purification (Agilent Zorbax SB C-18 5 μm 80 Å, 9.4×250 mm with 25-70% acetonitrile (0.1% TFA) in water (0.1% TFA) in a linear gradient over 45 min, 6 mL/min, Rₜ=9 min) with subsequent lyophilization, yielded primary amine (234) (25.7 mg, 21.9 μmol, 69% over two steps) as a colorless powder. mp=250° C. (decomposition). Rₜ=8.87 min (System A), purity=100%. ¹H NMR (400 MHz, DMSO-d₆) δ=9.74 (bs, 1H), 9.35 (bs, 1H), 9.10 (s, 1H), 9.06 (d, 4J=1.9 Hz, 1H), 8.97 (bs, 1H), 8.75 (t, ³J=5.1 Hz, 1H), 8.51 (s, 1H), 8.30 (s, 1H), 8.09-8.10 (m, 3H), 7.46-7.52 (m, 2H), 7.17-7.28 (m, 4H), 7.07 (d, ³J=7.6 Hz, 1H), 6.74 (d, ³J=7.4 Hz, 1H), 5.41-5.50 (m, 2H), 5.27-5.35 (m, 2H), 5.17-5.24 (m, 2H), 4.51 (t, ³J=6.7, 3H), 4.18-4.21 (m, 1H), 4.01 (bs, 3H), 3.55 (bs, 3H), 3.32-3.38 (m, 2H), 3.18 (bs, 2), 2.97-3.02 (m, 3H), 2.72-2.88 (m, 2H), 2.63 (t, ³J=6.5, 2H), 2.29 (bs, 2H), 2.02-2.03 (m, 3H), 1.80-1.81 ppm (s, 3H). ¹³C NMR (101 MHz, DMSO-d₆) δ=166.3, 165.3, 158.5, 158.2, 156.4, 156.2, 155.4, 155.4, 153.5, 147.1, 143.3, 142.3, 141.6, 136.9, 134.8, 134.8, 134.7, 134.5, 132.8, 129.3, 128.0, 126.4, 125.5, 124.5, 124.0, 121.8, 113.1, 113.1, 112.7, 112.7, 110.8, 100.1, 69.8, 67.3, 61.8, 56.4, 53.2, 50.8, 49.8, 49.7, 47.6, 46.7, 44.3, 43.6, 42.0, 36.2, 24.3, 1.54, 12.8 ppm. IR (ATR): \tilde{ν}=1672 (m), 1606 (w), 1574 (w), 1504 (w), 1445 (m), 1409 (w), 1305 (m), 1166 (s), 1145 (s), 1035 (s), 967 (w), 766 (w), 721 (w), 597 cm⁻¹ (w). MS (HR-ESI⁺): Exact mass calculated for [M+H]⁺: m/z=1170.2808, measured: m/z=1170.2803.

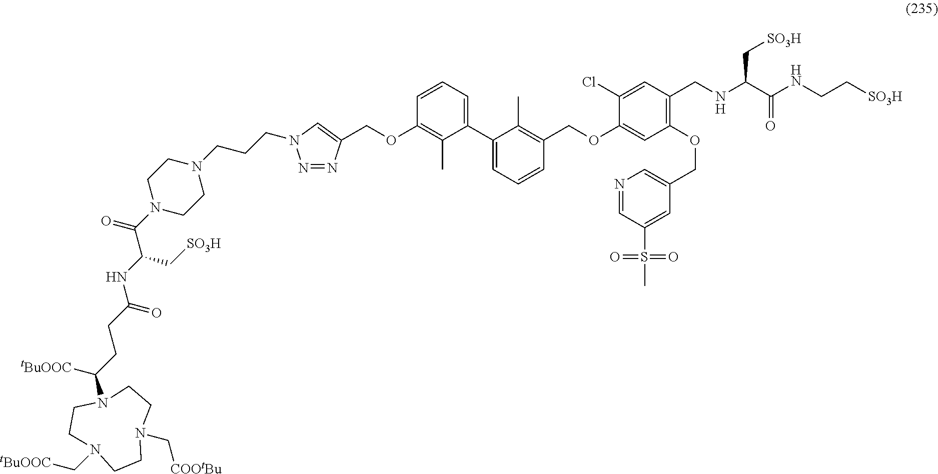

(R)-2-((R)-4-(4,7-Bis(2-(tert-butoxy)-2-oxoethyl)-1,4,7-triazonan-1-yl)-5-(tert-butoxy)-5-oxopentanamido)-3-(4-(3-(4-(((3′-((2-chloro-5-((5-(methylsulfonyl)pyridin-3-yl)methoxy)-4-((((R)-1-oxo-3-sulfo-1-((2-sulfoethyl)amino)propan-2-yl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-3-oxopropane-1-sulfonic acid (235). The amine (234) (20.0 mg, 17.1 μmol, 1.00 equiv.), (R)-NODAGA(t-Bu)₃ (18.6 mg, 34.2 μmol, 2.00 equiv.), HBTU (9.7 mg, 25.6 μmol, 1.50 equiv.), abs. DIPEA (6.0 μL, 34.2 μmol, 2.00 equiv.) and HOBt (2.3 mg, 17.1 μmol, 1.00 equiv.) reacted in abs. DMF (1 mL) according to GP-8. Semi-preparative purification was omitted and the residue was directly subjected to tBu-deprotection in the next step.

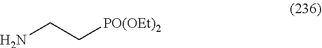

Diethyl (2-aminoethyl)phosphonate (236) was synthesized according to the literature.¹

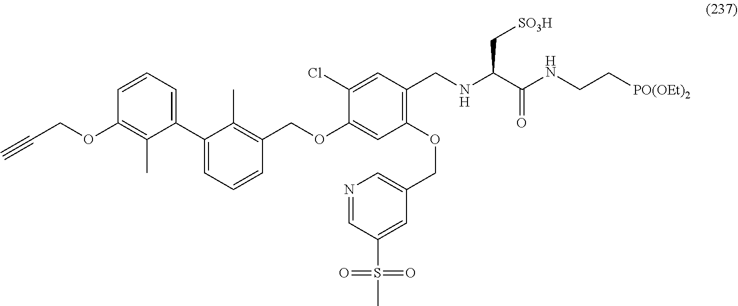

(R)-2-((5-Chloro-4-((2,2′-dimethyl-3′-(prop-2-yn-1-yloxy)-[1,1′-biphenyl]-3-yl)methoxy)-2-((5-(methylsulfonyl)pyridin-3-yl)methoxy)benzyl)amino)-3-((2-(diethoxyphosphoryl)ethyl)amino)-3-oxopropane-1-sulfonic acid (237). The carboxylic acid (231) (20.0 mg, 28.1 μmol, 1.00 equiv.), diethyl (2-aminoethyl)phosphonate (236) (7.6 mg, 42.2 μmol, 1.50 equiv.), HBTU (11.2 mg, 29.5 mmol, 1.05 equiv.), HOBt (3.8 mg, 28.1 mol, 1.00 equiv.) and abs. DIPEA (9.8 μL, 56.2 mmol, 2.00 equiv.) reacted in abs. DMF (1 mL) according to GP-8. Semi-preparative RP-HPLC (Agilent Zorbax SB C-18 5 μm 80 Å, 9.4×250 mm with 40-90% acetonitrile (0.1% TFA) in water (0.1% TFA) in a linear gradient over 45 min, 6 mL/min, Rₜ=8 min) with subsequent lyophilization yielded alkyne (237) (15.4 mg, 17.0 μmol, 60%) as a colorless powder. mp=117-120° C. Rₜ=12.10 min (System A), purity=100%. ¹H NMR (400 MHz, DMSO-d₆) δ=9.39 (bs, 1H), 9.11 (s, 1H), 9.07 (s, 1H), 8.98 (bs, 1H), 8.89 (t, 3J=5.4 Hz, 1H), 8.51 (s, 1H), 7.55 (s, 1H), 7.48-7.50 (m, 1H), 7.22-7.29 (m, 3H), 7.05-7.10 (m, 2H), 6.74 (d, ³J=7.5 Hz, 1H), 5.41-5.48 (m, 2H), 5.26-5.32 (m, 2H), 4.86 (d, 4J=1.9 Hz, 2H), 4.20-4.23 (m, 1H), 3.97-4.05 (m, 5H), 3.59 (s, 1H), 3.25-3.29 (m, 2H), 2.98-3.01 (m, 1H), 2.74-2.80 (m, 1H), 2.03 (s, 2H), 1.93-2.01 (m, 2H), 1.24 ppm (t, 3J=7.0 Hz, 6H). ¹³C NMR (101 MHz, DMSO-d₆) δ=165.6, 158.4, 158.0, 156.4, 155.5, 155.4, 153.5, 147.1, 142.3, 141.5, 136.9, 134.7, 134.6, 134.6, 134.4, 132.7, 129.3, 127.8, 126.2, 125.2, 124.1, 122.0, 113.1, 112.6, 110.9, 100.1, 79.5, 78.2, 69.7, 67.3, 61.2, 61.1, 56.3, 55.8, 49.7, 44.2, 43.6, 33.5, 25.4, 24.1, 16.3, 16.2, 15.3, 12.8 ppm. ³¹P NMR (162 MHz, DMSO-d₆) δ=28.2 ppm. IR (ATR): \tilde{ν}=1687 (w), 1606 (w), 1575 (w), 1505 (w), 1446 (w), 1409 (w), 1306 (m), 1163 (s), 1145 (s), 1091 (w), 1021 (m), 964 (m), 785 (w), 766 (w), 589 cm⁻¹ (w). MS (HR-ESI⁺): Exact mass calculated for [M+H]⁺: m/z=906.2262, measured: m/z=906.2256.

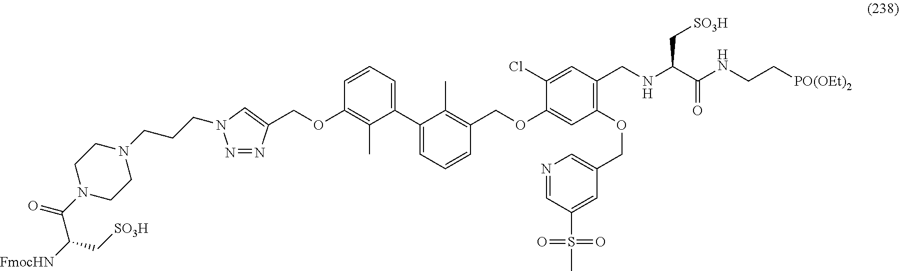

(R)-2-((4-((3′-((1-(3-(4-((((9H-Fluoren-9-yl)methoxy)carbonyl)(sulfo)-D-alanyl)piperazin-1-yl)propyl)-1H-1,2,3-triazol-4-yl)methoxy)-2,2′-dimethyl-[1,1′-biphenyl]-3-yl)methoxy)-5-chloro-2-((5-(methylsulfonyl)pyridin-3-yl)methoxy)benzyl)amino)-3-((2-(diethoxyphosphoryl)ethyl)amino)-3-oxopropane-1-sulfonic acid (238). Alkyne (237) (34.0 mg, 37.5 μmol, 1.00 equiv.) reacted with the linker structure (76) (18.3 mg, 33.8 μmol, 0.90 equiv.) with a premixed catalyst consisting of CuSO₄ (0.9 mg, 5.6 μmol, 0.15 equiv.), THPTA (1.6 mg, 3.8 μmol, 0.10 equiv.) and sodium ascorbate (37.2 mg, 188 μmol, 5.00 equiv.) in an 1:1 mixture of H₂O/t-BuOH (3 mL) at room temperature for 16 h. The solvent was removed in vacuo and the residue was used in the next step without further purification.

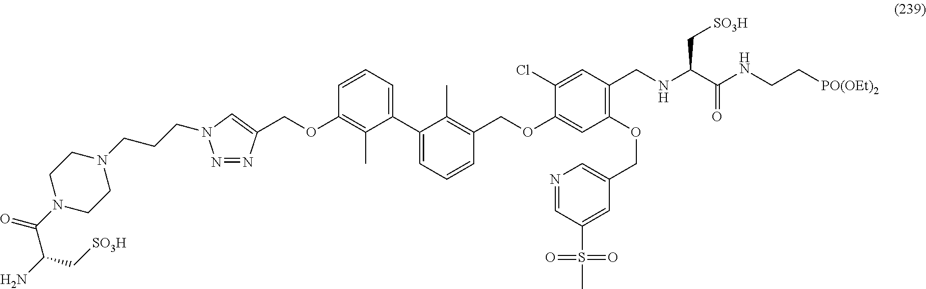

(R)-2-Amino-3-(4-(3-(4-(((3′-((2-chloro-4-((((R)-1-((2-(diethoxyphosphoryl)ethyl)amino)-1-oxo-3-sulfopropan-2-yl)amino)methyl)-5-((5-(methyl-sulfonyl)pyridin-3-yl)methoxy)phenoxy)methyl)-2,2′-dimethyl-[1,1′-biphenyl]-3-yl)oxy)methyl)-1H-1,2,3-triazol-1-yl)propyl)piperazin-1-yl)-3-oxopropane-1-sulfonic acid (239). The residue from the previous reaction was dissolved in abs. DMF (3 mL) and the Fmoc group was removed with sodium azide (12.2 mg, 188 μmol, 5.00 equiv.) according to GP-9. Semi-preparative RP-HPLC purification (Agilent Zorbax SB C-18 5 μm 80 Å, 9.4×250 mm with 33-88% acetonitrile (0.1% TFA) in water (0.1% TFA) in a linear gradient over 45 min, 6 mL/min, Rₜ=12 min) with subsequent lyophilization, yielded primary amine (239) (47.3 mg, 32.6 μmol, 87% over two steps) as a colorless powder. mp=190° C. (decomposition). Rₜ=9.48 min (System A), purity=100%. ¹H NMR (400 MHz, DMSO-d₆) δ=9.40 (bs, 1H), 9.11 (d, ⁴J=1.4 Hz, 1H), 9.07 (d, ⁴J=2.0 Hz, 1H), 8.97 (bs, 1H), 8.89 (t, ³J=5.5 Hz, 1H), 8.51 (t, 4J=1.9 Hz, 1H), 8.29 (s, 1H), 8.11-8.12 (m, 2H), 7.54 (s, 1H), 7.48 (d, ³J=7.5 Hz, 1H), 7.24-7.29 (m, 2H), 7.18-7.20 (m, 1H), 7.08 (d, ³J=7.3 Hz, 1H), 6.73 (d, 3J=7.3 Hz, 1H), 5.41-5.51 (m, 2H), 5.29-5.35 (m, 2H), 5.21-5.26 (m, 2H), 4.50 (t, 3J=6.8 Hz, 2H), 4.03-4.06 (m), 3.23-3.33 (m, 2H), 3.16-3.19 (m, 1H), 2.95 (m, 2H), 2.75-2.84 (m, 2H), 2.27-2.32 (m, 2H), 1.93-2.03 (m, 4H), 1.82 (s, 3H), 1.24 (t, 3J=7.0 Hz, 4H). ¹³C NMR (101 MHz, DMSO-d₆) δ=166.3, 165.6, 158.9, 158.5, 158.2, 157.8, 156.5, 156.2, 155.5, 153.5, 147.2, 143.2, 142.3, 141.6, 136.9, 134.7, 134.6, 134.5, 132.8, 132.7, 129.4, 127.9, 126.4, 125.5, 124.5, 123.9, 121.8, 113.1, 112.7, 110.7, 100.1, 69.8, 67.4, 61.7, 61.2, 61.2, 56.3, 53.2, 50.5, 49.8, 47.7, 46.7, 44.2, 43.6, 42.0, 33.5, 25.5, 24.3, 2.41, 16.3, 15.3, 12.9 ppm. ³¹P NMR (162 MHz, DMSO-d₆) δ=28.2 ppm. IR (ATR): \tilde{ν}=1673 (m), 1606 (w), 1574 (w), 1504 (w), 1445 (w), 1409 (w), 1306 (w), 1173 (s), 1145 (s), 1033 (m), 966 (m), 798 (w), 720 cm⁻¹ (w). MS (HR-ESI⁺): Exact mass calculated for [M+H]⁺: m/z=1226.3529, measured: m/z=1226.3517.

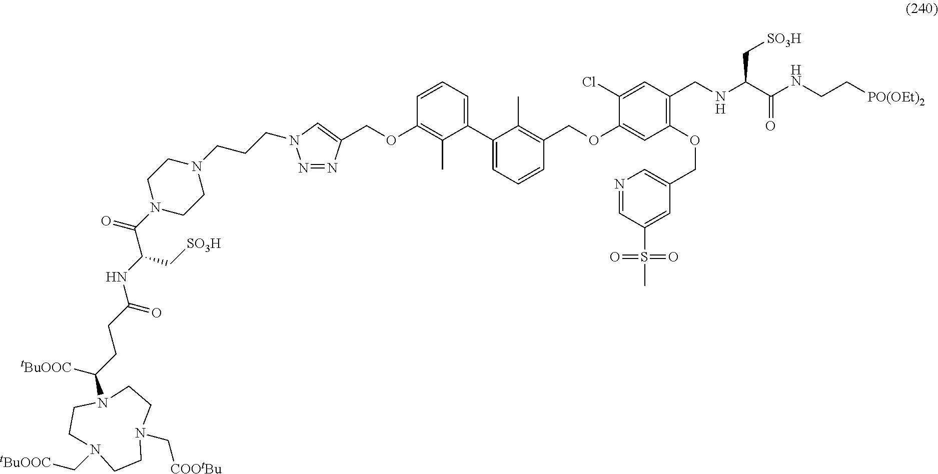

(R)-2-((R)-4-(4,7-Bis(2-(tert-butoxy)-2-oxoethyl)-1,4,7-triazonan-1-yl)-5-(tert-butoxy)-5-oxopentanamido)-3-(4-(3-(4-(((3′-((2-chloro-4-((((R)-1-((2-(diethoxyphosphoryl)ethyl)amino)-1-oxo-3-sulfopropan-2-yl)amino)methyl)-5-((5-(methylsulfonyl)pyridin-3-yl)methoxy)phenoxy)methyl)-2,2′-dimethyl-[1,1′-biphenyl]-3-yl)oxy)methyl)-1H-1,2,3-triazol-1-yl)propyl)piperazin-1-yl)-3-oxopropane-1-sulfonic acid (240). The amine (239) (10.0 mg, 8.2 μmol, 1.00 equiv.), (R)-NODAGA(t-Bu)₃ (8.9 mg, 16.3 μmol, 2.00 equiv.), HBTU (4.6 mg, 12.2 μmol, 1.50 equiv.), abs. DIPEA (2.8 μL, 16.3 μmol, 2.00 equiv.) and HOBt (1.1 mg, 8.2 μmol, 1.00 equiv.) reacted in abs. DMF (1 mL) according to GP-8. Semi-preparative purification (Agilent Zorbax SB C-18 5 μm 80 Å, 9.4×250 mm with 30-90% acetonitrile (0.1% TFA) in water (0.1% TFA) in a linear gradient over 45 min, 6 mL/min, Rₜ=13 min) with subsequent lyophilization, yielded NODA-GA-tris(t-Bu)₃-conjugate (240) (9.0 mg, 5.1 μmol, 63%) as a colorless oil. Rₜ=11.50 min (System A), purity=93.9%. IR (ATR): \tilde{ν}=1727 (m), 1674 (s), 1577 (w), 1455 (w), 1393 (w), 1370 (w), 1309 (w), 1200 (s), 1148 (s), 1040 (m), 696 (w), 834 (w), 799 (w), 720 cm⁻¹ (w). MS (HR-ESI⁺): Exact mass calculated for [M+H]⁺: m/z=1753.6943, measured: m/z=1753.6947.

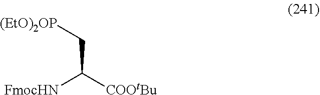

tert-Butyl (R)-2-((((9H-fluoren-9-yl)methoxy)carbonyl)amino)-3-(diethoxyphosphoryl)propanoate (234). Triethyl phosphite (3.98 mL, 23.2 mmol, 5.00 equiv.) was degassed with argon for 30 min and then the N-[(9H-Fluoren-9-ylmethoxy)carbonyl]-3-iodo-L-alanine 1,1-dimethylethyl ester (2.03 g, 4.64 mmol, 1.00 equiv.) was added. The solution was stirred at 140° C. for 16 h under argon. Subsequently, all volatiles were removed by vacuum destillation (90° C., 10-3 mbar) and the yellowish residue was used in the next step without further purification.

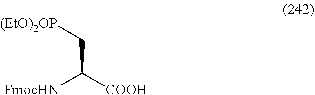

(R)-2-((((9H-Fluoren-9-yl)methoxy)carbonyl)amino)-3-(diethoxyphosphoryl)propanoic acid (242). The residue from the previous reaction was dissolved in an 1:1 mixture of TFA/DCM (20 mL). The solution was stirred at room temperature for 5 h and subsequently all solvents were removed under reduced pressure. DCM (200 mL) and sodium bicarbonate solution were added (400 mL) to reach pH 8. Phases were separated, the aqueous phase was washed with DCM (150 mL) and then the aqueous phase was adjusted to pH 2 with 1 M hydrochloric acid. The extraction of the aqueous phase was performed with DCM (3×200 mL), the combined organic extracts were dried over sodium sulfate, filtrated and concentrated under reduced pressure. Flash column chromatography on SiO₂ (EA:MeOH:AcOH, 93:7:1, R_{f}=0.27) was performed to yield the free carboxylic acid (242) (1.04 g, 2.33 mmol, 57% over two steps) as a light yellowish oil. Rₜ=10.90 min (System A), purity=100%. ¹H NMR (400 MHz, CD₂Cl₂) δ=7.73 (d, ³J=7.5 Hz, 2H), 7.54-7.56 (m, 2H), 7.35 (t, ³J=7.4 Hz, 2H), 7.24 (t, ³J=7.2 Hz, 2H), 4.02-4.46 (m, 7H), 1.20-1.22 ppm (m, 6H). ¹³C NMR (101 MHz, CD₂Cl₂) δ=176.7, 156.9, 144.4, 141.8, 128.2, 127.6, 125.8, 120.4, 67.8, 63.6 (d, ²J=33.5 Hz), 51.0, 25.5 (d, ¹J=150.2 Hz), 16.5 ppm (d, ³J=5.6 Hz). 31P NMR (162 MHz, CD₂Cl₂) δ=28.7 ppm. IR (ATR): G=1682 (s), 1607 (m), 1519 (w), 1448 (m), 1206 (s), 1137 (s), 1023 (s), 971 (w), 840 (w), 799 (w), 759 (w), 738 (m), 722 cm⁻¹ (m). MS (HR-ESI⁺): Exact mass calculated for [M+H]⁺: m/z=448.1480, measured: m/z=448.1518.

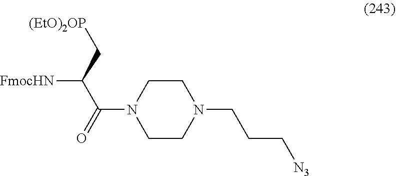

(9H-Fluoren-9-yl)methyl (R)-(1-(4-(3-azidopropyl)piperazin-1-yl)-3-(diethoxyphosphoryl)-1-oxopropan-2-yl)carbamate (243). The carboxylic acid (242) (50.0 mg, 111.8 μmol, 1.00 equiv.), azide linker (31) (28.4 mg, 167.6 μmol, 1.50 equiv.), HBTU (50.9 mg, 134.1 μmol, 1.20 equiv.), HOBt (15.1 mg, 111.8 mol, 1.00 equiv.) and abs. pyridine (18.2 μL, 223.5 μmol, 2.00 equiv.) reacted in abs. DMF (2 mL) according to GP-8. Semi-preparative RP-HPLC (Agilent Zorbax SB C-18 5 μm 80 Å, 9.4×250 mm with 30-90% acetonitrile (0.1% TFA) in water (0.1% TFA) in a linear gradient over 45 min, 6 mL/min, Rₜ=8 min) with subsequent lyophilization yielded linker (243) (38.0 mg, 63.5 μmol, 57%) as a colorless oil. Rₜ=10.40 min (System A), purity=86.8%. ¹H NMR (400 MHz, DMSO-d₆) δ=10.47 (bs, 1H), 7.89 (d, ³J=7.5 Hz, 2H), 7.70-7.72 (m, 2H), 7.42 (t, ³J=7.4 Hz, 2H), 7.33 (t, ³J=7.4 Hz, 2H), 4.65-4.73 (m, 1H), 4.32-4.33 (m, 2H), 4.20-4.23 (m, 1H), 3.94-3.99 (m, 4H), 3.46 (t, ³J=6.4 Hz, 2H), 3.11 (bs, 2H), 2.12-2.29 (m, 2H), 1.90 (bs, 2H), 1.20 ppm (q, ³J=6.8 Hz, 6H). ¹³C NMR (101 MHz, CD₂Cl₂) δ=169.1, 169.0, 158.5, 155.4, 143.7, 140.7, 127.6, 127.0, 125.2, 120.1, 65.8, 61.3 (d, ³J=6.2 Hz), 53.3, 50.8, 47.9, 46.6, 45.4, 42.2, 27.5 (d, J=139.2 Hz), 23.1, 16.2 (d, ³J=5.9 Hz). ³¹P NMR (162 MHz, DMSO-d₆) δ=27.1 ppm. IR (ATR): G=2101 (s), 1660 (s), 1531 (w), 1448 (m), 1248 (n), 1196 (s), 1130 (n), 1023 (s), 970 (n), 830 (w), 798 (w), 760 (w), 740 (m), 720 cm⁻¹ (m). MS (HR-ESI⁺): Exact mass calculated for [M+H]⁺: m/z=599.2747, measured: m/z=599.2745.

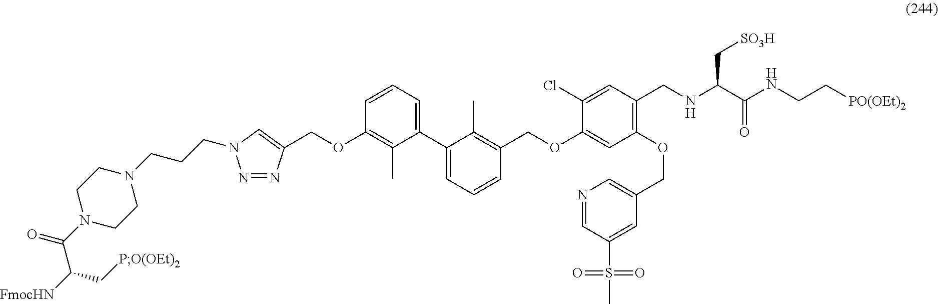

(R)-2-((4-((3′-((1-(3-(4-((R)-2-((((9H-Fluoren-9-yl)methoxy)carbonyl)amino)-3-(diethoxyphosphoryl)propanoyl)piperazin-1-yl)propyl)-1H-1,2,3-triazol-4-yl)methoxy)-2,2′-dimethyl-[1,1′-biphenyl]-3-yl)methoxy)-5-chloro-2-((5-(methylsulfonyl)pyridin-3-yl)methoxy)benzyl)amino)-3-((2-(diethoxyphosphoryl)ethyl)amino)-3-oxopropane-1-sulfonic acid (244). Alkyne (237) (60.0 mg, 66.2 μmol, 1.00 equiv.) reacted with the linker structure (243) (60.0 mg, 66.2 μmol, 0.90 equiv.) with a premixed catalyst consisting of CuSO₄ (1.6 mg, 9.9 μmol, 0.15 equiv.), THPTA (2.9 mg, 6.6 μmol, 0.10 equiv.) and sodium ascorbate (65.6 mg, 331 μmol, 5.00 equiv.) in an 1:1 mixture of H₂O/t-BuOH (3 mL) at room temperature for 16 h. The solvent was removed in vacuo and the residue was used in the next step without further purification.

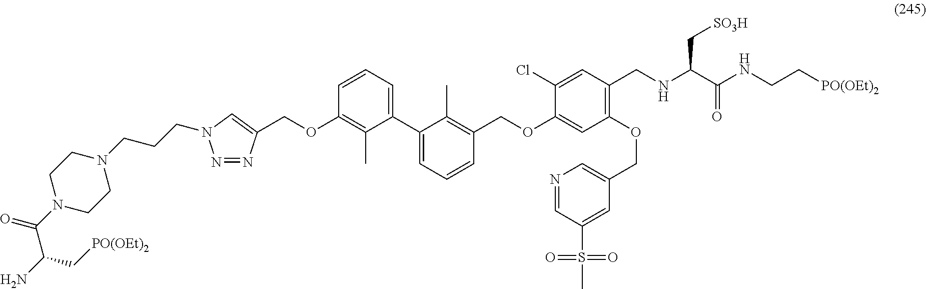

(R)-2-((4-((3′-((1-(3-(4-((R)-2-Amino-3-(diethoxyphosphoryl)propanoyl)piperazin-1-yl)propyl)-1H-1,2,3-triazol-4-yl)methoxy)-2,2′-dimethyl-[1,1′-biphenyl]-3-yl)methoxy)-5-chloro-2-((5-(methylsulfonyl)pyridin-3-yl)methoxy)benzyl)amino)-3-((2-(diethoxyphosphoryl)ethyl)amino)-3-oxopropane-1-sulfonic acid (245). The residue from the previous reaction was dissolved in abs. DMF (3 mL) and the Fmoc group was removed with sodium azide (21.5 mg, 331 μmol, 5.00 equiv.) according to GP-9. Semi-preparative RP-HPLC purification (Agilent Zorbax SB C-18 5 μm 80 Å, 9.4×250 mm with 30-70% acetonitrile (0.1% TFA) in water (0.1% TFA) in a linear gradient over 45 min, 6 mL/min, Rₜ=9 min) with subsequent lyophilization, yielded primary amine (245) (19.0 mg, 14.8 μmol, 50% over two steps) as a colorless powder. mp=225° C. (decomposition). Rₜ=9.74 min (System A), purity=98.0%. ¹H NMR (400 MHz, DMSO-d₆) δ=9.40 (bs, 2H), 9.11 (s, 1H), 9.07 (s, 1H), 8.98 (bs, 1H), 8.90 (t, ³J=5.3 Hz, 1H), 8.51 (s, 1H), 8.29-8.35 (m, 4H), 7.54 (s, 1H), 7.49 (d, ³J=7.4 Hz, 1H), 7.24-7.29 (m, 3H), 7.19 (d, ³J=8.2 Hz, 1H), 7.08 (d, ³J=7.5 Hz, 1H), 6.73 (d, ³J=7.3 Hz, 1H), 5.41-5.51 (m, 3H), 5.26-5.35 (m, 3H), 5.21 (s, 2H), 4.63 (bs, 2H), 4.50 (t, ³J=6.6 Hz, 3H), 4.20-4.23 (m, 1H), 3.98-4.02 (m), 3.25-3.31 (m, 3H), 3.14 (bs, 3H), 2.75-2.81 (m, 2H), 2.29-2.35 (m, 4H), 1.99 (s, 3H), 1.93-1.97 (m, 3H), 1.81 (s, 3H), 1.71-1.74 (m, 4H), 1.24 ppm (t, ³J=7.0 Hz, 12H). ¹³C NMR (101 MHz, DMSO-d₆) δ=166.5, 165.6, 160.8, 158.8, 158.7, 158.5, 158.2, 156.4, 156.2, 155.5, 153.5, 147.1, 143.2, 142.3, 141.6, 136.9, 134.7, 134.6, 134.5, 132.7, 132.7, 129.3, 127.8, 126.4, 125.5, 124.5, 123.9, 121.8, 118.3, 115.4, 113.0, 112.6, 110.7, 100.1, 69.8, 67.4, 62.1, 62.0, 61.7, 61.2, 61.1, 56.3, 53.2, 50.7, 49.7, 46.7, 45.9, 45.8, 44.2, 43.6, 42.6, 33.5, 27.4, 25.9, 25.9, 25.4, 24.5, 24.1, 16.3, 16.2, 16.1, 16.1, 15.3, 12.8 ppm. 31P NMR (162 MHz, DMSO-d₆) δ=28.2 (s, 1H), 26.8 ppm, (s, 1H). IR (ATR): \tilde{ν}=1673 (s), 1606 (w), 1575 (w), 1504 (w), 1445 (w), 1409 (w), 1307 (m), 1199 (s), 1174 (s), 1145 (s), 1021 (s), 967 (m), 830 (w), 798 (m), 720 cm⁻¹ (m). MS (HR-ESI⁺): Exact mass calculated for [M+H]⁺: m/z=1282.4250, measured: m/z=1282.4251.

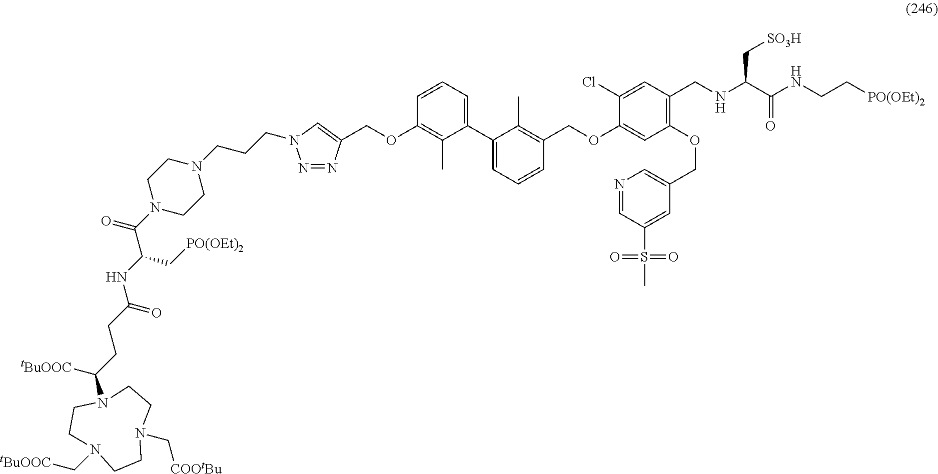

(R)-2-((4-((3′-((1-(3-(4-((R)-2-((R)-4-(4,7-Bis(2-(tert-butoxy)-2-oxoethyl)-1,4,7-triazonan-1-yl)-5-(tert-butoxy)-5-oxopentanamido)-3-(diethoxyphosphoryl)propanoyl)piperazin-1-yl)propyl)-1H-1,2,3-triazol-4-yl)methoxy)-2,2′-dimethyl-[1,1′-biphenyl]-3-yl)methoxy)-5-chloro-2-((5-(methylsulfonyl)pyridin-3-yl)methoxy)benzyl)amino)-3-((2-(diethoxyphosphoryl)ethyl)amino)-3-oxopropane-1-sulfonic acid (246). The amine (245) (20.0 mg, 15.6 μmol, 1.00 equiv.), (R)-NODAGA(t-Bu)₃ (17.0 mg, 31.2 μmol, 2.00 equiv.), HBTU (8.9 mg, 23.4 μmol, 1.50 equiv.), abs. DIPEA (5.4 μL, 31.2 μmol, 2.00 equiv.) and HOBt (4.2 mg, 15.6 μmol, 1.00 equiv.) reacted in abs. DMF (1 mL) according to GP-8. Semi-preparative purification (Agilent Zorbax SB C-18 5 μm 80 Å, 9.4×250 mm with 40-90% acetonitrile (0.1% TFA) in water (0.1% TFA) in a linear gradient over 45 min, 6 mL/min, Rₜ=8 min) with subsequent lyophilization, yielded NODA-GA-tris(t-Bu)₃-conjugate (246) (25.0 mg, 13.8 μmol, 89%) as a colorless powder. mp=140° C. (decomposition). Rₜ=11.70 min (System A), purity=100%. IR (ATR): G=1727 (w), 1678 (m), 1578 (w), 1454 (w), 1393 (w), 1369 (w), 1308 (w), 1251 (m), 1199 (m), 1148 (s), 1026 (m), 967 (w), 889 (w), 831 (w), 799 (m), 719 cm⁻¹ (m). MS (HR-ESI⁺): Exact mass calculated for [M+H]⁺: m/z=1808.7664, measured: m/z=1808.7684.

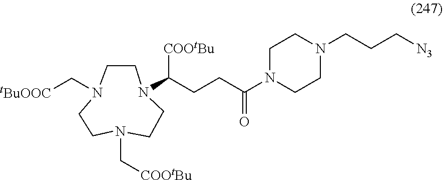

Di-tert-butyl 2,2′-(7-(5-(4-(3-azidopropyl)piperazin-1-yl)-1-(tert-butoxy)-1, 5-dioxopentan-2-yl)-1,4,7-triazonane-1,4-diyl)(R)-diacetate (247). (R)-NODA-GA(t-Bu)₃ (15.0 mg, 27.6 μmol, 1.00 equiv.), azide linker (31) (7.0 mg, 41.4 μmol, 1.50 equiv.), HBTU (12.6 mg, 33.1 μmol, 1.20 equiv.) and abs. DIPEA (7.3 μL, 55.2 μmol, 2.00 equiv.) reacted in abs. DMF (1 mL) according to GP-8. Semi-preparative RP-HPLC (Agilent Zorbax SB C-18 5 μm 80 Å, 9.4×250 mm with 30-80% acetonitrile (0.1% TFA) in water (0.1% TFA) in a linear gradient over 45 min, 6 mL/min, Rₜ=8 min, detection at 220 nm) with subsequent lyophilization yielded linker (247) (15.0 mg, 21.6 μmol, 78%) as a colorless oil. Rₜ=10.20 min (System A), purity=97.0%. ¹H NMR (400 MHz, CD₃OD) δ=3.97-3.98 (m, 3H), 2.59-3.66 (m), 1.91-2.13 (m, 5H), 1.45-1.50 ppm (m, 27H). ¹³C NMR (101 MHz, CD₃OD) δ=173.0, 171.2, 168.7, 162.7, 162.4, 119.4, 116.5, 84.5, 83.4, 64.7, 56.3, 56.3, 55.7, 52.9, 52.7, 51.6, 51.1, 47.0, 46.3, 43.5, 39.7, 31.0, 28.5, 28.5, 28.4, 26.7, 24.7 ppm. IR (ATR): G=2102 (m), 1727 (m), 1683 (s), 1456 (w), 11369 (m), 1251 (w), 1196 (m), 1149 (s), 1127 (s), 976 (w), 830 (w), 798 (w), 735 (m), 719 cm⁻¹ (m). MS (HR-ESI⁺): Exact mass calculated for [M+H]⁺: m/z=695.4820, measured: m/z=695.4824.

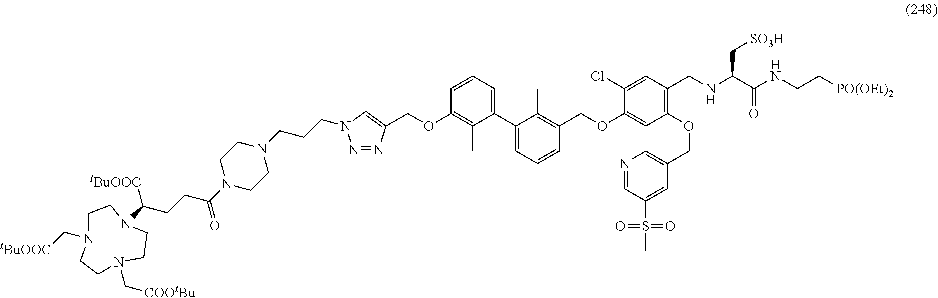

(R)-2-((4-((3′-((1-(3-(4-((R)-4-(4,7-Bis(2-(tert-butoxy)-2-oxoethyl)-1,4,7-triazonan-1-yl)-5-(tert-butoxy)-5-oxopentanoyl)piperazin-1-yl)propyl)-1H-1,2,3-triazol-4-yl)methoxy)-2,2′-dimethyl-[1,1′-biphenyl]-3-yl)methoxy)-5-chloro-2-((5-(methylsulfonyl)pyridin-3-yl)methoxy)benzyl)amino)-3-((2-(diethoxyphosphoryl)ethyl)amino)-3-oxopropane-1-sulfonic acid (248). Alkyne (237) (15.0 mg, 16.5 μmol, 1.00 equiv.) reacted with the linker structure (247) (9.8 mg, 14.1 μmol, 0.85 equiv.) with a premixed catalyst consisting of CuSO₄ (0.4 mg, 2.5 μmol, 0.15 equiv.), THPTA (0.7 mg, 1.7 μmol, 0.10 equiv.) and sodium ascorbate (16.4 mg, 82.7 μmol, 5.00 equiv.) in an 1:1 mixture of H₂O/t-BuOH (2 mL) at room temperature for 16 h. The solvent was removed in vacuo and the residue was purified by semi-preparative RP-HPLC purification (Agilent Zorbax SB C-18 5 μm 80 Å, 9.4×250 mm with 32-90% acetonitrile (0.1% TFA) in water (0.1% TFA) in a linear gradient over 45 min, 6 mL/min, Rₜ=15 min) and subsequent lyophilization yielded the NODA-GA-tris(t-Bu)₃-conjugate (248) (11.0 mg, 6.8 μmol, 41%) as a colorless powder. mp=155° C. (decomposition). Rₜ=11.90 min (System A), purity=95.7%. \tilde{ν}=1726 (w), 1681 (m), 1578 (w), 1455 (w), 1393 (w), 1369 (w), 1308 (w), 1251 (m), 1200 (m), 1147 (s), 1027 (m), 967 (w), 832 (w), 799 (m), 720 cm⁻¹ (m). MS (HR-ESI⁺): Exact mass calculated for [M+H]⁺: m/z=1600.7004, measured: m/z=1600.6986.

### FURTHER EXAMPLES

### Example 37—Compound 201

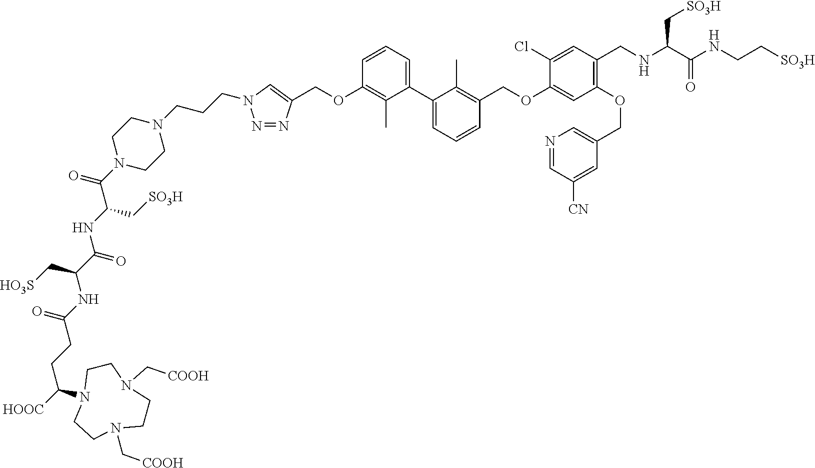

2,2′-(7-((R)-1-Carboxy-4-(((R)-1-(((R)-1-(4-(3-(4-(((3′-((2-chloro-5-((5-cyanopyridin-3-yl)methoxy)-4-((((R)-1-oxo-3-sulfo-1-((2-sulfoethyl)amino)propan-2-yl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-1-oxo-3-sulfopropan-2-yl)amino)-1-oxo-3-sulfopropan-2-yl)amino)-4-oxobutyl)-1,4,7-triazonane-1,4-diyl)diacetic acid (201). The tert-butyl ester deprotection was performed according to GP-10 in 500 μL of the deprotection cocktail with the residue of compound 230. Semi-preparative RP-HPLC purification (Agilent Zorbax SB C-18 5 μm 80 Å, 9.4×250 mm with 20-60% acetonitrile (0.1% TFA) in water (0.1% TFA) in a linear gradient over 45 min, 6 mL/min, Rₜ=13 min) and subsequent lyophilization yielded the NODAGA-conjugate (201) (9.2 mg, 5.6 μmol, 42% over two steps) as a colorless powder. mp=240° C. (decomposition). Rₜ=8.57 min (System A), purity=100%. \tilde{ν}=1651 (m), 1574 (w), 1505 (w), 1455 (w), 1410 (w), 1309 (w), 1166 (s), 1037 (s), 723 cm⁻¹ (w). MS (HR-ESI⁺): Exact mass calculated for [M+H]⁺: m/z=1627.4460, measured: m/z=1627.4447.

### Example 38—Compound 202

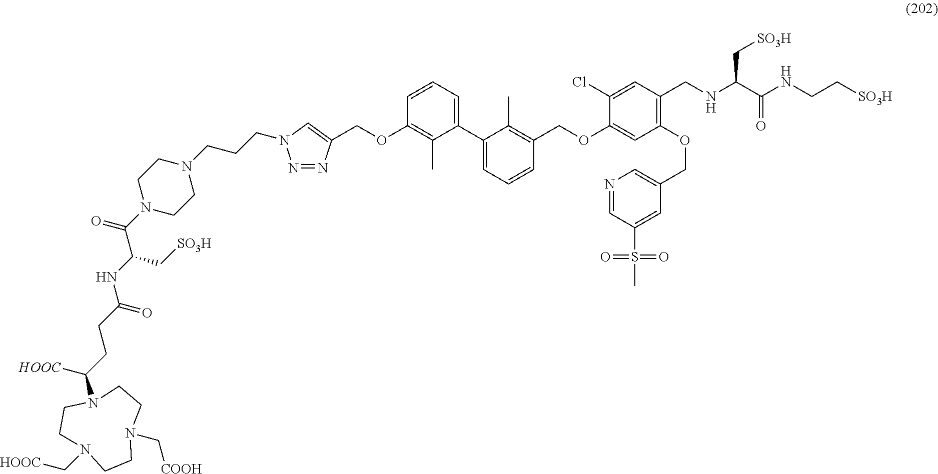 [SMILES: Cc1c(COc2cc(OCc3cncc(S(C)(=O)=O)c3)c(CN[C@@H](CS(=O)(=O)O)C(=O)NCCS(=O)(=O)O)cc2Cl)cccc1-c1cccc(OCc2cn(CCCN3CCN(C(=O)[C@H](CS(=O)(=O)O)NC(=O)CC[C@H](C(=O)O)N4CCN(CC(=O)O)CCN(CC(=O)O)CC4)CC3)nn2)c1C]

2,2′-(7-((R)-1-Carboxy-4-(((R)-1-(4-(3-(4-(((3′-((2-chloro-5-((5-(methylsulfonyl)pyridin-3-yl)methoxy)-4-((((R)-1-oxo-3-sulfo-1-((2-sulfoethyl)amino)propan-2-yl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-1-oxo-3-sulfopropan-2-yl)amino)-4-oxobutyl)-1,4,7-triazonane-1,4-diyl)diacetic acid (202). The tert-butyl ester deprotection was performed according to GP-10 in 400 μL of the deprotection cocktail with the residue of compound 235. Semi-preparative RP-HPLC purification (Agilent Zorbax SB C-18 5 μm 80 Å, 9.4×250 mm with 20-80% acetonitrile (0.1% TFA) in water (0.1% TFA) in a linear gradient over 45 min, 6 mL/min, Rₜ=11 min) and subsequent lyophilization yielded the NODAGA-conjugate (202) (13.3 mg, 8.7 μmol, 51% over two steps) as a colorless powder. mp=230° C. (decomposition). Rₜ=9.05 min (System A), purity=95.0%. IR (ATR): \tilde{ν}=1726 (w), 1651 (w), 1574 (w), 1505 (w), 1454 (w), 1410 (w), 1305 (m), 1166 (s), 1146 (s), 1036 (s), 768 (w), 727 cm⁻¹ (w). MS (HR-ESI⁺): Exact mass calculated for [M+H]⁺: m/z=1528.4377, measured: m/z=1528.4356.

### Example 39—Compound 203

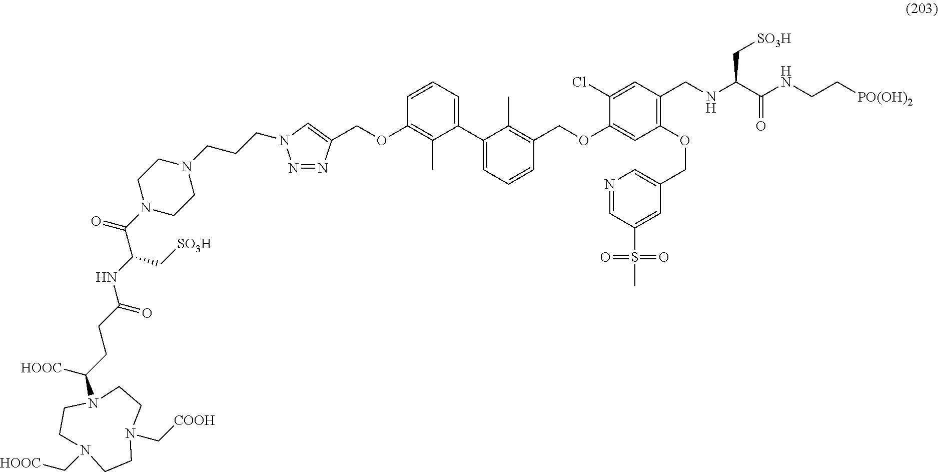

2,2′-(7-((R)-1-Carboxy-4-(((R)-1-(4-(3-(4-(((3′-((2-chloro-5-((5-(methylsulfonyl)pyridin-3-yl)methoxy)-4-((((R)-1-oxo-1-((2-phosphonoethyl)amino)-3-sulfopropan-2-yl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-1-oxo-3-sulfopropan-2-yl)amino)-4-oxobutyl)-1,4,7-triazonane-1,4-diyl)diacetic acid (203). Dealkylation of phosphonate esters and subsequent tert-butyl deprotection of NODA-GA tris(t-Bu)₃-conjugate (240) (9.0 mg, 5.1 μmol, 1.00 equiv.) was performed with trimethylsilyl bromide (8.1 μL, 66.8 μmol, 13.0 equiv.) and 400 μL of the deprotection cocktail respectively according to GP-11. Semi-preparative RP-HPLC purification (Agilent Zorbax SB C-18 5 μm 80 Å, 9.4×250 mm with 20-80% acetonitrile (0.1% TFA) in water (0.1% TFA) in a linear gradient over 45 min, 6 mL/min, Rₜ=12 min) and subsequent lyophilization yielded the NODAGA-conjugate (203) (4.6 mg, 3.0 μmol, 57% over two steps) as a colorless powder. mp=215° C. (decomposition). Rₜ=15.88 min (System E), purity=100%. IR (ATR): \tilde{ν}=1650 (m), 1573 (w), 1502 (w), 1451 (w), 1408 (w), 1305 (m), 1145 (s), 1036 (m), 768 (w), 719 cm⁻¹ (w). MS (HR-ESI⁺): Exact mass calculated for [M+H]⁺: m/z=1528.4472, measured: m/z=1528.4458.

### Example 40—Compound 204

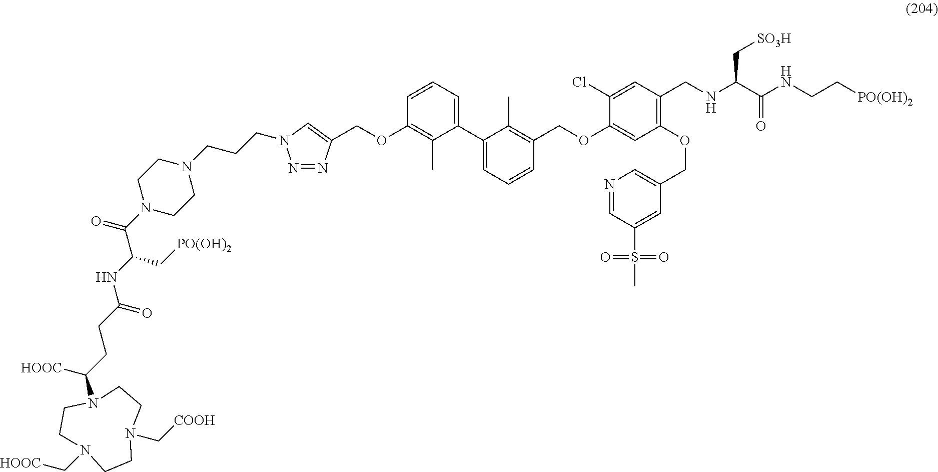

2,2′-(7-((R)-1-Carboxy-4-(((R)-1-(4-(3-(4-(((3′-((2-chloro-5-((5-(methylsulfonyl)pyridin-3-yl)methoxy)-4-((((R)-1-oxo-1-((2-phosphonoethyl)amino)-3-sulfopropan-2-yl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-1-oxo-3-phosphonopropan-2-yl)amino)-4-oxobutyl)-1,4,7-triazonane-1,4-diyl)diacetic acid (204). Dealkylation of phosphonate esters and subsequent tert-butyl deprotection of NODA-GA tris(t-Bu)₃-conjugate (246) (20.0 mg, 11.1 μmol, 1.00 equiv.) is performed with trimethylsilyl bromide (19.0 μL, 144 μmol, 13.0 equiv.) and 400 μL of the deprotection cocktail respectively according to GP-11. Semi-preparative RP-HPLC purification (Agilent Zorbax SB C-18 5 μm 80 Å, 9.4×250 mm with 25-80% acetonitrile (0.1% TFA) in water (0.1% TFA) in a linear gradient over 45 min, 6 mL/min, Rₜ=8 min) and subsequent lyophilization yielded the NODAGA-conjugate (204) (9.1 mg, 5.9 μmol, 53% over two steps) as a colorless powder. mp=220° C. (decomposition). Rₜ=16.09 min (System E), purity=100%. ν=1667 (m), 1651 (m), 1574 (w), 1505 (w), 1454 (w), 1409 (w), 1305 (m), 1170 (s), 1144 (s), 1036 (m), 797 (w), 719 cm⁻¹ (w). MS (HR-ESI⁺): Exact mass calculated for [M+H]⁺: m/z=1527.4534, measured: m/z=1527.4514. Example 41—Compound 205

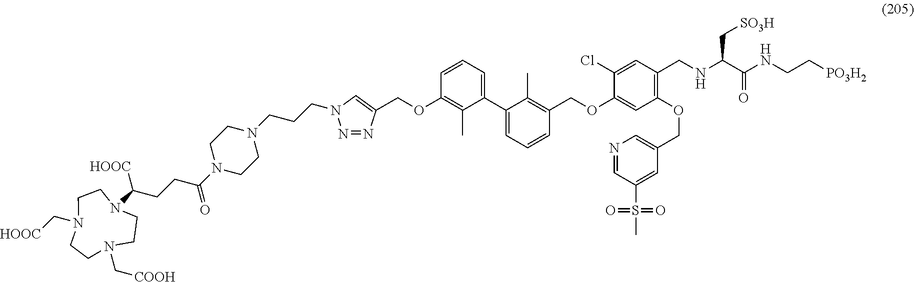

2,2′-(7-((R)-1-Carboxy-4-(4-(3-(4-(((3′-((2-chloro-5-((5-(methylsulfonyl)pyridin-3-yl)methoxy)-4-((((R)-1-oxo-1-((2-phosphonoethyl)amino)-3-sulfopropan-2-yl)amino)methyl)phenoxy)methyl)-2,2′-dimethyl-[1,1′-biphenyl]-3-yl)oxy)methyl)-1H-1,2,3-triazol-1-yl)propyl)piperazin-1-yl)-4-oxobutyl)-1,4,7-triazonane-1,4-diyl)diacetic acid (205). Dealkylation of phosphonate esters and subsequent tert-butyl deprotection of NODA-GA tris(t-Bu)₃-conjugate (248) (11.0 mg, 6.9 μmol, 1.00 equiv.) is performed with trimethylsilyl bromide (18.2 μL, 138 μmol, 20.0 equiv.) and 400 μL of the deprotection cocktail respectively according to GP-11. Semi-preparative RP-HPLC purification (Agilent Zorbax SB C-18 5 μm 80 Å, 9.4×250 mm with 25-80% acetonitrile (0.1% TFA) in water (0.1% TFA) in a linear gradient over 45 min, 6 mL/min, Rₜ=8 min) and subsequent lyophilization the NODAGA conjugate (205) (5.7 mg, 4.1 μmol, 54% over two steps) as a colorless powder. mp=220° C. (decomposition). Rₜ=16.09 min (System E), purity=100%. \tilde{ν}=1678 (m), 1643 (m), 1579 (w), 1443 (m), 1408 (m), 1307 (m), 1199 (s), 1173 (s), 1145 (s), 1039 (m), 890 (w), 799 (w), 720 (w). MS (HR-ESI⁺): Exact mass calculated for [M+H]⁺: m/z=1376.4500, measured: m/z=1376.4484.

### Example 42—Compounds 211, 212, 213, 214 und 215

### Labeling of Compounds 211, 212, 213, 214 und 215

The Ga-68 generator was eluted with approx. 1 M HCl (approx. 1500 MBq in 300 μL). 8 μL of 1 mM stock solution of the corresponding NODAGA-compound was added to a Protein LoBind Eppendorf tube containing 200 μL of an 1 M HEPES solution. 25 μL of the Ga-68 was added and the solution was shaken with 300 rpm at 50° C. for 10 min. Radio-TLC was performed with iTLC-SG as stationary phase and 0.1 M citrate solution (adjusted to pH 4 with 1 M NaOH) as mobile phase. The conversion was >95% and yielded approx. 90 MBq of the corresponding Ga-68 labelled NODAGA-compound.

### Example 43—Compounds 221, 222, 223, 224 und 225

### Labeling of Compounds 211, 212, 213, 214 und 215

8 μL of 1 mM stock solution of the corresponding NODAGA-compound was added to a Protein LoBind Eppendorf tube containing 200 μL of an approx. 1 M HEPES solution, which was prior adjusted with 1 M HCl to pH 4. The corresponding metal solution (both 0.01-0.1 M HCl) was added and the solution was shaken with 300 rpm at 50° C. for 10 min. Radio-TLC was performed with iTLC-SG as stationary phase and 0.1 M citrate solution (adjusted to pH 4 with 1 M NaOH) as mobile phase. The conversion was >99% and yielded the corresponding Cu-64 labelled NODAGA-compound.

### Example 44—Determining of Solubilities of Chelate Compounds

For assessing the water solubilities of compounds 115 to 124 and 221 to 225 the distribution coefficients log D were determined at the physiological pH-Wert of 7.4.

The following table shows the distribution coefficients log D of compounds 115 to 124 and 221 to 225

| Compound | Log D_{7.4} (n = 3) |
|---|---|
| 115 | −2.73 ± 0.04 |
| 116 | −3.03 ± 0.05 |
| 117 | −3.47 ± 0.10 |
| 118 | −2.75 ± 0.04 |
| 119 | −3.14 ± 0.02 |
| 120 | −3.50 ± 0.01 |
| 121 | −2.99 ± 0.02 |
| 122 | −3.48 ± 0.03 |
| 123 | −2.89 ± 0.02 |
| 124 | −4.00 ± 0.14 |
| 221 | −4.15 ± 0.09 |
| 222 | −3.80 ± 0.02 |
| 223 | −3.81 ± 0.08 |
| 224 | −4.28 ± 0.08 |
| 225 | −3.17 ± 0.02 |

In general, the more negative the values of log D the better the water solubility.

### Example 45—Determining of Solubilities of Fluorine Compounds

The water solubility of compounds 85-90 was determined by weighing 1 mg of the cold compound in an Eppendorf-tube and adding water in 1 μl steps. The addition was performed until everything was dissolved. The value in mg/μl was converted to g/1.

### Water Solubilities in Gram Per Liter

85: 80 g/l
86: 110 g/l
87: 115 g/l
88: 85 g/l
89: 120 g/l
90: 115 g/l

### Example 46—Inhibitory Constants on Tumor Cells of Compounds 85, 86 and 88 to 90

The binding affinities (Kᵢ values) of compounds 85, 86 and 88 to 90 were determined in a cell-based competitive assay using Lu-labelled a DOTA compound (128). The corresponding compound was incubated in 14 different concentrations between 5×10⁻⁹ M and 5×10⁻⁵ M with 1 nM of radiolabeled compound 128 together with PC3 hPSCA PD-L1 cells. After incubation at 4° C. for 90 min, the cells were washed with ice cold PBS. The accumulated activity on the cells was measured by a gamma counter. The used concentrations (x-axis) were plotted against the counts (y-axis) and graphical interpretation with a nonlinear regression analysis (GraphPad Software) yielded the Kᵢ value of the corresponding compounds 85, 86 and 88 to 90.

### LITERATURE

1. Musso, L., et al., Synthesis of 5,6-dihydro-4H-benzo [d]isoxazol-7-one and 5,6-dihydro-4H-isoxazolo[5,4-c]pyridin-7-one Derivatives as Potential Hsp90 Inhibitors. Chem. Biol. Drug. Des., 2015. 86: p. 1030-1035.
2. Yeung, K.-S., et al., Compounds Useful as Immunomodulators. Patent: WO 2017/066227 Å1, 2017.
3. Wünsch, S. and B. Breit, Probing o-Diphenylphosphanyl Benzoate (o-DPPB)Directed C D C Bond Formation: Total Synthesis of Dictyostatin. Chem. Eur. J., 2015. 21: p. 2358-2363.
4. Xiong, X. and Y.-Y. Yeung, Ammonium Salt-Catalyzed Highly Practical OrthoSelective Monohalogenation and Phenylselenation of Phenols: Scope and Applications. ACS Catal., 2018. 8: p. 4033-4043.
5. Ikeuchi, T., et al., Gold(I)-Catalyzed Cascade Cyclization Reactions of Allenynes for the Synthesis of Fused Cyclopropanes and Acenaphthenes. Angew. Chem. Int. Ed., 2019. 58: p. 7792-7796. 6th PD-L1 Antagonist Compound. Patent: WO 2020/169058 Å1.
7. Preparation of macrocyclic indole compounds as Mel-1 inhibitor. Patent: CN110845520.
8. Romieu, A., et al., Water solubilization of xanthene dyes by post-synthetic sulfonation in organic media. Tetrahedron Lett., 2010. 51: p. 3304-3308.
9. Borkin, D., et al., Property Focused Structure-Based Optimization of Small Molecule Inhibitors of the Protein-Protein Interaction between Menin and Mixed Lineage Leukemia (MLL). J. Med. Chem., 2016. 59: p. 892-913.
10. Fleming, P. E., et al., 2-(Amino-Substituted)-4-Aryl Pyrimidines and Related Compounds Useful for Treating Inflammatory Diseases. Patent: WO 2005/066139 Å2, 2005.
11. Egbertson, M. S., et al., Non-Peptide Fibrinogen Receptor Antagonits. 2. Optimization of a Tyrosine Template as a Mimic for Arg-Gly-Asp. J. Med. Chem., 1994. 37: p. 2537-2551.
12. Pretze, M. and C. Mamat, Automated preparation of [18F]AFP and [18F]BFP: Two novel bifunctional 18F-labeling building blocks for Huisgen-Click. J Fluorine Chem, 2013. 150: p. 25-35.
13. Mier, W., et al., Conjugation of DOTA Using Isolated Phenolic Active Esters: The Labeling and Biodistribution of Albumin as Blood Pool Marker. Bioconjugate Chem., 2005. 16: p. 237-240.
14. Liu, Z., G. Yoon, and S. H. Cheon, An enantioselective total synthesis of (S)-(−)licochalcone E: determination of the absolute configuration. Tetrahedron, 2010. 66(17): p. 3165-3172.
15. Enders, D., et al., Asymmetric total synthesis of smyrindiol employing an organocatalytic aldol key step. Beilstein J Org Chem, 2012. 8: p. 1112-7.
16. Maschauer, S. and O. Prante, A series of 2—O-trifluoromethylsulfonyl-d-mannopyranosides as precursors for concomitant 18F-labeling and glycosylation by click chemistry. Carbohydrate Research, 2009. 344(6): p. 753-761.
17. Maschauer, S., et al., 18F-Glyco-RGD Peptides for PET Imaging of Integrin Expression: Efficient Radiosynthesis by Click Chemistry and Modulation of Biodistribution by Glycosylation. Molecular Pharmaceutics, 2014. 11(2): p. 505-515.
18. Pretze, M., et al., An efficient bioorthogonal strategy using CuAAC click chemistry for radiofluorinations of SNEW peptides and the role of copper depletion. ChemMedChem, 2013. 8(6): p. 935-45.
19. Aktoudianakis, E. A., Todd; Cho, Aesop; Du, Zhimin; Graupe, Michael, PD1/PD-Inhibitors. WO 2018/195321 Å1, 2018.
20. Parcella; Mark George Saulnier; Li-Qiang Sun; Alan Xiangdong Wang; Ningning Xu; Juliang Zhu; Paul Michael Scala, K.-S.Y.T.P.C.D.B.F.K.A.G.Y.P.H.D.R.L.Z.M.E.M.K.E.,Compounds useful as immunomodulators. US 2017/0107202 A1, 2017.
21. Chupak, L. S. Z., Xiaofang, Compounds useful as Immunomodulators. WO 2015/034820 A1, 2015.
22. Feng, Z. C., Xiaoguang; Yang, Yang; Zhou, Chuan; Ji, Ming; Jin, Xiaofeng; Xue, Nina; Zheng, Yi; Chen, Hao; Li, Ung, Phenylate Derivative, Preparation Method therefore, and pharmaceutical composition, and uses thereof WO 2017/202276 A 1, 2017.
23. Guzik, K., et al., Development of the Inhibitors that Target the PD-1/PD-L1 Interaction-A Brief Look at Progress on Small Molecules, Peptides and Macrocycles. Molecules, 2019. 24(11).
24. Bridoux, J., et al., Anti-Human PD-L1 Nanobody for Immuno-PET Imaging: Validation of a Conjugation Strategy for Clinical Translation. Biomolecules, 2020. 10(10): p. 1388.
25. Fu, R., et al., Antibody Fragment and Affibody ImmunoPET Imaging Agents: Radiolabelling Strategies and Applications. ChemMedChem, 2018. 13(23): p. 2466-2478.
26. Niemeijer, A. N., et al., Whole body PD-1 and PD-L1 positron emission tomography in patients with non-small-cell lung cancer. Nature Communications, 2018. 9(1): p. 4664.
27. Bensch, F., et al., 89Zr-atezolizumab imaging as a non-invasive approach to assess clinical response to PD-L1 blockade in cancer. Nature Medicine, 2018.
28. Kuan, H., et al., Developing native peptide-based radiotracers for PD-L1 PET imaging and improving imaging contrast by pegylation. Chemical Communications, 2019. 55(29): p. 4162-4165.
29. Lesniak, W. G., et al., Development of [(18)F]FPy-WL12 as a PD-L1 Specific PET Imaging Peptide. Molecular Imaging, 2019. 18: p. 1536012119852189-1536012119852189.
30. De Silva, R. A., et al., Peptide-Based (68)Ga-PET Radiotracer for Imaging PD-L1 Expression in Cancer. Mol Pharm, 2018. 15(9): p. 3946-3952.
31. Chatterjee, S., et al., Rapid PD-L1 detection in tumors with PET using a highly specific peptide. Biochemical and Biophysical Research Communications, 2017. 483(1): p. 258-263.
32. Kumar, D., et al., Peptide-based PET quantifies target engagement of PD-L1 therapeutics. J Clin Invest, 2018.
33. Miao, Y., et al., One-Step Radiosynthesis and Initial Evaluation of a Small Molecule PET Tracer for PD-L1 Imaging. Bioorganic & Medicinal Chemistry Letters, 2020: p. 127572.
34. Feng, Z. Y., Yang, Iodine isotope-labeled benzyl phenyl ether derivative, and preparation method and medicine thereof Composition and use. CN 111662270 A, 2020.
35. Huck, B. R., L. Kötzner, and K. Urbahns, Small Molecules Drive Big Improvements in Immuno- Oncology Therapies. Angewandte Chemie International Edition, 2018. 57(16): p. 4412-4428.
36. Weekes, D. M.; Ramogida, C. F.; Jaraquemada-Pelaez, M. d. G.; Patrick, B. O.; Apte, C.; Kostelnik, T. I.; Cawthray, J. F.; Murphy, L.; Orvig, C. Dipicolinate complexes of gallium (III) and lanthanum (III). Inorganic chemistry 2016, 55, 12544-12558.
37. Song, X. P.; Bouillon, C.; Lescrinier, E.; Herdewijn, P. Iminodipropionic Acid as the Leaving Group for DNA Polymerization by HIV-1 Reverse Transcriptase. ChemBioChem 2011, 12, 1868-1880.

### LIST OF ABBREVIATIONS

THF tetrahydrofurane
DIPEA diisopropylethylamine
MEM methoxymethyl
MOM (2-methoxyethoxy)methyl
MeOH ethanol
EtOH ethanol
BuOH butanol
PPh₃ triphenylphosphane
CBr₄ tetrabrommethane
CCl₄ tetrachlormethan
DMF dimethylformamid
DCM dichlormethan
TFA trifluoressigsaure
TBDMS tert-butyldimethylsilyl
TBAF tetrabutylammoniumfluoride
THPTA tris(3-hydroxypropyltriazolylmethyl)amine
HBTU hexafluorophosphate benzotriazole tetramethyl uronium

1-15. (canceled)

16. A compound of general formula I 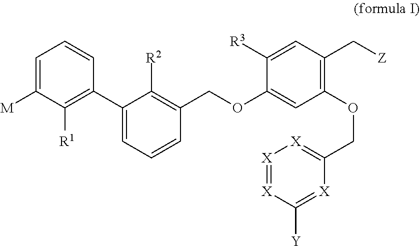
wherein
R¹ is selected from the group consisting of hydrogen, a substituted or unsubstituted C₁-C₆ alkyl group and bromine;
R² is selected from the group consisting of a substituted or unsubstituted C₁-C₆ alkyl group and bromine or iodine;
R³ is selected from the group consisting of chlorine, bromine, iodine and a first leaving group AG¹;
M is hydrogen or a group R⁵;
X is C—H or N, with the provision that at most two of the moieties X are N and the remaining moieties X are C—H;
Y is selected from the group consisting of —CN, —S(O)₂—CH₃, fluorine, iodine and a second leaving group AG²;
Z is selected from the group consisting of a group of general formula II, a group of general formula III, a group of general formula IV, a group of general formula V, a group of general formula VI and a group of general formula VIa 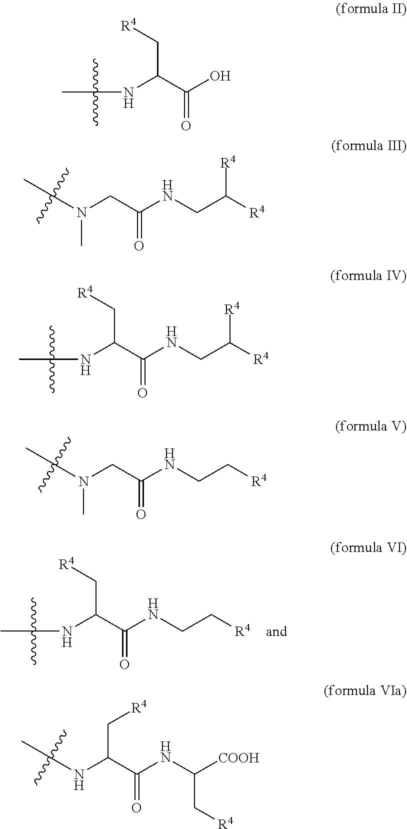
AG¹ is selected from the group consisting of an organotin group, a boronic acid and a boronic acid ester;
AG² is selected from the group consisting of an organotin group, a fluorosulphate group, a boronic acid and a boronic acid ester;
R⁴ is a sulphonic acid group or a phosphonic acid group;
R⁵ is selected from a first group or a second group, wherein
the first group consists of a group of general formula VII or a group of general formula VIII: 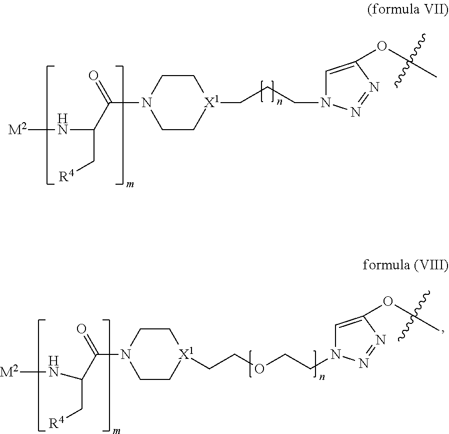
X¹ is C—H or N,
M² is a chelate ligand or a chelate complex consisting of the chelate ligand and a central atom, wherein the central atom is selected from the group consisting of Sc-43, Sc-44, Sc-47, Cu-61, Cu-62, Cu-64, Cu-67, Ga-68, Y-86, Zr-89, Y-90, Tc-99m, In-111, La-133, Gd-153, Gd-155, Gd-157, Tb-149, Tb-152, Tb-155, Tb-161, Lu-177, Pd-197m, Pb-209, Pb-212, Bi-213, and Ac-225, m is an integer of 0 to 3, n is an integer of 1 to 7, and p is an integer of 1 to 4;
the second group consists of a group of general formula IX, a group of general formula X, a group of general formula XI, and a group of general formula XII: 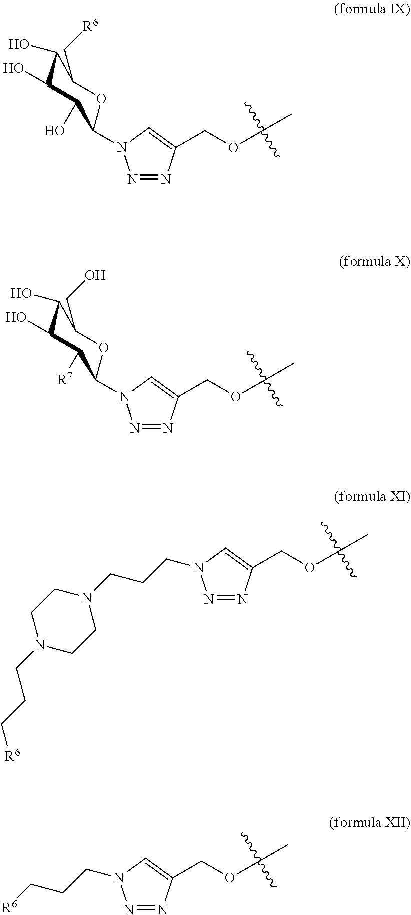
wherein
R⁶ is O-Ts or fluorine and R⁷ is O-Tf or fluorine, Ts designates a tosyl group and Tf designates a triflyl group.

17. The compound according to claim 16, wherein R⁶ and R⁷ are [¹⁸F]fluorine.

18. The compound according to claim 16, wherein Z is a group of general formula III or IV

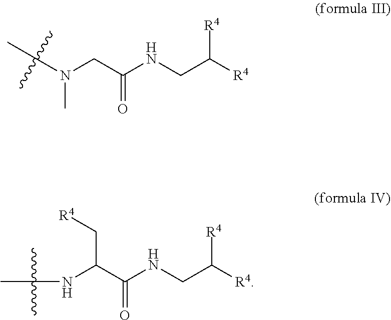

19. The compound according to claim 18, wherein R⁴ is a sulphonic acid group.

20. The compound according to claim 16, wherein R⁵ is selected from a group of general formula IX, X, or XII:

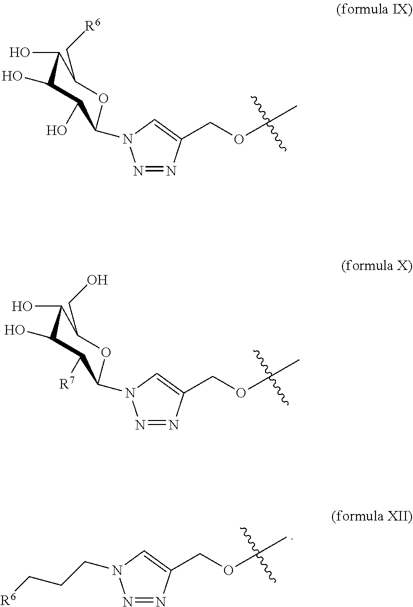

21. The compound according to claim 20, wherein R⁶ and R⁷ are fluorine.

22. The compound according to claim 20, wherein R⁶ and R⁷ are [¹⁸F]fluorine.

23. The compound according to claim 16, wherein R⁵ is selected from the group of general formula VII or VIII:

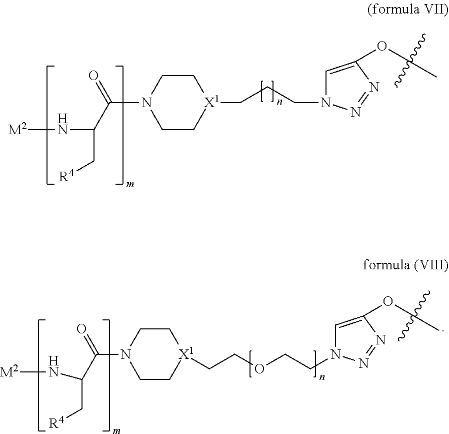

24. The compound according to claim 23, wherein m is 0 or 1, n is 1 or 2, and p is 1 or 2.

25. The compound according to claim 23, wherein M² is a chelate ligand that is selected from the group consisting of a 1,4,7,10-tetraazacyclododecane-1,4,7,10-tetra acetic acid group (DOTA group), a 1,4,7-triazacyclononane-1-glutaric acid-1,4,7-acetic acid group (NODAGA group), and a 6,6′-((1,4,10,13-tetraoxa-7,16-diazacyclooctadecane-7,16-diyl)bis(methylene))dipicolinic acid group (MACROPA group).

26. The compound according to claim 23, wherein M² is a chelate complex the central atom of which is selected from the group consisting of Cu-64, Ga-68, and Lu-177.

27. The compound according to claim 16 for use as a medicament.

28. The compound according to claim 16 for use as a medicament for the diagnostics and therapy of diseases in which the protein programmed cell death 1 ligand 1 (PD-Li) is involved.

29. The compound according to claim 27, wherein the medicament is for the diagnostics and/or therapy of cancer diseases and COVID-19 infections.

30. A medicament containing a compound according to claim 16 or a pharmaceutically acceptable salt thereof.
